SEQUENCE LISTING (40 sequences)

SEQ ID NO: 1 - DNA, 3385 bp, Homo sapiens
ccacctctccagccactcatctctgcccagctgctgccctccccaggaggcctccatggcttcacctacctccaccaacccagcgcatgcccactttgagagcttcctgcaggcccagctgtgccaggacgtgctgagcagcttccaggagctgtgtggggccctggggctggaacccggtggggggctgccccagtaccacaagatcaaggaccagctcaactactggagcgccaagtcactgtggaccaagctggacaagcgagcaggccagcctgtctaccagcagggccgggcctgcaccagcaccaagtgcctggtggtgggtgctggaccttgcgggctgcgggtcgctgtggagctggcgctgctgggggcccgagtggtgctggtggaaaagcgcaccaagttctctcgccacaacgtgctccacctctggcccttcaccatccacgacctgcgggcactcggtgctaagaagttctacgggcgcttctgcaccggcaccctggaccacatcagcatcaggcagctccagctgcttctgctgaaggtagcattgctgctgggggtggaaattcactggggtgtcactttcactggcctccagccccctcctaggaaggggagtggctggcgtgcccagctccaacccaacccccctgcccagctggccaactatgaatttgacgtccttatctcggctgcaggaggtaaattcgtccctgaaggcttcaaagttcgagaaatgcgaggcaaactggccattggcatcacagccaactttgtgaatggacgcaccgtggaggagacacaggtgccggagatcagtggtgtagccaggatctacaaccagagcttcttccagagccttctcaaagccacaggcattgatctggagaacattgtgtactacaaggacgacacccactactttgtgatgacagccaagaagcagtgcctgctgcggctgggggtgctgcgccaggactggccagacaccaatcggctgctgggcagtgccaatgtggtgcccgaggctctgcagcgctttacccgggcagctgctgactttgccacccatggcaagctcgggaaactagagtttgcccaggatgcccatgggcagcctgatgtctctgcctttgacttcacgagcatgatgcgggcagagagttctgctcgtgtgcaagagaagcatggcgcccgcctgctgctgggactggtgggggactgcctggtggagcccttctggcccctgggcactggagtggcacggggcttcctggcagcctttgatgcagcctggatggtgaagcggtgggcagagggcgctgagtccctagaggtgttggctgagcgtgagagcctgtaccagcttctgtcacagacatccccagaaaacatgcatcgcaatgtggcccagtatgggctggacccagccacccgctaccccaacctgaacctccgggcagtgacccccaatcaggtacgagacctgtatgatgtgctagccaaggagcctgtgcagaggaacaacgacaagacagatacagggatgccagccaccgggtcggcaggcacccaggaggagctgctacgctggtgccaggagcagacagctgggtacccgggagtccacgtctccgatttgtcttcctcctgggctgatgggctagctctgtgtgccctggtgtaccggctgcagcctggcctgctggaaccctcagagctgcaggggctgggagctctggaagcaactgcttgggcactaaaggtggcagagaatgagctgggcatcacaccggtggtgtctgcacaggccgtggtagcagggagtgacccactgggcctcattgcctacctcagccacttccacagtgccttcaagagcatggcccacagcccaggccctgtcagccaggcctccccagggacctccagtgctgtattattccttagtaaacttcagaggaccctgcagcgatcccgggccaaggaaaatgcagaggatgctggtggcaagaagctgcgcttggagatggaggccgagaccccaagtactgaggtgccacctgacccagagcctggtgtacccctgacacccccatcccaacaccaggaggccggtgctggggacctgtgtgcactttgtggggaacacctctatgtcctggaacgcctctgtgtcaacggccatttcttccaccggagctgcttccgctgccatacctgtgaggccacactgtggccaggtggctacgagcagcacccaggagatggacatttctactgcctccagcacctgccccagacagaccacaaagcggaaggcagcgatagaggccctgagagtccggagctccccacaccaagtgagaatagcatgccaccaggcctctcaactcccacagcctcgcaggagggggccggtcctgttccagatcccagccagcccacccgtcggcagatccgcctctccagcccggagcgccagcggttgtcctcccttaaccttacccctgacccggaaatggagcctccacccaagcctccccgcagctgctccgccttggcccgccacgccctggagagcagctttgtgggctggggcctgccagtccagagccctcaagctcttgtggccatggagaaggaggaaaaagagagtcccttctccagtgaagaggaagaagaagatgtgcctttggactcagatgtggaacaggccctgcagacctttgccaagacctcaggcaccatgaataactacccaacatggcgtcggactctgctgcgccgtgcgaaggaggaggagatgaagaggttctgcaaggcccagaccatccaacggcgactaaatgagattgaggctgccttgagggagctagaggccgagggcgtgaagctggagctggccttgaggcgccagagcagttccccagaacagcaaaagaaactatgggtaggacagctgctacagctcgttgacaagaacaacagcctggtggctgaggaggccgagctcatgatcacggtgcaggaattgaatctggaggagaaacagtggcagctggaccaggagctacgaggctacatgaaccgggaagaaaacctaaagacagctgctgatcggcaggctgaggaccaggtcctgaggaagctggtggatttggtcaaccagagagatgccctcatccgcttccaggaggagcgcaggctcagcgagctggccttggggacaggggcccagggctagacgagggtgggccgtctgctttcgttcccacaaagaaagcacctcaccccagcacagtgccacccctgttcatctgggctgcctggcagagagccttgctgtttacaattaaaatgtttctgccac

SEQ ID NO: 2 - protein, 863 aa, Homo sapiens
MAGPRGALLAWCRRQCEGYRGVEIRDLSSSFRDGLAFCAILHRHRPDLLDFDSLSKDNVFENNRLAFEVAEKELGIPALLDPNDMVSMSVPDCLSIMTYVSQYYNHFCSPGQAGVSPPRKGLAPCSPPSVAPTPVEPEDVAQGEELSSGSLSEQGTGQTPSSTCAACQQHVHLVQRYLADGRLYHRHCFRCRRCSSTLLPGAYENGPEEGTFVCAEHCARLGPGTRSGTRPGPFSQPKQQHQQQLAEDAKDVPGGGPSSSAPAGAEADGPKASPEARPQIPTKPRVPGKLQELASPPAGRPTPAPRKASESTTPAPPTPRPRSSLQQENLVEQAGSSSLVNGRLHELPVPKPRGTPKPSEGTPAPRKDPPWITLVQAEPKKKPAPLPPSSSPGPPSQDSRQVENGGTEEVAQPSPTASLESKPYNPFEEEEEDKEEEAPAAPSLATSPALGHPESTPKSLHPWYGITPTSSPKTKKRPAPRAPSASPLALHASRLSHSEPPSATPSPALSVESLSSESASQTAGAELLEPPAVPKSSSEPAVHAPGTPGNPVSLSTNSSLASSGELVEPRVEQMPQASPGLAPRTRGSSGPQPAKPCSGATPTPLLLVGDRSPVPSPGSSSPQLQVKSSCKENPFNRKPSPAASPATKKATKGSKPVRPPAPGHGFPLIKRKVQADQYIPEEDIHGEMDTIERRLDALEHRGVLLEEKLRGGLNEGREDDMLVDWFKLIHEKHLLVRRESELIYVFKQQNLEQRQADVEYELRCLLNKPEKDWTEEDRAREKVLMQELVTLIEQRNAIINCLDEDRQREEEEDKMLEAMIKKKEFQREAEPEGKKKGKFKTMKMLKLLGNKRDAKSKSPRDKS

SEQ ID NO: 3 - DNA, 6008 bp, Homo sapiens
ccgggccgcctcgctcgctcccagctctgtcagtggcccgcggggcccgatcgctgcgcccgcggccagggccgaggcaggcctgacccggggccgggcagcccgcgcgactttcggaacatggcaacccgtgtgtgtctcatcccagaaagagaagactttaaccactgtgatgcctgagaatccagtgtgacgtttctccagatacttcatgctgttcacctgtgtcctcgccgcaccactgccgcacacgactcctgaaccatgggggaaaacgaggatgagaagcaggcccaggcggggcaggtttttgagaactttgtccaggcatccacgtgcaaaggtaccctccaggccttcaacattctcacacgacacctggacctagaccctctggaccacagaaacttttattccaagctcaagtccaaggtgaccacctggaaagccaaagccctgtggtacaaattggataagcgtggttcccacaaagagtataagcgagggaagtcgtgcacgaacaccaagtgtctcatagttgggggaggaccctgtggcttgcgcactgccattgaacttgcctacctgggagccaaagtggtcgtggtggagaagagggactccttctcccggaacaacgtgctacacctctggcctttcaccatccatgaccttcggggcctgggagccaagaagttctatgggaagttctgtgctggctccatcgaccatatcagtattcgccaactacagctcatcctattcaaggtggccctgatgctgggagttgaaatccatgtgaatgtggagttcgtgaaggttctagagcctcctgaagatcaagaaaatcaaaaaattggctggcgggcagaatttctccctacagaccattctctgtcggagtttgagtttgacgtcatcattggtgccgatggccgcaggaacaccctggaagggttcagaagaaaagaattccgtgggaagctggcgattgccatcaccgccaacttcataaacagaaacagcacagcggaagccaaggtggaagagattagtggtgtggctttcatcttcaatcagaaattttttcaggaccttaaagaagaaacaggcatagatcttgagaacattgtttactacaaggactgcacccactattttgtaatgacagccaagaagcagagcctgctcgacaaaggtgtcatcattaacgactacatcgacacagagatgctgctgtgtgcggagaacgtgaaccaagacaacctgctatcctatgcccgggaagctgcagactttgccaccaactaccagctgccatccttagactttgccatgaaccactatgggcagcctgatgtggccatgtttgactttacctgcatgtatgcctcagagaacgcggccctggtgcgggagcggcaggcgcaccagctgctcgtggcccttgtgggtgacagcttgcttgagccattttggcccatgggtacaggctgtgcccgtggcttcctggcagcctttgacacggcatggatggtgaagagctggaaccagggcacccctcccctggagctgctggctgaaagggaaagtctctaccggctgttacctcagacaaccccggagaacatcaacaagaactttgagcagtacacgttggacccagggacacggtacccaaacctcaactcacactgtgtcaggccccatcaggtgaagcatttgtatatcactaaggagctggagcactaccctctcgagagactgggctcggtgaggagatctgtcaacctctccaggaaggagtcagatatccggcccagcaagctcctgacctggtgccagcagcagacagagggctaccagcatgtcaacgtcaccgacctgaccacatcctggcgcagtgggttggccctgtgtgccatcatccaccgcttccggcctgagctcatcaactttgactctttgaatgaagatgatgctgtggagaacaaccagctcgcatttgatgtggccgagcgagagtttgggatccctccagtgaccacgggcaaagagatggcatctgcccaggagcctgacaagctcagcatggtcatgtacctctccaagttctacgagctcttccggggcaccccactgaggcccgtggattcttggcgcaaaaactatggagaaaatgctgacctcagcttggccaaatcatccatttctaataactatctcaacctcacatttccaaggaagaggactccacgggtggatggtcaaaccggagagaatgacatgaacaaacggagacggaagggcttcaccaacctggacgagccttcaaacttttccagccgtagcttgggctccaatcaagagtgtgggagcagtaaggaaggtggaaatcagaacaaagtcaagtccatggcgaatcagctgctggccaagtttgaggagagcactcggaacccctcactcatgaagcaggaacgccgtgtctcagggataggtaagccggtcctgtgctcttcctccggccctcctgttcactcttgctgccccaagccggaggaggccacacccagcccatcacctcctctgaaaaggcagttcccctctgtggtcgtgacggggcacgtgctcagagagctcaagcaagtgtctgctggcagtgagtgcctgagcagaccttggagagccagagccaagtctgacctacagctgggtgggacagaaaatttcgctaccctgccttctacccgcccgagggcgcaggctctttccggggtgctgtggcggctgcagcaagtggaggaaaagattctccagaagagggctcagaacttggccaacagggaatttcacacaaagaacattaaggagaaggcggctcaccttgcctccatgtttggacacggggatttcccgcagaataaactgctctctaaaggcctgtctcatactcatcctccatctcctccctctcgccttccgtctcctgatccagctgcttcttcctctccatcaactgttgactctgcttctcctgccagaaaggaaaagaagtcaccttcagggttccattttcatcccagccatttgagaacagtgcatcctcagctgacggtagggaaagtgtccagcggaataggggctgcagctgaagtcctggtcaatctgtacatgaatgatcacagacctaaggcccaggccacctctccagacctggaatctatgcgaaagtcatttccccttaacctgggaggcagcgacacgtgttacttctgtaagaaacgtgtgtacgtgatggaacggctgagcgccgagggccacttcttccaccgggagtgtttccgctgcagcatctgtgccaccaccttgcgcctggccgcctacacctttgactgcgatgaaggcaaattttactgcaagcctcacttcattcactgtaaaaccaatagcaaacaacggaagagacgggcagagttgaagcaacaaagagaggaggaggcaacatggcaagagcaggaagcccctcggagagacactcccaccgaaagttcttgcgcagtggccgccattggcaccctggaaggcagccccccagttcatttcagccttccagtgctacacccacttcttggccaccccatctgggggaaggacaggagctggacaggccaagagctatctcccttggctggagaagaccgggaaaaagggagtactggagccaggaaggaagaagagggagggccagtgctggtaaaggagaagttgggcctgaagaagttagtcctcacccaggagcagaagaccatgttgttggattggaatgactccatccctgagagtgtgcacctcaaagctggggagcgaatttcccagaaaagtgctgagaatggtagaggaggccgtgtgctaaaaccagtccgccccctgctgctccctagggcagcaggagagcccctgccaacccagagaggggctcaggagaagatggggacccctgcggaacaagctcaaggggagcgaaacgtgcctccacccaagtccccactgcggctcatagccaatgccatccgaaggtctctagagcccctcctttccaactctgaaggcgggaagaaggcctgggccaagcaagaatccaaaactttgcccacacaggcctgcactcgctcattcagccttcggaaaaccaattccaataaagacggggaccagcattcccctgggagaaaccagtcctcagcctttagccctcctgaccctgccctccgcacccacagtttgcccaatcggccatccaaggtctttcctgcacttaggtccccaccctgcagcaagattgaagaagtccccacactcctcgagaaagtgagtttgcaagagaacttcccagatgcttctaagcctccaaagaaaagaatctcacttttttcctccctcagactcaaagacaaatcttttgagagtttcctccaagaatccagacaaagaaaggacatcagggacctctttggcagccccaagaggaaggtgctgcctgaagatagtgcgcaggccctggagaagctgctgcagcctttcaaaagcacctccctgcgccaggcagctcctcctcctcctcctcctcctcctcctcctcctcctcctcctcctccccctacagcgggaggtgcagactccaagaactttcccctcagagcacaggtaacagaggcttcctcttctgcctcttcaacctcctcctcctctgcagatgaagaatttgatccccagctttccttgcagttaaaggagaagaagacacttagaagaagaaagaagctagaaaaagcaatgaagcagttggttaagcaagaagaattgaaaagactctataaggctcaggccatccagaggcagctggaggaggtggaggagcggcagagggcttctgagatccagggtgtgaggctggagaaggcgttgcgaggagaagcagattcaggcacacaggatgaagcacagcttttgcaggaatggtttaagctggttctggagaagaataaattaatgcgatatgagtcggagctcctaatcatggcccaggaactggaattagaagatcatcaaagcagactggagcagaaactgagagagaaaatgctcaaggaggagagccagaaagatgagaaggatctaaacgaagagcaagaagtattcaccgagctgatgcaagtgattgagcaaagggacaaactcgtcgattccttagaggaacaacgcatcagagaaaaagccgaggaccagcactttgaaagcttcgtattctccagaggctgtcagctgagcaggacttgaggaggcccgtagtccctctccctggctgcacgttgggaccggatcaggccaagtgcaccacacaccctcatgggtctttctgcaggatttatcatccctggaacttaagttataccttactgcattttttaaaattaaaattctcttgcacgcatggcagcttcccaaggttcttccagagattcaaatgaagaaaacccaaagactctttggcaattggcagtcaacttcagccaggctctcagactggaggtgttgttggcagatgaccagcattgttttccctagaaagtgacacaaagacttgactttcctgctacttttatcattttccttcccaattcattgagttacatactttaagatttttgagaagctgccttttcattaatatatccatatttgcctttttttgtatggatgaccagtttccaaatgtcagaaagaagcagccgcagtttaaagattaggttaatatttaaattgtgtttccagagaaagaggagaaaccttgagattactgattacataaagcaaataactcatatagcaggtgttaattcaatccagggtgaatttaatttaccaggtgcatttataagccttaatataatatacataagcaatgagagcttaatagaacatttgagccttaattttattttaag

SEQ ID NO: 4 - protein, 1633 aa, Homo sapiens
MGENEDEKQAQAGQVFENFVQASTCKGTLQAFNILTRHLDLDPLDHRNFYSKLKSKVTTWKAKALWYKLDKRGSHKEYKRGKSCTNTKCLIVGGGPCGLRTAIELAYLGAKVVVVEKRDSFSRNNVLHLWPFTIHDLRGLGAKKFYGKFCAGSIDHISIRQLQLILFKVALMLGVEIHVNVEFVKVLEPPEDQENQKIGWRAEFLPTDHSLSEFEFDVIIGADGRRNTLEGFRRKEFRGKLAIAITANFINRNSTAEAKVEEISGVAFIFNQKFFQDLKEETGIDLENIVYYKDCTHYFVMTAKKQSLLDKGVIINDYIDTEMLLCAENVNQDNLLSYAREAADFATNYQLPSLDFAMNHYGQPDVAMFDFTCMYASENAALVRERQAHQLLVALVGDSLLEPFWPMGTGCARGFLAAFDTAWMVKSWNQGTPPLELLAERESLYRLLPQTTPENINKNFEQYTLDPGTRYPNLNSHCVRPHQVKHLYITKELEHYPLERLGSVRRSVNLSRKESDIRPSKLLTWCQQQTEGYQHVNVTDLTTSWRSGLALCAIIHRFRPELINFDSLNEDDAVENNQLAFDVAEREFGIPPVTTGKEMASAQEPDKLSMVMYLSKFYELFRGTPLRPVDSWRKNYGENADLSLAKSSISNNYLNLTFPRKRTPRVDGQTGENDMNKRRRKGFTNLDEPSNFSSRSLGSNQECGSSKEGGNQNKVKSMANQLLAKFEESTRNPSLMKQERRVSGIGKPVLCSSSGPPVHSCCPKPEEATPSPSPPLKRQFPSVVVTGHVLRELKQVSAGSECLSRPWRARAKSDLQLGGTENFATLPSTRPRAQALSGVLWRLQQVEEKILQKRAQNLANREFHTKNIKEKAAHLASMFGHGDFPQNKLLSKGLSHTHPPSPPSRLPSPDPAASSSPSTVDSASPARKEKKSPSGFHFHPSHLRTVHPQLTVGKVSSGIGAAAEVLVNLYMNDHRPKAQATSPDLESMRKSFPLNLGGSDTCYFCKKRVYVMERLSAEGHFFHRECFRCSICATTLRLAAYTFDCDEGKFYCKPHFIHCKTNSKQRKRRAELKQQREEEATWQEQEAPRRDTPTESSCAVAAIGTLEGSPPVHFSLPVLHPLLGMLLDWNDSIPESVHLKAGERISQKSAENGRGGRVLKPVRPLLLPRAAGEPLPTQRGAQEKMGTPAEQAQGERNVPPPKSPLRLIANAIRRSLEPLLSNSEGGKKAWAKQESKTLPTQACTRSFSLRKTNSNKDGDQHSPGRNQSSAFSPPDPALRTHSLPNRPSKVFPALRSPPCSKIEEVPTLLEKVSLQENFPDASKPPKKRISLFSSLRLKDKSFESFLQESRQRKDIRDLFGSPKRKVLPEDSAQALEKLLQPFKSTSLRQAAPPPPPPPPPPPPPPPPPTAGGADSKNFPLRAQVTEASSSASSTSSSSADEEFDPQLSLQLKEKKTLRRRKKLEKAMKQLVKQEELKRLYKAQAIQRQLEEVEERQRASEIQGVRLEKALRGEADSGTQDEAQLLQEWFKLVLEKNKLMRYESELLIMAQELELEDHQSRLEQKLREKMLKEESQKDEKDLNEEQEVFTELMQVIEQRDKLVDSLEEQRIREKAEDQHFESFVFSRGCQLSRT

SEQ ID NO: 5 - DNA, 8977 bp, Homo sapiens
atggaggagaggaagcatgagaccatgaacccagctcatgtcctctttgaccggtttgtccaggccaccacctgcaagggaaccctcaaggctttccaggagctctgtgaccacctggaactaaagccaaaggactaccgctccttctatcacaagctcaagtccaagcttaactactggaaagccaaagccctctgggcaaaattggacaaacggggcagtcacaaagactacaaaaagggaaaagcgtgcactaacaccaagtgtctcatcattggggctggcccctgtggtctccgtacagccatcgacttatccttactgggggccaaggtggttgttattgagaaacgagatgccttctcccgcaacaacgtcttgcatctctggccattcaccatacatgatctacgaggtctgggtgccaagaagttctatggcaagttctgtgctggagccatcgaccatatcagtatccgtcagctccaactaatacttttgaaagtagccttgatcctaggcattgaaatccacgtcaatgtggaattccaaggacttatacagcctcctgaggaccaagagaatgaacggataggctggcgggcactggtgcaccccaagactcatcctgtgtcagagtatgaatttgaagtgatcatcggtggggatggtcggaggaacaccttggaagggtttcgtcggaaagaattccgtggcaaactggccatcgccatcacggcaaattttatcaaccgaaatacaacagcagaagctaaagtggaagagatcagtggtgtggcttttatattcaaccaaaaatttttccaggaactgagggaagccacaggtattgacttggagaacatcgtttactacaaagatgacacacactatttcgttatgacagccaaaaagcagagtttgctggacaaaggagtgatactacatgactacgccgacacagagctcctgctttcccgagaaaacgtggaccaggaggctctgctcagctatgccagggaggcggcagacttctctacccagcagcagctgccgtctctggattttgccatcaatcactatgggcagcccgatgtggccatgtttgacttcacttgtatgtatgcctccgagaacgccgccttggtgcgggagcagaacggacaccagttactagtggctctggtcggggacagcctcctagagcctttctggccaatgggaacaggaatagcccggggctttctagctgctatggactctgcctggatggtccgaagttggtctctaggaacgagccctttggaagtgctggcagagagggaaagtatttacaggttgctgcctcagaccacccctgagaatgtgagtaagaacttcagccagtacagtatcgaccctgtcactcggtatcccaatatcaacgtcaacttcctccggccaagccaggtgcgccatttatatgatactggcgaaacaaaagatattcacctggaaatggagagcctggtgaattcccgaaccacccccaaattgactcgcaatgagtctgtagctcgttcaagcaaactgctgggttggtgccagaggcagacagatggctatgcaggggtaaacgtgacagatctcaccatgtcctggaaaagtggcttggccctttgtgcaattatccatagataccgccctgacctgatagattttgattctttggatgagcaaaatgtggagaagaataaccaactggcctttgacattgctgagaaggaattgggcatttctcccatcatgacaggcaaagaaatggcctccgtgggggagcctgataagctgtccatggtgatgtacctgactcagttctacgagatgtttaaggactccctcccctctagcgacaccttggacctaaatgccgaggagaaagcagtcctgatagccagcaccagatcccctatctccttcctaagcaaacttggccagaccatctctcggaagcgttctcccaaggataaaaaggaaaaggacttggatggtgctgggaagaggagaaagaccagtcaatcagaggaggaggaagctcctcggggccacagaggagaaagaccgaccctggtgagcactctgacagacaggaggatggacgttgccgttgggaaccagaacaaagtgaagtacatggcgacccagctgctggccaaatttgaagagaatgcgcccgcacagtccatcggcatacggagacagctgacacaagagcgtggggccagccagccgtcctgctgcctgcctgggcaggttcgccctgcccccaccccccggtggaaacagggctccatgaagaaggagttcccgcagaacctgggaggcagcgacacatgctacttctgccagaagcgggtctacgtgatggagaggctgagtgccgagggcaagttcttccaccggagctgcttcaagtgcgagtactgcgccaccaccctgcgcctctcggcctacgcctacgacatcgaggatggtaaattctactgtaagccacactactgctatcgactctctggctacgcacaaaggaagagaccggcagtggctcccctgtctggaaaggaggccaaaggacccctgcaggatggcgccaccacagatgcaaacggacgggccaacgccgtggccagctccactgagagaaccccaggttcaggcgtgaacggcctggaggagcccagcatcgccaagcgactgaggggcaccccagagcggatcgagctggagaactaccgcctgtccctgaggcaggctgaggcactgcaggaggtaccggaggagactcaggccgagcacaacctgagcagcgtgctggacacgggcgccgaggaggacgtcgccagcaggtcagcacgcagggctgcagggcgcccacccgccacacggcccgaagagtccagtgaagccgggaaccagaggctccagcaggtcatgcacgcggcggatcctctggagatccaggctgacgtgcactggactcatatccgtgagagagaggaggaagagaggatggcgccggcctctgagtcctctgcttccggagccccattggatgagaatgacctagaggaagatgtggactcagaaccagccgagatagaaggggaggcagcagaggatggggacccaggggacactggtgctgagctggatgatgatcagcactggtctgacagcccgtcggatgctgacagagagctgcgtttgccgtgcccagctgagggggaagcagagctggagctgagggtgtcggaagatgaggagaagctgcccgcctcaccgaagcaccaagagagaggtccctcccaagccaccagccccatccggtctccccaggaatcagctcttctgttcattccagtccacagcccctcaacagaggggccccaactcccacctgtccctgccgccacccaggagaaatcacctgaggagcgccttttccctgagcctttgctccccaaagagaagcccaaagctgatgccccctcggatctgaaagctgtgcactctcccatccgatcacagccagtgaccctgccagaagctaggactcctgtctcaccagggagcccgcagccccggccacccgtggcggcctccacgcccccacccagcccactccccatctgctcccagccccagccttccaccgaggccactgtcccatcccctacccagtcccccatacgcttccagcctgccccggccaaaacatccaccccactggcccctctccctgtccaaagccaaagtgacaccaaggacagactgggcagcccccttgctgtggatgaggccctcagacggagcgacctggtggaggagttctggatgaagagtgcggagatccgccgcagcctcgggctcacacctgtggaccgcagcaaggggcccgagcccagcttccccacgcctgccttcaggccagtgtccctcaaatcctattccgttgaaaagtccccccaggatgagggactccaccttctcaagcctctgtccatccccaaaaggctgggcctgccaaagccggaaggcgagccgttgtccctgccaaccccccggtccccgtccgacagagagctacgcagcgcccaggaggagcgcagggagctgtccagcagctctggcctgggcctgcacgggagctcctccaacatgaagacactgggcagccagagcttcaacacctcggactccgccatgctcacgcccccctccagcccgcccccaccgccacccccgggcgaggagcccgccaccttgcggaggaagctcagggaggccgagcccaatgcctcggtggtcccgccgcccttgcccgccacctggatgcggcccccccgggagcctgctcagccccccagagaggaggtgcggaagtcgtttgtggagagtgtggaggagattccctttgctgatgatgtggaggacacctatgacgacaagactgaggactcaagcctgcaggagaaattcttcacgcccccgtcctgctggccgcgccccgagaagcctcgccacccgcccctggccaaggagaacgggaggctgcctgctctggaggggacgctgcagccacagaagagggggctgcccttggtgtccgcggaagccaaggagttggccgaggagcgcatgcgagccagggagaagtccgtgaagagccaggcgctgcgggatgccatggccaggcagctgagcaggatgcagcagatggagctggcctcaggcgcccccaggccccgcaaggcgtcctcagcaccctcccagggcaaggagcgccggcctgactcccccacacgccccactctcaggggctccgaggagcccaccctgaagcatgaagccaccagcgaggaggtcctctccccgccgtcggactcagggggcccagatggctctttcacttcatccgagggctccagtgggaagagcaagaagaggtcgtcactcttctccccccgcagaaacaagaaggagaagaagtccaaaggcgagggccggcccccggagaagcccagctccaacctcctggaagaagccgccgccaaacccaagtccctgtggaagtccgtcttctccgggtacaagaaggacaagaagaagaaggccgacgacaagtcctgccccagcaccccctccagcggggccacggtggactctggaaagcacagggtgcttcccgtcgtaagggcagagctgcagctccggcgccagctgagcttctccgaggactcagacctctccagcgacgatgtccttgagaagtcctcacagaagtcccggcgagagccaagaacctacacggaggaggaactgaatgccaagctgacccggcgtgtgcaaaaggcagctcggagacaggccaagcaggaggagcttaagcggctgcatcgagcccagatcatccagcggcagctgcagcaggtggaggagaggcagcggcggctggaggaaaggggcgtggctgtggagaaggcgctccggggcgaagcaggcatgggcaagaaggacgaccccaagctgatgcaggagtggttcaagctagtgcaggagaagaacgccatggtgcgctacgagtcggagctgatgatctttgcccgggagctggagctggaagaccggcagagtcgactgcagcaggagctccgggaacgcatggcagtggaagatcaccttaagactgaggaggagctgtcagaagagaagcagattctcaatgagatgctggaggtggtggagcagagagactcactggtggcgctgctggaggagcagcggctccgggagagagaggaggacaaggacctggaggctgccatgctgtccaagggcttcagccttaactggtcctgagctcccacccaacgctccattttctgttggcatctgcctggccaggcagtggcatccaaaccacccggagccgcgatctgaggaggcctggcacctccttggagtttacgctcagatgcccgtgtgctgcttggaaagtggtcgagtcccgcgtgcagtggggagccccaggtgacagtggttatctgagacggctccacctcctgggaggaggcccacctggacctcccactcagaggaggagcacggcgtgtatggcatgacgcaggggaccaccccgcgcgctccctgaggatgtgctggctgtgccccttttttccactggcacatttggtaagagagggaagctgctccccgtcagaaccacagtgcgccgtgcgaggggcactgtcttcttcatgctccctggagcaccaccaaagaaacgtaaacaataccccacgaaagcagggtcaggggtcagggtgcgatcgagacccaggatgggggcgtccagtcatgcccaccccagcatcacaggagacatggaggtgcgggcaggctcctgaattattatgcaaattaggaggacgcaggaggggtctgccctccagccgaacaccacacactggaccctaagtggccaaatgcctgggccgcttgctggctgtggcctgaggcttgtgggttgctgcattttgcttgtagttcacaaccattttgacactggaaaatgctgactttgggggacaggatgaggccctacattctaagcccccagttggcagacaggcattgtccctgttccacatttatgtcgggacaggagatgaccttttcctccgtgtttttcctgtgtttgcacgttgaaatgaagctgacaacctggcaagacgctcagccgcttcaaaccctttttgtcaattaacttattttttaatacttgaaaagaagtaacttcgtttgtgtatctttactagaggaactgatcacctgcgcccgggtgcgggagccacagcggcatctggtgcgtcctacgcgacctggtccggggctgcccggtgctcctcacgtgcatctatttattagcctttctcttcgtatcactggcctggctggcatcagggagctgcccagaacccctgtgtgggtcctcctcacagctttctgtcccctcctccacccggtgcctgcctcaccctggcgctagaccatctggaccactcatgtgatgagggtgcatttccgttctgttttgggccaggccaacagcagagctgccactctcaccctcccagtgagaattccggctctgcagaactcgcccttgtctcagtttgggggccagggcatcaccttcctccgcacatatgttaaagaaggtttcaggatgggccctcatccacacaggccaagagggtgcaaggtgggaccctggaatcatgtggctggtgagaattccctcctcccagcctaagattcacccagacggaaacgcgagtgctgcagtggatgctgggatgcaggctggtgcctctcaagcagatcagcgactccccccttccttccgaaggtgacgggcacctgccttgtgccggatcttcatggggacataaagggcgagccccgaatcactagctcctatagccaaactgttcctttctcacggttccgcaccagcctggctgtgtacagctcatcaagccactgagctaatcggggtggggtgcttgtccatcaaagcagtcagcacatagcccagcgaggagccatccggaccagacccgcctgccaggggcgctccagctgcctgccgcctccgtgggctggggccagctgggagcagaggcctggcccctccaaggcgtcccgcagtggagcaggctgagtggctgtgctgaccttgggctttccatgggaaccacactgtgcttcaacttgaacattcatcccagctgcaaaggagcaaagaacctgtgtcatccttgtctgtccagaagctgccatctctctcccatgcacatccgggagataacgccgctagtggccgccacagcgctgattctccacctgttctcacttgaggcaaaggtctccttttcttttctctttacttaaaaaaggggagaggagggtttctagattccatcttcaaaccccagtcgtcccataaaattggacgtgggaaaagaccttcactgcctgcgtggcctttcccagacctctcctcgaatgccacaaaaccggtccagccccggcagagccgccttcggcccttgtagctcctgctggccccacaacagggaaacagtttgcaaagtggcttggaaggagtgtggcctagggatctgctgtaagtggccagacgtagcaggagagcccagtgtcaccttctggtcctggtcagccttaacactgtggcatcctccacagcacacatggcagcctgcagtctggaaggtggacccgagcctttgcagagaggcgccgcagagccgcaggcctgcgccccagccttctgctcccgactgtgaggacacccagttctagtagagcactttttttaaagctcccattttgtaaccactagtttgcggttgacttgagtactctggtgacttcctgcgtcaagcgttctcaagctgtgagaatgtgcgcagctccaggcaggttttctctcggagagttaagtcttcccttgaaggcagggaagcaggatggatacacatatatcacacacataaaacaccaggtgcgggagcagcccagactcaaggctgactaaactggaggctgaataccgtggaggtccacatgcagcttccctggagggcaggccggaggcgctcccgcccctgggcttgaggatgctgcaccccgtgggcttccaggcctgcccagatgatgccttcaggcctctgtccctggcggccatcctcaggccgattttgaccagcaatgatagactcttcttaaccctttcaaaataaatttttcagtgggacagaaaggagagttaaaaaacatttttttaaaggtggtaacatctgacccacaaagggaatgggtctgttttatgcaaaataaaagtttttcaaat

SEQ ID NO: 6 - protein, 1965 aa, Homo sapiens
MEERKHETMNPAHVLFDRFVQATTCKGTLKAFQELCDHLELKPKDYRSFYHKLKSKLNYWKAKALWAKLDKRGSHKDYKKGKACTNTKCLIIGAGPCGLRTAIDLSLLGAKVVVIEKRDAFSRNNVLHLWPFTIHDLRGLGAKKFYGKFCAGAIDHISIRQLQLILLKVALILGIEIHVNVEFQGLIQPPEDQENERIGWRALVHPKTHPVSEYEFEVIIGGDGRRNTLEGFRRKEFRGKLAIAITANFINRNTTAEAKVEEISGVAFIFNQKFFQELREATGIDLENIVYYKDDTHYFVMTAKKQSLLDKGVILHDYADTELLLSRENVDQEALLSYAREAADFSTQQQLPSLDFAINHYGQPDVAMFDFTCMYASENAALVREQNGHQLLVALVGDSLLEPFWPMGTGIARGFLAAMDSAWMVRSWSLGTSPLEVLAERESIYRLLPQTTPENVSKNFSQYSIDPVTRYPNINVNFLRPSQVRHLYDTGETKDIHLEMESLVNSRTTPKLTRNESVARSSKLLGWCQRQTDGYAGVNVTDLTMSWKSGLALCAIIHRYRPDLIDFDSLDEQNVEKNNQLAFDIAEKELGISPIMTGKEMASVGEPDKLSMVMYLTQFYEMFKDSLPSSDTLDLNAEEKAVLIASTRSPISFLSKLGQTISRKRSPKDKKEKDLDGAGKRRKTSQSEEEEAPRGHRGERPTLVSTLTDRRMDVAVGNQNKVKYMATQLLAKFEENAPAQSIGIRRQLTQERGASQPSCCLPGQVRPAPTPRWKQGSMKKEFPQNLGGSDTCYFCQKRVYVMERLSAEGKFFHRSCFKCEYCATTLRLSAYAYDIEDGKFYCKPHYCYRLSGYAQRKRPAVAPLSGKEAKGPLQDGATTDANGRANAVASSTERTPGSGVNGLEEPSIAKRLRGTPERIELENYRLSLRQAEALQEVPEETQAEHNLSSVLDTGAEEDVASRSARRAAGRPPATRPEESSEAGNQRLQQVMHAADPLEIQADVHWTHIREREEEERMAPASESSASGAPLDENDLEEDVDSEPAEIEGEAAEDGDPGDTGAELDDDQHWSDSPSDADRELRLPCPAEGEAELELRVSEDEEKLPASPKHQERGPSQATSPIRSPQESALLFIPVHSPSTEGPQLPPVPAATQEKSPEERLFPEPLLPKEKPKADAPSDLKAVHSPIRSQPVTLPEARTPVSPGSPQPRPPVAASTPPPSPLPICSQPQPSTEATVPSPTQSPIRFQPAPAKTSTPLAPLPVQSQSDTKDRLGSPLAVDEALRRSDLVEEFWMKSAEIRRSLGLTPVDRSKGPEPSFPTPAFRPVSLKSYSVEKSPQDEGLHLLKPLSIPKRLGLPKPEGEPLSLPTPRSPSDRELRSAQEERRELSSSSGLGLHGSSSNMKTLGSQSFNTSDSAMLTPPSSPPPPPPPGEEPATLRRKLREAEPNASVVPPPLPATWMRPPREPAQPPREEVRKSFVESVEEIPFADDVEDTYDDKTEDSSLQEKFFTPPSCWPRPEKPRHPPLAKENGRLPALEGTLQPQKRGLPLVSAEAKELAEERMRAREKSVKSQALRDAMARQLSRMQQMELASGAPRPRKASSAPSQGKERRPDSPTRPTLRGSEEPTLKHEATSEEVLSPPSDSGGPDGSFTSSEGSSGKSKKRSSLFSPRRNKKEKKSKGEGRPPEKPSSNLLEEAAAKPKSLWKSVFSGYKKDKKKKADDKSCPSTPSSGATVDSGKHRVLPVVRAELQLRRQLSFSEDSDLSSDDVLEKSSQKSRREPRTYTEEELNAKLTRRVQKAARRQAKQEELKRLHRAQIIQRQLQQVEERQRRLEERGVAVEKALRGEAGMGKKDDPKLMQEWFKLVQEKNAMVRYESELMIFARELELEDRQSRLQQELRERMAVEDHLKTEEELSEEKQILNEMLEVVEQRDSLVALLEEQRLREREEDKDLEAAMLSKGFSLNWS

SEQ ID NO: 7 - DNA, 14172 bp, Drosophila
atgagccgccaacaccagcggcaccaccagcagcatcaccacctgccgccgcaccagcaaccgcagcagcagatgccgcaacaacagcagcagctgacggcgcagcagcagcaacaacagcagctgctgatggcggagcacgcggcggccgcggaggcggcggagctattcgacctgctgtgcgtggccacaacgatgcgccagatcctggcgctccatcgggccatgtgcgaggctgtgggattgagaccctcgcctctgaacgacttctacccacggctaaaggccaaggtgcgttcgtggaaggcgcaggccctgtggaagaagttcgacgccagagctgcccatagagtctacggcaagggagctgcctgtactggcacacgcgtcctggtcatcggagcagggccctgtggactgcgcaccgccatcgaggcccaactgctgggcgccaaggtggtggtgctggagaaacgcgatcgcatcacccggaacaatgtgctccatctgtggccattcgtcatcacggatctgcgcaacttgggcgcaaagaagttctacggcaagttttgcgccggctccatcgatcacatctccattcggcagctgcagtgcatgctgctcaaggtggcgctgctcctgggcgtagagatccacgagggagtcagttttgatcacgctgtagagccctctggcgatggcggcggatggagggcagctgttactcccgcagatcatcctgtatctcactacgaattcgatgtgttgatcggagcggatggcaagcggaatatgctggactttaggaggaaggagttccgcgggaagctggccatcgctattacagcgaactttatcaacaagaagacggaggcggaggctaaagtagaggagatcagtggggtggctttcatcttcaaccaggccttcttcaaggagctgtacgggaagacgggcatcgacctggaaaacatcgtctactacaaggacgagacgcactacttcgtgatgacggccaagaagcacagtctaattgacaagggcgttattatcgaggatatggccgatcccggcgagcttctcgccccagccaatgtggatacacaaaagctgcacgactatgcacgcgaggctgcggagttctccacccaataccaaatgccaaacctggagttcgctgttaatcactacggcaaaccagatgtggccatgttcgacttcacatcgatgtttgccgccgagatgtcctgtcgggtgattgtgcgcaaaggagctcgcctgatgcagtgcctcgtgggtgacagtctgctcgagccgttttggcccactggatcgggttgtgcccgtggattcttatccagcatggatgctgcctatgccatcaagctttggtccaacccgcagaacagcacacttggcgttctggcgcagcgcgaaagcatctaccggctgcttaaccagaccacgccggacaccctgcagcgggacatcagtgcctataccgtggatccggccacgcgctatccgaatctgaacagggagtcggtcaatagctggcaggtcaaacatctggtcgacacggacgacccgtccattctggagcagaccttcatggacacgcatgctctgcagaccccgcatttggacacaccgggcagacgcaagcgacgcagtggagacttgctgccccagggtgccacgttgctgagatggataagtgcccagctgcattcctatcagtttattcccgaactcaaggaggcttcggatgtgttccggaatggacgcgttctgtgtgcgcttatcaatcgctatcgtcctgatctcatcgactacgctgccaccaaggacatgagtcccgtggagtgcaatgagctgtcattcgccgtcctagagcgcgaactccacatcgatcgcgtcatgagtgccaaacagtcgctggacttgaccgagctggagtcgcgaatctggctcaactatttggaccagatctgcgacttgtttcgcggcgagatcccccatatcaagcaccccaagatggactttagcgatttgcgccagaagtatcgtatcaaccatacgcatgcccaacccgacttctccaagctgctggcaacgaaacccaaggccaagtcgccgatgcaggatgctgtggacatacccacgacagtgcagcggcgctcggtgctcgaggaggagcgagccaagcggcagcgtcgccacgagcagcttcttaacatcggtggaggggcagcaggagccgccgccggagttgccggaagcgggacaggaaccacaacgcagggtcaaaacgatacgccacgccggtccaagaagcgccgtcaggttgacaaaaccgccaatattgaggagcgccagcagcgcttgcaggagatcgaggagaatcggcaggagcggatgagcaagcggcgccagcagcgctgtcaccagacgcagaatttctacaagagccttcagctcctgcaggcgggcaagctcttgagggagggtggtgaggcgggagtggccgaggatggcaccccattcgaggactactcgatattcctctaccgccagcaggcgcccgtattcaatgatcgcgtcaaggacctcgagcgaaagcttctgtttcccgatcgcgaacggggagatattccgtcggcattgccgcgcacggcggacgagcagttcagcgatcggattaaaaacatggagcagcggatgacgggacgtggtggccttggtggtgacaagaagcccaaggatctgatgcgggctatcggcaagatcgactcgaacgattggaatgtacgcgagatcgagaagaagatcgagctgtcgaagaagacggagatccacgggcctaagggccgcgagaaggttcccaagtggagcaaggagcagttccaggcgcgacagcacaagatgtccaagccgcaacgccaggattcgcgtgaggcggaaaagttcaaggacatcgaccagactatccgcaacttggacaagcagctaaaggagggccacaatctggatgtgggcgagcggggacgcaacaaagtggcctccattgccggtcagtttggcaaaaaggatgaggccaattcggatgagaagaacgcgggcagcagcaatgccaccaccaacaccaacaacacagtcatacccaaatctagttccaaggtggcgctggcctttaaaaagcaggctgcctccgaaaagtgccgcttctgtaagcaaaccgtctacccgatggagaagaccaccgtggagggattggttctgcatcgcaattgccttaagtgccaccactgccacaccaacttgcgtctgggaggctacgcctttgatcgggacgatccgcagggccgactttactgcacccaacacttccggttgccacccaaaccgctgccgcagcgcaccaacaaagccaggaaatccgctgccgctcaacccgcctcgcctgctgtaccaccaactgcgggatccgtacccactgcagctgccacatcggagcatatggacaccactccacccagggaccaggtggatctactgcagacctcgcgagcaaatgcctctgccgatgccatgtccgatgatgaagccaatgttatcgatgagcacgaatggtctggtcgcaacttcttgcccgagtccaacaacgattcccaatcagagctatccagttcagatgaatcggatacggaatcggattcggagatgtttgaggaggcggatgattcgccgtttggtgctcagaccctccagctggcgtcggattggattggaaagcaatactgtgaggacagtgatgattctgacgatttctacgactcaagtgaagatgacggcaaagatgacaccgagggtgaggaattcaagaaggcccgcgaattgaggcgccaggaagttcgcctgcagccgttgcccgccaatctgcccacagatacggagaccgagaaactcaagttaaacgtagacaataaagaaaacatggcggacggaagctctctgaaatcgggcaattcctttgagtccgcgcgcagccagccgtctacgcccctgtccacgcccactcgtgtcgagatggagcaactggagcgagatgctccacgcaagtttagcagcgaaatcgaggcaatcagcgagaaactttaccacatgaacaacatggtaaagatgaacaaggacctcgaggtgctggccaaggaaaatctggtcaagagcggcatcctgcgcaagctcacgctgaaggagaagtggctggcggagaatgcggccatagcagcaggccaaaaagtgactccgactcctagtgcgactgctcctgggcttcaacccaagtcgaagttcgatgaaaagttcgaaaaggtagtgagtccacctcagccggttgtcgaacccaaacccaagcccgtaatcgatttcaatttggatgagttaaaaccacgtaaaccgaactttgaggagcgacccaaggagcagctgcccaggcctgaaagtttgaaaaaacctccacaacaaaaaccgaagggcagcagcacaaacgtgagccgctccaacagcttgaagagtaatgccagcaacgggagtccaaaggtgaaaaaagctcctgtttcaaataacagcaaaatgcaaattgaaggaatccttgataccttgagaaagattcagagccagaacagtagcgatcaggatgaagatatggatgtagatgaggatgtggaaagaaagccaaataaagagctcaacagcaagttgaaggaaatccaagccagcagctttgccggcacaatggaccatattaaatcccagctgactatgccaacggttagtgcgcaggcaccaccctccatggatctttccaagtactttcccaaccagaagcaagagaagagctccaccagcagcacaaacaagaatcaggttaccctgaaggatgtaaatctggccaagtacttccccagcagtccagctccacaacggagaaccgtggaaacggtggccgatcgactaaagaagtcacagactgaggctgctctagctaaaaccaagctactggaggatcaggccaataaccaggccgaaaagaccaagaaagaggtcgaaaaggaaggagagtctaaaaaaatcacgaaaaaggtggcagactccaaagcagtacctccaaaacggcaggcctcgttagacacctttagcttaagggagcaccaaatggatggagcattagacctgaccaaaaagaagggtcccaccaaagcgagcgctggtgttaaaaaaccagccaaatcagggagtaccacatccgtgacaaaagccacggccacatccgaaggaaagaccattaagattgtaaagaaaatcgtacccaaaggaaccaaagctaaaaaggcagcagaagcggcccaagaatccgcagtagtagaggctccgccagaaaagaaacctccaaaagatgaagcagagcggatattggacgaaattctgggagatggtgagtatcgttcacccagctccgagtatcagcgcttgttccaggacgaaaagtcgcctagcgatttgtccgataacatagatcggatactcgaggaatccgagttggatgtggaattgggtctgccaaaacgcagcagtaagaaactggtaaaaaccaaatccctgggcgagggtgactttgacatgaagccttcaaaagaaagacttactggagtacagaacatacttaagcgatttgagtcaatgagttcagtcacctcacagaacagtgatgaacaggcggggttcaaattacgtcgcatggagtccaccaccagtaatctgagcagcttgacccgttccagggaatcacttgtctccgtcagcgattcaatgagtgatttggagaaaaccatggactacctccgtaatgaatggcgcaatgaggccaccaattttctgcaaaagaagcgtgataaattctatgccaaaaaggaggagcaagaaaaggaggctaaaattctggctaaacctgatcccttagacaatcttcctgttcaatatcgcgactccaagttggccaagttctttggcttggccgctagcaagtcaccagaaaaccgaaagtcccctattaagaagaagaaatctccctctaaaacgccaaaggtgaccaaagcaaacaattcactggaggagctggccaagattagtaatgttcgccagaccaagaaggcacagccgaagactctaaagcctgtagaagtaaaacctttaaagccagccagtccagttccagatgattttgagattcttgatctgctggaaaaagccacggaggccaaagaactggaacgctcgaagaccaagagtccagccgtagaatctatcagtcagacgcccaaagaagctatagtagaaatttccttaccagtggaagacattaaaaatctacccaaaaccgggtgtgataagtcctcaaacagctctcgccgtggttcccaatctagtttgatcatgtcgcgacgacactcggagatttctctaaacgagaaactcaaccaggacgcactcgcagccttgaaccaaatagaaaaagaaagagaggcggagcaagtggacgaacttttccagagcatggtggaggaaatggagcaggaaccgcagcccacagctattgtcgaacctccagaagaagacatcgatgcagattccttgtgcacaacaattagcaagagtcccagtgcacagcccgttacggtggtcaaacgtggcagttccgaggatcagagcatagagaagcttttcagccacttttccgacgagatgctggtgaatgtggagtttgactctaacgatgagcttgtaggaattacaccaagggcaacactcgtttcccgaaacacagaagatcgggactatctggataaactagagtctttggagcgcgatgaggagactttccaaccggttgttggggaaaaattcatacaggaaaatgtccaggacgaagtggacggattgcatttcccatcacgcccacaacggagacccaaaagcagttcatcttccagtgaaccatcacttcctgtggctcctcaaaggttggaaaaaaagctatcgaaattagatccggaggatatgcctccttctgtacaggatctattacaacaggtttaccagaagaacatccaacctgaattagtggaagtaattccagttgaaggcaaacaaactttaaggttccccagcatgttggcggaagaagatgtagacgaagtggatcactctaaagaaggaatcaagaaaattgaaacggctccagaagaggttcgtaaggtaaccgagccggaggatgttgctcgggtaattccaagtccaatcaaaccgtcaataagtcagagcaactccctcaagagcgaaaattcctctggcagcagtttagtggaaataccaaaaataattgcaccacccaagtcgagtagcaaggaaaactcttcggattgggacagggagaagctgcccgcatcacccatgccacgacgaagacttctgccaaatcaaacgccatataaagccccaagcgtggctagtaaggagagctccttagagtgggacatggagaagctacccaacagtcccatgttgccaaggaggaacaagatgcgagcaatctcacctagtaccaaccctgtgcaacttctcaacaatttaccctccgatgtagatgacgaggcggcacagagacgccttatcgaggatttcgagcaggagagacgtcaggctctgatcaagcgggatgagaacttcgaggccattgcggcggagcaacgcaggcgcgactccttacagagcagcagtaactcgagcagcaaacgcagtttaccaccgcccacgccgcccatgatggcatcgcgacgcggtacaactcaagacacaaatcgcacccaggataccgcatcgcggcacgagggaacgccgcccatgttcaagaagctggatgtcgatggcagcggtacatcaatggattccacctcctgctccacgcggcgcagttcgtttgcatttatagagttacaggataacaaaccagtgattgtgcccatgcccaagaaactgaagttgccaaagccggagccacctaggttcgtacccgagccagtggccactgatgagcctgtgcccgaggtttttcagggtcgcgcttggcccaagacacagttggaaggagaggtcgatctaggcgattcggataatgaagatgaaacagaaaagctaaagaaacagctgcccgaatacgctcgttcggactctcctccttcggctgcattcaagaatcgcaagtggccggatgggaaaacagtttttgataaacgagccgaatctcttgaggaggaagatatcttcgaaggattaccgtccccaaggaaaagaggttctcaaagattcatggacaagccgcgctctcaatcgccacagcctttcaaaccgctggccaacagctccaggaaaagctcaaagtcttttagcgaccttaagaaaggaccctccttgcaatctctgtcggcgcaatccagccaggacacggacacactgtccaccaccacaacagtagccacagctcgtcctgctagctatgccaactacgaggaccccatggatgccagtacccaagctttgctggatcgaagcaaacggttacacaaccgcaaaagagatttcgtaaacgagcgagtagtggagcgcaacccctatatgagggatgtgcttaggagcacggatcgccgtgattacgacgacgtggatgaagatctgactagctacaggccaagacattatgccagctccacgctaaatcgtttccccaacaccacaataagaaaaagcaacaactacgattacctcagtcccagcagtgattatttgagcaggagaagctacataccgagcgccagtgcaaccagcagttactatccatcgaccacgcgtagctcccatttgagtgacctgttccgacgacgcagccccgccagcggaactgtatccgcactttccggctacggcaacaaagagtcgtgcgttatctcaatcgggctggccttagatcgagttggccacttgattgaaagtaaatgcacctgggtacgatctacgaaggttcaaaccgagtccgagagcacttcacccgacgaagtggagctcaattctgccactgagatatccaccgactctgagtttgacaacgatgagattatacgccaggcgcccaaaatcttcatcgatgacacccatctaaggaagcccaccaaggttcagatcaagtccaccatgatcggacccaatgcagcttccgccggactccatcagaagcagttggcggcgcgtgagaagggcggcagctacctccagaagtaccaaccacaaccgccactgccacagtttagaccgttggtccaggtggatcccaccctgctcattggcagccagcgcgctcctcttcagaatccacggccaggagactacttgctaaacaagacggccagtacggagggtatcgcctcaaaaaagagcctggggctaaaaaagcgctatctgctgggtgagccggccaatggcaataagatccagaagtccggatccacttcagtgctggattcacgcattcgcagcttccagtcgaacatatcggagtgccagaagcttttgaatcccagcagcgacataagtgccggcatgcgaaccttcctcgatcgcacaaagttgggcgaaggcagccagacgacacccggacagacgaacgaactaatccgttccgccaccagcaatgtgattaacgatctgcgcgtggagcttcggatacagaaaactggctccagccactccacggacaacgagaaggaaaacgttttcgtgaactgtaagaacgagctgaacaaggggatggaatacacggatgcggtcaatgccacgctgctggaccagctggccagaaaaagttcacccaccacgccgacgaataagacggtggtcgaggttattgacctggttacacctgagaagccaattgacattatcgatctaacggcactggaaacgccgaaaaagcagttggtcgatggtagcgccatggatgtagatgaacgcctcacacccgatagcaacaaaatcagcgaactgcagcaggaagtgaaggaggaacccaagccggatgtctctagggatgtgaaagaatgcataccagatatactgggacacattaaggagggaacgggatcgaaggagccaggtggagaggaccaacagagcctgctggagcagtcggacgaagagaagcgcgactcaccggaaaaggatgtggccgaacatgagctttatgaaccggacagtgtgcagatccaggtgcccaatatcccatgggaaaaaagcaagccggaggtcatgtctaccaccggcagcagtggctccatctgctcaagctcagactcttctagtattgaagacatccagcactacattttggagtccacaactagtccagatactcagacagttggcggaaagcacaatgtgccccgtttggaggtgcacgacacaagtggtgccctgatgcaggtggacagcctgatgattgtgaacggaaagtatattggggatcccgaggatgtcaagttcttggatatgccggccaatgttattgttccgccagcaccggcgcttaaaacgaatgagctggatatggaggatgaccaagaggcggaggcggaaccagtaactgctactccggagccggtggaatgtacggtcatcgaggctgagcgccgtgttactgctccccctcctttgccagagatgggtccacccaaactgaagttcgatagcaaaaatgagaacaagatcgagagcttgaagaatcttccgttgatcgtagagagcaatgtggagcacagtcaggcagtgaaacccattactcttaacttaagcaatctggccaggacgccggatacaccaaccacgcccacggcgcacgatagcgataaaacacccactggggaaattctctcgcgaggatctgactcagaaaccgagcacactggcactggtcaggtactaacggagacggaactctccgactggacggccgacgactgtatctcggagaactttgttgacttggagttcgcgcttaactctaacaagggtacgataaaacggcgcaaggatcgacgacgcagtggagcaagcaaacttcccagtggcaacgaggtaatccacgagctggccaggcaggcgccagtggtgcaaatggatggaattcttagtgccatcgacattgatgacatagagttcatggacacgggttcggagggttcttgtgctgaagcttatcccgcaacaaatacagctctcattcagaatagaggttacatggagtacatcgaggcggagccgaaaaagacgacccgcaaggcagctccaccatccagttacccaggaaatttaccgcctttaatgacgaagcgggacgagaaactgggcgttgattacattgagcagggggcgtacataatgcacgatgatgcaaagacgcctgtgaatgaggtggctcctgccatgacccagtcgctaactgactcaatcacgctcaatgaactggatgatgacagtatgataatatcccaaacccagccaacgacaacggaggaaagtgaggcactgacggtggtcaccagtccacttgacacgtcctcgcccagggttctcgatcaatttgcatccatgttggcggcgggaaaaggtgactccacacccagtagctcagagcaacaaccaaagacgtctacggtgacgagcagcagcactgggcccaactcctcgacaacaggaaacgtctcgaaggagccgcaggaggaggacctgcaaatccagtttgagtatgttcgagcactgcagcagcggatatcgcagatcagcacccaacggcgtaagagctctaagggagaggcacctaacctgcagctaaacagtagcgcacctgtgatagaatcagccgaggatccggccaagcccgcagaggagcctctggtctcaatgcgaccgcggaccaccagcatttccggaaaggtaccggagatacccacacttagcagcaagctggaagagataaccaaagaacgcactaagcaaaaggatctgattcacgacctagtcatggacaagttgcagtcgaagaagcagctaaacgctgagaagcgtctgcaccggagtcgacagcgcagtttgctgaccagtggctatgccagtggctccagccttagtccgacgcccaagctggctgctgcttgcagtccgcaggattccaactgctctagccaagcgcactaccacgcctccacggcggaggaggccccgaagccgccggcggaaaggccgttgcagaagtccgccacgtccacctatgtgtcgccttatcgcactgtccaagcgcccacacgtagtgctgatctctataagccgcgccccttcagcgaacacatcgattcgaacgctctggcgggttacaagctcggcaagacggcctcgtttaatggcggcaagttgggcgactttgcgaaacccattgccccggcgagagttaaccgaggaggaggtgtcgcgaccgcggatatagccaatatttccgcgtcgacggagaacctaagaagcgaggccagggccagggctcgtcttaagtctaacacagagctgggccttagtcccgaggaaaagatgcagctaatacgttcaagattgcactacgaccaaaacagatctctgaagccgaagcaactggaggagatgccatccggggatctggcggcacgtgcccgcaaaatgagtgcctcgaagagcgtcaatgatctggcctacatggtgggacagcagcagcagcagcaggttgagaaggatgccgtgctccaagccaaggcggctgactttacatccgatcccaatttggcgtccggtggtcaggagaaggcaggcaaaactaagtccggacgcaggccaaaggatccggagcggcgtaagagtctcatacagtcgttgtccagcttcttccaaaagggatctggatccgcggcctccagttccaaggagcagggcggcgctgtggctgccgtccactctgaacagtcagagcgaccaggcaccagcagcagcggcacgcccacaatatcggatgcggcgggtggaggcggaggaggaggtggcgtcttcagcagattccgcatctcgcccaagtccaaggagaagtcaaagtcttgctttgatctgaggaatttcggttttggtgacaaggatatgctggtctgcaatgcagcatctccagcaggagccacatccgcatcacagaaaaatcactcgcaagagtatctgaacaccacgaacaacagtcgctatcgaaagcaaacgaacactgcgaaaccgaaacccgaatcgttctcttcatccagtccgcagctctatatacacaagccccaccacctggccgcagctcatcccagtgccctggacgaccagacaccaccacccataccgcctcttccactgaattatcagagatccgatgatgagagctacgctaacgagacacgagagcataagaagcaacgtgccatatcgaaggcttcacgacaagctgagctcaagcgattgcgaatcgctcaagagattcagcgggaacaggaggagatcgaggtgcaactgaaggatctggaggcacgcggcgtgcttattgagaaggccttgcgaggcgaggcgcagaatattgaaaacctggatgcgacaaaggacaacgacgagaagctacttaaggaacttttggagatttggcgcaacatcacagcactcaagaaacgcgatgaggaactgactataaggcaacaggaactgcaactggagtatcggcatgcccagctgaaggaagagctcaatctgcgcttgtcctgcaacaaactggacaaaagctctgccgatgtggccgccgagggagcaattctcaacgagatgctggaaattgtcgccaagcgagccgccctacgacccacagcctcccagctcgacctcacggcagcgggatcagcatccacgtccgccgaggcaacgggcattaagctgacgggacaaccgcatgaccacgaagaatcgatcatttga

SEQ ID NO: 8 - protein, 4723 aa, Drosophila
MSRQHQRHHQQHHHLPPHQQPQQQMPQQQQQLTAQQQQQQQLLMAEHAAAAEAAELFDLLCVATTMRQILALHRAMCEAVGLRPSPLNDFYPRLKAKVRSWKAQALWKKFDARAAHRVYGKGAACTGTRVLVIGAGPCGLRTAIEAQLLGAKVVVLEKRDRITRNNVLHLWPFVITDLRNLGAKKFYGKFCAGSIDHISIRQLQCMLLKVALLLGVEIHEGVSFDHAVEPSGDGGGWRAAVTPADHPVSHYEFDVLIGADGKRNMLDFRRKEFRGKLAIAITANFINKKTEAEAKVEEISGVAFIFNQAFFKELYGKTGIDLENIVYYKDETHYFVMTAKKHSLIDKGVIIEDMADPGELLAPANVDTQKLHDYAREAAEFSTQYQMPNLEFAVNHYGKPDVAMFDFTSMFAAEMSCRVIVRKGARLMQCLVGDSLLEPFWPTGSGCARGFLSSMDAAYAIKLWSNPQNSTLGVLAQRESIYRLLNQTTPDTLQRDISAYTVDPATRYPNLNRESVNSWQVKHLVDTDDPSILEQTFMDTHALQTPHLDTPGRRKRRSGDLLPQGATLLRWISAQLHSYQFIPELKEASDVFRNGRVLCALINRYRPDLIDYAATKDMSPVECNELSFAVLERELHIDRVMSAKQSLDLTELESRIWLNYLDQICDLFRGEIPHIKHPKMDFSDLRQKYRINHTHAQPDFSKLLATKPKAKSPMQDAVDIPTTVQRRSVLEEERAKRQRRHEQLLNIGGGAAGAAAGVAGSGTGTTTQGQNDTPRRSKKRRQVDKTANIEERQQRLQEIEENRQERMSKRRQQRCHQTQNFYKSLQLLQAGKLLREGGEAGVAEDGTPFEDYSIFLYRQQAPVFNDRVKDLERKLLFPDRERGDIPSALPRTADEQFSDRIKNMEQRMTGRGGLGGDKKPKDLMRAIGKIDSNDWNVREIEKKIELSKKTEIHGPKGREKVPKWSKEQFQARQHKMSKPQRQDSREAEKFKDIDQTIRNLDKQLKEGHNLDVGERGRNKVASIAGQFGKKDEANSDEKNAGSSNATTNTNNTVIPKSSSKVALAFKKQAASEKCRFCKQTVYPMEKTTVEGLVLHRNCLKCHHCHTNLRLGGYAFDRDDPQGRLYCTQHFRLPPKPLPQRTNKARKSAAAQPASPAVPPTAGSVPTAAATSEHMDTTPPRDQVDLLQTSRANASADAMSDDEANVIDEHEWSGRNFLPESNNDSQSELSSSDESDTESDSEMFEEADDSPFGAQTLQLASDWIGKQYCEDSDDSDDFYDSSEDDGKDDTEGEEFKKARELRRQEVRLQPLPANLPTDTETEKLKLNVDNKENMADGSSLKSGNSFESARSQPSTPLSTPTRVEMEQLERDAPRKFSSEIEAISEKLYHMNNMVKMNKDLEVLAKENLVKSGILRKLTLKEKWLAENAAIAAGQKVTPTPSATAPGLQPKSKFDEKFEKVVSPPQPVVEPKPKPVIDFNLDELKPRKPNFEERPKEQLPRPESLKKPPQQKPKGSSTNVSRSNSLKSNASNGSPKVKKAPVSNNSKMQIEGILDTLRKIQSQNSSDQDEDMDVDEDVERKPNKELNSKLKEIQASSFAGTMDHIKSQLTMPTVSAQAPPSMDLSKYFPNQKQEKSSTSSTNKNQVTLKDVNLAKYFPSSPAPQRRTVETVADRLKKSQTEAALAKTKLLEDQANNQAEKTKKEVEKEGESKKITKKVADSKAVPPKRQASLDTFSLREHQMDGALDLTKKKGPTKASAGVKKPAKSGSTTSVTKATATSEGKTIKIVKKIVPKGTKAKKAAEAAQESAVVEAPPEKKPPKDEAERILDEILGDGEYRSPSSEYQRLFQDEKSPSDLSDNIDRILEESELDVELGLPKRSSKKLVKTKSLGEGDFDMKPSKERLTGVQNILKRFESMSSVTSQNSDEQAGFKLRRMESTTSNLSSLTRSRESLVSVSDSMSDLEKTMDYLRNEWRNEATNFLQKKRDKFYAKKEEQEKEAKILAKPDPLDNLPVQYRDSKLAKFFGLAASKSPENRKSPIKKKKSPSKTPKVTKANNSLEELAKISNVRQTKKAQPKTLKPVEVKPLKPASPVPDDFEILDLLEKATEAKELERSKTKSPAVESISQTPKEAIVEISLPVEDIKNLPKTGCDKSSNSSRRGSQSSLIMSRRHSEISLNEKLNQDALAALNQIEKEREAEQVDELFQSMVEEMEQEPQPTAIVEPPEEDIDADSLCTTISKSPSAQPVTVVKRGSSEDQSIEKLFSHFSDEMLVNVEFDSNDELVGITPRATLVSRNTEDRDYLDKLESLERDEETFQPVVGEKFIQENVQDEVDGLHFPSRPQRRPKSSSSSSEPSLPVAPQRLEKKLSKLDPEDMPPSVQDLLQQVYQKNIQPELVEVIPVEGKQTLRFPSMLAEEDVDEVDHSKEGIKKIETAPEEVRKVTEPEDVARVIPSPIKPSISQSNSLKSENSSGSSLVEIPKIIAPPKSSSKENSSDWDREKLPASPMPRRRLLPNQTPYKAPSVASKESSLEWDMEKLPNSPMLPRRNKMRAISPSTNPVQLLNNLPSDVDDEAAQRRLIEDFEQERRQALIKRDENFEAIAAEQRRRDSLQSSSNSSSKRSLPPPTPPMMASRRGTTQDTNRTQDTASRHEGTPPMFKKLDVDGSGTSMDSTSCSTRRSSFAFIELQDNKPVIVPMPKKLKLPKPEPPRFVPEPVATDEPVPEVFQGRAWPKTQLEGEVDLGDSDNEDETEKLKKQLPEYARSDSPPSAAFKNRKWPDGKTVFDKRAESLEEEDIFEGLPSPRKRGSQRFMDKPRSQSPQPFKPLANSSRKSSKSFSDLKKGPSLQSLSAQSSQDTDTLSTTTTVATARPASYANYEDPMDASTQALLDRSKRLHNRKRDFVNERVVERNPYMRDVLRSTDRRDYDDVDEDLTSYRPRHYASSTLNRFPNTTIRKSNNYDYLSPSSDYLSRRSYIPSASATSSYYPSTTRSSHLSDLFRRRSPASGTVSALSGYGNKESCVISIGLALDRVGHLIESKCTWVRSTKVQTESESTSPDEVELNSATEISTDSEFDNDEIIRQAPKIFIDDTHLRKPTKVQIKSTMIGPNAASAGLHQKQLAAREKGGSYLQKYQPQPPLPQFRPLVQVDPTLLIGSQRAPLQNPRPGDYLLNKTASTEGIASKKSLGLKKRYLLGEPANGNKIQKSGSTSVLDSRIRSFQSNISECQKLLNPSSDISAGMRTFLDRTKLGEGSQTTPGQTNELIRSATSNVINDLRVELRIQKTGSSHSTDNEKENVFVNCKNELNKGMEYTDAVNATLLDQLARKSSPTTPTNKTVVEVIDLVTPEKPIDIIDLTALETPKKQLVDGSAMDVDERLTPDSNKISELQQEVKEEPKPDVSRDVKECIPDILGHIKEGTGSKEPGGEDQQSLLEQSDEEKRDSPEKDVAEHELYEPDSVQIQVPNIPWEKSKPEVMSTTGSSGSICSSSDSSSIEDIQHYILESTTSPDTQTVGGKHNVPRLEVHDTSGALMQVDSLMIVNGKYIGDPEDVKFLDMPANVIVPPAPALKTNELDMEDDQEAEAEPVTATPEPVECTVIEAERRVTAPPPLPEMGPPKLKFDSKNENKIESLKNLPLIVESNVEHSQAVKPITLNLSNLARTPDTPTTPTAHDSDKTPTGEILSRGSDSETEHTGTGQVLTETELSDWTADDCISENFVDLEFALNSNKGTIKRRKDRRRSGASKLPSGNEVIHELARQAPVVQMDGILSAIDIDDIEFMDTGSEGSCAEAYPATNTALIQNRGYMEYIEAEPKKTTRKAAPPSSYPGNLPPLMTKRDEKLGVDYIEQGAYIMHDDAKTPVNEVAPAMTQSLTDSITLNELDDDSMIISQTQPTTTEESEALTVVTSPLDTSSPRVLDQFASMLAAGKGDSTPSSSEQQPKTSTVTSSSTGPNSSTTGNVSKEPQEEDLQIQFEYVRALQQRISQISTQRRKSSKGEAPNLQLNSSAPVIESAEDPAKPAEEPLVSMRPRTTSISGKVPEIPTLSSKLEEITKERTKQKDLIHDLVMDKLQSKKQLNAEKRLHRSRQRSLLTSGYASGSSLSPTPKLAAACSPQDSNCSSQAHYHASTAEEAPKPPAERPLQKSATSTYVSPYRTVQAPTRSADLYKPRPFSEHIDSNALAGYKLGKTASFNGGKLGDFAKPIAPARVNRGGGVATADIANISASTENLRSEARARARLKSNTELGLSPEEKMQLIRSRLHYDQNRSLKPKQLEEMPSGDLAARARKMSASKSVNDLAYMVGQQQQQQVEKDAVLQAKAADFTSDPNLASGGQEKAGKTKSGRRPKDPERRKSLIQSLSSFFQKGSGSAASSSKEQGGAVAAVHSEQSERPGTSSSGTPTISDAAGGGGGGGGVFSRFRISPKSKEKSKSCFDLRNFGFGDKDMLVCNAASPAGATSASQKNHSQEYLNTTNNSRYRKQTNTAKPKPESFSSSSPQLYIHKPHHLAAAHPSALDDQTPPPIPPLPLNYQRSDDESYANETREHKKQRAISKASRQAELKRLRIAQEIQREQEEIEVQLKDLEARGVLIEKALRGEAQNIENLDATKDNDEKLLKELLEIWRNITALKKRDEELTIRQQELQLEYRHAQLKEELNLRLSCNKLDKSSADVAAEGAILNEMLEIVAKRAALRPTASQLDLTAAGSASTSAEATGIKLTGQPHDHEESII

SEQ ID NO: 9 - DNA, 9009 bp, Drosophila
atgagccgccaacaccagcggcaccaccagcagcatcaccacctgccgccgcaccagcaaccgcagcagcagatgccgcaacaacagcagcagctgacggcgcagcagcagcaacaacagcagctgctgatggcggagcacgcggcggccgcggaggcggcggagctattcgacctgctgtgcgtggccacaacgatgcgccagatcctggcgctccatcgggccatgtgcgaggctgtgggattgagaccctcgcctctgaacgacttctacccacggctaaaggccaaggtgcgttcgtggaaggcgcaggccctgtggaagaagttcgacgccagagctgcccatagagtctacggcaagggagctgcctgtactggcacacgcgtcctggtcatcggagcagggccctgtggactgcgcaccgccatcgaggcccaactgctgggcgccaaggtggtggtgctggagaaacgcgatcgcatcacccggaacaatgtgctccatctgtggccattcgtcatcacggatctgcgcaacttgggcgcaaagaagttctacggcaagttttgcgccggctccatcgatcacatctccattcggcagctgcagtgcatgctgctcaaggtggcgctgctcctgggcgtagagatccacgagggagtcagttttgatcacgctgtagagccctctggcgatggcggcggatggagggcagctgttactcccgcagatcatcctgtatctcactacgaattcgatgtgttgatcggagcggatggcaagcggaatatgctggactttaggaggaaggagttccgcgggaagctggccatcgctattacagcgaactttatcaacaagaagacggaggcggaggctaaagtagaggagatcagtggggtggctttcatcttcaaccaggccttcttcaaggagctgtacgggaagacgggcatcgacctggaaaacatcgtctactacaaggacgagacgcactacttcgtgatgacggccaagaagcacagtctaattgacaagggcgttattatcgaggatatggccgatcccggcgagcttctcgccccagccaatgtggatacacaaaagctgcacgactatgcacgcgaggctgcggagttctccacccaataccaaatgccaaacctggagttcgctgttaatcactacggcaaaccagatgtggccatgttcgacttcacatcgatgtttgccgccgagatgtcctgtcgggtgattgtgcgcaaaggagctcgcctgatgcagtgcctcgtgggtgacagtctgctcgagccgttttggcccactggatcgggttgtgcccgtggattcttatccagcatggatgctgcctatgccatcaagctttggtccaacccgcagaacagcacacttggcgttctggcgcagcgcgaaagcatctaccggctgcttaaccagaccacgccggacaccctgcagcgggacatcagtgcctataccgtggatccggccacgcgctatccgaatctgaacagggagtcggtcaatagctggcaggtcaaacatctggtcgacacggacgacccgtccattctggagcagaccttcatggacacgcatgctctgcagaccccgcatttggacacaccgggcagacgcaagcgacgcagtggagacttgctgccccagggtgccacgttgctgagatggataagtgcccagctgcattcctatcagtttattcccgaactcaaggaggcttcggatgtgttccggaatggacgcgttctgtgtgcgcttatcaatcgctatcgtcctgatctcatcgactacgctgccaccaaggacatgagtcccgtggagtgcaatgagctgtcattcgccgtcctagagcgcgaactccacatcgatcgcgtcatgagtgccaaacagtcgctggacttgaccgagctggagtcgcgaatctggctcaactatttggaccagatctgcgacttgtttcgcggcgagatcccccatatcaagcaccccaagatggactttagcgatttgcgccagaagtatcgtatcaaccatacgcatgcccaacccgacttctccaagctgctggcaacgaaacccaaggccaagtcgccgatgcaggatgctgtggacatacccacgacagtgcagcggcgctcggtgctcgaggaggagcgagccaagcggcagcgtcgccacgagcagcttcttaacatcggtggaggggcagcaggagccgccgccggagttgccggaagcgggacaggaaccacaacgcagggtcaaaacgatacgccacgccggtccaagaagcgccgtcaggttgacaaaaccgccaatattgaggagcgccagcagcgcttgcaggagatcgaggagaatcggcaggagcggatgagcaagcggcgccagcagcgctgtcaccagacgcagaatttctacaagagccttcagctcctgcaggcgggcaagctcttgagggagggtggtgaggcgggagtggccgaggatggcaccccattcgaggactactcgatattcctctaccgccagcaggcgcccgtattcaatgatcgcgtcaaggacctcgagcgaaagcttctgtttcccgatcgcgaacggggagatattccgtcggcattgccgcgcacggcggacgagcagttcagcgatcggattaaaaacatggagcagcggatgacgggacgtggtggccttggtggtgacaagaagcccaaggatctgatgcgggctatcggcaagatcgactcgaacgattggaatgtacgcgagatcgagaagaagatcgagctgtcgaagaagacggagatccacgggcctaagggccgcgagaaggttcccaagtggagcaaggagcagttccaggcgcgacagcacaagatgtccaagccgcaacgccaggattcgcgtgaggcggaaaagttcaaggacatcgaccagactatccgcaacttggacaagcagctaaaggagggccacaatctggatgtgggcgagcggggacgcaacaaagtggcctccattgccggtcagtttggcaaaaaggatgaggccaattcggatgagaagaacgcgggcagcagcaatgccaccaccaacaccaacaacacagtcatacccaaatctagttccaaggtggcgctggcctttaaaaagcaggctgcctccgaaaagtgccgcttctgtaagcaaaccgtctacctgatggagaagaccaccgtggagggattggttctgcatcgcaattgccttaagtgccaccactgccacaccaacttgcgtctgggaggctacgcctttgatcgggacgatccgcagggccgattttactgcacccaacacttccggttgccacccaaaccgctgccgcagcgcaccaacaaagccaggaaatccgctgccgctcaacccgcctcgcctgctgtaccaccaactgcgggatccgtacccactgcagctgccacatcggagcatatggacaccactccacccagggaccaggtggacctactggagacctcgcgagcaaatgcctctgccgatgccatgtccgatgatgaagccaatgttatcgatgagcacgaatggtctggtcgcaacttcttgcccgagtccaacaacgattcccaatcagagctatccagttcagatgagtcggatacggaatcggattcggagatgtttgaggaggcggatgattcgccgtttggtgctcagaccctccagctggcgtcggattggattggaaagcaatactgtgaggacagtgatgattctgacgatttctacgactcaagtgaaggtattgcggatgacggcaaagatgacaccgagggtgaggaattcaagaaggcccgcgaattgaggcgccaggaagttcgcctgcagccgttgcccgccaatctgcccacagatacggagaccgaggttcaaaccgagtccgagagcacttcacccgacgaagtggagctcaattctgccactgagatatccaccgactctgagtttgacaacgatgagattatacgccaggcgcccaaaatcttcatcgatgacacccatctaaggaagcccaccaaggttcagatcaagtccaccatgatcggacccaatgcagcttccgccggactccatcagaagcagttggcggcgcgtgagaagggcggcagctacctccagaagtaccaaccacaaccgccactgtcacagtttaaaccgttggtccaggtggatcccaccctgctcattggcagccagcgcgctcctcttcagaatccacggccaggagactacttgctaaacaagacggccagtacggagggtatcgcctcaaaaaagagcctggagctaaaaaagcgctatctgctgggtgagccggccaatggcgataagatccagaagtccggatccacttcagtgctggattcacgcattcgcagcttccagtcgaacatatcggagtgccagaagcttttgaatcccagcagcgacataagtgccggcatgcgaaccttcctcgatcgcacaaagttgggcgaaggcagccagacgacacccggacagacgaacgaactaatccgttccgccaccagcaatgtgattaacgatctgcgcgtggagcttcggatacagaaaactgactccagccactccacggacaacgagaaggaaaacgttttcgtgaactgtaagaacgagctgaacaaggggatggaatacacggatgcggtcaatgccacgctgctggaccagctggccagaaaaagttcacccaccacgccgacgaataagacggtggtcgaggttattgacctggttacacctgagaagccaattgacattatcgatctaacggcactggaaacgccgaaaaagcagttggtcgatggtagcgccatggatgtagatgaacgcctcacacccgatagcaacaaaatcagcgaactgcagcaggaagtgaaggaggaacccaagccggatgtctctagggatgtgaaagaatgcataccagatatactgggacacattaaggagggaacgggatcgaaggagccaggtggagaggaccaacagagcctgctggagcagtcggacgaagagaagcgcgactcaccggaaaaggatgtggccgaacatgagctttatgaaccggacagtgtgcagatccaggtgcccaatatcccatgggaaaaaagcaagccggaggtcatgtctaccaccggcagcagtggctccatctgctcaagctcagactcttctagtattgaagacatccagcactacattttggagtccacaactagtccagatactcagacagttggcggaaagcacaatgtgccccgtttggaggtgcacgacacaagtggtgccctgatgcaggtggacagcctgatgattgtgaacggaaagtatattggggatcccgaggatgtcaagttcttggatatgccggccaatgttattgttccgccagcaccggcgcttaaaacgaatgagctggatatggaggatgaccaagaggcggaggcggaaccagtaactgctactccggagccggtggaatgtacggtcatcgaggctgagcgccgtgttactgctccccctcctttgccagagatgggtccacccaaactgaagttcgatagcaaaaatgagaacaagatcgagagcttgaagaatcttccgttgatcgtagagagcaatgtggagcacagtcaggcagtgaaacccattactcttaacttaagcaatctggccaggacgccggatacaccaaccacgcccacggcgcacgatagcgataaaacacccactggggaaattctctcgcgaggatctgactcagaaaccgagcacactggcactggtcaggtactaacggagacggaactctccgactggacggccgacgactgtatctcggagaactttgttgacttggagttcgcgcttaactctaacaagggtacgataaaacggcgcaaggatcgacgacgcagtggagcaagcaaacttcccagtggcaacgaggtaatccacgagctggccaggcaggcgccagtggtgcaaatggatggaattcttagtgccatcgacattgatgacatagagttcatggacacgggttcggagggttcttgtgctgaagcttatcccgcaacaaatacagctctcattcagaatagaggttacatggagtacatcgaggcggagccgaaaaagacgacccgcaaggcagctccaccatccagttacccaggaaatttaccgcctttaatgacgaagcgggacgagaaactgggcgttgattacattgagcagggggcgtacataatgcacgatgatgcaaagacgcctgtgaatgaggtggctcctgccatgacccagtcgctaactgactcaatcacgctcaatgaactggatgatgacagtatgataatatcccaaacccagccaacgacaacggaggaaagtgaggcactgacggtggtcaccagtccacttgacacgtcctcgcccagggttctcgatcaatttgcatccatgttggcggcgggaaaaggtgactccacacccagtagctcagagcaacaaccaaagacgtctacggtgacgagcagcagcactgggcccaactcctcgacaacaggaaacgtctcgaaggagccgcaggaggaggacctgcaaatccagtttgagtatgttcgagcactgcagcagcggatatcgcagatcagcacccaacggcgtaagagctctaagggagaggcacctaacctgcagctaaacagtagcgcacctgtgatagaatcagccgaggatccggccaagcccgcagaggagcctctggtctcaatgcgaccgcggaccaccagcatttccggaaaggtaccggagatacccacacttagcagcaagctggaagagataaccaaagaacgcactaagcaaaaggatctgattcacgacctagtcatggacaagttgcagtcgaagaagcagctaaacgctgagaagcgtctgcaccggagtcgacagcgcagtttgctgaccagtggctatgccagtggctccagccttagtccgacgcccaagctggctgctgcttgcagtccgcaggattccaactgctctagccaagcgcactaccacgcctccacggcggaggaggccccgaagccgccggcggaaaggccgttgcagaagtccgccacgtccacctatgtgtcgccttatcgcactgtccaagcgcccacacgtagtgctgatctctataagccgcgccccttcagcgaacacatcgattcgaacgctctggcgggttacaagctcggcaagacggcctcgtttaatggcggcaagttgggcgactttgcgaaacccattgccccggcgagagttaaccgaggaggaggtgtcgcgaccgcggatatagccaatatttccgcgtcgacggagaacctaagaagcgaggccagggccagggctcgtcttaagtctaacacagagctgggccttagtcccgaggaaaagatgcagctaatacgttcaagattgcactacgaccaaaacagatctctgaagccgaagcaactggaggagatgccatccggggatctggcggcacgtgcccgcaaaatgagtgcctcgaagagcgtcaatgatctggcctacatggtgggacagcagcagcagcagcaggttgagaaggatgccgtgctccaagccaaggcggctgactttacatccgatcccaatttggcgtccggtggtcaggagaaggcaggcaaaactaagtccggacgcaggccaaaggatccggagcggcgtaagagtctcatacagtcgttgtccagcttcttccaaaagggatctggatccgcggcctccagttccaaggagcagggcggcgctgtggctgccgtccactctgaacagtcagagcgaccaggcaccagcagcagcggcacgcccacaatatcggatgcggcgggtggaggcggaggaggaggtggcgtcttcagcagattccgcatctcgcccaagtccaaggagaagtcaaagtcttgctttgatctgaggaatttcggttttggtgacaaggatatgctggtctgcaatgcagcatctccagcaggagccacatccgcatcacagaaaaatcactcgcaagagtatctgaacaccacgaacaacagtcgctatcgaaagcaaacgaacactgcgaaaccgaaacccgaatcgttctcttcatccagtccgcagctctatatacacaagccccaccacctggccgcagctcatcccagtgccctggacgaccagacaccaccacccataccgcctcttccactgaattatcagagatccgatgatgagagctacgctaacgagacacgagagcataagaagcaacgtgccatatcgaaggcttcacgacaagctgagctcaagcgattgcgaatcgctcaagagattcagcgggaacaggaggagatcgaggtgcaactgaaggatctggaggcacgcggcgtgcttattgagaaggccttgcgaggcgaggcgcagaatattgaaaacctggatgcgacaaaggacaacgacgagaagctacttaaggaacttttggagatttggcgcaacatcacagcactcaagaaacgcgatgaggaactgactataaggcaacaggaactgcaactggagtatcggcatgcccagctgaaggaagagctcaatctgcgcttgtcctgcaacaaactggacaaaagctctgccgatgtggccgccgagggagcaattctcaacgagatgctggaaattgtcgccaagcgagccgccctacgacccacagcctcccagctcgacctcacggcagcgggatcagcatccacgtccgccgaggcaacgggcattaagctgacgggacaaccgcatgaccacgaagaatcgatcatttga

SEQ ID NO: 10 - protein, 3002 aa, Drosophila
MSRQHQRHHQQHHHLPPHQQPQQQMPQQQQQLTAQQQQQQQLLMAEHAAAAEAAELFDLLCVATTMRQILALHRAMCEAVGLRPSPLNDFYPRLKAKVRSWKAQALWKKFDARAAHRVYGKGAACTGTRVLVIGAGPCGLRTAIEAQLLGAKVVVLEKRDRITRNNVLHLWPFVITDLRNLGAKKFYGKFCAGSIDHISIRQLQCMLLKVALLLGVEIHEGVSFDHAVEPSGDGGGWRAAVTPADHPVSHYEFDVLIGADGKRNMLDFRRKEFRGKLAIAITANFINKKTEAEAKVEEISGVAFIFNQAFFKELYGKTGIDLENIVYYKDETHYFVMTAKKHSLIDKGVIIEDMADPGELLAPANVDTQKLHDYAREAAEFSTQYQMPNLEFAVNHYGKPDVAMFDFTSMFAAEMSCRVIVRKGARLMQCLVGDSLLEPFWPTGSGCARGFLSSMDAAYAIKLWSNPQNSTLGVLAQRESIYRLLNQTTPDTLQRDISAYTVDPATRYPNLNRESVNSWQVKHLVDTDDPSILEQTFMDTHALQTPHLDTPGRRKRRSGDLLPQGATLLRWISAQLHSYQFIPELKEASDVFRNGRVLCALINRYRPDLIDYAATKDMSPVECNELSFAVLERELHIDRVMSAKQSLDLTELESRIWLNYLDQICDLFRGEIPHIKHPKMDFSDLRQKYRINHTHAQPDFSKLLATKPKAKSPMQDAVDIPTTVQRRSVLEEERAKRQRRHEQLLNIGGGAAGAAAGVAGSGTGTTTQGQNDTPRRSKKRRQVDKTANIEERQQRLQEIEENRQERMSKRRQQRCHQTQNFYKSLQLLQAGKLLREGGEAGVAEDGTPFEDYSIFLYRQQAPVFNDRVKDLERKLLFPDRERGDIPSALPRTADEQFSDRIKNMEQRMTGRGGLGGDKKPKDLMRAIGKIDSNDWNVREIEKKIELSKKTEIHGPKGREKVPKWSKEQFQARQHKMSKPQRQDSREAEKFKDIDQTIRNLDKQLKEGHNLDVGERGRNKVASIAGQFGKKDEANSDEKNAGSSNATTNTNNTVIPKSSSKVALAFKKQAASEKCRFCKQTVYLMEKTTVEGLVLHRNCLKCHHCHTNLRLGGYAFDRDDPQGRFYCTQHFRLPPKPLPQRTNKARKSAAAQPASPAVPPTAGSVPTAAATSEHMDTTPPRDQVDLLQTSRANASADAMSDDEANVIDEHEWSGRNFLPESNNDSQSELSSSDESDTESDSEMFEEADDSPFGAQTLQLASDWIGKQYCEDSDDSDDFYDSSEGIADDGKDDTEGEEFKKARELRRQEVRLQPLPANLPTDTETEVQTESESTSPDEVELNSATEISTDSEFDNDEIIRQAPKIFIDDTHLRKPTKVQIKSTMIGPNAASAGLHQKQLAAREKGGSYLQKYQPQPPLSQFKPLVQVDPTLLIGSQRAPLQNPRPGDYLLNKTASTEGIASKKSLELKKRYLLGEPANGDKIQKSGSTSVLDSRIRSFQSNISECQKLLNPSSDISAGMRTFLDRTKLGEGSQTTPGQTNELIRSATSNVINDLRVELRIQKTDSSHSTDNEKENVFVNCKNELNKGMEYTDAVNATLLDQLARKSSPTTPTNKTVVEVIDLVTPEKPIDIIDLTALETPKKQLVDGSAMDVDERLTPDSNKISELQQEVKEEPKPDVSRDVKECIPDILGHIKEGTGSKEPGGEDQQSLLEQSDEEKRDSPEKDVAEHELYEPDSVQIQVPNIPWEKSKPEVMSTTGSSGSICSSSDSSSIEDIQHYILESTTSPDTQTVGGKHNVPRLEVHDTSGALMQVDSLMIVNGKYIGDPEDVKFLDMPANVIVPPAPALKTNELDMEDDQEAEAEPVTATPEPVECTVIEAERRVTAPPPLPEMGPPKLKFDSKNENKIESLKNLPLIVESNVEHSQAVKPITLNLSNLARTPDTPTTPTAHDSDKTPTGEILSRGSDSETEHTGTGQVLTETELSDWTADDCISENFVDLEFALNSNKGTIKRRKDRRRSGASKLPSGNEVIHELARQAPVVQMDGILSAIDIDDIEFMDTGSEGSCAEAYPATNTALIQNRGYMEYIEAEPKKTTRKAAPPSSYPGNLPPLMTKRDEKLGVDYIEQGAYIMHDDAKTPVNEVAPAMTQSLTDSITLNELDDDSMIISQTQPTTTEESEALTVVTSPLDTSSPRVLDQFASMLAAGKGDSTPSSSEQQPKTSTVTSSSTGPNSSTTGNVSKEPQEEDLQIQFEYVRALQQRISQISTQRRKSSKGEAPNLQLNSSAPVIESAEDPAKPAEEPLVSMRPRTTSISGKVPEIPTLSSKLEEITKERTKQKDLIHDLVMDKLQSKKQLNAEKRLHRSRQRSLLTSGYASGSSLSPTPKLAAACSPQDSNCSSQAHYHASTAEEAPKPPAERPLQKSATSTYVSPYRTVQAPTRSADLYKPRPFSEHIDSNALAGYKLGKTASFNGGKLGDFAKPIAPARVNRGGGVATADIANISASTENLRSEARARARLKSNTELGLSPEEKMQLIRSRLHYDQNRSLKPKQLEEMPSGDLAARARKMSASKSVNDLAYMVGQQQQQQVEKDAVLQAKAADFTSDPNLASGGQEKAGKTKSGRRPKDPERRKSLIQSLSSFFQKGSGSAASSSKEQGGAVAAVHSEQSERPGTSSSGTPTISDAAGGGGGGGGVFSRFRISPKSKEKSKSCFDLRNFGFGDKDMLVCNAASPAGATSASQKNHSQEYLNTTNNSRYRKQTNTAKPKPESFSSSSPQLYIHKPHHLAAAHPSALDDQTPPPIPPLPLNYQRSDDESYANETREHKKQRAISKASRQAELKRLRIAQEIQREQEEIEVQLKDLEARGVLIEKALRGEAQNIENLDATKDNDEKLLKELLEIWRNITALKKRDEELTIRQQELQLEYRHAQLKEELNLRLSCNKLDKSSADVAAEGAILNEMLEIVAKRAALRPTASQLDLTAAGSASTSAEATGIKLTGQPHDHEESII

SEQ ID NO: 11 - DNA, 8205 bp, Drosophila
atgagccgccaacaccagcggcaccaccagcagcatcaccacctgccgccgcaccagcaaccgcagcagcagatgccgcaacaacagcagcagctgacggcgcagcagcagcaacaacagcagctgctgatggcggagcacgcggcggccgcggaggcggcggagctattcgacctgctgtgcgtggccacaacgatgcgccagatcctggcgctccatcgggccatgtgcgaggctgtgggattgagaccctcgcctctgaacgacttctacccacggctaaaggccaaggtgcgttcgtggaaggcgcaggccctgtggaagaagttcgacgccagagctgcccatagagtctacggcaagggagctgcctgtactggcacacgcgtcctggtcatcggagcagggccctgtggactgcgcaccgccatcgaggcccaactgctgggcgccaaggtggtggtgctggagaaacgcgatcgcatcacccggaacaatgtgctccatctgtggccattcgtcatcacggatctgcgcaacttgggcgcaaagaagttctacggcaagttttgcgccggctccatcgatcacatctccattcggcagctgcagtgcatgctgctcaaggtggcgctgctcctgggcgtagagatccacgagggagtcagttttgatcacgctgtagagccctctggcgatggcggcggatggagggcagctgttactcccgcagatcatcctgtatctcactacgaattcgatgtgttgatcggagcggatggcaagcggaatatgctggactttaggaggaaggagttccgcgggaagctggccatcgctattacagcgaactttatcaacaagaagacggaggcggaggctaaagtagaggagatcagtggggtggctttcatcttcaaccaggccttcttcaaggagctgtacgggaagacgggcatcgacctggaaaacatcgtctactacaaggacgagacgcactacttcgtgatgacggccaagaagcacagtctaattgacaagggcgttattatcgaggatatggccgatcccggcgagcttctcgccccagccaatgtggatacacaaaagctgcacgactatgcacgcgaggctgcggagttctccacccaataccaaatgccaaacctggagttcgctgttaatcactacggcaaaccagatgtggccatgttcgacttcacatcgatgtttgccgccgagatgtcctgtcgggtgattgtgcgcaaaggagctcgcctgatgcagtgcctcgtgggtgacagtctgctcgagccgttttggcccactggatcgggttgtgcccgtggattcttatccagcatggatgctgcctatgccatcaagctttggtccaacccgcagaacagcacacttggcgttctggcgcagcgcgaaagcatctaccggctgcttaaccagaccacgccggacaccctgcagcgggacatcagtgcctataccgtggatccggccacgcgctatccgaatctgaacagggagtcggtcaatagctggcaggtcaaacatctggtcgacacggacgacccgtccattctggagcagaccttcatggacacgcatgctctgcagaccccgcatttggacacaccgggcagacgcaagcgacgcagtggagacttgctgccccagggtgccacgttgctgagatggataagtgcccagctgcattcctatcagtttattcccgaactcaaggaggcttcggatgtgttccggaatggacgcgttctgtgtgcgcttatcaatcgctatcgtcctgatctcatcgactacgctgccaccaaggacatgagtcccgtggagtgcaatgagctgtcattcgccgtcctagagcgcgaactccacatcgatcgcgtcatgagtgccaaacagtcgctggacttgaccgagctggagtcgcgaatctggctcaactatttggaccagatctgcgacttgtttcgcggcgagatcccccatatcaagcaccccaagatggactttagcgatttgcgccagaagtatcgtatcaaccatacgcatgcccaacccgacttctccaagctgctggcaacgaaacccaaggccaagtcgccgatgcaggatgctgtggacatacccacgacagtgcagcggcgctcggtgctcgaggaggagcgagccaagcggcagcgtcgccacgagcagcttcttaacatcggtggaggggcagcaggagccgccgccggagttgccggaagcgggacaggaaccacaacgcagggtcaaaacgatacgccacgccggtccaagaagcgccgtcaggttgacaaaaccgccaatattagttccaaggtggcgctggcctttaaaaagcaggctgcctccgaaaagtgccgcttctgtaagcaaaccgtctacctgatggagaagaccaccgtggagggattggttctgcatcgcaattgccttaagtgccaccactgccacaccaacttgcgtctgggaggctacgcctttgatcgggacgatccgcagggccgattttactgcacccaacacttccggttgccacccaaaccgctgccgcagcgcaccaacaaagccaggaaatccgctgccgctcaacccgcctcgcctgctgtaccaccaactgcgggatccgtacccactgcagctgccacatcggagcatatggacaccactccacccagggaccaggtggacctactggagacctcgcgagcaaatgcctctgccgatgccatgtccgatgatgaagccaatgttatcgatgagcacgaatggtctggtcgcaacttcttgcccgagtccaacaacgattcccaatcagagctatccagttcagatgagtcggatacggaatcggattcggagatgtttgaggaggcggatgattcgccgtttggtgctcagaccctccagctggcgtcggattggattggaaagcaatactgtgaggacagtgatgattctgacgatttctacgactcaagtgaaggtattgcggatgacggcaaagatgacaccgagggtgaggaattcaagaaggcccgcgaattgaggcgccaggaagttcgcctgcagccgttgcccgccaatctgcccacagatacggagaccgaggttcaaaccgagtccgagagcacttcacccgacgaagtggagctcaattctgccactgagatatccaccgactctgagtttgacaacgatgagattatacgccaggcgcccaaaatcttcatcgatgacacccatctaaggaagcccaccaaggttcagatcaagtccaccatgatcggacccaatgcagcttccgccggactccatcagaagcagttggcggcgcgtgagaagggcggcagctacctccagaagtaccaaccacaaccgccactgtcacagtttaaaccgttggtccaggtggatcccaccctgctcattggcagccagcgcgctcctcttcagaatccacggccaggagactacttgctaaacaagacggccagtacggagggtatcgcctcaaaaaagagcctggagctaaaaaagcgctatctgctgggtgagccggccaatggcgataagatccagaagtccggatccacttcagtgctggattcacgcattcgcagcttccagtcgaacatatcggagtgccagaagcttttgaatcccagcagcgacataagtgccggcatgcgaaccttcctcgatcgcacaaagttgggcgaaggcagccagacgacacccggacagacgaacgaactaatccgttccgccaccagcaatgtgattaacgatctgcgcgtggagcttcggatacagaaaactgactccagccactccacggacaacgagaaggaaaacgttttcgtgaactgtaagaacgagctgaacaaggggatggaatacacggatgcggtcaatgccacgctgctggaccagctggccagaaaaagttcacccaccacgccgacgaataagacggtggtcgaggttattgacctggttacacctgagaagccaattgacattatcgatctaacggcactggaaacgccgaaaaagcagttggtcgatggtagcgccatggatgtagatgaacgcctcacacccgatagcaacaaaatcagcgaactgcagcaggaagtgaaggaggaacccaagccggatgtctctagggatgtgaaagaatgcataccagatatactgggacacattaaggagggaacgggatcgaaggagccaggtggagaggaccaacagagcctgctggagcagtcggacgaagagaagcgcgactcaccggaaaaggatgtggccgaacatgagctttatgaaccggacagtgtgcagatccaggtgcccaatatcccatgggaaaaaagcaagccggaggtcatgtctaccaccggcagcagtggctccatctgctcaagctcagactcttctagtattgaagacatccagcactacattttggagtccacaactagtccagatactcagacagttggcggaaagcacaatgtgccccgtttggaggtgcacgacacaagtggtgccctgatgcaggtggacagcctgatgattgtgaacggaaagtatattggggatcccgaggatgtcaagttcttggatatgccggccaatgttattgttccgccagcaccggcgcttaaaacgaatgagctggatatggaggatgaccaagaggcggaggcggaaccagtaactgctactccggagccggtggaatgtacggtcatcgaggctgagcgccgtgttactgctccccctcctttgccagagatgggtccacccaaactgaagttcgatagcaaaaatgagaacaagatcgagagcttgaagaatcttccgttgatcgtagagagcaatgtggagcacagtcaggcagtgaaacccattactcttaacttaagcaatctggccaggacgccggatacaccaaccacgcccacggcgcacgatagcgataaaacacccactggggaaattctctcgcgaggatctgactcagaaaccgagcacactggcactggtcaggtactaacggagacggaactctccgactggacggccgacgactgtatctcggagaactttgttgacttggagttcgcgcttaactctaacaagggtacgataaaacggcgcaaggatcgacgacgcagtggagcaagcaaacttcccagtggcaacgaggtaatccacgagctggccaggcaggcgccagtggtgcaaatggatggaattcttagtgccatcgacattgatgacatagagttcatggacacgggttcggagggttcttgtgctgaagcttatcccgcaacaaatacagctctcattcagaatagaggttacatggagtacatcgaggcggagccgaaaaagacgacccgcaaggcagctccaccatccagttacccaggaaatttaccgcctttaatgacgaagcgggacgagaaactgggcgttgattacattgagcagggggcgtacataatgcacgatgatgcaaagacgcctgtgaatgaggtggctcctgccatgacccagtcgctaactgactcaatcacgctcaatgaactggatgatgacagtatgataatatcccaaacccagccaacgacaacggaggaaagtgaggcactgacggtggtcaccagtccacttgacacgtcctcgcccagggttctcgatcaatttgcatccatgttggcggcgggaaaaggtgactccacacccagtagctcagagcaacaaccaaagacgtctacggtgacgagcagcagcactgggcccaactcctcgacaacaggaaacgtctcgaaggagccgcaggaggaggacctgcaaatccagtttgagtatgttcgagcactgcagcagcggatatcgcagatcagcacccaacggcgtaagagctctaagggagaggcacctaacctgcagctaaacagtagcgcacctgtgatagaatcagccgaggatccggccaagcccgcagaggagcctctggtctcaatgcgaccgcggaccaccagcatttccggaaaggtaccggagatacccacacttagcagcaagctggaagagataaccaaagaacgcactaagcaaaaggatctgattcacgacctagtcatggacaagttgcagtcgaagaagcagctaaacgctgagaagcgtctgcaccggagtcgacagcgcagtttgctgaccagtggctatgccagtggctccagccttagtccgacgcccaagctggctgctgcttgcagtccgcaggattccaactgctctagccaagcgcactaccacgcctccacggcggaggaggccccgaagccgccggcggaaaggccgttgcagaagtccgccacgtccacctatgtgtcgccttatcgcactgtccaagcgcccacacgtagtgctgatctctataagccgcgccccttcagcgaacacatcgattcgaacgctctggcgggttacaagctcggcaagacggcctcgtttaatggcggcaagttgggcgactttgcgaaacccattgccccggcgagagttaaccgaggaggaggtgtcgcgaccgcggatatagccaatatttccgcgtcgacggagaacctaagaagcgaggccagggccagggctcgtcttaagtctaacacagagctgggccttagtcccgaggaaaagatgcagctaatacgttcaagattgcactacgaccaaaacagatctctgaagccgaagcaactggaggagatgccatccggggatctggcggcacgtgcccgcaaaatgagtgcctcgaagagcgtcaatgatctggcctacatggtgggacagcagcagcagcagcaggttgagaaggatgccgtgctccaagccaaggcggctgactttacatccgatcccaatttggcgtccggtggtcaggagaaggcaggcaaaactaagtccggacgcaggccaaaggatccggagcggcgtaagagtctcatacagtcgttgtccagcttcttccaaaagggatctggatccgcggcctccagttccaaggagcagggcggcgctgtggctgccgtccactctgaacagtcagagcgaccaggcaccagcagcagcggcacgcccacaatatcggatgcggcgggtggaggcggaggaggaggtggcgtcttcagcagattccgcatctcgcccaagtccaaggagaagtcaaagtcttgctttgatctgaggaatttcggttttggtgacaaggatatgctggtctgcaatgcagcatctccagcaggagccacatccgcatcacagaaaaatcactcgcaagagtatctgaacaccacgaacaacagtcgctatcgaaagcaaacgaacactgcgaaaccgaaacccgaatcgttctcttcatccagtccgcagctctatatacacaagccccaccacctggccgcagctcatcccagtgccctggacgaccagacaccaccacccataccgcctcttccactgaattatcagagatccgatgatgagagctacgctaacgagacacgagagcataagaagcaacgtgccatatcgaaggcttcacgacaagctgagctcaagcgattgcgaatcgctcaagagattcagcgggaacaggaggagatcgaggtgcaactgaaggatctggaggcacgcggcgtgcttattgagaaggccttgcgaggcgaggcgcagaatattgaaaacctggatgcgacaaaggacaacgacgagaagctacttaaggaacttttggagatttggcgcaacatcacagcactcaagaaacgcgatgaggaactgactataaggcaacaggaactgcaactggagtatcggcatgcccagctgaaggaagagctcaatctgcgcttgtcctgcaacaaactggacaaaagctctgccgatgtggccgccgagggagcaattctcaacgagatgctggaaattgtcgccaagcgagccgccctacgacccacagcctcccagctcgacctcacggcagcgggatcagcatccacgtccgccgaggcaacgggcattaagctgacgggacaaccgcatgaccacgaagaatcgatcatttga

SEQ ID NO: 12 - protein, 2734 aa, Drosophila
MSRQHQRHHQQHHHLPPHQQPQQQMPQQQQQLTAQQQQQQQLLMAEHAAAAEAAELFDLLCVATTMRQILALHRAMCEAVGLRPSPLNDFYPRLKAKVRSWKAQALWKKFDARAAHRVYGKGAACTGTRVLVIGAGPCGLRTAIEAQLLGAKVVVLEKRDRITRNNVLHLWPFVITDLRNLGAKKFYGKFCAGSIDHISIRQLQCMLLKVALLLGVEIHEGVSFDHAVEPSGDGGGWRAAVTPADHPVSHYEFDVLIGADGKRNMLDFRRKEFRGKLAIAITANFINKKTEAEAKVEEISGVAFIFNQAFFKELYGKTGIDLENIVYYKDETHYFVMTAKKHSLIDKGVIIEDMADPGELLAPANVDTQKLHDYAREAAEFSTQYQMPNLEFAVNHYGKPDVAMFDFTSMFAAEMSCRVIVRKGARLMQCLVGDSLLEPFWPTGSGCARGFLSSMDAAYAIKLWSNPQNSTLGVLAQRESIYRLLNQTTPDTLQRDISAYTVDPATRYPNLNRESVNSWQVKHLVDTDDPSILEQTFMDTHALQTPHLDTPGRRKRRSGDLLPQGATLLRWISAQLHSYQFIPELKEASDVFRNGRVLCALINRYRPDLIDYAATKDMSPVECNELSFAVLERELHIDRVMSAKQSLDLTELESRIWLNYLDQICDLFRGEIPHIKHPKMDFSDLRQKYRINHTHAQPDFSKLLATKPKAKSPMQDAVDIPTTVQRRSVLEEERAKRQRRHEQLLNIGGGAAGAAAGVAGSGTGTTTQGQNDTPRRSKKRRQVDKTANISSKVALAFKKQAASEKCRFCKQTVYLMEKTTVEGLVLHRNCLKCHHCHTNLRLGGYAFDRDDPQGRFYCTQHFRLPPKPLPQRTNKARKSAAAQPASPAVPPTAGSVPTAAATSEHMDTTPPRDQVDLLQTSRANASADAMSDDEANVIDEHEWSGRNFLPESNNDSQSELSSSDESDTESDSEMFEEADDSPFGAQTLQLASDWIGKQYCEDSDDSDDFYDSSEGIADDGKDDTEGEEFKKARELRRQEVRLQPLPANLPTDTETEVQTESESTSPDEVELNSATEISTDSEFDNDEIIRQAPKIFIDDTHLRKPTKVQIKSTMIGPNAASAGLHQKQLAAREKGGSYLQKYQPQPPLSQFKPLVQVDPTLLIGSQRAPLQNPRPGDYLLNKTASTEGIASKKSLELKKRYLLGEPANGDKIQKSGSTSVLDSRIRSFQSNISECQKLLNPSSDISAGMRTFLDRTKLGEGSQTTPGQTNELIRSATSNVINDLRVELRIQKTDSSHSTDNEKENVFVNCKNELNKGMEYTDAVNATLLDQLARKSSPTTPTNKTVVEVIDLVTPEKPIDIIDLTALETPKKQLVDGSAMDVDERLTPDSNKISELQQEVKEEPKPDVSRDVKECIPDILGHIKEGTGSKEPGGEDQQSLLEQSDEEKRDSPEKDVAEHELYEPDSVQIQVPNIPWEKSKPEVMSTTGSSGSICSSSDSSSIEDIQHYILESTTSPDTQTVGGKHNVPRLEVHDTSGALMQVDSLMIVNGKYIGDPEDVKFLDMPANVIVPPAPALKTNELDMEDDQEAEAEPVTATPEPVECTVIEAERRVTAPPPLPEMGPPKLKFDSKNENKIESLKNLPLIVESNVEHSQAVKPITLNLSNLARTPDTPTTPTAHDSDKTPTGEILSRGSDSETEHTGTGQVLTETELSDWTADDCISENFVDLEFALNSNKGTIKRRKDRRRSGASKLPSGNEVIHELARQAPVVQMDGILSAIDIDDIEFMDTGSEGSCAEAYPATNTALIQNRGYMEYIEAEPKKTTRKAAPPSSYPGNLPPLMTKRDEKLGVDYIEQGAYIMHDDAKTPVNEVAPAMTQSLTDSITLNELDDDSMIISQTQPTTTEESEALTVVTSPLDTSSPRVLDQFASMLAAGKGDSTPSSSEQQPKTSTVTSSSTGPNSSTTGNVSKEPQEEDLQIQFEYVRALQQRISQISTQRRKSSKGEAPNLQLNSSAPVIESAEDPAKPAEEPLVSMRPRTTSISGKVPEIPTLSSKLEEITKERTKQKDLIHDLVMDKLQSKKQLNAEKRLHRSRQRSLLTSGYASGSSLSPTPKLAAACSPQDSNCSSQAHYHASTAEEAPKPPAERPLQKSATSTYVSPYRTVQAPTRSADLYKPRPFSEHIDSNALAGYKLGKTASFNGGKLGDFAKPIAPARVNRGGGVATADIANISASTENLRSEARARARLKSNTELGLSPEEKMQLIRSRLHYDQNRSLKPKQLEEMPSGDLAARARKMSASKSVNDLAYMVGQQQQQQVEKDAVLQAKAADFTSDPNLASGGQEKAGKTKSGRRPKDPERRKSLIQSLSSFFQKGSGSAASSSKEQGGAVAAVHSEQSERPGTSSSGTPTISDAAGGGGGGGGVFSRFRISPKSKEKSKSCFDLRNFGFGDKDMLVCNAASPAGATSASQKNHSQEYLNTTNNSRYRKQTNTAKPKPESFSSSSPQLYIHKPHHLAAAHPSALDDQTPPPIPPLPLNYQRSDDESYANETREHKKQRAISKASRQAELKRLRIAQEIQREQEEIEVQLKDLEARGVLIEKALRGEAQNIENLDATKDNDEKLLKELLEIWRNITALKKRDEELTIRQQELQLEYRHAQLKEELNLRLSCNKLDKSSADVAAEGAILNEMLEIVAKRAALRPTASQLDLTAAGSASTSAEATGIKLTGQPHDHEESII

SEQ ID NO: 13 - DNA, 5411 bp, Homo sapiens
ggccaagccggggccccgaagccagagccggagccgggcgggccgcggggtcatggctgggccgcggggcgcgctgctggcctggtgccgccgccagtgcgagggctaccgcggcgtggagatccgcgacctgagcagctccttccgggacggcctggccttctgcgccatcctgcaccggcaccggcccgacctgctagattttgattcgctttccaaggacaatgtcttcgagaataaccgtttggcctttgaagtggctgagaaggagctggggatccccgctctcctggaccccaatgacatggtctccatgagcgtccctgactgcctcagcatcatgacctatgtgtcccagtattacaaccacttctgcagtcctggccaagctggtgtctcgccacccagaaagggccttgcaccctgttccccgccgtctgtagcacccactccagtggaaccagaagatgtggctcagggcgaggagctctcctcaggcagcctgtcagagcagggcaccggccagacccccagcagcacgtgcgcagcctgccagcagcatgtgcacttggtgcagcgctacctggctgacggcaggctgtaccatcgccactgcttccggtgtcggcggtgctccagcaccctgctccctggggcttatgagaatgggcctgaggagggcacctttgtgtgtgcagaacactgtgccaggctgggcccggggacacggtcggggaccaggcctgggcccttctcacagccaaagcagcagcaccagcagcaactcgcagaagatgccaaggatgttccaggaggcggccccagctccagtgctcctgcaggggctgaggccgatggacccaaggccagccctgaggcccggccgcagatccctaccaagccccgggttcctggcaaactacaggagctggccagcccccctgcgggccgccccacccctgcccccaggaaggcctctgagagcaccaccccagcaccccccacgccccggccccgctccagtctgcagcaggagaacctggtggagcaggctggcagcagcagcctggtgaacgggagactgcacgaactgcctgtccccaagccgagggggacaccgaagccgtccgaggggacaccagcccccaggaaggaccccccatggatcacgctggtgcaggcagaaccaaagaagaagccagccccacttcccccaagcagcagcccggggccaccaagccaggacagcaggcaggtggagaatggaggcaccgaggaggtggcccagccgagcccaacggccagcctggagtccaaaccctataacccctttgaggaggaggaggaggacaaggaggaagaggctccagctgcacccagcctggccaccagccctgccctgggccacccggagtccacacccaagtccctgcacccctggtacggcatcacccctaccagcagccccaagacaaagaagcgccctgccccgcgcgcacccagcgcgtccccactggctctccacgcctcccgcctctcgcactcggagccgccctcggccacaccatcgccagcgctcagcgtggagagcctgtcgtctgagagcgccagccagactgcaggtgcagagcttctggagccgccagctgtgcccaagagctcctcagagcctgctgtccatgcccctggtacccctggaaaccctgtcagcctctctaccaactcctccctggcctcctctggggaactagtggagcctagagtggaacaaatgcctcaagccagccctggccttgcccccaggaccaggggcagctcaggtccccagccagccaagccctgcagtggcgccaccccaacgcctctcttgttggttggagacaggagcccggtgccttcccctggaagctcgtccccacagctgcaggtaaagtcctcctgcaaggagaatccttttaaccggaagccatcacctgcagcgtccccagccacaaagaaggccaccaagggatccaagccagtgaggccacctgcccctggacacggctttccactcatcaaacgcaaggtccaggctgaccagtacatccctgaggaggacatccatggagagatggataccattgagcgccggctggatgccctggagcaccgtggggtgctgctggaggagaagctgcgtggcggcctgaatgagggccgtgaggatgacatgctggtggactggttcaagctcatccacgagaagcacctactggtgcggcgagagtccgagctcatctatgtcttcaagcagcagaacctggagcagcgccaggctgatgtcgagtatgagctccggtgcctcctcaataagccagaaaaggactggacggaggaggaccgggcccgggagaaggtgctgatgcaggagcttgtgaccctcattgagcagcgcaacgctatcatcaactgcctggatgaggaccggcagagggaggaagaggaagacaagatgttggaagccatgatcaagaagaaagagttccagagggaggctgaacctgagggcaagaagaaggggaagttcaagaccatgaagatgttgaaactgctaggaaacaaacgtgatgccaagagcaagtcccccagagacaagagctaacagcacgagaagccagttggggactgccccctcctggagcagctcctgggctgtgctctgtttgaagggggcgccctgctcccctcagatcagtcaggaggaagatgactaaggggagggatcctctgggtgatggcctcttcctcctcagggacctctgactgctctgggccaaagaatctcttgtttcttctccgagccccaggcagcggtgattcagccctgcccaacctgattctgatgactgcggatgctgtgacggacccaaggggcaaatagggtcccagggtccagggaggggcgcctgctgagcacttccgcccctcaccctgcccagcccctgccatgagctctgggctgggtctccgcctccagggttctgctcttccaggcaggccagcaagtggcgctgggccacactggcttcttcctgccccatccctggctctgagtctctgtcttcctgtcctgtgcaggcgcccttggatctcagtttccctcactcaggaactctgtttctgaagtcttcagttaagtttgagtttatgactgagtggcctgtactgtcagacgtgaatgggcctgacgggcaaatccatccctctctccctcacagttccaggagcggcttccctcgtctccccttactccacagggagcctcccttgccaggaccagggctgcgacggccatgctggggcaggtgagtgctctgttagctgctcccagtgctgtccccaggctgcagttctggtccctggttgtcaggtaggaagggtgcacttgaagcaggtgctcatctcggttccttaacgtttatagtctgacccctcacttaggctttcctctgccaccccggtccagggaagaggctcgctcccgcccatggtcatcactggtctgtctgctctgttgtctgttctttccctgactccctcccaccgaaggcctgatggctactcacccctctgggatggctatgggagaggaggagtgatggggaccgccaccttttctgcaggaaatgtgcccagcagctcttggtcaaagcactgttgctataagctatctctgggatgcctctaggcccccttccctctacacacctctgggaaaagattacactgtattaactctcgaggagtttcctcaccaataaacagacaacctcaactgccagtgccctgcagcctcgggccacagcggcagccttgtttgccttcccacctgcctctgccacacctggtggctgaacatctctggtcgcccagaggccatgttggggccatcctccaagagggatctctgccctcaccgcctgccactgggcaggatccctttcctctgcagggagaggtggctcctcggccatgcagcccctggcaggctccttctaaacatgcctgttgacctggagctggcgccaccaactccagggcctttccagggccagacaggtaacacgcatgaacccgagtgacagctctgacgggctgtttcggtgtcaggagacaaagctggcaggggcaggggtgaactggaggcaagtcaagtcacctgtggcctgtggggctgaatgtgggcccggtgttgccagatcctttgtcataagaagctagaaatccagattttatgtgtgtgtaatttgtaaatgctgaaagctagcctgaattttttttttttttttttgagacagagtctcgctctgtcgcccaggctggagtgcagtggcgcgatctcagctcactgcaagctccgcctcctgggttcacgccatcctcctgcctcggcctcctgagcagctgggactacaggcgcatgctacgacgcctggctaattttttgtatttttagtagagacggggtttcaccgtgttaaccaggatggtctcgatctcctgaccttgtgatccacccaccttggcctcccaaagtgctgggattacaggcgtgagccaccacgcccggccactagcctgaatttcaatcaagggttggctgatactgtgtgtccagggtggactggatttgtcctggggggttctctggtttgctgcctcctgaccacatgatggggccttcgaggtcgaggacaactgttcccattagattgcaccctctgccctcaggttcttgagggtgtgtggacacagaggctttccatgggatgtccctgagccggcccttgattggggcctcaccatttacagggccgttttattctgcaaaccgaaacttgggtcatgtgacctgatgggattatgggactccctccaggtgcccgagacaaggttgatatttccaaaatattttggtgatttagtgggacaagcaaatgacagaataccggagaaggcagggatcgtgggtgtcaggagccagaggggagggggacagatgtgctgtgtacaggacaaggtgtcaggtgactccttcccagcagggcctcgcagatgcacaagcacggagctggtgggttttgcccaagaaaggtcacgcggcacatgcagggattggaactcccaggccagggctctaggtcgctcccaccttttcatgtttctttctgtggccatgggtatagtggaaagacataaagctaaagccaacttttaatcctgaatgcactgcttgccaggtaaatgcccttggttgtggtatcttgttgagacttagttttcacagagggataatgaaccgttgcagaggtttattgagatcattaacagagtggaattcagcacccgccacagcagccagccatggg

SEQ ID NO: 14 - protein, 863 aa, Homo sapiens; identical to SEQ ID NO: 2

SEQ ID NO: 15 - DNA, 2685 bp, Homo sapiens
atggcggccatcagggcgctgcaacagtggtgccggcagcagtgcgagggctaccgcgacgtgaatatctgcaacatgaccacgtcgttccgcgacggcctggctttctgcgccatcctgcaccgccaccggcccgacctcataaacttcagtgctctcaagaaggaaaatatttatgaaaacaataaactggccttccgcgtggccgaggagcacttgggcatcccagccttgctggatgccgaggacatggtggccttgaaggtgcctgaccggctgagcatcttgacctacgtgtcccagtattacaactacttccacggccgctcccccattgggggcatggcaggcgtgaagagggcctcggaggactctgaggaggagccgtcagggaagaaggctccagtccaggcggccaagctgccctcgcccgccccagcccggaagcctccactatctccagcccagacaaaccctgtggtccagaggaggaatgagggtgcagggggcccgccccccaagactgaccaggcattggcgggcagcttggtcagcagcacctgcggggtctgcggcaagcacgtgcacctggtacagcggcacctggccgacgggaggctttaccaccggagctgcttcaggtgtaagcagtgctcctgcacgctgcactcgggggcctacaaggccacaggagagccgggcaccttcgtctgcaccagccacctccccgcagccgcctctgcaagccccaagttgacgggtctggtcccccgacagccaggggccatgggtgtggattccaggacctcctgttccccacagaaggcccaggaggcaaacaaggccagaccgttggcctgggagcctgctgcgggcaactcgcctgccagggcttccgttccagctgcacccaaccctgcagccaccagcgccacgtccgtccacgtgaggagcccagccaggccctctgagagccgcctggcccccactcccacggaggggaaagtccgccctcgtgtgaccaatagctccccgatgggctggtcgtcagctgccccgtgcacagcagcggctgcctcccatcccgccgtgcccccgagtgccccagaccctcgcccggccacaccccagggcgggggagccccccgagtggcagctcctcaaaccacactcagttcaagctccacatctgcagccacggtggaccccccagcctggaccccgtccgcctccaggacccagcaggcccggaataagtttttccaaacatcagcagtgccccccggcaccagcctttctggcagaggtcccaccccgtcacttgttctatccaaggacagcagcaaggagcaggcgcggaacttcctcaagcaggccctctcagcgctggaagaggctggcgctccggcgcctggcaggccctccccagccactgccgctgttcccagttctcagcccaaaactgaagcaccacaagcaagtcccttagccaagccgttacagtcctcgtctccccgggtgcttggcctcccttcgaggatggaaccgccagccccgctgagcacgagcagtacctctcaggcatccgcgttgcccccggcaggcaggaggaacttggctgaatcctcaggggtcggcagggtgggtgctggctccaggccgaagccagaggccccgatggcaaagggtaaaagcaccaccttaacgcaggacatgagcaccagcctccaggaaggccaggaggacgggccggcaggatggagagcgaatctgaagcccgtggacaggaggagcccagctgagaggactctgaagcccaaggaaccacgggccctggcagagccgagggcgggggaggcccccaggaaggtctcaggcagctttgctgggagtgtccacatcaccctgacccccgtgaggcctgacaggaccccacgcccagccagcccaggacccagcctcccagccaggtccccctccccaccccgccgcaggagactggccgtccctgccagcctcgacgtttgtgacaactggcttcggccggagccccctggccaggaagcccgagtgcagagctggaaggaggaggagaagaaacctcaccttcagggcagaccagggagacccttgtccccggccaatgtccctgctctgcctggcgagacggtgacctccccagtcaggctgcaccccgactacctctccccggaggagatacagaggcagctgcaggacatcgagaggcggctggacgccctggagctccgcggcgtggagctggagaagcgactgcgggcggccgagggagatgacgctgaggatagcctcatggtggactggttctggctcattcacgagaagcagcttctgctgagacaggagtcagagctgatgtacaagtccaaggcccagcgtctggaggagcagcagctggacatcgagggcgagctgcgccggctcatggccaagcccgaggctctgaagtcactgcaggagcggcggcgggagcaggagctgctggagcagtacgtgagcaccgtgaacgaccgcagtgacatcgtggactcgctggacgaggaccggctccgggaacaagaggaggatcagatgctgcgggacatgattgagaagctgggcctccagaggaagaagtccaagttccgcttgtccaagatctggtca

SEQ ID NO: 16 - protein, 904 aa, Homo sapiens
MAAIRALQQWCRQQCEGYRDVNICNMTTSFRDGLAFCAILHRHRPDLINFSALKKENIYENNKLAFRVAEEHLGIPALLDAEDMVALKVPDRLSILTYVSQYYNYFHGRSPIGGMAGVKRASEDSEEEPSGKKAPVQAAKLPSPAPARKPPLSPAQTNPVVQRRNEGAGGPPPKTDQALAGSLVSSTCGVCGKHVHLVQRHLADGRLYHRSCFRCKQCSCTLHSGAYKATGEPGTFVCTSHLPAAASASPKLTGLVPRQPGAMGVDSRTSCSPQKAQEANKARPLAWEPAAGNSPARASVPAAPNPAATSATSVHVRSPARPSESRLAPTPTEGKVRPRVTNSSPMGWSSAAPCTAAAASHPAVPPSAPDPRPATPQGGGAPRVAAPQTTLSSSSTSAATVDPPAWTPSASRTQQARNKFFQTSAVPPGTSLSGRGPTPSLVLSKDSSKEQARNFLKQALSALEEAGAPAPGRPSPATAAVPSSQPKTEAPQASPLAKPLQSSSPRVLGLPSRMEPPAPLSTSSTSQASALPPAGRRNLAESSGVGRVGAGSRPKPEAPMAKGKSTTLTQDMSTSLQEGQEDGPAGWRANLKPVDRRSPAERTLKPKEPRALAEPRAGEAPRKVSGSFAGSVHITLTPVRPDRTPRPASPGPSLPARSPSPPRRRRLAVPASLDVCDNWLRPEPPGQEARVQSWKEEEKKPHLQGRPGRPLSPANVPALPGETVTSPVRLHPDYLSPEEIQRQLQDIERRLDALELRGVELEKRLRAAEGDDAEDSLMVDWFWLIHEKQLLLRQESELMYKSKAQRLEEQQLDIEGELRRLMAKPEALKSLQERRREQELLEQYVSTVNDRSDIVDSLDEDRLREQEEDQMLRDMIEKLGLQRKKSKFRLSKIWSPKSKSSPSQ

SEQ ID NO: 17 - DNA, 3351 bp, Drosophila
caccgttttgccttgtttacatttaatgtaatttgtaccagataaatcttatttgattttactcaagtcaagtggcacgatgtgcttttgttgttcacttagtttacagtttgtgactgctattttcgtttagttatattcgccaataagccagcaattaacccactaaaatgtctgatcgtcgtggcacaaaagttggaactggtacgaaggctttggagtattggtgccgagttgtgacccaaggatataatggggtcaaggtggagaacatgaccacttcctggcgaaatggacttgccttctgcgccataatacaccactttcggccggatcttatagatttcgaccgacttaaagcagacgatatctatgagaacaacgatttggcctttacaacggccgagaaatatttgggaattcccgcactgctcgatgcagctgacatggtttcgtatgaagtacctgatagactctccatactaacgtatttatcccagttttacaaggtgctcggcaagagcctgaagcacccgaagccagaggagccacttggcgaagagagtgagccaccgcaaaaggtgatgcacattgttgggatgcctcgacgtgataagtgccagaaatgcaaccttcctgtttttctcgccgaaagggtccttgtcggcaaacgggcttatcatcgcacatgcttaaaatgtgcaagatgttcgtcattgctcacacccgggagtttttatgaaacggaggtcaataacatatactgttgcgagacttgtcccgatgaagaaagtgaaccggaatctgacattttaaagttaaagacaaccactactgattctccgaatgataaacaaatggtggcacaaagttctgattactctgaagctgaagataaacaagaagacctggaagataatgatatacgtactactgataagcctgaaaacttccaaccgccgtccaacaaagatgaacaaaataatgaactaactattaatccggtaaaccctatattatcagaggagcgtaaaatctcttttattccactagacgaagaagatggtggccttattgaacaatataataaatcaacaactcctgtaaagcccgctataccagagaaaccaaaagtctctactcttccactagacgatgaacaacatgctggcgttgaacaaaacaatgatctggcggttagtccggaaaatgacattccaaaagagaaacttaagatttcttcggtctcaatatatcttgaagatgaccgccttgttgtagatgcaatacatccggataatttagataaacaagaagctttgaacaatacatcagacgcactcattccagaatcacaggaagcaccgattcctgaaaataacactcaagtagccattaaaccagaagaccatataagcccgcgtaaagaaaataaaattttctcaaacacagaaagctgctctaagcaggaaggtgttctcccaaaacaaatggatctcgagtctcccaaggacaaggtaattgagacaaaagcatctgaaactgactatccagaggatctaaatccctttaaagatgatgactcatctaaaggtgccaacccattcgacagctctgatgatgaagtggagcttcttaaagccattccagcccaacaaagcaaaggaaaagttgtaccgccccgtccaccgccacctaaaattggcctttcatccatctccaatccgtcagagaaaccacattccagccctacactttcgcatgggaaaaaaatgccgatgccaacaccgagaatatctatatcaaaaacccagactccagcaaaaccaatgacgcatcaaggccagaaaagtagtatttcctcttcgtcttcggagcatttgaatagcataagaacgttcgatcgcggagcagatgaccgtggctcaagcatctccttgccatctgcaaatgggcctcgcaagccacttcgcgcctcagtggggagtccgctgcgaagcgaagaatcgagtcccactaccagtcttagctctattacctctccgatgcggaagaagcgccaggcacctttgcccccaatacaaacggactttgacagtgatcctggattttcaaaattgtccgacgaacaaaaggcattgttgcacactcagcttaaggccccaaatctgggtgattcaaccagaagactaataccacttgaccagagtctgctatccgatgaggcaaccgagtcgagtaattacgacgagagcttaagtacatccaatgccgacgaggaggtaaatgtggtataccgacgcattttggtgccgccaactcaacccgaaaacactgttgaacgtagcaaggaggatcaaaaatcgcctatcgtgtataacgacttcgatagaaacgtaagcccattggggcacaataaatccactcatgggaaatggaagaggcgaaagggaccagcaccagcggttccaataccaccgcgcaaagtcttacaaaggcttccgctgcaagaaattcgccacgagttcgaaattattgcggtacagcagctgggtcttgagaaacaaggcgttattctggagaaaatgatcagggatcgctgtgagcgttccttagatgccaccgatactgatggtcccgaatccgcagaagtgctaaccaactcaaaggaagtggaagatcttatattgcaactgttcgagctggtcaacgagaaaaacgaattgttccgcagacaagcggaactgatgtaccttcgacgtcagcatcgcctggagcaggagcaggcggacatagagcatgagatacgagtattaatggggcaaccggagcacaacaaaaccgattcggataaagcccatgaggaagtattaatcaatcgccttgtgaaggtggttgaaatgcgtaacgaagtaattgatagcctagagactgaccgagttcgcgaggcgcgagaggatatgagcatcaagaatcggcttcatatatacaattctgagcgcgaggaaccaccagcacatccgagaagcgctgacaaatcatccaaaaagctgtctaaaaaggaacgaaaaaaacttaaggaggaaaataagctgggcaagggcaaaaagtcggacctcgacaaggacgtcgatgagtccgaacaggcaccagctttggaaaaggttaaaaagaagcgaaacttgttctttttgaaaatgtagttgggggctggttgagttgaagtgtttacgcatcgaacaattgaaatttattttatataaatttgtaacctattaagtttctattgccatatgtgagcatttcaataaataaagtatatattttcctttaaaaaaaaaaaaaaaaaaa

SEQ ID NO: 18 - protein, 1010 aa, Drosophila
MSDRRGTKVGTGTKALEYWCRVVTQGYNGVKVENMTTSWRNGLAFCAIIHHFRPDLIDFDRLKADDIYENNDLAFTTAEKYLGIPALLDAADMVSYEVPDRLSILTYLSQFYKVLGKSLKHPKPEEPLGEESEPPQKVMHIVGMPRRDKCQKCNLPVFLAERVLVGKRAYHRTCLKCARCSSLLTPGSFYETEVNNIYCCETCPDEESEPESDILKLKTTTTDSPNDKQMVAQSSDYSEAEDKQEDLEDNDIRTTDKPENFQPPSNKDEQNNELTINPVNPILSEERKISFIPLDEEDGGLIEQYNKSTTPVKPAIPEKPKVSTLPLDDEQHAGVEQNNDLAVSPENDIPKEKLKISSVSIYLEDDRLVVDAIHPDNLDKQEALNNTSDALIPESQEAPIPENNTQVAIKPEDHISPRKENKIFSNTESCSKQEGVLPKQMDLESPKDKVIETKASETDYPEDLNPFKDDDSSKGANPFDSSDDEVELLKAIPAQQSKGKVVPPRPPPPKIGLSSISNPSEKPHSSPTLSHGKKMPMPTPRISISKTQTPAKPMTHQGQKSSISSSSSEHLNSIRTFDRGADDRGSSISLPSANGPRKPLRASVGSPLRSEESSPTTSLSSITSPMRKKRQAPLPPIQTDFDSDPGFSKLSDEQKALLHTQLKAPNLGDSTRRLIPLDQSLLSDEATESSNYDESLSTSNADEEVNVVYRRILVPPTQPENTVERSKEDQKSPIVYNDFDRNVSPLGHNKSTHGKWKRRKGPAPAVPIPPRKVLQRLPLQEIRHEFEIIAVQQLGLEKQGVILEKMIRDRCERSLDATDTDGPESAEVLTNSKEVEDLILQLFELVNEKNELFRRQAELMYLRRQHRLEQEQADIEHEIRVLMGQPEHNKTDSDKAHEEVLINRLVKVVEMRNEVIDSLETDRVREAREDMSIKNRLHIYNSEREEPPAHPRSADKSSKKLSKKERKKLKEENKLGKGKKSDLDKDVDESEQAPALEKVKKKRNLFFLKM

SEQ ID NO: 19 - protein, 202 aa, Artificial sequence
  Drosophila truncated mutant
MNYQRSDDESYANETREHKKQRAISKASRQAELKRLRIAQEIQREQEEIEVQLKDLEARGVLIEKALRGEAQNIENLDATKDNDEKLLKELLEIWRNITALKKRDEELTIRQQELQLEYRHAQLKEELNLRLSCNKLDKSSADVAAEGAILNEMLEIVAKRAALRPTASQLDLTAAGSASTSAEATGIKLTGQPHDHEESII

SEQ ID NO: 20 - protein, 3002 aa, Artificial sequence
  Drosophila G-W mutant.  G residues 134, 136, 139 of Drosophila MI CAL changed to W residues
MSRQHQRHHQQHHHLPPHQQPQQQMPQQQQQLTAQQQQQQQLLMAEHAAAAEAAELFDLLCVATTMRQILALHRAMCEAVGLRPSPLNDFYPRLKAKVRSWKAQALWKKFDARAAHRVYGKGAACTGTRVLVIWAWPCWLRTAIEAQLLGAKVVVLEKRDRITRNNVLHLWPFVITDLRNLGAKKFYGKFCAGSIDHISIRQLQCMLLKVALLLGVEIHEGVSFDHAVEPSGDGGGWRAAVTPADHPVSHYEFDVLIGADGKRNMLDFRRKEFRGKLAIAITANFINKKTEAEAKVEEISGVAFIFNQAFFKELYGKTGIDLENIVYYKDETHYFVMTAKKHSLIDKGVIIEDMADPGELLAPANVDTQKLHDYAREAAEFSTQYQMPNLEFAVNHYGKPDVAMFDFTSMFAAEMSCRVIVRKGARLMQCLVGDSLLEPFWPTGSGCARGFLSSMDAAYAIKLWSNPQNSTLGVLAQRESIYRLLNQTTPDTLQRDISAYTVDPATRYPNLNRESVNSWQVKHLVDTDDPSILEQTFMDTHALQTPHLDTPGRRKRRSGDLLPQGATLLRWISAQLHSYQFIPELKEASDVFRNGRVLCALINRYRPDLIDYAATKDMSPVECNELSFAVLERELHIDRVMSAKQSLDLTELESRIWLNYLDQICDLFRGEIPHIKHPKMDFSDLRQKYRINHTHAQPDFSKLLATKPKAKSPMQDAVDIPTTVQRRSVLEEERAKRQRRHEQLLNIGGGAAGAAAGVAGSGTGTTTQGQNDTPRRSKKRRQVDKTANIEERQQRLQEIEENRQERMSKRRQQRCHQTQNFYKSLQLLQAGKLLREGGEAGVAEDGTPFEDYSIFLYRQQAPVFNDRVKDLERKLLFPDRERGDIPSALPRTADEQFSDRIKNMEQRMTGRGGLGGDKKPKDLMRAIGKIDSNDWNVREIEKKIELSKKTEIHGPKGREKVPKWSKEQFQARQHKMSKPQRQDSREAEKFKDIDQTIRNLDKQLKEGHNLDVGERGRNKVASIAGQFGKKDEANSDEKNAGSSNATTNTNNTVIPKSSSKVALAFKKQAASEKCRFCKQTVYLMEKTTVEGLVLHRNCLKCHHCHTNLRLGGYAFDRDDPQGRFYCTQHFRLPPKPLPQRTNKARKSAAAQPASPAVPPTAGSVPTAAATSEHMDTTPPRDQVDLLQTSRANASADAMSDDEANVIDEHEWSGRNFLPESNNDSQSELSSSDESDTESDSEMFEEADDSPFGAQTLQLASDWIGKQYCEDSDDSDDFYDSSEGIADDGKDDTEGEEFKKARELRRQEVRLQPLPANLPTDTETEVQTESESTSPDEVELNSATEISTDSEFDNDEIIRQAPKIFIDDTHLRKPTKVQIKSTMIGPNAASAGLHQKQLAAREKGGSYLQKYQPQPPLSQFKPLVQVDPTLLIGSQRAPLQNPRPGDYLLNKTASTEGIASKKSLELKKRYLLGEPANGDKIQKSGSTSVLDSRIRSFQSNISECQKLLNPSSDISAGMRTFLDRTKLGEGSQTTPGQTNELIRSATSNVINDLRVELRIQKTDSSHSTDNEKENVFVNCKNELNKGMEYTDAVNATLLDQLARKSSPTTPTNKTVVEVIDLVTPEKPIDIIDLTALETPKKQLVDGSAMDVDERLTPDSNKISELQQEVKEEPKPDVSRDVKECIPDILGHIKEGTGSKEPGGEDQQSLLEQSDEEKRDSPEKDVAEHELYEPDSVQIQVPNIPWEKSKPEVMSTTGSSGSICSSSDSSSIEDIQHYILESTTSPDTQTVGGKHNVPRLEVHDTSGALMQVDSLMIVNGKYIGDPEDVKFLDMPANVIVPPAPALKTNELDMEDDQEAEAEPVTATPEPVECTVIEAERRVTAPPPLPEMGPPKLKFDSKNENKIESLKNLPLIVESNVEHSQAVKPITLNLSNLARTPDTPTTPTAHDSDKTPTGEILSRGSDSETEHTGTGQVLTETELSDWTADDCISENFVDLEFALNSNKGTIKRRKDRRRSGASKLPSGNEVIHELARQAPVVQMDGILSAIDIDDIEFMDTGSEGSCAEAYPATNTALIQNRGYMEYIEAEPKKTTRKAAPPSSYPGNLPPLMTKRDEKLGVDYIEQGAYIMHDDAKTPVNEVAPAMTQSLTDSITLNELDDDSMIISQTQPTTTEESEALTVVTSPLDTSSPRVLDQFASMLAAGKGDSTPSSSEQQPKTSTVTSSSTGPNSSTTGNVSKEPQEEDLQIQFEYVRALQQRISQISTQRRKSSKGEAPNLQLNSSAPVIESAEDPAKPAEEPLVSMRPRTTSISGKVPEIPTLSSKLEEITKERTKQKDLIHDLVMDKLQSKKQLNAEKRLHRSRQRSLLTSGYASGSSLSPTPKLAAACSPQDSNCSSQAHYHASTAEEAPKPPAERPLQKSATSTYVSPYRTVQAPTRSADLYKPRPFSEHIDSNALAGYKLGKTASFNGGKLGDFAKPIAPARVNRGGGVATADIANISASTENLRSEARARARLKSNTELGLSPEEKMQLIRSRLHYDQNRSLKPKQLEEMPSGDLAARARKMSASKSVNDLAYMVGQQQQQQVEKDAVLQAKAADFTSDPNLASGGQEKAGKTKSGRRPKDPERRKSLIQSLSSFFQKGSGSAASSSKEQGGAVAAVHSEQSERPGTSSSGTPTISDAAGGGGGGGGVFSRFRISPKSKEKSKSCFDLRNFGFGDKDMLVCNAASPAGATSASQKNHSQEYLNTTNNSRYRKQTNTAKPKPESFSSSSPQLYIHKPHHLAAAHPSALDDQTPPPIPPLPLNYQRSDDESYANETREHKKQRAISKASRQAELKRLRIAQEIQREQEEIEVQLKDLEARGVLIEKALRGEAQNIENLDATKDNDEKLLKELLEIWRNITALKKRDEELTIRQQELQLEYRHAQLKEELNLRLSCNKLDKSSADVAAEGAILNEMLEIVAKRAALRPTASQLDLTAAGSASTSAEATGIKLTGQPHDHEESII

SEQ ID NO: 21 - protein, 1048 aa, Mouse
MASPASTNPAHDHFETFVQAQLCQDVLSSFQGLCRALGVESGGGLSQYHKIKAQLNYWSAKSLWAKLDKRASQPVYQQGQACTNTKCLVVGAGPCGLRAAVELALLGARVVLVEKRIKFSRHNVLHLWPFTIHDLRALGAKKFYGRFCTGTLDHISIRQLQLLLLKVALLLGVEIHWGVKFTGLQPPPRKGSGWRAQLQPNPPAQLASYEFDVLISAAGGKFVPEGFTIREMRGKLAIGITANFVNGRTVEETQVPEISGVARIYNQKFFQSLLKATGIDLENIVYYKDETHYFVMTAKKQCLLRLGVLRQDLSETDQLLGKANVVPEALQRFARAAADFATHGKLGKLEFAQDARGRPDVAAFDFTSMMRAESSARVQEKHGARLLLGLVGDCLVEPFWPLGTGVARGFLAAFDAAWMVKRWAEGAGPLEVLAERESLYQLLSQTSPENMHRNVAQYGLDPATRYPNLNLRAVTPNQVQDLYDMMDKEHAQRKSDEPDSRKTTTGSAGTEELLHWCQEQTAGFPGVHVTDFSSSWADGLALCALVHHLQPGLLEPSELQGMGALEATTWALRVAEHELGITPVLSAQAVMAGSDPLGLIAYLSHFHSAFKNTSHSSGLVSQPSGTPSAILFLGKLQRSLQRTRAKVDEETPSTEEPPVSEPSMSPNTPELSEHQEAGAEELCELCGKHLYILERFCVDGHFFHRSCFCCHTCEATLWPGGYGQHPGDGHFYCLQHLPQEDQKEADNNGSLESQELPTPGDSNMQPDPSSPPVTRVSPVPSPSQPARRLIRLSSLERLRLSSLNIIPDSGAEPPPKPPRSCSDLARESLKSSFVGWGVPVQAPQVPEAIEKGDDEEEEEEEEEEEEEPLPPLEPELEQTLLTLAKNPGAMTKYPTWRRTLMRRAKEEEMKRFCKAQAIQRRLNEIEATMRELEAEGTKLELALRKESSSPEQQKKLWLDQLLRLIQKKNSLVTEEAELMITVQELDLEEKQRQLDHELRGYMNREETMKTEADLQSENQVLRKLLEVVNQRDALIQFQEERRLREMPA

SEQ ID NO: 22 - protein, 1480 aa, Mouse
MGENEDEKQAQASQVFENFVQATTCKGTLQAFNILTCLLDLDPLDHRNFYSQLKSKVNTWKAKALWHKLDKRGSHKEYKRGKACSNTKCLIVGGGPCGLRTAIELAYLGAKVVVVEKRDTFSRNNVLHLWPFTIHDLRGLGAKKFYGKFCAGSIDHISIRQLQLILFKVALMLGVEVHVNVEFVRVLEPPEDQENQKVGWRAEFLPADHALSDFEFDVIIGADGHRNTLEGFRRKEFRGKLAIAITANFINRNSTAEAKVEEISGVAFIFNQKFFQDLKEETGIDLENIVYYKDSTHYFVMTAKKQSLLDKGVILNDYIDTEMLLCSENVNQDNLLSYAREAADFATNYQLPSLDFAINHNGQPDVAMFDFTSMYASENAALMRERQAHQLLVALVGDSLLEPFWPMGTGCARGFLAAFDTAWMVKSWDQGTPPLEVLAERESLYRLLPQTTPENINKNFEQYTLDPATRYPNLNLHCVRPHQVKHLYITKEMDRFPLERWGSVRRSVSLSRRESDIRPNKLLTWCQQQTKGYQHVRVTDLTTSWRSGLALCAIIHSFRPELINFDSLNEDDAVENNQLAFDVAKREFGILPVTTGKEMASTQEPDKLSMVMYLSKFYELFRGTPLRPMDSWRKNYGENADFGLGKTFIQNNYLNLTLPRKRTPRVDTQTEENDMNKRRRQGFNHLEELPSFSSRSLGSSQEYAKESGSQNKVKHMANQLLAKFEENTRNPSVVKQESPRKAFPLSLGGRDTCYFCKKRVYMIERLSAEGHFFHQECFRCSVCSATLRLAAYAFDCDEGKFYCKPHFVHCKTSSKQRKRRAELNQQREEEGTWQEQEAPRRDVPTESSCAVAAISTPEGSPPGTSTSFFRKALSWPLRLTRGLLNLPQSLLRWMQGLQEAAGHHVRDNAHNYCFMFELLSLGLLLLWAFSKVLAAMYRESEESLENIRSWLLRFIPVKLQMGQPGGPELSKERKLGLKKLVLTEEQKNKLLDWSDCTQEHKTGEQLSQESAENIRGGSLKPTCSSTLSQAVKEKLLSQKKALGGMRTPAVKAPQEREVPPPKSPLKLIANAILRSLLHNSEAGKKTSPKPESKTLPRGQPHARSFSLRKLGSSKDGDQQSPGRHMAKKASAFFSLASPTSKVAQASDLSLPNSILRSRSLPSRPSKMFFSTTPHSKVEDVPTLLEKVSLQDATHSPKTGASHISSLGLKDKSFESFLQECKQRKDIGDFFNSPKEEGPPGNRVPSLEKLVQPVGSTSMGQVAHPSSTGQDAHPVAPVTEATSSPTSSSAEEEADSQLSLRIKEKILRRRRKLEKQSAKQEELKRLHKAQAIQRQLEEVEERQRTLAIQGVKLEKVLRGEAADSGTQDEAQLLQEWFKLVLEKNKLMRYESELLIMAQELELEDHQSRLEQKLRQKMLKDEGQKDENDLKEEQEIFEEMMQVIEQRNKLVDSLEEQRVKERTQDQHFENFVLSRGCQLSRT

SEQ ID NO: 23 - protein, 1026 aa, Mouse
  MISC_FEATURE (1016)..(1016): Xaa is any amino acid
MEERKQETTNQAHVLFDRFVQATTCKGTLRAFQELCDHLELKPKDYRSFYHKLKSKLNYWKAKALWAKLDKRGSHKDYKKGKACTNTKCLIIGAGPCGLRTAIDLSLLGAKVVVIEKRDAFSRNNVLHLWPFTIHDLRGLGAKKFYGKFCAGAIDHISIRQLQLILLKVALILGIEIHVNVEFQGLVQPPEDQENERIGWRALVHPKTHPVSEYEFEVIIGGDGRRNTLEGFRRKEFRGKLAIAITANFINRNTTAEAKVEEISGVAFIFNQKFFQELRETTGIDLENIVYYKDDTHYFVMTAKKQSLLDKGVILHDYTDTELLLSRENVDQEALLNYAREAADFSTQQQLPSLDFAINHYGQPDVAMFDFTCMYASENAALVREQNGHQLLVALVGDSLLEPFWPMGTGIARGFLAAMDSAWMVRSWSLGTSPLEVLAERESIYRLLPQTTPENVSKNFSQYSIDPVTRYPNININFLRPSQVRHLYDSGETKDIHLEMENMVNPRTTPKLTRNESVARSSKLLGWCQRQTEGYSGVNVTDLTMSWKSGLALCAIIHRYRPDLIDFDSLDEQNVEKNNQLAFDIAEKELGISPIMTGKEMASVGEPDKLSMVMYLTQFYEMFKDSLSSSDTLDLNAEEKAVLIASTKSPISFLSKLGQTISRKRSPKDKKEKDSDGAGKRRKTSQSEEEEPPRSYKGERPTLVSTLTDRRMDAAVGNQNKVKYMATQLLAKFEENAPAQSTGVRRQGSIKKEFPQNLGGSDTCYFCQKRVYVMERLSAEGKFFHRSCFKCEYCATTLRLSAYAYDIEDEFSPNFWCSAHYHVPVALPATVMPMCLLYHPSQVLVCLEGGPAFMSPVLFNDTNSRQAKQEELKRLHRAQIIQRQLEQVEEKQRQLEERGVAVEKALRGEAGMGKKDDPKLMQEWFKLVQEKNAMVRYESELMIFARELELEDRQSRLQQELRERMAVEDHLKTEGELSEEKKILNEMLEVVEQRDSLVALLEEQRLREKEEDKDLXAAMLCKGFSL

SEQ ID NO: 24 - protein, 476 aa, Anopheles gambiae
EMFLHFCAATTMKQIRGLYWNMLDTIGLRPGPLEEFYPKMKAAIRDWRAQALFKKFDARAAHKVYCKGRAASKTRVLIVGAGPCGLRTAIDAQLLGAKVVVVVEKRDRISRNNVLHLWPFIIHDLKALGAKKFYGKFCAGSIDHISIRQLQCILLKVALLLGVEMHEGVSFVKEIEPGDGYGWRASVSPEDHAVSHYEFDVLIGADGKRNTLEGFQRKEFRGKLAIAITANFINKRTEAEAMVEEISGVAFIFDQPFFKALYEKTGCDLENIVYYKDDTHYFVMTAKKHSLLHRGVIIKDLSDPAELLAPSNVDKPKLYEYARDAANFATKYQMPNLEFAVNHYGTPDVAVFDFTSIFAAHNSCKVTVRKNYRLLSCLVGDSLLEPFWPTGSGCARGFLSSMDAAYAIKLFANPKNSLLATIAQRESVYRLLGQTTPENLNRAFGAYTLDPSTRYKNLNKASVQIGQVKHLLDTDD

SEQ ID NO: 25 - protein, 211 aa, Ciona intestinalis
NIVYYKGETHYFVMTAKKHSLVSKGVLKQDYDNTNELLCYNNIDQEELMKYAKQAADFSTRHQLPHLDFAINQYGQSDIALFDFTCMYAAENAALFRETYRQKLLCCLVGDSLLEPFWPMGTGCARGFLAAFDLVWMTKQLALKRKCSNYDPNDNKVELAVLAERESIYRVLHQTTPQNTMKNHQDYTIAPSTRYANLNLKAVTPSQVKPL

SEQ ID NO: 26 - protein, 252 aa, Danio rerio
FRRKEFRGKLAIAITANFINRNTTAEAKVEEISGVAFIFNQKFFQDLREATGIDLENIVYYKDDTHYFVMTAKKQSLLEKGVILDYADTEMLLSRANVDQKALLSYAREAADFSTNHQLPKLDFAINHYGQPDVAMFDFTCMYASENAALVRQRNGHKLLVALVGDSLLEPFWPMGTGIARGFLAAMDSAWMVRSWAHGSSPLEVLAERESIYRLLPQTTPENVSKNFSQYSVDPTTRYPNISLHQVRPNQV

SEQ ID NO: 27 - protein, 154 aa, Danio rerio
ADHPVADYDFDVVVGADGRRNSLEGFRRKEFRGKLAIAITANFTNRNTTAEAKVEEISGVAFIFNQKFFQDLRQETGIDLENIVYYKDNTHYFVMTAKKQSLLDKGVIIHDYIDTEALLNSENVNQEALLVYAREAADYATHYQLPTLDYAMNH

SEQ ID NO: 28 - protein, 230 aa, Gallus gallus
LFDRFVQASTCKGTLKAFQELCDYLELKPKDYRSFYHKLKSKLNYWKAKALWAKLDKRGSHKDYKKGKACANTKCLIIGAGPCGLRTAIDLSFLGAKVVVIEKRDAFSRNNVLHLWPFTIHDLRGLGAKKFYGKFCAGSIDHISIRQLQLILLKVALILGIEIHVNVEFQGLVYPPEDQENERIGWRALVHPKTHPVSEYEFEVIIGGDGRRNTLEGFRRKEFRGKLAIA

SEQ ID NO: 29 - protein, 227 aa, Gallus gallus
LFEHFIRARQCQEVLSCFAELCHQLGLRGNGLQLYHSLKAALNFWSAKALWIKLDKKAGHKDYDQGTACASTKCLVVGAGPCGLRTAIELALLGARVVVLEKRDSFSRNNVLHLWPFTIHDLRALGAKKFYGRFCTGTLDHISIRQLQLILLKVALLLGVEVHTKVQFKGLHPPTGKAAGQGGWRAVLQPSSSPLSHYEFDVLISAGGGKFVPEDFKRKEMRGKLAI

SEQ ID NO: 30 - protein, 467 aa, Rattus norvegicus
QVFENFVQATTCKGTLQAFNILTCLLDLDPLDHRNFYTQLKSKVNTWKAKALWHKLDKRGSHKEYKRGKACSNTKVLIVGGGPCGLRTAIELAYLGAKVVVVEKRDTFSRNNVLHLWPFTIHDLRGLGAKKFYGKFCAGSIDHISIRQLQLILFKVALMLGVEIHVNVEFVRVREPPKDGWRAEFLPADHALSNFEFDVIIGADGHRNTLEFRRKEFRGKLAIAITANFINRNSTAEAKVEEISGVAFIFNQKFFQDLKEETGIDLENIVYYKDSTHYFVMTAKKQSLLDKGVILQDYIDTEMLLCAENVNQDNLLSYAREAADFATNYQLPSLDFAINHNGQPDVAMFDFTSMYASENAALMRERQAHQLLVALVGDSLLEPFWPMGTGCARGFLAAFDTAWMVKSWDQGTPPLEVLAERESLYRLLPQTTPENINKNFEQYTLDPATRYPNLNVHCVRPHQVSAL

SEQ ID NO: 31 - protein, 467 aa, Rattus norvegicus
FETFVQAQLCQDVLSSFQGLCRALGVESGGGLPQYHKIKAQLNYWSAKSLWAKLDKRASQPAYQQGQACTNTKVLVVGAGPCGLRAAVELALLGARVVLVEKRTKFSRHNVLHLWPFTIHDLRALGAKKFYGRFCTGTLDHISIRQLQLLLLKVALLLGVEIHWGFTFTGLQPPPKKGGSGWRARIQPSPPAQLASYEFDVLISAGGGKFVLGFTIREMRGKLAIGITANFVNGRTVEETQVPEISGVARIYNQKFFQSLLKATGIDLENIVYYKDDTHYFVMTAKKQCLLRLGVLRQDLPETDQLLGKANVVPEALQQFARAAADFATQGKLGKLEFAQDARGRPDVAAFDFTSMMRSESSARIQEKHGARLLLGLVGDCLVEPFWPLGTGVARGFLAAFDAAWMVKRWAEGTGPLELLAERESLYQLLSQTSPENMHRNVAQYGLDPATRYPNLNLRAVTPNQVR

SEQ ID NO: 32 - protein, 468 aa, Rattus norvegicus
LFDRFVQATTCKGTLRAFQELCDHLELKPKDYRSFYHKLKSKLNYWKAKALWAKLDKRGSHKDYKKGKACTNTKVLIIGAGPCGLRTAIDLSLLGAKVVVIEKRDAFSRNNVLHLWPFTIHDLRGLGAKKFYGKFCAGAIDHISIRQLQLILLKVALILGIEIHVNVEFQGLVQPPEDGIGWRALVHPKTHPVSEYEFEVIIGGDGRRNTLEFRRKEFRGKLAIAITANFINRNTTAEAKVEEISGVAFIFNQKFFQELREATGGIDLENIVYYKDDTHYFVMTAKKQSLLDKGVILQDYTDTELLLSRENVDQEALLNYAREAADFSTQQQLPSLDFAINHYGQPDVAMFDFTCMYASENAALVREQNGHQLLVALVGDSLLEPFWPMGTGIARGFLAAMDSAWMVRSWSLGTSPLEVLAERRESIYRLLPQTTPENVSKNFSQYSIDPVTRYPNININFLRPSQVR

SEQ ID NO: 33 - protein, 428 aa, Bos taurus
LFDRFVQATTCKGTLKAFQELCDHLELKPKDHRSFYHKLKSKLNYWKAKALWAKLDKRGSHKDYKKGKVCTNTKVLIIGAGPCGLRTAIDLSLLGAKVVVIEKRDAFSRNNVLHLWPFTIHDLRGLGAKKFYGKFCAGAIDHISRQLQLILLKVALILGIEIHVNVEFRGLVEPPEDGIGWRALVHPKTHPVSEYEFEVIIGGDGRRNTLEFRRKEFRGKLAIAITANFINRNTTAEAKVEEISGVAFIFNQKFFQELREATGGIDLENIVYYKDDTHYFVMTAKKQSLLDKGVILQDYADTELLLSRENVDQEALLSYAREAADFSTQQQLPSLDFAINHYGQPDVAMFDFTCMYASENAALVREHNGHQLAWWLWVGGDSLRESIYRLLPQTTPENVSKNFSQYSIDPVTRYPNVNVNFLRPSQVR

SEQ ID NO: 34 - protein, 177 aa, Bos taurus
ITANFVNGRTVEETQVPEISGVARIYNQSFFQSLLKATGIDLENIVYYKDDTHYFVMTAKKQCLLRLGVLHKDWPDTERLLGSANVVPEALQRFARAAADFATHGKLGKLEFARDAHGRPDVSAFDFTSMMRAESSARVQERHGTRLLLGLVGDCLVEPFWPLGTGVARGFLAAFDA

SEQ ID NO: 35 - protein, 169 aa, Sus scrofa
AKVVVVEKRDTFSRNNVLHLWPFTIHDLRGLGAKKFYGKFCAGSIDHISIRQLQLILFKVALLLGVEIHVNVEFVKVLEPPEDQENQKIGWRAEFLPADHSLSEFEFDVIIGADGRRNTLEGFRRKEFRGKLAIAITANFINRNSTAEAKVEEISGVAFIFNQKFFQDL

SEQ ID NO: 36 - protein, 468 aa, Pan troglodytes
  MISC_FEATURE (298)..(298): Xaa is any amino acid
LFDRFVQATTCKGTLKAFQELCDHLELKPKDYRSFYHKLKSKLNYWKAKALWAKLDKRGSHKDYKKGKACANTKVLIIGAGPCGLRTAIDLSLLGAKVVVIEKRDAFSRNNVLHLWPFTIHDLRGLGAKKFYGKFCAGAIDHISIRQLQLILLKVALILGIEIHVNVEFQGLIQPPEDGIGWRALVHPKTHPVSEYEFEVIIGGDGRRNTLEFRRKEFRGKLAIAITANFINRNTTAEAKVEEISGVAFIFNQKFFQELREATGGIDLENIVYYKDDTHYFVMTAKKQSLLDKGVILXDYADTELLLSRENVDQEALLSYAREAADFSTQQQLPSLDFAINHYGQPDVAMFDFTCMYASENAALVREQNGHQLLVALVGDSLLEPFWPMGTGIARGFLAAMDSAWMVRSWSLGTSPLEVLAERRESIYRLLPQTTPENVSKNFSQYSIDPVTRYPNINVNFLRPSQVR

SEQ ID NO: 37 - DNA, 18 bp, Artificial sequence
  Amplification primer
ggagcagggccctgtgga

SEQ ID NO: 38 - DNA, 18 bp, Artificial sequence
  Amplification primer
tgggcatggccctgttgg

SEQ ID NO: 39 - protein, 7 aa, Drosophila
GAGPCGL

SEQ ID NO: 40 - protein, 7 aa, Artificial sequence
  FAD-binding domain mutant
WAWPCWL

(19) United States
(12) Patent Application Publication
(10) Pub. No.: US 2003/0232419 A1
(43) Pub. Date: Dec. 18, 2003

(54) Molecules interacting with CASL (MICAL) polynucleotides, polypeptides, and methods of using the same

(76) Inventors: Alex L. Kolodkin, Baltimore, MD (US); Jon R. Terman, Baltimore, MD (US); Tiany Mao, Parkville, MD (US); Ronald J. Pasterkamp, Baltimore, MD (US); Hung-Hsiang Yu, Lynnwood, WA (US)

Correspondence Address: LISA A. HAILE, J.D., PH.D., GRAY CARY WARE & FREIDENRICH LLP, Suite 1100, 4365 Executive Drive, San Diego, CA, 92121-2133, US

(21) Appl. No.: 10/359,012

(22) Filed: Feb. 4, 2003

Related U.S. Application Data
(60) Provisional application No. 60/354,178, filed on Feb. 4, 2002.
(60) Provisional application No. 60/384,302, filed on May 30, 2002.
(60) Provisional application No. 60/388,325, filed on Jun. 13, 2002.

(51) Int. Cl. C12Q 1/68; G01N 33/53; G01N 33/567; C12N 9/06; C12P 21/02; C12N 5/06; C07K 16/40; C07H 21/04

(52) U.S. Cl. 435/191; 435/69.1; 435/320.1; 435/325; 530/388.26; 435/6; 435/7.2; 536/23.2

(57) ABSTRACT

The present invention provides MICAL and MICAL-Like polypeptides and polynucleotides. Also provided are methods that for identifying agents that affect axon growth and placement. Furthermore, provided herein are methods for affecting axon growth and placement.

### RELATED APPLICATION DATA

This application claims the benefit of priority under 35 U.S.C. §119(e) of U.S. Serial No. 60/354,178, filed Feb. 4, 2002; U.S. Serial No. 60/384,302, filed May 30, 2002, and No. 60/388,325, filed Jun. 13, 2002, the entire contents of which are incorporated herein by reference.

### STATEMENT OF GOVERNMENT SUPPORT

This invention was made in part with government support under Grant Nos. NRSA-NS11055 and NS15165 awarded by the National Institutes of Health. The United States government may have certain rights in this invention.

### BACKGROUND OF THE INVENTION

1. Field of the Invention

This invention relates generally to polynucleotides encoding a family of polypeptides, and more specifically to polynucleotides encoding polypeptides having oxygenase activity and methods of use thereof.

2. Background Information

A great deal of research has focused on identifying factors that underlie human pathological conditions and disease states. This research has focused on molecules that are important for normal human development to identify those factors whose function is compromised in human disorders. For example, during development, neurons form connections with one another and with other targets by extending processes called axons.

In order to make developmental connections, axons navigate over long distances selecting their correct pathway, finding their appropriate target area, and establishing the proper connections with their target. The means by which axons accomplish this, remains largely unknown. It is clear however, that identifying the molecular signals that enable axons to form these connections is important for developing treatments for many neurological disorders including spinal cord injury.

Many of the same molecules implicated as functioning to guide the growing axon have been shown to function in cell adhesion, cell proliferation, cytoskeletal integrity, and other aspects of normal cell migration both in the nervous system and outside it. Characterization of these molecules will identify strategies for treatment of cell migration disorders including tumorigenesis. Therefore, there remains a need to identify molecules that function to guide growing axons.

Following spinal cord injury in humans, axons fail to reestablish their connections, which results in paralysis and loss of sensation of the affected area. The factors that inhibit axons from reestablishing their connections are not known. It is interesting, however, that during development, inhibition of axon growth plays a role in forming the nervous system. Axons are guided to their targets by molecules that attract them as well as by those that inhibit (i.e., repel) them. These molecules help channel axons into appropriate areas, as well as prevent them from entering unwanted regions. However, these molecules remain largely unidentified. Therefore, there remains a need to identify molecules that inhibit or repel axon growth.

### SUMMARY OF THE INVENTION

The present invention relates to a family of proteins, called MICALs, that are large, multidomain proteins expressed in axons, that interact with the neuronal plexin A receptor and are required for semaphorin 1a-PlexA-mediated repulsive axon guidance. In addition to containing several domains known to interact with cytoskeletal components, MICALs have a flavoprotein monooxygenase domain, the integrity of which is required for Sema-1a-PlexA repulsive axon guidance. The presence of these domains suggest a previously unknown role for oxidoreductases in repulsive neuronal guidance.

In one embodiment, the present invention provides an isolated polypeptide that includes a plexin interacting region and one or more of an N-terminal MICAL domain, a calponin homology domain, a LIM domain, and a proline rich region, wherein the polypeptide has monooxygenase activity, plexin interacting activity, and/or axon guidance regulatory activity. The polypeptide can also include a first variable MICAL region and a second variable MICAL region, which can form part of the proline rich region. The polypeptide can include, for example, from N-terminal to C-terminal, an N-terminal MICAL domain, a calponin homology doxmain, a first variable MICAL region, a LIM domain, a proline rich region, and a plexin interacting region.

In one aspect, the polypeptide is a mammalian polypeptide. For example, the isolated polypeptide can be human MICAL-1, human MICAL-2, or human MICAL-3. Accordingly, the polypeptide can include an amino acid sequence as set forth in SEQ ID NO:2 (human MICAL-1), SEQ ID NO:4 (human MICAL-2), or SEQ ID NO:6 (human MICAL 3).

In another aspect, the polypeptide is a Drosophila MICAL polypeptide. For example, the polypeptide can have an amino acid sequence as set forth in SEQ ID NO:8 (Drosophila long variant) SEQ ID NO: 10 (Drosophila medium variant), or SEQ ID NO: 12 (Drosophila short variant).

In another embodiment, the present invention provides a MICAL-Like polypeptide. Accordingly, the isolated polypeptide includes a plexin interacting region and alternatively one or more of a calponin homology domain, a LIM domain, and a proline rich region, and wherein the polypeptide interacts with a plexin. The polypeptide can also include a first variable region and a second variable region, which can form part of the proline rich region. For example, the polypeptide includes, from N-terminal to C-terminal, a calponin homology domain, a first variable MICAL region, a LIM domain, a proline rich region, and a plexin interacting region.

In another aspect, the isolated polypeptide is a Drosophila MICAL-Like polypeptide. For example, the polypeptide can have an amino acid sequence as set forth in SEQ ID NO: 18 (Drosophila MICAL-Like polypeptide).

In another embodiment, the present invention provides an isolated polynucleotide encoding a MICAL polypeptide or a MICAL-Like polypeptide of the present invention. The polynucleotide can encode a mammalian MICAL polypeptide, for example, human MICAL-1, human MICAL-2, human MICAL-3, human MICAL-Like 1, or human MICAL-Like 2. Accordingly, in one aspect, the polynucleotide is the coding sequence portion of SEQ ID NO:1 (human MICAL-1 cDNA), the coding sequence portion of SEQ ID NO:3 (human MICAL-2 cDNA), the coding sequence portion of SEQ ID NO:5 (human MICAL 3 cDNA), the coding sequence portion of SEQ ID NO: 13 (human MICAL-Like 1 cDNA), or the coding sequence portion of SEQ ID NO: 15 (human MICAL-Like 2 cDNA).

The present invention also provides an isolated polynucleotide that selectively hybridizes to a polynucleotide encoding a MICAL polypeptide or a MICAL-Like polypeptide.

In yet another embodiment, the present invention provides a method for identifying an agent that affects axonal guidance regulatory activity. The method includes contacting a polypeptide of the present invention that has axonal guidance regulatory activity, or a cell expressing the polypeptide, for example recombinantly expressing the polypeptide, with a candidate agent. Next, axonal guidance regulatory activity or expression of the polypeptide is compared in the presence versus absence of the agent. A difference in activity or expression is indicative of an agent that affects axonal guidance regulatory activity.

In another embodiment, the present invention provides a method for affecting axonal guidance regulatory activity. The method includes contacting a cell, for example, a neuron, that expresses a polypeptide of the invention such as a MICAL polypeptide, with an agent that alters MICAL activity and, thereby, affects axonal guidance regulatory activity. In one aspect, the method is performed in vivo and includes inhibiting axonal guidance regulatory activity by contacting the cell with an antioxidant that inhibits MICAL activity. The axonal guidance activity is a semaphorin-mediated axonal repulsion. As such, in another embodiment, the present invention provides a method for affecting a semaphorin-mediated process by contacting a cell that expresses a MICAL polypeptide of the invention with an effective amount of an agent that modulates MICAL activity and, thereby, affects axonal guidance regulatory activity. An agent is, for example, a small molecule, a polypeptide or fragment thereof, a peptidomimetic, or an antisense polynucleotide.

In another embodiment, the present invention provides a method for treating a neurological condition in a subject, that includes contacting in the subject, a cell of the central nervous system or the peripheral nervous system, having a disrupted axonal connection or a cell that affects axonal growth of the central nervous system or peripheral nervous system cell, with an amount of an agent that modulates the activity or expression of a MICAL polypeptide, the amount being effective to modulate axon regulatory activity, monooxygenase activity, and/or plexin interacting activity. In one aspect, the neurological condition is a spinal cord injury.

The present invention identifies exemplary flavonoids as agents that are used in methods of various embodiments of the present invention to inhibit axonal guidance regulatory activity. A variety of flavonoid anti-oxidants are known and are candidate inhibitors MICAL activity and, thereby, of axonal guidance regulatory activity such as semaphorin-mediated axonal repulsion. In one aspect of the invention, the flavonoids ECGC and EC and related gallic acid derivatives are inhibitors of semaphorin-mediated axonal repulsion.

In another aspect, the present invention provides a method for inducing regrowth of an injured process of a neuron, that includes altering the levels of reactive oxygen species or other oxidation products in the milieu of the neuron.

### BRIEF DESCRIPTION OF THE DRAWINGS

FIG. 1 provides a molecular characterization of Drosophila MICAL, MICAL expression in Drosophila embryonic motor axons, and co-immunoprecipitation of MICAL with neuronal PlexA.

FIG. 1A provides a schematic diagram of the MICAL locus. Variable exons are indicated with asterisks and produce: 1) a “long” isoform (4723 aa); 2) a “medium” isoform (3002 aa) (spliced out exon is shown with “X” though lines); and 3) a “short” isoform (2734 aa) (spliced out exons shown by thick angled exon connector lines). The regions corresponding to clones 23 and 151 are shown.

FIG. 1B provides the domain organization of the Drosophila MICAL gene. MICAL is characterized by flavin adenine dinucleotide (FAD) consensus binding motifs (GXGXXG, DG, and GD motifs), a calponin homology domain, a LIM domain, a Proline rich region, and a coiled-coil motif.

FIG. 2 provides schematic representations illustrating that the MICALs are a family of neuronally-expressed plexin-interacting proteins conserved from flies to mammals.

FIG. 2A is a schematic representation of the organization of the MICAL family of proteins. Amino acid identities are indicated among vertebrate MICALs and Drosophila MICAL (%s within domains) and between vertebrate MICALs (%s in arrows). The black regions indicate sequence that is not well-conserved among family members and variable in length (//). Regions encoded by an ORF situated in close proximity in the genome (10 kb) but for which cDNA sequence connecting them has not yet been identified are indicated (dots).

FIG. 2B provides a schematic representation of the domain organization of the MICAL-Like proteins. MICAL-like proteins have a similar domain organization as the MICALs but lack the N terminal 500 amino acid domain. Domain alignment and amino acid identity between Drosophila MICAL and MICAL-like proteins is indicated (within domains) and between MICAL-like proteins (within arrows). Available D-MICAL-L cDNA and genomic DNA sequence information suggests that the D-MICAL-L protein begins just N-terminal to the CH domain. Human MICAL-L1 and MICAL-L2 are similar in overall domain organization to D-MICAL-L and do not contain the highly conserved ˜500 amino acid MICAL N-terminal domain (dots indicate where molecular analysis is required to conclusively define the structural features of mammalian MICAL-L proteins; P, proline rich region; cc, coiled-coil).

FIG. 3 illustrates that the MICALs contain flavoprotein monooxygenase domains required for MICAL function in Drosophila.

FIG. 3A provides a schematic representation of three sequence motifs that define MICALs as flavoprotein monooxygenases. An alignment of MICALs with members of the flavoprotein monooxygenase family is shown in which (+) indicates that MICALs match the consensus, (*) indicates that MICALs match the highly important conserved residues, and (.) indicates the conserved spacing of these residues within these motifs. Shading of sequences is based on ClustalX: conserved hydrophobic residues, cysteine residues, acids, and bases are shaded in dark gray; conserved proline and glycine residues are indicated with light gray shading. MICALs contain a 100% match with the consensus ADP binding region of FAD binding proteins (FAD Fingerprint 1), a well-conserved GD sequence (FAD Fingerprint 2), and a well-conserved DG motif: distinguishing features of flavoprotein monooxygenases. The proline ((*)) in the FAD fingerprint 2 is also likely to be conserved. In the upper consensus line, uppercase indicates an amino acid; h is a hydrophobic residue, s is a small residue (i.e., compact, zero, or few side chains); c is a charged residue, and x is any residue.

FIG. 3B is a spectral analysis of MICAL that illustrates that MICAL is an FAD binding protein. A bacterial fusion protein consisting of the Drosophila MICAL flavoprotein monooxygenase (FM) domain has an absorption peak at 452 nm and a shoulder at 358 nm (dashed line), consistent with that of 50 μM free FAD (solid line) and similar in shape to spectra from other flavoproteins.

FIG. 4 illustrates that flavoprotein monooxygenase inhibitors attenuate vertebrate semaphorin axonal repulsion. Inhibitors of flavoprotein monooxygenases (EGCG and EC) as well as specific inhibitors of other oxidation/reduction enzymes including nitric oxide synthase (L-NAME), xanthine oxidase (allopurinol; Allo), and mitochondrial electron transport (NADH dehydrogenase; rotenone; Rote) were tested for their ability to inhibit semaphorin-dependent repulsive axon guidance in vertebrates.

FIG. 4A provides a schematic diagram of the rat D E14/15 Rat DRG explants were co-cultured with 293 cells expressing Sema 3A and grown for 48 hours in the presence of an inhibitor or vehicle. Axonal outgrowth was determined by measuring proximal (P) and distal (D) axon lengths.

FIG. 4B provides a graph illustrating that redox inhibitors do not have adverse effects on expression of Sema 3A or its biological activity. Media was collected from untransfected 293 cells (No Sema3A) or cells transfected with AP-Sema3A and grown in the presence of vehicle (Sema3A), 25 μm EGCG (3A/EGCG), 500 μm EC (3A/EC), 500 μm L-NAME (3A/L-NAME), 500 μm Allo (3A/Allo), or 0.1 μm Rote (3A/Rote) and ligand concentration (AP activity) was determined. The media was then diluted to 1 nM (to remove the active concentration of the inhibitor) and its biological activity was assayed in a growth cone collapse assay (% Collapse; n>60 growth cones per condition). The AP activity and percentage of growth cones collapsed were similar in the presence of all compounds.

FIG. 4C provides a graph that quantitates the effects of oxidation/reduction enzyme inhibitors on Sema 3A repulsion scored as the ratio of the axon lengths on the proximal and distal sides of the explant (P/D ratio), and on Sema3A-mediated growth cone collapse indicated as % collapsed growth cones (gray). In repulsion assays, outgrowth of DRG axons on the side distal to the 293 cells appeared normal. Attenuation of Sema 3A-mediated axonal repulsion was observed with the flavoprotein monooxygenase inhibitors (EGCG, and EC) in a dose-dependent manner but not with specific inhibitors of other oxidation/reduction enzymes. n's=number of DRG explants (repulsion assays) or number of growth cones scored (collapse assays; distributed over 4 different explants/condition). For Rote, n=4, however only 4 out of 12 explants survived. (**=p<0.0001; *=p<0.001; paired t-test). Scale bar=550 mm.

FIG. 5 shows results of a yeast interaction assay and identification of Drosophila MICAL. FIG. 5A provides a diagram of the Plexin A polypeptide. FIG. 5B illustrates that clones 23 and 151 of the yeast interaction assay encode a novel PlexA interacting protein. FIG. 5C provides a Northern blot of Drosophila total RNA using a probe that included a portion of clone 151.

FIG. 6 provides a series of graphic representations that illustrate the generation and characterization of MICAL Loss-of-Function mutants. FIG. 6A Schematic of the screen to remove the MICAL locus by generating a small deletion between two P elements that flank MICAL. FIG. 6B provides a table of summarizing genetic complementation analyses of lines exhibiting the stretch wing phenotype. FIG. 6C provides a diagram that summarizes complementation analyses and genetic organization of the MICAL locus. Sizes are in kilobases (kb); non-continuous sequence is indicated by “//”. FIG. 6D provides a Western blot that illustrates that Df(₃R)swp2^{MICAL} is a MICAL null allele that produces no MICAL protein. Prominent bands are observed at 530 kD, 330 kD, 300 kD, 200 kDa, and 125 kDa in wild type and at stronger intensity in MICAL duplication embryo; none of these bands are observed in Df(3R)swp2^{MICAL} embryos. Arrows indicate bands predicted from MICAL cDNA analysis (see text; FIG. 1A).

FIG. 7 identifies various domains of the Drosophila MICAL medium variant polypeptide (SEQ ID NO:10). Flavoprotein Monooxygenase domain is indicated by a squiggly underline; Calponin Homology domain is indicated by gray highlighting; MICAL Homology Region of Unknown Function is indicated by italics; LIM Domain is indicated by black highlighting; Proline Rich (Putative SH3 Ligands) are indicated by single underlining; Putative IQ (calcium) Binding Domain is indicated by dashed underlining; Putative Ena (Ena-like Proteins) binding Domain (Renfranz and Beckerle, Curr. Opin. Cell. Biol. 14:88, (2002)) (proline rich region) is indicated by underlining and italics; Plexin Interacting Region is indicated by bold; PDZ Ligand is indicated by double underline.

FIG. 8 provides a diagram that indicates the various domains of MICAL polypeptides and their amino acid residue numbers. Note: The MICAL 2 and 3 plexin interacting region is numbered backwards due to the lack of the intervening sequence denoted “ . . . ”.

FIG. 9 provides a diagram that indicates the various domains of MICAL-Like polypeptides and their amino acid residue numbers.

FIG. 10 provides identifies various domains of the mouse MICAL-1 polypeptide (SEQ ID NO:21). Flavoprotein Monooxygenase domain is indicated by a squiggly underline; Calponin Homology domain is indicated by gray highlighting; MICAL Homology Region of Unknown Function is indicated by italics; LIM Domain is indicated by black highlighting; Proline Rich (Putative SH3 Ligands) are indicated by single underlining; Putative IQ (calcium) Binding Domain is indicated by dashed underlining; Putative Ena (Ena-like Proteins) (Renfranz and Beckerle, Curr. Opin. Cell. Biol. 14:88, (2002)) binding Domain (proline rich region) is indicated by underlining and italics; Plexin Interacting Region is indicated by bold; PDZ Ligand is indicated by double underline.

FIG. 11 provides the cDNA (SEQ ID NO:1) and encoded polypeptide (SEQ ID NO:2) sequence of human MICAL-1.

FIG. 12 provides the cDNA (SEQ ID NO:3) and encoded polypeptide (SEQ ID NO:4) sequence of human MICAL-2.

FIG. 13 provides the cDNA (SEQ ID NO:5) and encoded polypeptide (SEQ ID NO:6) sequence of human MICAL-3.

FIG. 14 provides the cDNA (SEQ ID NO:7) and encoded polypeptide (SEQ ID NO:8) sequence of the Drosophila MICAL long isoform.

FIG. 15 provides the cDNA (SEQ ID NO:9) and encoded polypeptide (SEQ ID NO: 10) sequence of the Drosophila MICAL medium isoform.

FIG. 16 provides the cDNA (SEQ ID NO:11) and encoded polypeptide (SEQ ID NO: 12) sequence of the Drosophila MICAL short isoform.

FIG. 17 provides the cDNA (SEQ ID NO:13) and encoded polypeptide (SEQ ID NO: 14) sequence of human MICAL-Like 1.

FIG. 18 provides the cDNA (SEQ ID NO: 15) and encoded polypeptide (SEQ ID NO: 16) sequence of human MICAL-Like 2.

FIG. 19 provides the cDNA (SEQ ID NO: 17) and encoded polypeptide (SEQ ID NO: 18) sequence of Drosophila MICAL-Like.

FIG. 20 provides MICALs in other species. The amino acid sequence through the Flavoprotein Monooxygenase Domain is shown for the species indicated. The numbers indicate the amino acid number for which it aligns with Drosophila MICA medium isoform)(e.g., 53 aligns to Drosophila MICAL amino acid 53). Percent amino acid identity to the corresponding region of Drosophila MICAL is shown.

### DETAILED DESCRIPTION OF THE INVENTION

The present invention is based on the identification of a family of flavoprotein monooxygenases. This family of proteins is involved in the regulation of repulsive axon guidance. While not wanting to be limited by a particular theory, as illustrated herein, this protein family appears to regulate repulsive axon guidance by directly associating with plexins. Through this association, “MICALS” appear to be required for semaphorin-mediated repulsive axon guidance. Furthermore, MICAL proteins contain multiple domains that are known to be important for interactions with actin, intermediate filaments, and cytoskeletal-associated adaptor proteins. Therefore, MICALs are excellent candidates for directly mediating the cytoskeletal alterations characteristic of semaphorin signaling and provide novel targets for the attenuation of axonal repulsion.

In one embodiment, the present invention provides an isolated polypeptide that includes one or more of an N-terminal MICAL domain, a calponin homology domain, a LIM domain, a proline rich region, and a plexin interacting region, wherein the polypeptide has monooxygenase activity, plexin interacting activity, and/or axon guidance regulatory activity. The polypeptide can also include a first variable MICAL region and a second variable MICAL region, typically surrounding the LIM domain. The second variable region in certain aspects forms a portion of the proline rich region and can include the LIM domain. Accordingly, in one aspect, the polypeptide is a MICAL polypeptide. A MICAL polypeptide includes the following domain organization from N-terminal to C-terminal: an N-terminal MICAL domain, a calponin homology domain, a first variable MICAL region, a LIM domain, a proline rich region, and a plexin interacting region. Furthermore, a MICAL polypeptide has monooxygenase activity and interacts with a plexin, typically plexin A.

The polypeptide can be a mammalian MICAL polypeptide. For example, the isolated polypeptide can be human MICAL-1, human MICAL-2, or human MICAL-3. Accordingly, the polypeptide can include an amino acid sequence as set forth in SEQ ID NO:2 (human MICAL-1), SEQ ID NO:4 (human MICAL-2), or SEQ ID NO:6 (human MICAL 3).

The isolated polypeptide can be a Drosophila MICAL polypeptide. For example, the polypeptide can have an amino acid sequence as set forth in SEQ ID NO:8 (Drosophila long variant), SEQ ID NO:10 (Drosophila long variant), or SEQ ID NO: 12 (Drosophila long variant).

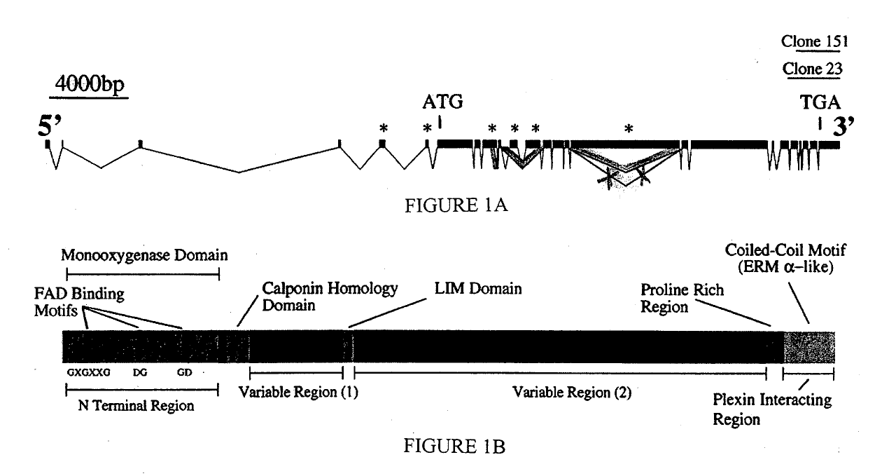

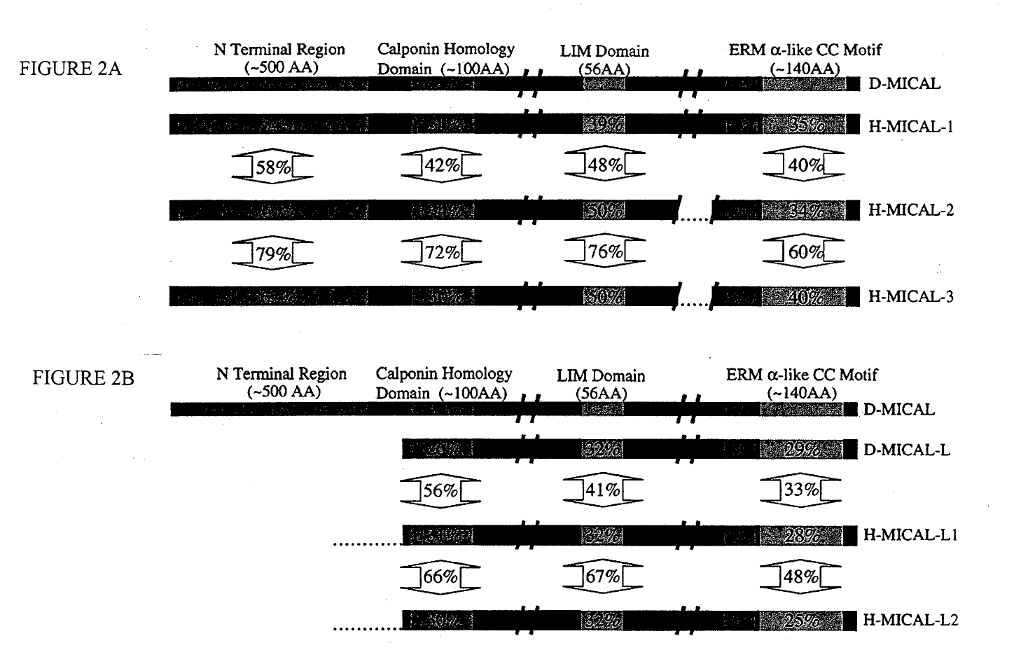

MICALs are also referred to as 151 proteins or Zephyrins. The arrangement of domains within a typical MICAL polypeptide are shown in FIG. 1A. As indicated above, a MICAL polypeptide of the present invention typically includes the following domain organization from N-terminal to C-terminal: an N-terminal MICAL domain, a calponin homology domain, a first variable MICAL region, a LIM domain, a proline rich region, and a plexin interacting region. Furthermore, a MICAL polypeptide has monooxygenase activity and interacts with a plexin, typically plexin A. The MICALs appear unique with respect to containing both calponin homology (CH) and LIM domains, in addition to their conserved N- and C-terminal regions (FIG. 2A).

In certain aspects, the present invention provides a polypeptide that includes a calponin homology domain. In fact, a MICAL polypeptide of the present invention includes a calponin homology domain. A calponin homology domain is a domain that has at least 30% amino acid sequence identity to the calponin homology domain of SEQ ID NO:2, residues 508 to 612, SEQ ID NO:4, residues 516 to 622, SEQ ID NO:6, residues 518 to 624, SEQ ID NO:8, residues 562 to 669, SEQ ID NO:10, residues 562 to 669, and/or SEQ ID NO: 12, residues 562 to 669. In certain aspects, the polypeptide through the calponin homology domain can interact with actin. The calponin homology domain in certain aspects has at least 40%, 50%, 70%, 75%, 80%, 90%, 95%, or 99% sequence identity to SEQ ID NO:2, residues 508 to 612, SEQ ID NO:4, residues 516 to 622, SEQ ID NO:6, residues 518 to 624, SEQ ID NO:8, residues 562 to 669, SEQ ID NO:10, residues 562 to 669, and/or SEQ ID NO:12, residues 562 to 669.

A polypeptide of the present invention in certain aspects includes a LIM domain. In fact, A MICAL polypeptide of the present invention includes a LIM domain. A LIM domain (Bach (2000), supra) is a domain that has at least 30% amino acid sequence identity to a LIM domain of SEQ ID NO:2, residues 697 to 750, SEQ ID NO:4, residues 1002 to 1056, SEQ ID NO:6, residues 792 to 851, SEQ ID NO:8, residues 1074 to 1129, SEQ ID NO:10, residues 1074 to 1129, and/or SEQ ID NO:12, residues 806 to 861. The LIM domain in certain aspects has at least 40%, 50%, 70%, 75%, 80%, 90%, 95%, or 99% sequence identity to the LIM domain of SEQ ID NO:2, residues 697 to 750, SEQ ID NO:4, residues 1002 to 1056, SEQ ID NO:6, residues 792 to 851, SEQ ID NO:8, residues 1074 to 1129, SEQ ID NO:10, residues 1074 to 1129, and/or SEQ ID NO:12, residues 806 to 861. LIM domains mediate protein/protein interactions with other LIM domain-containing proteins (See Bach (2000), supra).

MICAL polypeptide of the present invention includes a proline rich region. The proline rich region includes the proline rich region indicated in FIG. 1B as well as variable region 2 indicated in FIG. 1B. Accordingly, the proline rich region extends between the LIM domain and the Plexin-interacting region, and includes a variable proline rich domain and a conserved proline rich domain. Thus, the proline rich region extends from the first residue of the N terminal of the Plexin interacting domain to the last C terminal residue of the LIM domain. The proline rich region is defined by the PXXP motifs (the SH3 binding domains).A proline rich region is a region that has at least 1 PXXP SH3 binding domain. In certain aspects, the proline rich region has at least 5, 6, 7, 8, 9, 10, 12, 11, 12, 13, 14, 15, 16, 17, 18, 19, or 20 SH3 binding domains. The proline rich region in certain aspects of the invention has at least 1%, 2%, 3%, 4%, 5%, 6%, or 7% proline residues. The proline rich region in certain aspects has at least 40%, 50%, 70%, 75%, 80%, 90%, 95%, or 99% sequence identity to the proline rich region of SEQ ID NO:2, residues 751-909, SEQ ID NO:4, residues 1057 to a residue that is 230 amino acids from the C terminus, SEQ ID NO:6, residues 852 to a residue that is 190 amino acids from the C terminus, SEQ ID NO:8, residues 1130-4522, SEQ ID NO:10, residues 1130-2801, and/or SEQ ID NO:12, residues 862-2533. The proline rich region includes numerous potential SH3 binding domains (See e.g., Wages et al., J. Virol. 66(4): 1866-74 (1992)) and can include at least one Ena binding domain. For example, there are 18 putative SH3 binding domains in the medium isoform (within the variable region 2) of Drosophila MICAL.

A MICAL polypeptide of the present invention includes a plexin interacting region at its C-terminus. This domain is typically immediately C-terminal to the proline rich region. Typically, the plexin interacting region contains a predicted heptad-repeat, coiled-coil structure (FIG. 1B), a motif thought to be involved in protein-protein interactions (Burkhard et al., 2001). Interestingly, this region of a MICAL of the present invention typically shares amino acid similarity with several other coiled-coil domain-containing proteins including a portion of the alpha domain found in the Ezrin, Radixin, and Moesin (ERM) proteins (˜22% identity; Bretscher et al., 2000).

A plexin interacting region is a region that has at least 30% amino acid sequence identity to the plexin interacting region of SEQ ID NO:2, residues 910 to 1067, SEQ ID NO:4, residues 348 to 509 after the missing intervening sequence (labeled “ . . . ”), SEQ ID NO:6, residues 800 to 989 after the missing intervening sequence (labeled “ . . . ”), SEQ ID NO:8, residues 4522 to 4723, SEQ ID NO:10, residues 2802 to 3002, and/or SEQ ID NO: 12, residues 2534 to 2734. The proline rich region in certain aspects has at least 40%, 50%, 70%, 75%, 80%, 90%, 95%, or 99% sequence identity to the proline rich region of SEQ ID NO:2, residues 910 to 1067, SEQ ID NO:4, residues 348 to 509 after the missing intervening sequence (labeled “ . . . ”), SEQ ID NO:6, residues 800 to 989 after the missing intervening sequence (labeled “ . . . ”), SEQ ID NO:8, residues 4522 to 4723, SEQ ID NO:10, residues 2802 to 3002, and/or SEQ ID NO: 12, residues 2534 to 2734.

In certain aspect, the last four amino acids of MICAL (ESII) are a PDZ protein binding motif (Harris and Lim, 2001).

Typically, MICAL polypeptides of the present invention have two regions of varying length (See e.g., FIG. 1B), a first variable MICAL region and a second variable MICAL region, that have no significant similarity to any other proteins, and that appear to determine the size of the different MICAL proteins (FIG. 1B). The second variable region includes a high concentration of proline residues, and as indicated above, forms a portion of the proline rich region. For example, the second variable region of Drosophila MICAL medium isoform has 124 proline residues out of 1663 (i.e., 7.5% proline). The variable region and the proline rich region in FIG. 1B, for the Drosophila medium isoform has 130 prolines out of 1671 residues (i.e., 7.8% proline).

Interposed between the first and the second variable regions, MICALs typically have a LIM domain as discussed above (FIG. 1B), a protein-protein interaction module found in a variety of proteins involved in signal transduction cascades and in cytoskeletal organization (Bach, 2000), and also a calponin homology (CH) domain as discussed above (FIG. 1B), a domain also found in cytoskeletal and signal transduction proteins and known to be involved in actin filament binding (Gimona et al., 2002).

The present invention also provides an isolated polypeptide as disclosed above, wherein the polypeptide includes an N-terminal MICAL domain having at least about 40%, 45%, 50%, 55%, 60%, 65%. 70%, 75%, 80%, 85%, 90%, 95%, 97%, 98%, or 99% sequence identity to the N-terminal MICAL domains of SEQ ID NOS:2, 4, 6, 8, 10, or 12, and has monooxygenase activity.

The MICAL N-terminal domain of ˜500 amino acids is highly conserved among MICAL-related proteins, but is unique over its entire length in comparison to other proteins. This N-terminal region is referred to herein as the N-terminal MICAL domain. In certain aspects, the N-terminal MICAL domain includes the portion of a MICAL polypeptide that is N-terminal to the Calponin Homology domain. The N-terminal domain can be, for example about 500 to about 561 amino acids in length. N-terminal MICAL domains include residues 1-484 in SEQ ID NO:2, residues 1-492 in SEQ ID NO:4, residues 1-492 in SEQ ID NO:6, and residues 44-529 of SEQ ID Nos:8, 10, and 12.

The N-terminal MICAL domain typically includes a consensus dinucleotide binding sequence, GxGxxG (FIGS. 1B and 2A) which is distinct from the sequence present in classical mononucleotide binding motifs (Eggink et al., 1990; Eppink et al., 1997; Schulz, 1992; Wierenga et al., 1986). The N-terminal MICAL domain also typically includes three separate sequence motifs spaced throughout this domain that define them as flavoprotein monooxygenases (also called hydroxylases), a subclass of oxidoreductases (Eggink et al., 1990; Eppink et al., 1997; Wierenga et al., 1986). The amino acid sequence surrounding the GXGXXG motif typically match the consensus sequence for the ADP binding region of flavin adenine dinucleotide (FAD) binding proteins (Rossmann fold or FAD Fingerprint 1, FIGS. 1B and 2A), and distinguishes this region from consensus NAD, or NADP binding folds (Vallon, 2000; Wierenga et al., 1986). The N-terminal MICAL domain also typically has a well-conserved GD motif (FAD Fingerprint 2; FIGS. 1B and 2A) C-terminal to the FAD Fingerprint 1 region, which is important for binding the ribose moiety of FAD (Eggink et al., 1990; Eppink et al., 1997). Finally, N-terminal MICAL domains typically have the conserved DG motif (“Conserved Motif”; FIGS. 1B and 2A) between the FAD Fingerprint 1 and 2 motifs that has been reported to be involved in binding the pyrophosphate moiety of FAD (Eppink et al., 1997).

Proteins with the consensus FAD binding regions of MICALs bind FAD and use FAD in the catalysis of oxidation-reduction reactions. Flavoprotein monooxygenases are oxidoreductases (enzymes that catalyze oxidation and reduction reactions) and catalyze the insertion of one atom of molecular oxygen into their substrate using nucleotides as electron donors (Massey, 1995). Accordingly, MICAL polypeptides of the present invention typically have flavoprotein monooxygenase activity. Like other monooxygenases of this type, polypeptides of the present invention typically use FAD as a co-enzyme. Apart from the three consensus regions reported above, monooxygenases typically vary significantly, reflecting the wide range of enzymes in this family and their variable substrate binding pockets also encompassed within this domain (Eppink et al., 1997). However, MICALs and other monooxygenases show significant similarity within these three FAD binding regions and also similar spacing of these regions within the monooxygenase domain. Therefore, polypeptides of the present invention typically have a high degree of identity with the three FAD binding regions (e.g., greater than 50%, 75%, 80%, 85%, 90%, 95%. 98% and 99% sequence identity) and similar spacing of these regions.

As indicated above, polypeptides of the present invention have monooxygenase activity and bind FAD through their N-terminal MICAL domain. Methods are known in the art for determining whether a polypeptide binds FAD. For example, a solution of a polypeptide of the present invention, including a solution of a polypeptide that includes the N-terminal MICAL domain but not the other domains typically present on MICALs, such as MICAL-FM (see the Examples section herein), is yellow in color, a characteristic of flavoproteins.

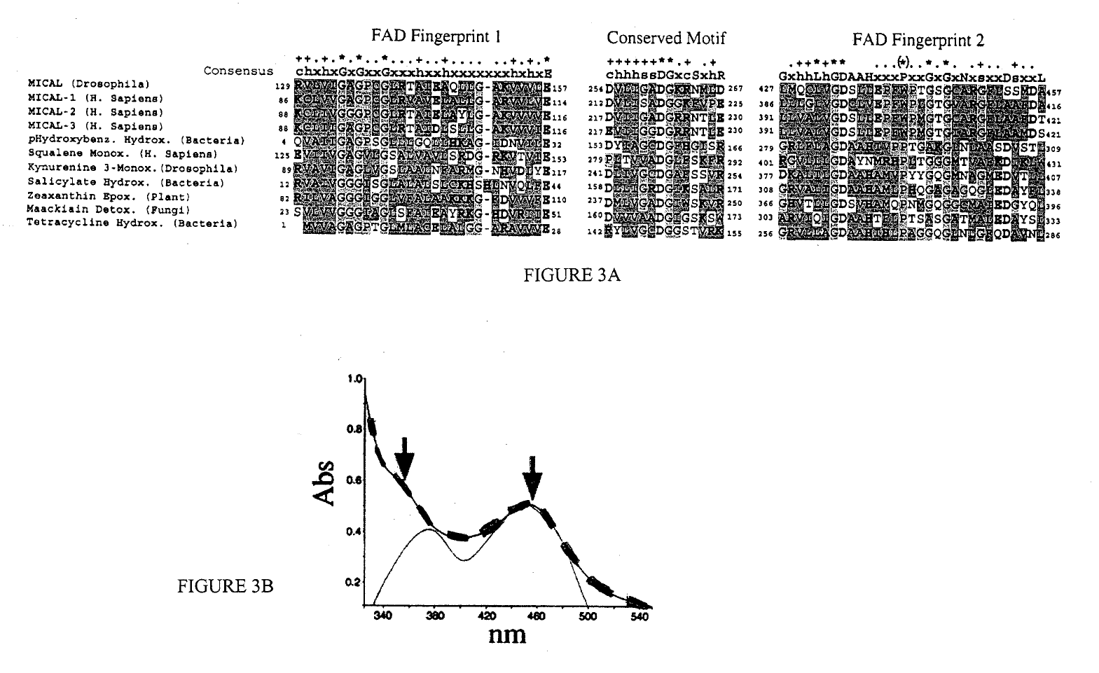

Accordingly, spectral analysis of a polypeptide of the present invention can be used to further identify the polypeptide as flavin binding polypeptides, and therefore to further determine whether a polypeptide includes an N-terminal MICAL domain. For example, a polypeptide that includes an N-terminal MICAL domain, especially a polypeptide that includes none of the other MICAL domains, typically has distinctive spectral properties. For example, a polypeptide that includes the N-terminal MICAL domain but none of the other MICAL domains can show an absorption peak at around 450 nm and a shoulder at around 360 nm (FIG. 3B). This is similar to the absorption spectra of FAD itself (˜450 nm and ˜360 nm; Macheroux, 1999), and to other related flavoproteins (e.g., p-Hydroxybenzoate Hydroxylase, Hosokawa and Stanier, 1966; and GidA, White et al., 2001). Monooxygenase activity and other flavoprotein activities can also be determined using other standard protocols as listed in Flavoprotein Protocols, Methods in Molecular Biology, Vol. 131, S. K. Chapman and G. A. Reid (Eds), Humana Press, 1999, which is incorporated by reference herein in its entirety.

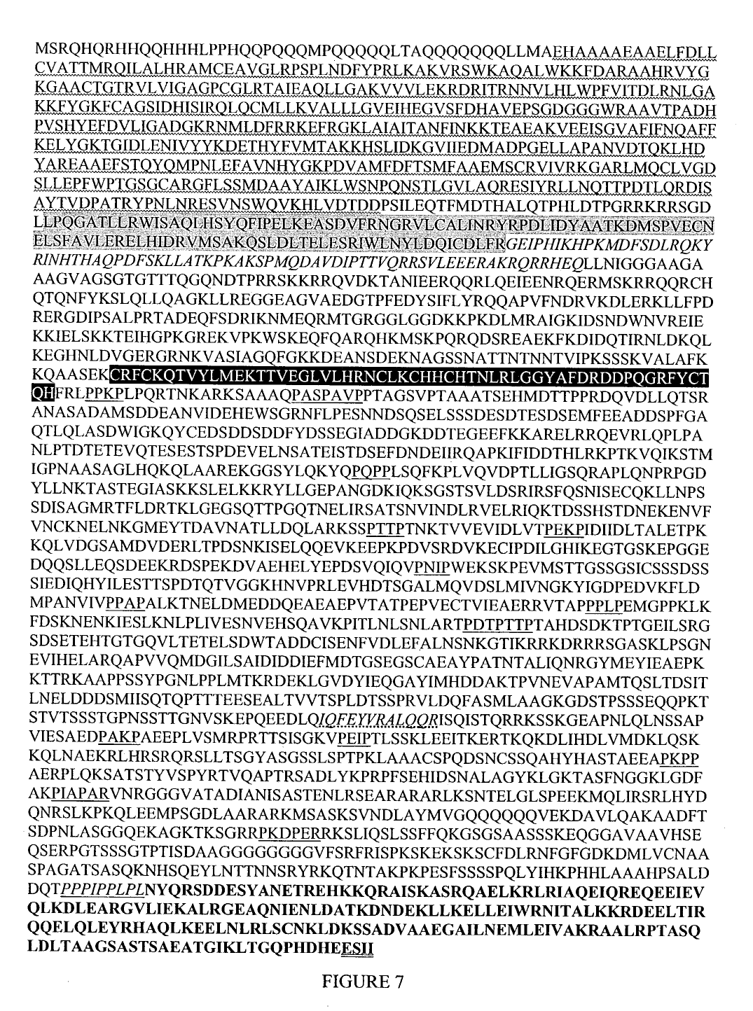

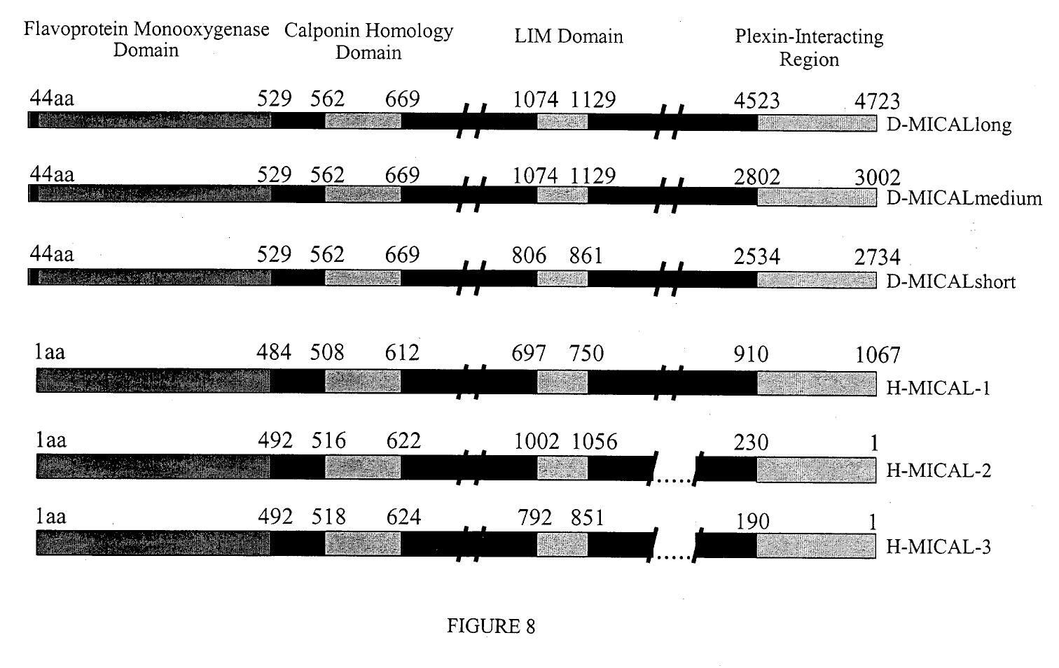

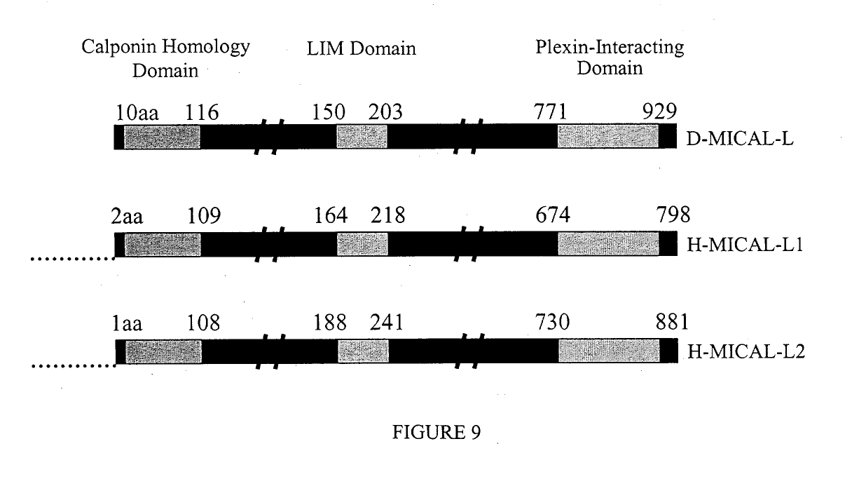

A MICAL polypeptide of the present invention can include additional functional domains observed on MICAL polypeptides (See FIG. 7). These additional domains include for example, a MICAL homology region of unknown function (FIG. 7), a putative IQ (calcium) binding domain and a PDZ ligand. Accordingly, in certain aspects, a MICAL polypeptide of the present invention includes, optionally from N-terminal to C-terminal, a flavoprotein monooxygenase domain (N-terminal MICAL domain), a calponin homology domain, a MICAL homology region of unknown function; a LIM Domain; a proline rich region (Putative SH3 Ligands), a putative IQ (calcium) binding domain; a putative Ena binding domain (Ena-like Proteins) (Renfranz and Beckerle, Curr. Opin. Cell. Biol. 14:88, (2002)), and a plexin interacting region.

A MICAL polypeptide of the present invention can include a vimentin interacting region.

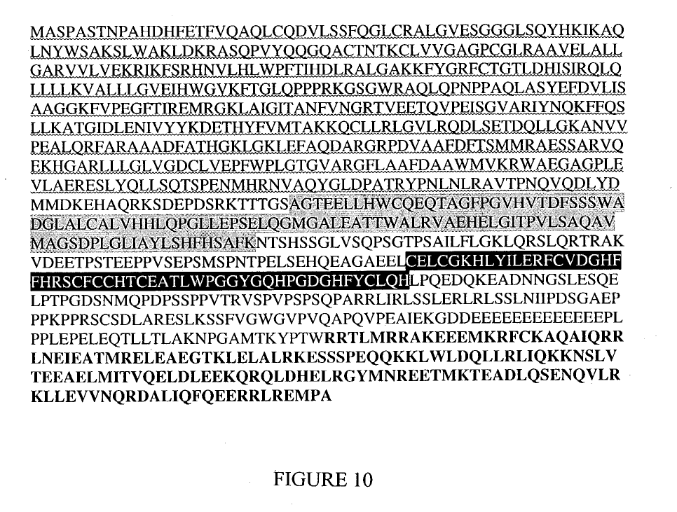

As discussed in more detail herein, the disclosed sequences provided in this specification can be used to identify MICALs or MICAL-Like polypeptides of other species. For example, the sequences disclosed herein were used to search public EST and genomic databases to identify the sequence of mouse MICAL-1 (SEQ ID NO:21) (FIG. 10) (see below). Accordingly, in another aspect, the present invention provides a mouse MICAL polypeptide, such as a polypeptide according to SEQ ID NO:21, SEQ ID NO:22, or SEQ ID NO:23.

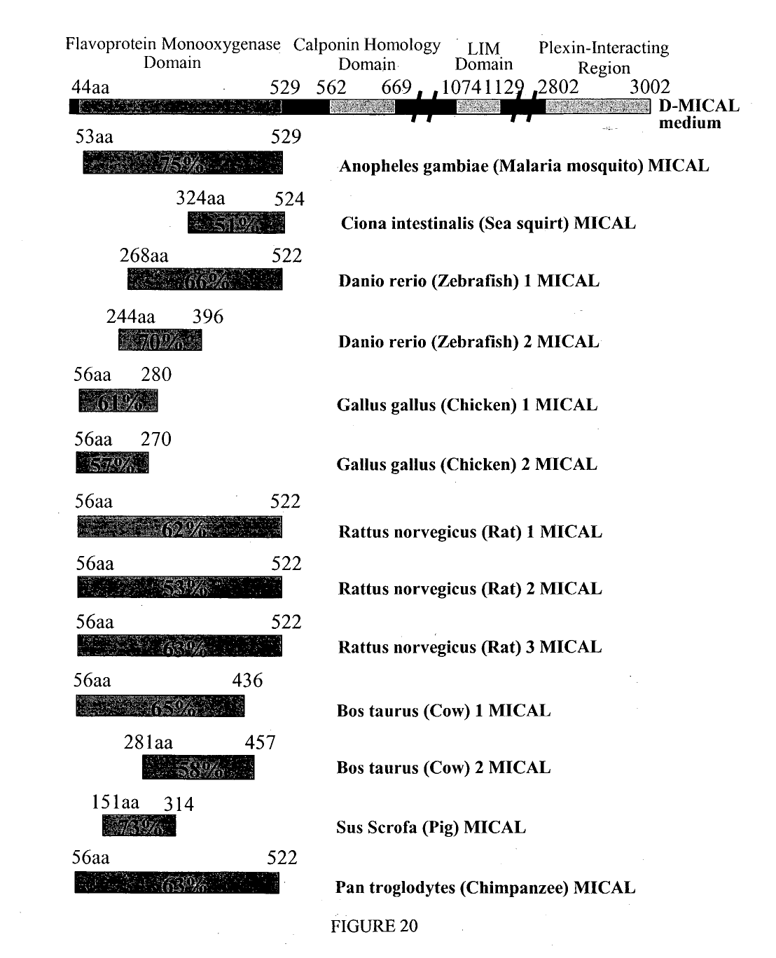

Using the Drosophila MICAL amino acid sequence (or MICALs from human or any other species) the publicly available EST and genomic databases can be searched to identify MICAL proteins in other species. The MICAL N terminal flavoprotein monooxygenase domain identified herein, is extremely highly conserved between MICALs in all species (>50% identity). Furthermore, the N terminal flavoprotein monooxygenase domain (i.e., the N-terminal MICAL domain) is not very similar (<<<20% identity) to anything else in the data base (in the genome) over the MICAL ˜500 amino acid flavoprotein monooxygenase stretch. Therefore, using this domain to search is extremely fast and accurate to find MICAL sequences. The searching strategy can be a standard, routinely used search strategy. The degree of similarity between MICALs in all species is so high that by searching a public database with the Drosophila MICAL flavoprotein monooxygenase amino acid sequence using the publicly available Blast searching program's TBLASTN command one can pull out the coding exons from ordered or unordered (rough draft) genomic sequence. TBLASTN searches a nucleotide database for DNA that is translated by the program into amino acid residues. The alignments can be observed on a web browser. The aligned sequence can then be assembled to identify the coding sequence for the MICAL(s) for any species of interest. An EST nucleotide database can also be searched in a similar manner to a genomic database and as quickly. The sequences provided herein, including the sequences of FIG. 20 and the mouse MICAL sequences (SEQ ID NO:21, SEQ ID NO:22, and SEQ ID NO:23), provide examples of sequences that were obtained in this manner. This strategy would effectively and unequivocally identify all of the raw sequences in the database that code for a MICAL protein.

Furthermore, the rest of the MICAL protein is also highly conserved. Therefore, the other portions of the Drosophila MICAL amino acid sequence and the amino acid sequence from those portions of the flavoprotein monooxygenase domain previously identified for a particular species, can then be used to assemble publicly available sequences to identify the full MICAL protein. This can efficiently be done by searching similar databases and using a similar strategy and piecing together the aligned sequence to get a full MICAL sequence.

In certain aspects, a polypeptide of the present invention is at least 100, 200, 300, 400, 500, 1000, 1500, 2000, 2500, or 3000 amino acids in length.

In another aspect, a polypeptide of the present invention is a functional portion of a MICAL polypeptide. A functional portion of a MICAL polypeptide is a polypeptide that includes at least an N-terminal MICAL domain that retains monooxygenase activity and/or a functional MICAL plexin interacting region.

In another embodiment, the present invention provides an isolated polypeptide that includes an N-terminal MICAL domain, but not one or more of the other domains and regions typically found on a MICAL polypeptide. In one aspect, the polypeptide has an N-terminal MICAL domain but no other domain or region of a MICAL polypeptide. The N-terminal MICAL domain for example, is at least 40%, 50%, 75%, 80%, 90%, 95%, 98%, 99%, or 100% identical to an N-terminal MICAL domain of a naturally-occurring MICAL, such as Drosophila MICAL or human MICAL 1, 2, or 3. The polypeptide typically retains monooxygenase activity and typically includes the consensus dinucleotide binding sequence and three motifs found in flavoprotein monooxygenases disclosed above.

The MICAL polypeptides of the present invention have axon guidance regulatory activity. Axon guidance regulatory activity is the ability to affect the positioning, steering, and/or outgrowth of an axon in vivo or in vitro. Not to be limited by theory, it is believed that MICAL polypeptides of the present invention regulate axon guidance by associating with plexins, thereby being involved in semaphorin-plexin mediated repulsive axon guidance, especially Semaphorin 1a (Sema-1a)-PlexA-mediated repulsive axon guidance, as discussed in more detail hereinbelow.

The examples section herein illustrates several methods that can be used to identify axon guidance regulatory activity, referred to herein as axon guidance regulatory activity assays. For example, where the polypeptide being analyzed, or an ortholog thereof, is encoded for by a Drosophila gene, Drosophila mutants can be generated that are loss of function or gain of function mutants. For example, by deleting all or part of the gene encoding the polypeptide, a loss of function mutant can be generated. If the polypeptide has axon guidance regulatory activity, Drosophila loss of function mutants devoid of the function of the polypeptide should exhibit motor axon guidance defects similar to the distinct and highly penetrant defects seen in Sema1a and PlexA loss of function mutants and seen in the MICAL loss of function mutants discussed in the Examples section.

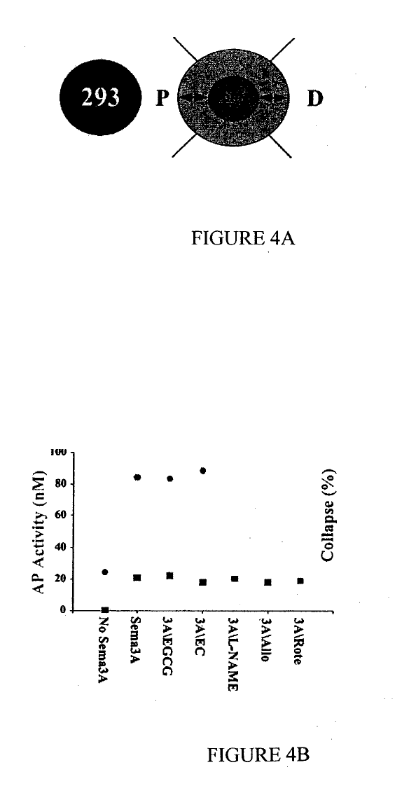

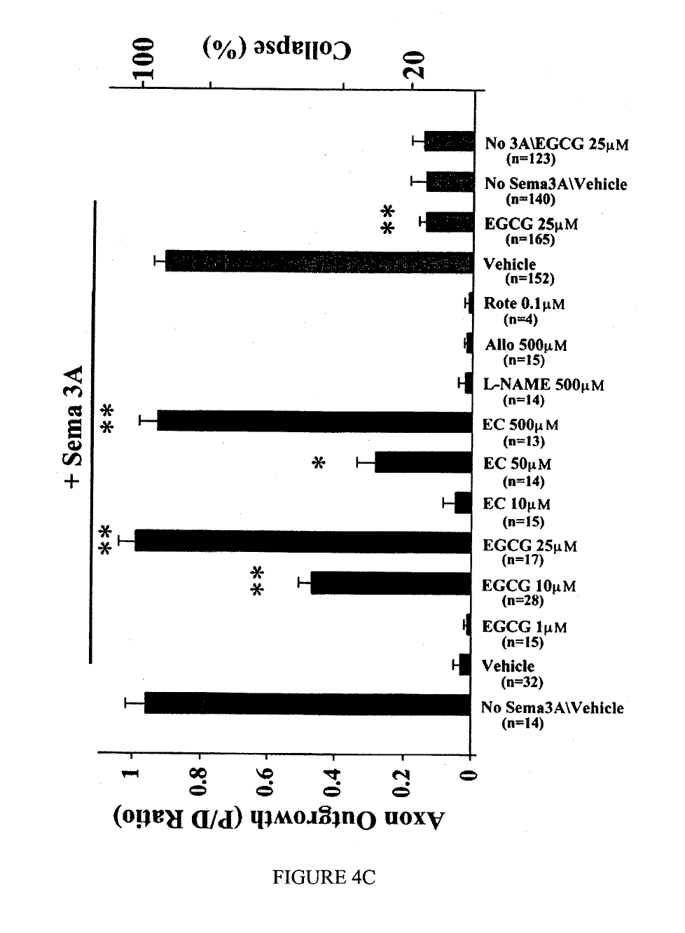

As another example, axon guidance regulatory activity can be identified by employing an in vitro rat DRG growth cone repulsion assay (Messersmith et al., 1995). The method involves co-culturing E14/15 rat DRG explants with 293 or COS cells expressing Sema3A in the presence of an inhibitor of an on-test polypeptide, as illustrated in the Examples section. NGF-dependent DRG axons exhibit little to no outgrowth toward Sema3A-secreting 293 cell aggregates. If the on-test polypeptide has axon guidance activity then inhibitors of the activity of the polypeptide will inhibit axon repulsion (See e.g., FIG. 4). Axon guidance regulatory activity can also be determined, for example, using single cell turning assays as described by Poo et al. (Neuron, 19, 1225-35 (1997)), growth cone collapse assays as described by Raper et al. (See e.g., Luo et al, Cell 75, 217-27 (1993)), and mouse knock-out genetic approaches where phenotypes can be observed that must originate from loss of a repulsive response (based on expression data of the ligand, etc . . . ) (See e.g., Giger et al., Neuron, 25, 29 (2000)).

It will be recognized that strategies used herein to identify MICALs and MICAL-like proteins can be used to identify additional MICAL polypeptides, such as MICAL polypeptides of other mammalian species. For example, as illustrated in the Examples, a yeast 2 hybrid system that uses the terminal highly conserved “C2” portion of the PlexA cytoplasm domain can be used to screen cDNA libraries prepared from any organism. Additionally, MICAL polypeptides can be identified by the ability to rescue mutant organisms, such as mutant Drosophila prepared using methods disclosed herein, which lack MICAL function. Finally, recombinant DNA technologies can be used to identify and/or develop polynucleotides that encode MICAL or MICAL-Like polypeptides, that are related to, but distinct from those disclosed herein as discussed in more detail hereinbelow.

Accordingly, the present invention provides an isolated polypeptide as disclosed above, wherein the isolated polypeptide is at least, for example, 75%, 80%, 85%, 90%, 95%, 97%, 98%, 99%, or 99.9% identical to SEQ ID NO:2, SEQ ID NO:4, SEQ ID NO:6, SEQ ID NO:8, SEQ ID NO:10, or SEQ ID NO:12.

A polypeptide of the present invention can be a MICAL variant, ortholog, isoform, or mutant. Provided herein are sequences of certain human and Drosophila MICALs. However, it will be recognized that additional variants are likely to exist in a population, which have different sequences than the disclosed MICALs. These alleles will likely be highly related in sequence to the disclosed MICALs, and can be further identified as a MICAL by their axonal guidance regulation activity, and by the position in the genome of the polynucleotide sequence encoding the allele. Therefore, based on the presently disclosed MICAL sequences, orthologous sequences in other species besides human or Drosophila, such as rat or mouse, can be identified using methods well known in the art, as illustrated herein. Methods for identifying MICAL polynucleotides of other species are discussed in detail below.

Furthermore, additional MICAL polypeptides and polynucleotides encoding the MICAL polypeptides, can be identified using protein alignment tools, such as those reported in the Examples included herein. As will be recognized, these tools include, for example, PFAM, BLAST, PRINTS, JALVIEW, AND ClustalX, some of which are discussed in more detail herein.

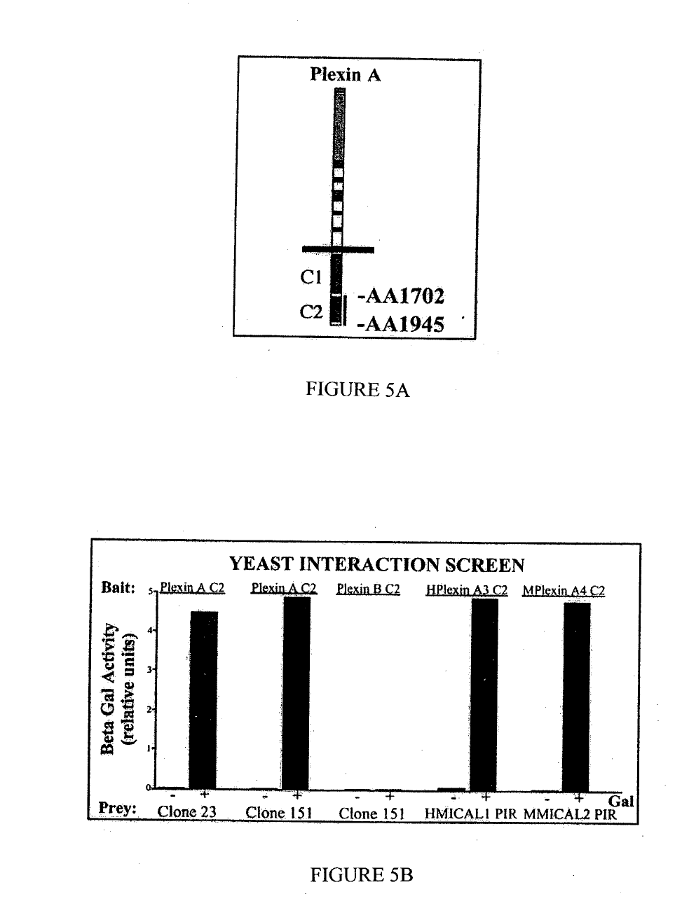

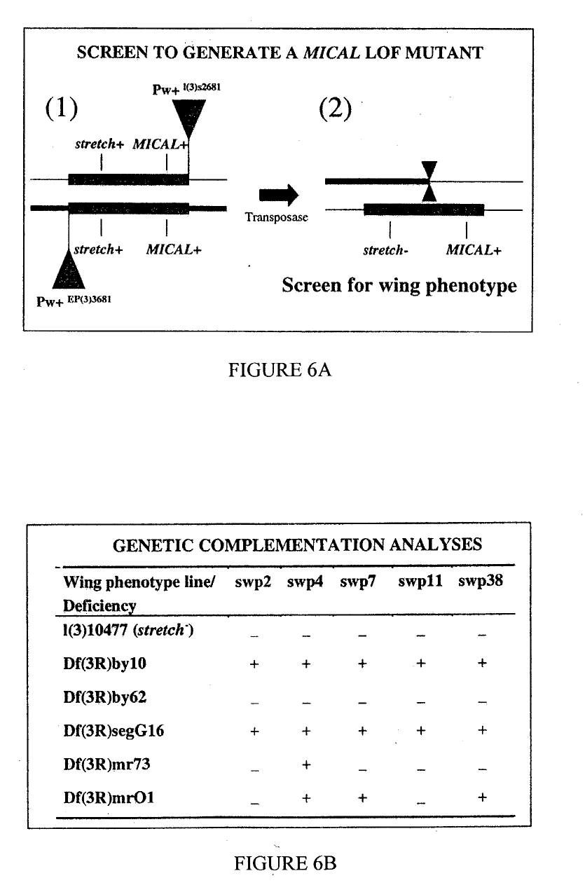

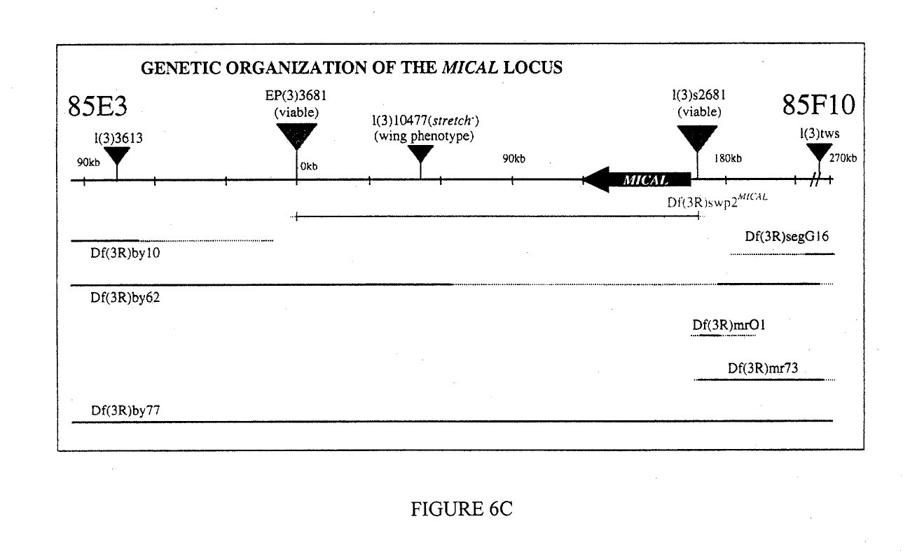

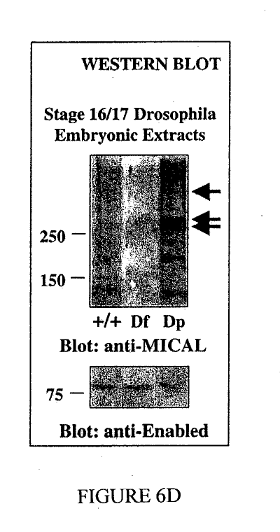

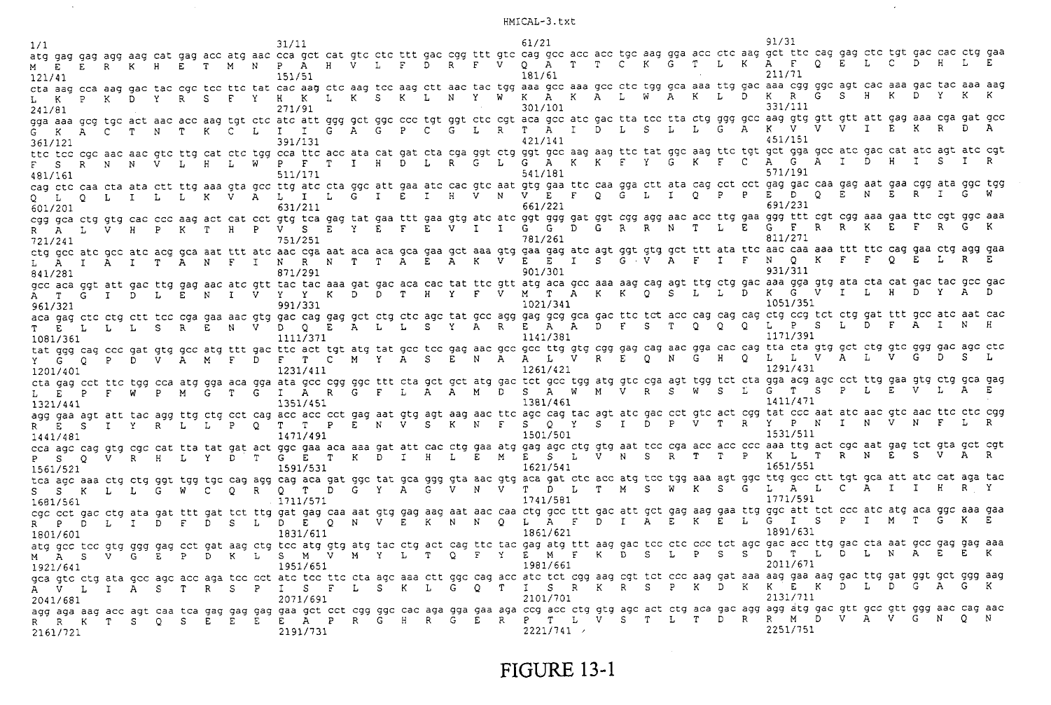

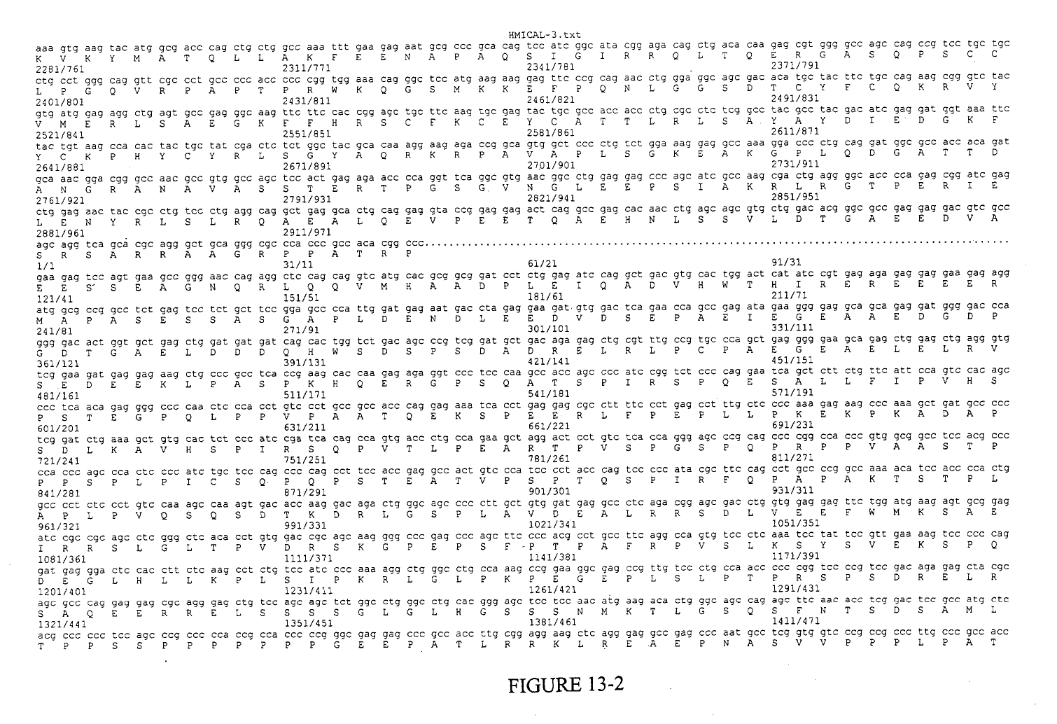

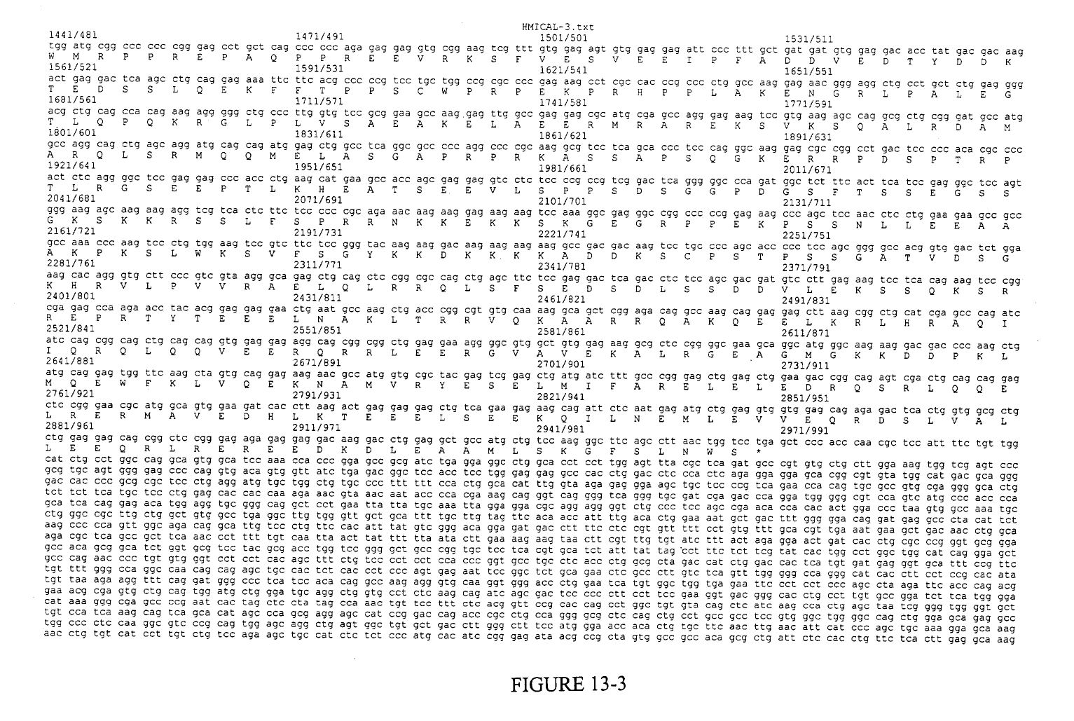

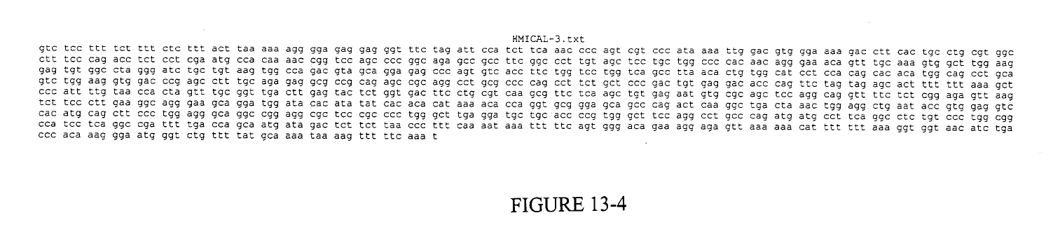

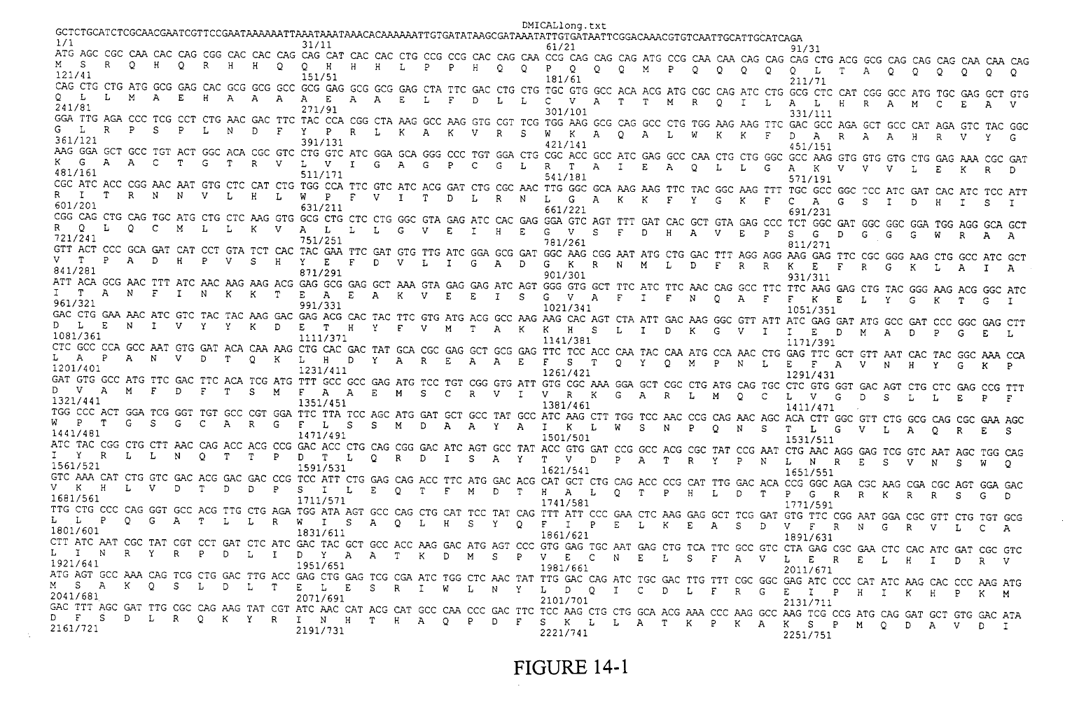

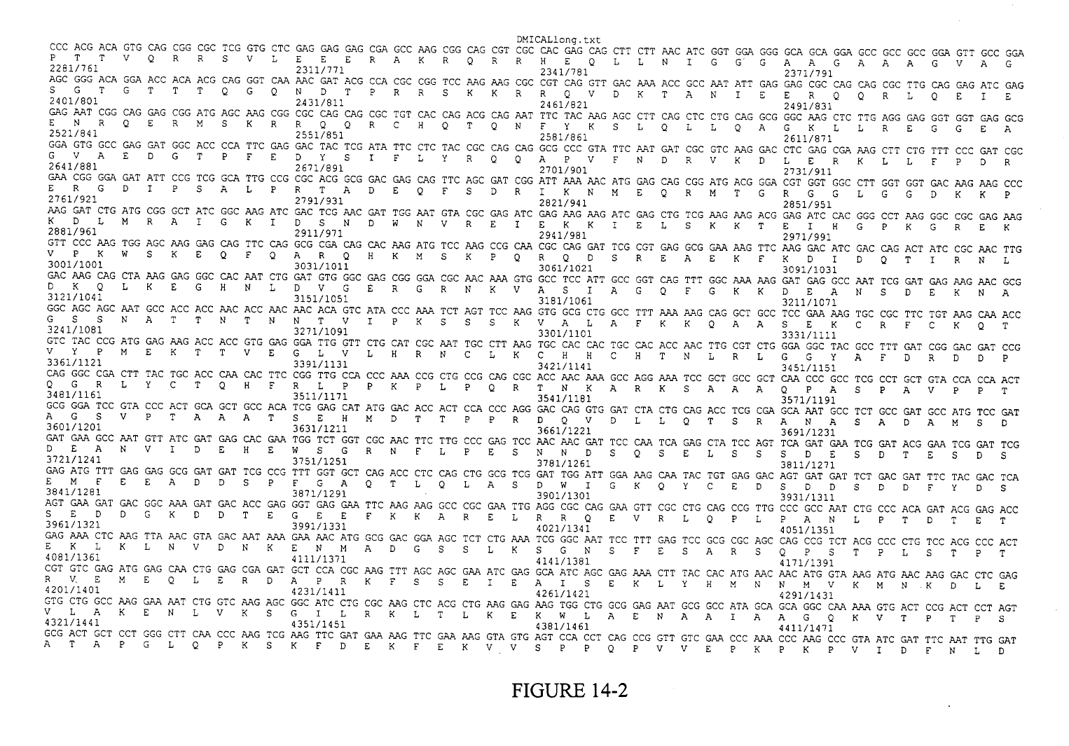

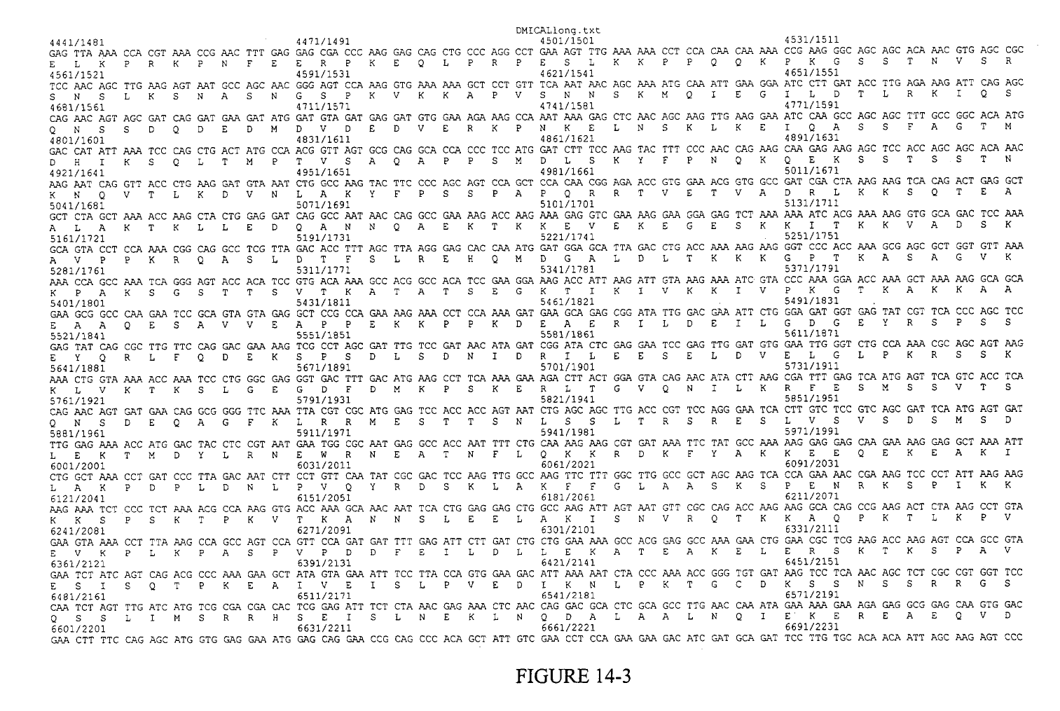

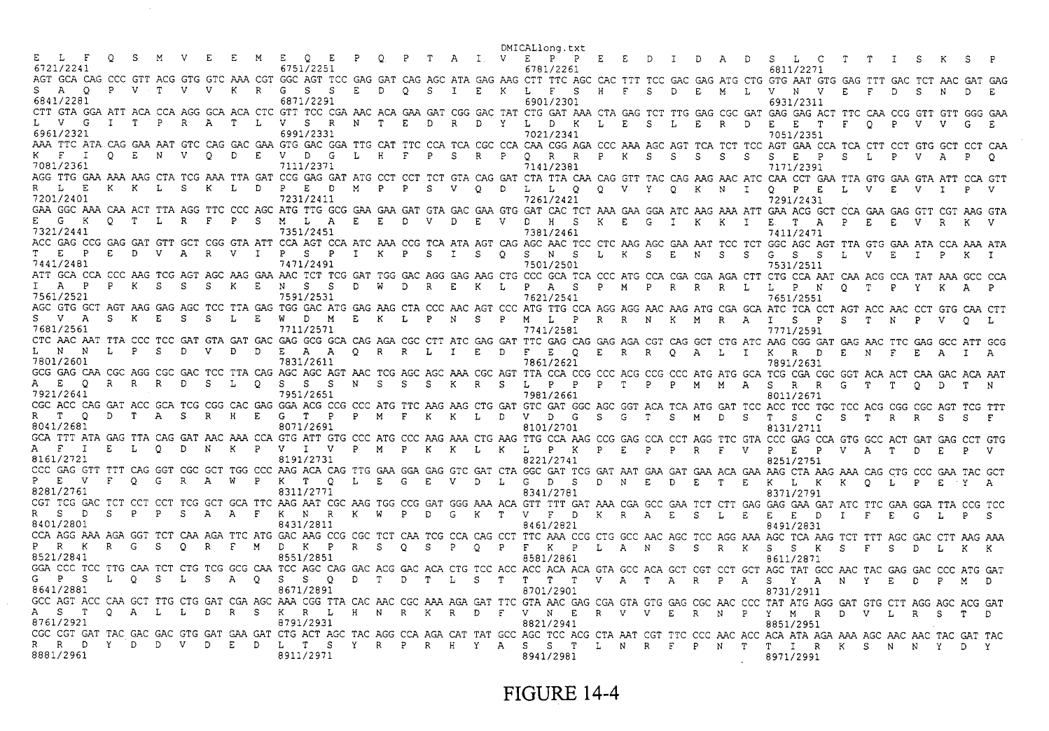

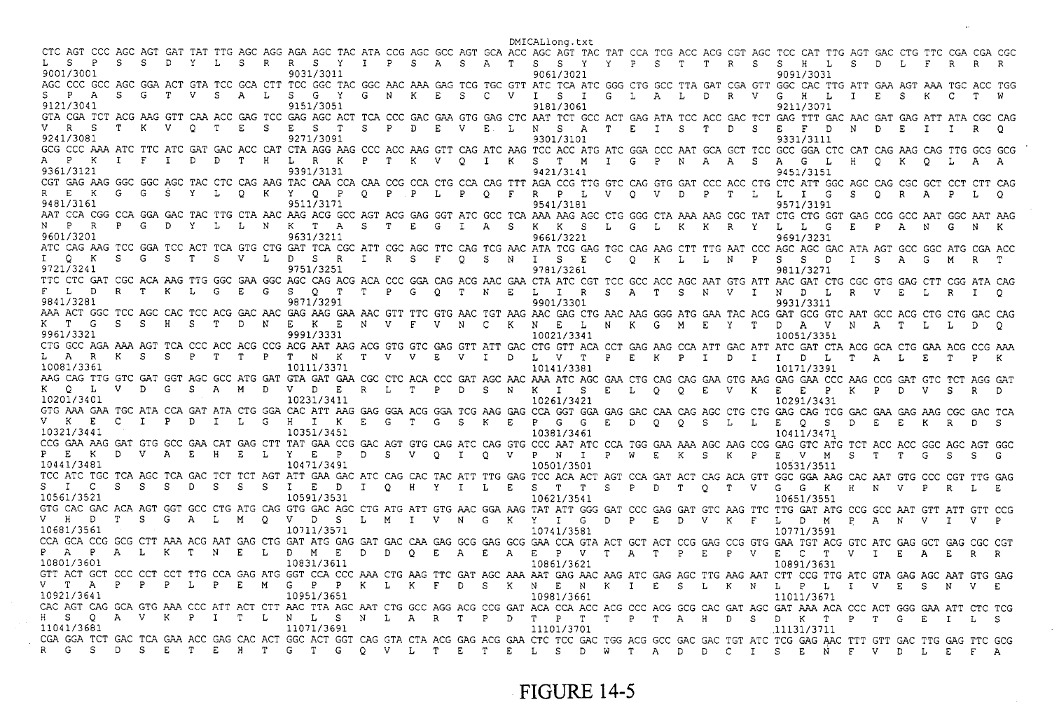

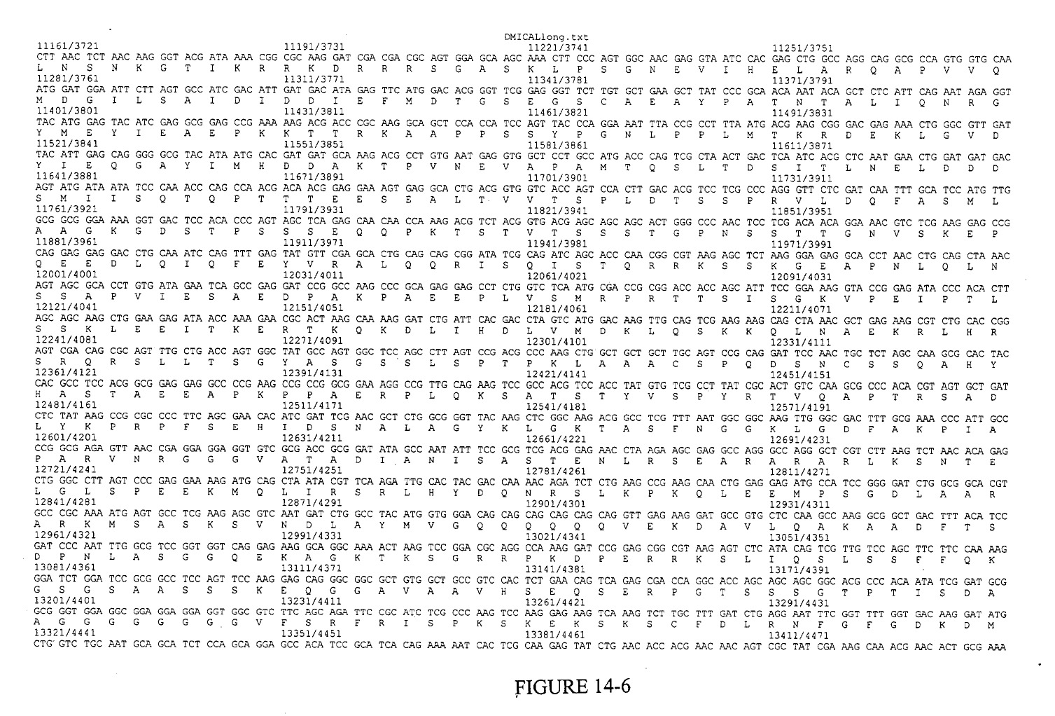

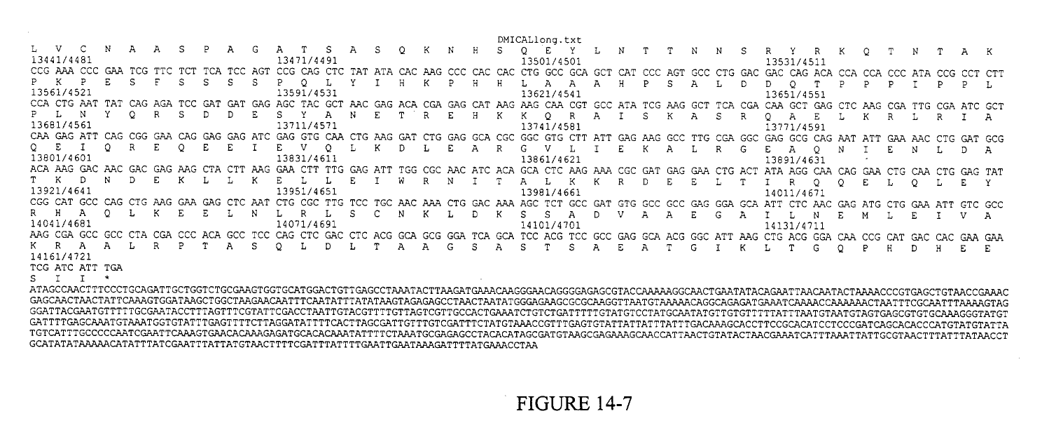

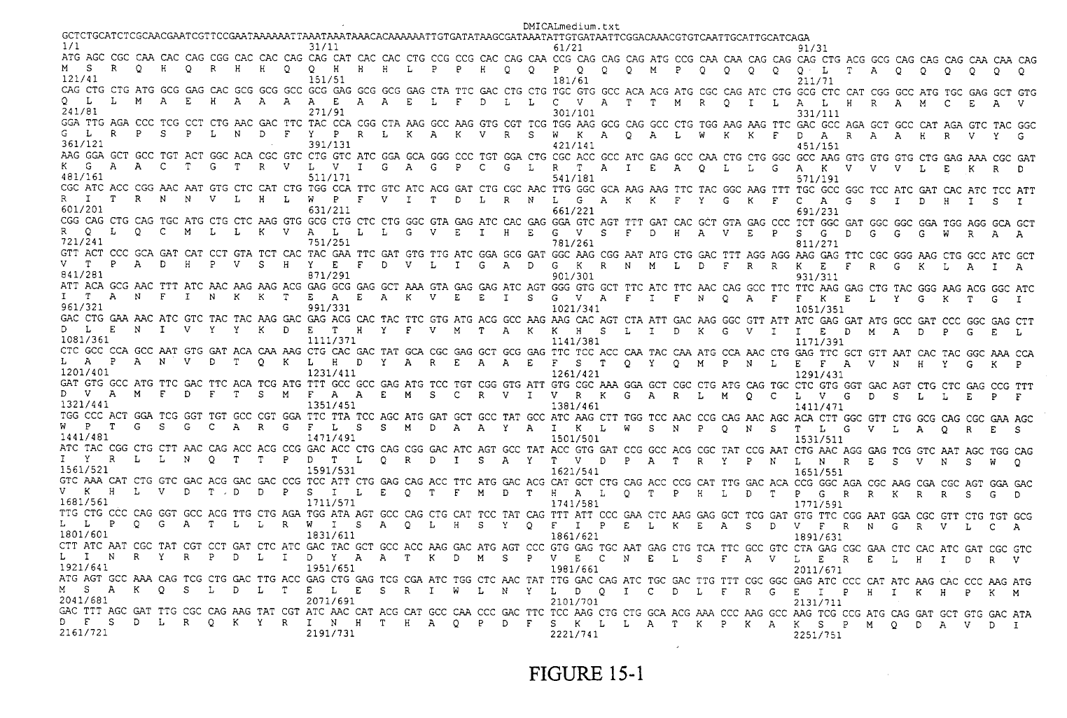

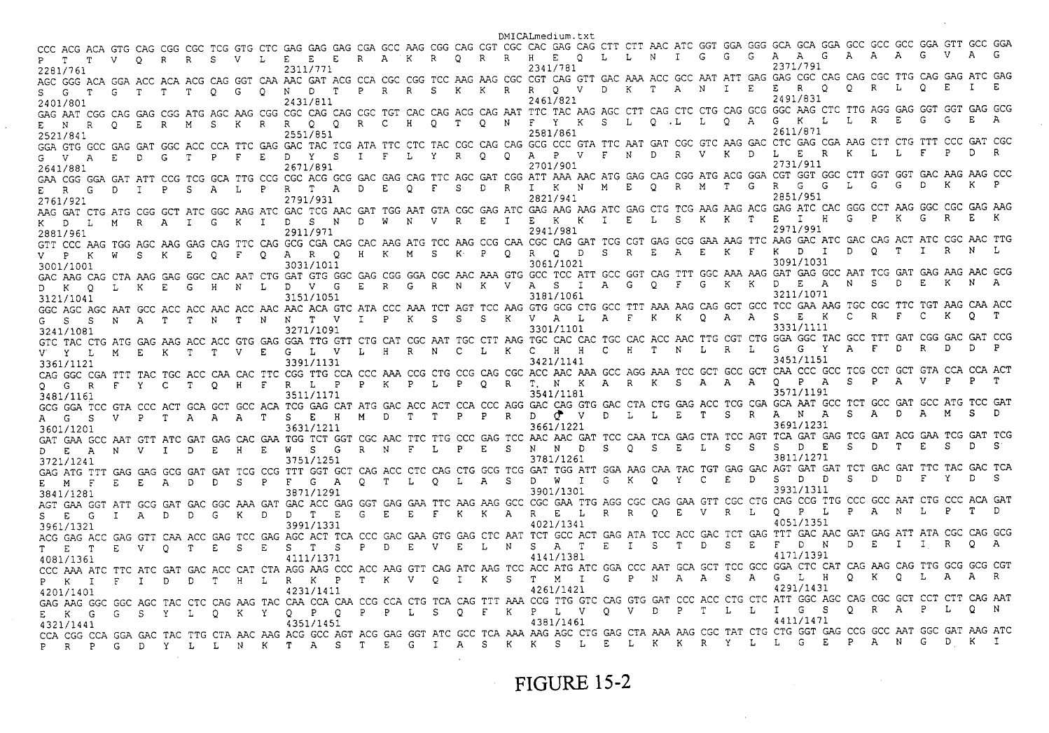

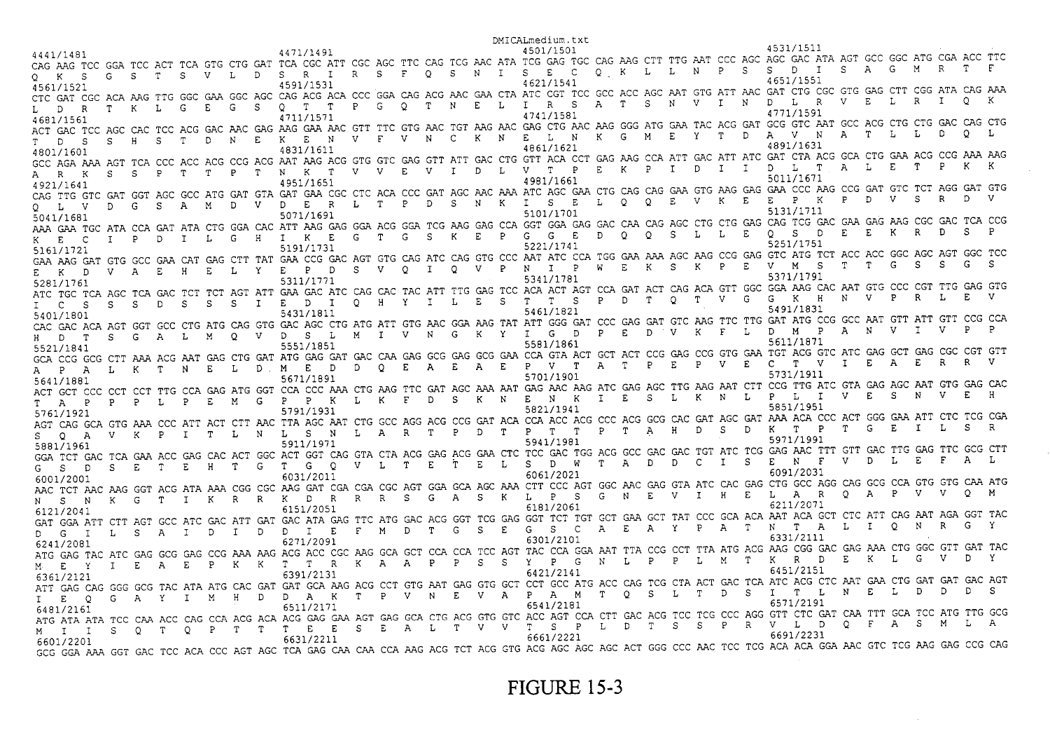

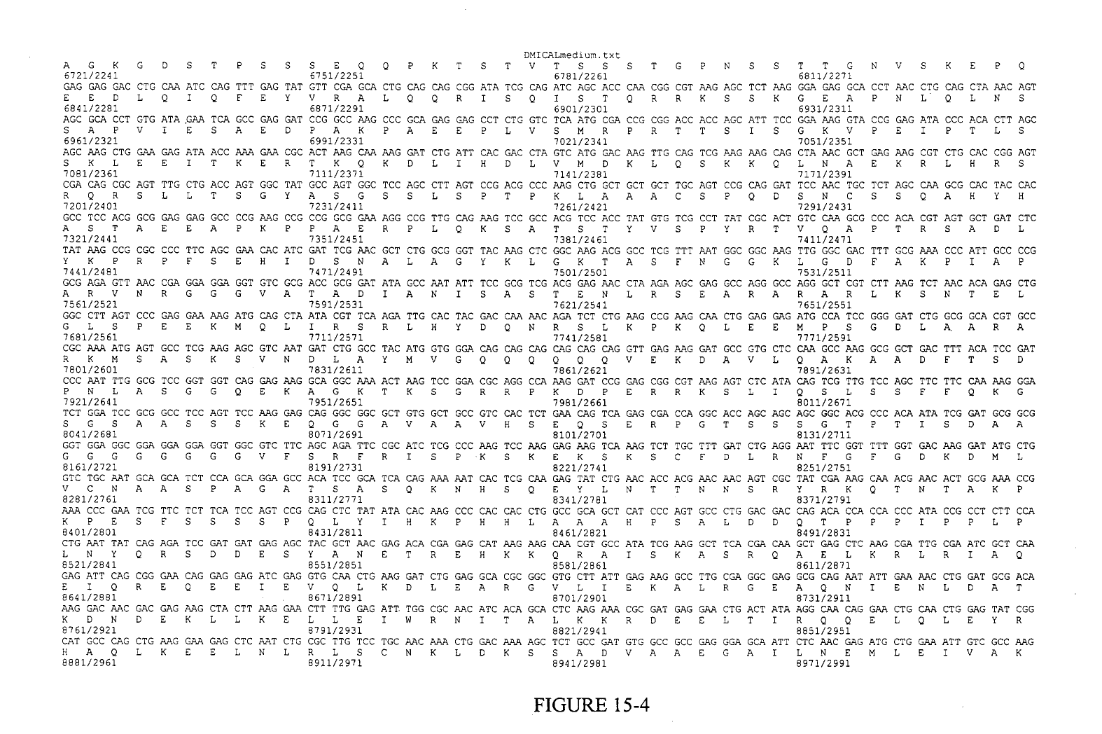

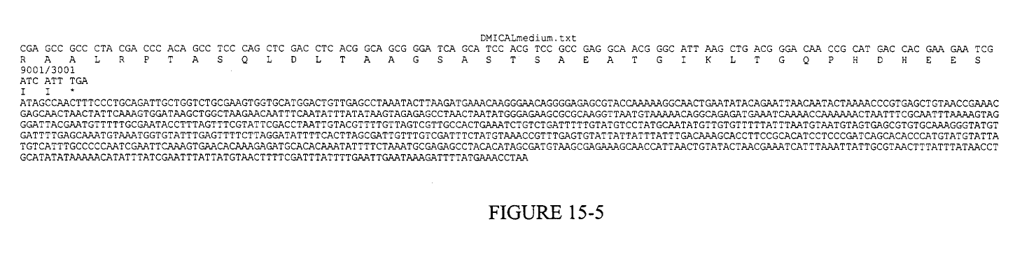

Finally, as disclosed herein, MICAL transcripts in at least some species are alternately spliced so as to give rise to different polypeptide isoforms from the same MICAL gene. These polypeptide variants or isoforms are examples of MICAL polypeptides of the present invention. MICAL genes cover greater than 40 kb of genomic sequences and have at least 25 exons (See FIG. 1A and FIG. 5). Based on analysis of isolated cDNAs and Western analysis (See FIG. 6D), there are at least three Drosophila MICAL isoforms, “long” (See e.g. SEQ ID NO:8, “medium” (SEQ ID NO: 10), and “short” (SEQ ID NO:12) (FIG. 1A and FIGS. 13-15).

In another embodiment, the present invention provides a MICAL consensus polypeptide. A MICAL consensus polypeptide is a polypeptide that includes at least 50%, 75%, 80%, 85%, 90%, 95%, 99%, or 100% of the most prevalent amino acid occurrences of all MICALs, or of MICALs of a species, such as human MICALs. It will be recognized that amino acid sequences of polypeptides of a protein family such as MICALs, can be aligned and an amino acid sequence of a polypeptide with axonal guidance regulatory activity can be identified which is different than all of the naturally-occurring family members, but which includes at least some of the most common amino acid occurrences of the naturally-occurring family members.

Experimental results presented in the Examples section herein demonstrate that at least some MICALs directly associate with plexins and are required for semaphorin-mediated repulsive axon guidance. Furthermore, as disclosed above, MICALs contain multiple domains that are known to be important for interactions with actin, intermediate filaments, and cytoskeletal-associated adaptor proteins. Therefore MICALs are excellent candidates for directly mediating the cytoskeletal alterations characteristic of semaphorin signaling and provide novel targets for the attenuation of axonal repulsion.

During neural development axons reach their appropriate targets by interpreting a myriad of guidance cues present in their environment. Semaphorin proteins, one of the largest families of guidance cues, are known to influence axon pathfinding, fasciculation, branching, and neuronal cell migration (He et al., 2002; Raper, 2000). A chemorepulsive role in axon guidance has been extensively demonstrated both in vitro and in vivo for many semaphorins, but they also mediate attractive neuronal guidance.

The 7 classes of semaphorins include both transmembrane and secreted proteins and are evolutionarily conserved, structurally and in many cases functionally, from invertebrates to vertebrates (Semaphorin Nomenclature Committee, 1999). For example, the transmembrane semaphorin Sema-1a in Drosophila is present on developing motor axons and acts as a repellent to regulate motor axon fasciculation in vivo (Yu et al., 1998). The related vertebrate transmembrane semaphorin Sema6A also functions as a repellent for axons of sympathetic neurons in vitro (Xu et al., 2000). Sema 3A, a well-characterized vertebrate secreted semaphorin, is a potent axonal repellent for a variety of neurons in vitro, and in vivo serves as a chemorepellent essential for the establishment of many axonal pathways (Raper, 2000). Similarly, the related Drosophila secreted semaphorin Sema-2a is expressed on developing muscles and regulates motor axon pathfinding as a target-derived chemorepellent (Matthes et al., 1995; Winberg et al., 1998a).

Insight into how semaphorins signal repulsive guidance comes from work showing that plexins, a large family of evolutionarily conserved transmembrane proteins, serve as signal transducing receptors for both membrane-bound and secreted semaphorins (Tamagnone and Comoglio, 2000). The four classes of plexins have been found to associate directly with members of five different semaphorin classes. In the Drosophila nervous system plexin A (PlexA) is a functional receptor in vivo for Sema la-mediated motor axon repulsion (Winberg et al., 1998b). In vertebrates, repulsion mediated by class 3 secreted semaphorins is dependent on plexin function both in vitro and in vivo (Cheng et al., 0.2001; reviewed in Tamagnone and Comoglio, 2000). However, repulsive guidance mediated by class 3 semaphorins, including Sema3A and Sema3F, requires a holoreceptor complex which includes a ligand-binding obligate co-receptor, neuropilin-1 or neuropilin-2, and a class A plexin. Plexin cytoplasmic domains are highly conserved and, for certain A class plexins, are responsible for signaling semaphorin-mediated repulsive axon guidance (Cheng et al., 2001; Takahashi and Strittmatter, 2001).

The repulsive nature of semaphorin signaling mediated by plexin receptors is due to the modification of the growth cone cytoskeleton. For example, following exposure to secreted Sema3A, growth cones undergo rapid collapse which is accompanied by the depolymerization of F-actin and decreased ability to polymerize new F-actin (Fan et al., 1993). Several modulators of cytoskeletal dynamics have been implicated in this process including Rho family GTPases, p21-activated kinase (PAK), and LIM kinase (Liu and Strittmatter, 2001; Whitford and Ghosh, 2001). In addition, members of the collapsin response mediator protein (CRMP) family, the Ig superfamily protein LI, intracellular levels of cGMP, and the catalytically inactive receptor tyrosine kinase family member offtrack (OTK), have also been implicated in transducing semaphorin repulsive guidance (He et al., 2002). It remains unknown, however, how plexins directly regulate the activity of these signaling molecules in order to modulate cytoskeletal dynamics.

In another embodiment, the present invention provides an isolated polypeptide that includes a plexin interacting region. The plexin interacting region is typically at least 40%, 50%, 75%, 80%, 90%, 95%, 98%, 99%, or 100% identical to a plexin interacting region of a naturally-occurring MICAL, such as Drosophila MICAL or human MICAL 1, 2, or 3, and retains the ability to interact with a plexin. The plexin interacting region for example, can be at least 90% identical to a plexin interacting region of Drosophila MICAL (SEQ ID NO:8, SEQ ID NO:10, or SEQ ID NO:12), or to a plexin interacting region of human MICAL 1 (SEQ ID NO:2), human MICAL 2 (SEQ ID NO:4), human MICAL 3 (SEQ ID NO:6), or a conservative variant thereof. A conservative variant is a polypeptide that is identical to another polypeptide except for conservative amino acid substitutions, as discussed hereinbelow.

A polypeptide of this embodiment of the invention typically retains the ability to specifically interact with all or part of a plexin either directly or indirectly. For example, the isolated polypeptide can directly interact with the C2 domains of a PlexA, such as PlexA3 and PlexA4. As disclosed in the Examples herein, human MICAL-1 and mouse MICAL-2 specifically interact with the C2 domains of human PlexA3 and mouse PlexA4, respectively. Indirect interactions can be identified, for example, using genetic approaches illustrated in the Examples herein.

Methods for determining whether an on-test polypeptide is capable of interacting with a plexin are well-known in the art. For example, traditional methods of identifying specific protein interactions can be used. Accordingly, immunoprecipitation can be used to identify whether a polypeptide interacts with a plexin by determining whether the on-test polypeptide and a plexin coimmunoprecipitate, as illustrated in the Examples section. Furthermore, for example, a plexin protein can be isolated on a protein gel, and binding of a labeled on-test polypeptide can be determined. Alternatively, for example, a yeast interaction assay can be used, as disclosed in the Examples herein (See FIG. 5).

The isolated polypeptide that includes a plexin interacting region, in certain aspects, does not have one or more other domains and/or activities typically present in a MICAL polypeptide. For example, the polypeptide in certain aspects does not have monooxygenase activity. The polypeptide of this embodiment of the invention can be a mutant MICAL polypeptide that acts as a dominant negative mutant with respect to MICAL activity. For example, as illustrated in the Examples section, the polypeptide can be a truncated MICAL polypeptide that includes at least one, but not all, functional domains typically present on a MICAL polypeptide. Alternatively, the mutant can include mutations that alter, for example by destroying, certain MICAL functions.

The examples section provided herein provides a MICAL^{G→W} mutant (SEQ ID NO:20) that is mutated in the three glycine residues within the FAD fingerprint 1 motif of MICAL to tryptophan, a mutation known in other proteins to disrupt FAD binding without altering the overall structure of the protein (Kubo et al., 1997; Lawton and Philpot, 1993; Wierenga et al., 1986). As illustrated in the Examples section, the MICAL^{G→W} mutant which includes an intact plexin-interacting domain but is functionally inactive, exerts a dominant-negative effect on motor axon guidance in a wild-type genetic background. Not to be limited by theory, it is believed that MICAL^{G→W} exerts its dominant negative effect by binding to a plexin, thereby competing for binding of wild-type MICAL to the plexin target.

As illustrated in the Examples herein, a polypeptide according to this embodiment of the invention can be a truncated mutant that only includes a plexin-interacting region. Accordingly, the polypeptide can have an amino acid sequence as set forth in SEQ ID NO: 19. The polypeptide can be targeted to the membrane by including a membrane targeting sequence such as an N-terminal myristoylation sequence as illustrated in the Examples section herein (see mutant MICAL^{Myr→CT}). Other membrane targeting sequences such as, for example, a palmitylation sequence, can be used.

A polypeptide of this embodiment of the invention, can include, for example, a MICAL or a MICAL-Like plexin interacting domain, since both of these protein families include a plexin interacting domain.

In another embodiment the present invention relates to a family of MICAL-like (MICAL-L) proteins, members of which have a similar organization to MICALs but lack the region N-terminal to the CH domain (FIG. 2B). MICAL-L proteins include at least one MICAL-L protein in Drosophila (D-MICAL-L) and at least two family members in humans. D-MICAL-L cDNA and genomic DNA sequence information suggest that D-MICAL-L Plexin interacting domain begins just N-terminal to the CH domain of a MICAL protein. Analysis of publicly available mammalian cDNA and genomic sequences suggests that human MICAL-L1 and MICAL-L2 are similar in overall domain organization to D-MICAL-L and do not contain the highly conserved ˜500 amino acid MICAL N-terminal domain.

Accordingly, the present invention provides an isolated polypeptide that includes a plexin interacting region and alternatively one or more of a calponin homology domain, a LIM domain, and a proline rich region. The polypeptide can also include a first variable region and a second variable region. Accordingly, in one aspect, the polypeptide is or includes a MICAL-like polypeptide. A MICAL-Like polypeptide includes, from N-terminal to C-terminal, a calponin homology domain, a first variable MICAL region, a LIM domain, a second variable MICAL region, a proline rich region, and a plexin interacting region.

A polypeptide according to this aspect of the invention typically specifically interacts with a plexin, as discussed above for MICAL polypeptides of the present invention.

The polypeptide, for example, includes a calponin homology domain, followed by a first variable region, followed by a LIM domain, followed by a second variable region, followed by a proline rich region, and followed by a plexin interacting region. Such polypeptides include a variant, ortholog, isoform, or mutant of a MICAL-Like protein disclosed herein.

In one aspect, the polypeptide is a mammalian MICAL-Like polypeptide. For example, the isolated polypeptide can be human MICAL-Like 1 or human MICAL-Like 2. Accordingly, the isolated polypeptide can have an amino acid sequence that is at least 40%, 50%, 75%, 80%, 90%, 95%, 98%, 99%, or 100% identical to a an amino acid sequence as set forth in SEQ ID NO: 14 (human MICAL-Like 1) or SEQ ID NO: 16 (human MICAL-like 2).

In another aspect, the isolated polypeptide is a Drosophila MICAL-L polypeptide. For example, the polypeptide can have an amino acid sequence as set forth in SEQ ID NO: 18 (Drosophila MICAL-Like), or a variant, ortholog, isoform, or mutant thereof.

In another aspect, the polypeptide of the present invention is a functional portion of a MICAL-Like polypeptide. A functional portion of a MICAL polypeptide is a polypeptide that includes at least a functional domain, for example a functional MICAL plexin interacting region.

A functional peptide portion of a MICAL or MICAL-Like polypeptide for example, can be obtained by examining peptide portions of a MICAL or MICAL-Like polypeptide using methods as provided herein or other standard methods, to identify fragments that retain at least one of the activities of a wild-type MICAL including the ability to interact with a plexin, particularly Plexin A, and monooxygenase activity.

A functional peptide portion of a MICAL or MICAL-Like polypeptide that specifically interacts with plexin can be identified using any of various assays known to be useful for identifying specific protein-protein interactions. Such assays include, for example, methods of gel electrophoresis, affinity chromatography, the two hybrid system of Fields and Song (Nature 340:245-246, 1989; see, also, U.S. Pat. No. 5,283,173; Fearon et al., Proc. Natl. Acad. Sci., USA 89:7958-7962, 1992; Chien et al., Proc. Natl. Acad. Sci. USA 88:9578-9582, 1991; Young, Biol. Reprod. 58:302-311(1998), each of which is incorporated herein by reference), the reverse two hybrid assay (Leanna and Hannink, Nucl. Acids Res. 24:3341-3347, 1996, which is incorporated herein by reference), the repressed transactivator system (U.S. Pat. No. 5,885,779, which is incorporated herein by reference), the phage display system (Lowman, Ann. Rev. Biophys. Biomol. Struct. 26:401-424, 1997, which is incorporated herein by reference), GST/HIS pull down assays, mutant operators (WO 98/01879, which is incorporated herein by reference), the protein recruitment system (U.S. Pat. No. 5,776,689, which is incorporated herein by reference), and the like (see, for example, Mathis, Clin. Chem. 41:139-147, 1995 Lam, Anticancer Drug Res. 12:145-167, 1997; Phizicky et al., Microbiol. Rev. 59:94-123, 1995; each of which is incorporated herein by reference).

A functional peptide portion of a MICAL or MICAL-Like polypeptide also can be identified using methods of molecular modeling. For example, an amino acid sequence of a MICAL or MICAL-Like polypeptide can be entered into a computer system having appropriate modeling software, and a three dimensional representation of the MICAL or MICAL-Like polypeptide (“virtual MICAL” or “virtual MICAL-Like polypeptide”) can be produced. A MICAL or MICAL-Like polypeptide amino acid sequence also can be entered into the computer system, such that the modeling software can simulate portions of the MICAL or MICAL-Like polypeptide sequence, and can identify those peptide portions that can interact specifically, for example, with the virtual plexin.

It should be recognized that such methods, including two hybrid assays and molecular modeling methods, also can be used to identify other specifically interacting molecules encompassed within the present invention. For example, the methods can be used to identify other proteins to which MICALS and/or MICAL-Like proteins bind, as revealed by the various domains of these proteins.

Modeling systems useful for the purposes disclosed herein can be based on structural information obtained, for example, by crystallographic analysis or nuclear magnetic resonance analysis, or on primary sequence information (see, for example, Dunbrack et al., “Meeting review: the Second meeting on the Critical Assessment of Techniques for Protein Structure Prediction (CASP2) (Asilomar, Calif., Dec. 13-16, 1996). Fold Des. 2(2): R27-42, (1997); Fischer and Eisenberg, Protein Sci. 5:947-55, 1996; (see, also, U.S. Pat. No. 5,436,850); Havel, Prog. Biophys. Mol. Biol. 56:43-78, 1991; Lichtarge et al., J. Mol. Biol. 274:325-37, 1997; Matsumoto et al., J. Biol. Chem. 270:19524-31, 1995; Sali et al., J. Biol. Chem. 268:9023-34, 1993; Sali, Molec. Med. Today 1:270-7, 1995a; Sali, Curr. Opin. Biotechnol. 6:437-51, 1995b; Sali et al., Proteins 23: 318-26, 1995c; Sali, Nature Struct. Biol. 5:1029-1032, 1998; U.S. Pat. No. 5,933,819; U.S. Pat. No. 5,265,030, each of which is incorporated herein by reference).

The crystal structure coordinates of a MICAL or MICAL-Like polypeptide can be used to design compounds that bind to the protein and alter its physical or physiological properties in a variety of ways. The structure coordinates of the protein can also be used to computationally screen small molecule databases for agents that bind to the polypeptide to develop modulating or binding agents, which can act as agonists or antagonists of MICAL axon guidance regulatory activity. Such agents can be identified by computer fitting kinetic data using standard equations (see, for example, Segel, “Enzyme Kinetics” (J. Wiley & Sons 1975), which is incorporated herein by reference).

Methods of using crystal structure data to design inhibitors or binding agents are known in the art. For example, MICAL or MICAL-Like polypeptide coordinates can be superimposed onto other available coordinates of similar proteins, including proteins having a bound inhibitor, to provide an approximation of the way the inhibitor interacts with the receptor. Computer programs employed in the practice of rational drug design also can be used to identify compounds that reproduce interaction characteristics similar to those found, for example, between a MICAL or MICAL-Like polypeptide and a co-crystallized plexin. Detailed knowledge of the nature of the specific interactions allows for the modification of compounds to alter or improve solubility, pharmacokinetics, and the like, without affecting binding activity.

Computer programs for carrying out the activities necessary to design agents using crystal structure information are well known. Examples of such programs include, Catalyst Databases™—an information retrieval program accessing chemical databases such as BioByte Master File, Derwent WDI and ACD; Catalyst/HYPO™—generates models of compounds and hypotheses to explain variations of activity with the structure of drug candidates; Ludi™—fits molecules into the active site of a protein by identifying and matching complementary polar and hydrophobic groups; and Leapfrog™—“grows” new ligands using a genetic algorithm with parameters under the control of the user.

Various general purpose machines can be used with such programs, or it may be more convenient to construct more specialized apparatus to perform the operations. Generally, the embodiment is implemented in one or more computer programs executing on programmable systems each comprising at least one processor, at least one data storage system (including volatile and non-volatile memory and/or storage elements), at least one input device, and at least one output device. The program is executed on the processor to perform the functions described herein.

Each such program can be implemented in any desired computer language, including, for example, machine, assembly, high level procedural, or object oriented programming languages, to communicate with a computer system. In any case, the language may be a compiled or interpreted language. The computer program will typically be stored on a storage media or device, for example, a ROM, CD-ROM, magnetic or optical media, or the like, that is readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein.

Embodiments of the invention include systems, for example, internet based systems, particularly computer systems which store and manipulate coordinate information obtained by crystallographic or NMR analysis, or amino acid or nucleotide sequence information, as disclosed herein. As used herein, the term “computer system” refers to the hardware components, software components, and data storage components used to analyze coordinates or sequences as set forth herein. The computer system typically includes a processor for processing, accessing and manipulating the sequence data. The processor can be any well known type of central processing unit, for example, a Pentium II or Pentium III processor from Intel Corporation, or a similar processor from Sun, Motorola, Compaq, Advanced MicroDevices or International Business Machines.

Typically the computer system is a general purpose system that comprises the processor and one or more internal data storage components for storing data, and one or more data retrieving devices for retrieving the data stored on the data storage components. A skilled artisan can readily appreciate that any one of the currently available computer systems are suitable.

Where it is desired to identify a chemical entity that interacts specifically with MICAL or MICAL-Like polypeptide, any of several methods to screen chemical entities or fragments for their ability to interact specifically with the molecule can be used. This process may begin by visual inspection, for example, of MICAL or MICAL-Like polypeptide on the computer screen. Selected peptide portions of MICAL or MICAL-Like polypeptides, or chemical entities that can act as mimics, then can be positioned in a variety of orientations, or docked, within an individual binding site of the MICAL or MICAL-Like polypeptides. Docking can be accomplished using software such as Quanta and Sybyl, followed by energy minimization and molecular dynamics with standard molecular mechanics forcefields, such as CHARM and AMBER.

Specialized computer programs can be particularly useful for selecting peptide portions of a prodomain, or chemical entities useful, for example, as a MICAL or MICAL-Like polypeptide agonist or antagonist. Such programs include, for example, GRID (Goodford, J. Med. Chem., 28:849-857,1985; available from Oxford University, Oxford, UK); MCSS (Miranker and Karplus, Proteins: Structure. Function and Genetics 11:29-34, 1991, available from Molecular Simulations, Burlington Mass.); AUTODOCK (Goodsell and Olsen, Proteins: Structure. Function, and Genetics 8:195-202, 1990, available from Scripps Research Institute, La Jolla Calif.); DOCK (Kuntz, et al., J. Mol. Biol. 161:269-288, 1982, available from University of California, San Francisco Calif.), each of which is incorporated herein by reference.

Suitable peptides or agents that have been selected can be assembled into a single compound or binding agent. Assembly can be performed by visual inspection of the relationship of the fragments to each other on the three-dimensional image displayed on a computer screen, followed by manual model building using software such as Quanta or Sybyl. Useful programs to aid one of skill in the art in connecting the individual chemical entities or fragments include, for example, CAVEAT (Bartlett et al, Special Pub., Royal Chem. Soc. 78:182-196, 1989, available from the University of California, Berkeley Calif.); 3D Database systems such as MACCS-3D (MDL Information Systems, San Leandro Calif.; for review, see Martin, J. Med. Chem. 35:2145-2154, 1992); HOOK (available from Molecular Simulations, Burlington, Mass.), each of which is incorporated herein by reference.

In another embodiment, the present invention provides an isolated polynucleotide encoding a polypeptide of the present invention disclosed hereinabove. Accordingly, a polynucleotide of the present invention can encode a MICAL polypeptide or a MICAL-like polypeptide of the present invention. A polynucleotide of the present invention that encodes a MICAL polypeptide encodes an isolated polypeptide that includes one or more of an N-terminal MICAL domain, a calponin homology domain, a LIM domain, a proline rich region, and a plexin interacting region, wherein the polypeptide has monooxygenase activity, plexin interacting activity, and/or axon guidance regulatory activity. The encoded polypeptide can also include a first variable region and a second variable region surrounding the LIM domain.

A polynucleotide of the present invention that encodes a MICAL-Like polypeptide encodes an isolated polypeptide that includes one or more of a calponin homology domain, a LIM domain, a proline rich region, and a plexin interacting region, wherein the polypeptide has plexin interacting activity. The encoded polypeptide can also include a first variable region and a second variable region surrounding the LIM domain.

In one aspect the polynucleotide encodes a mammalian MICAL polypeptide, or a functional portion thereof, or MICAL-like polypeptide, or a functional portion thereof. For example, the polynucleotide can encode all or a portion of human MICAL-1, human MICAL-2, human MICAL-3, human MICAL-Like 1, or human MICAL-like 2. As such the polynucleotide can include all or a portion (e.g. a cDNA, or a coding region) of a human MICAL-1 gene, human MICAL-2 gene, human MICAL-3 gene, human MICAL-Like 1 gene, or human MICAL-like 2 gene. The polynucleotide, for example, can include a coding region or an entire transcript. Accordingly, the polynucleotide can encode a polypeptide that includes an amino acid sequence as set forth in SEQ ID NO:2 (human MICAL-1), SEQ ID NO:4 (human MICAL-2), SEQ ID NO:6 (human MICAL-3), SEQ ID NO: 14 (human MICAL Like 1), or SEQ ID NO:16 (human MICAL-Like 2), or an isoform thereof.

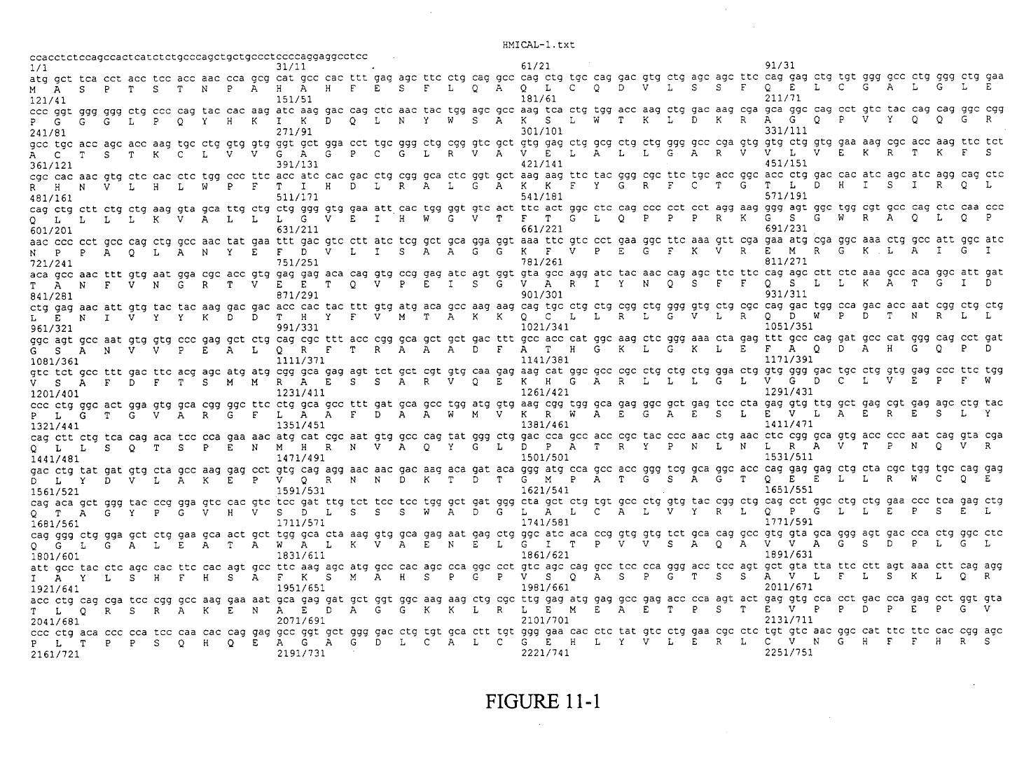

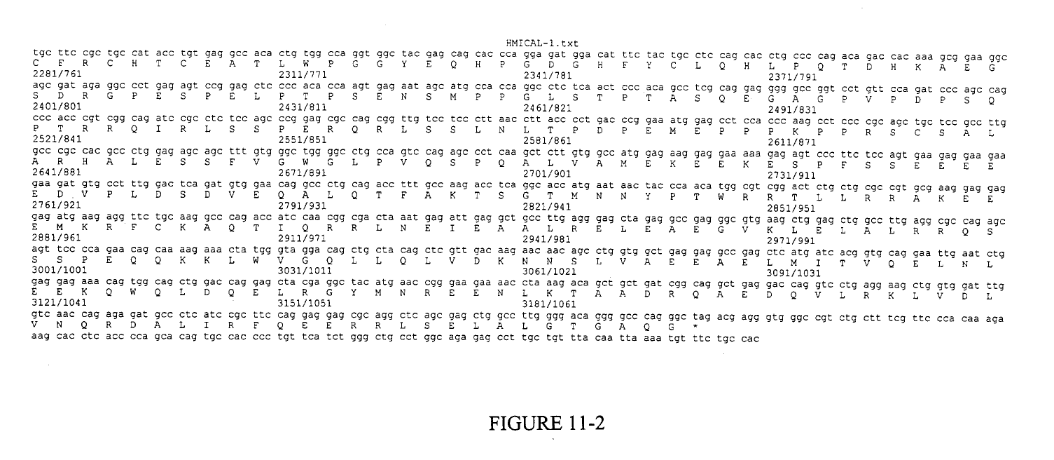

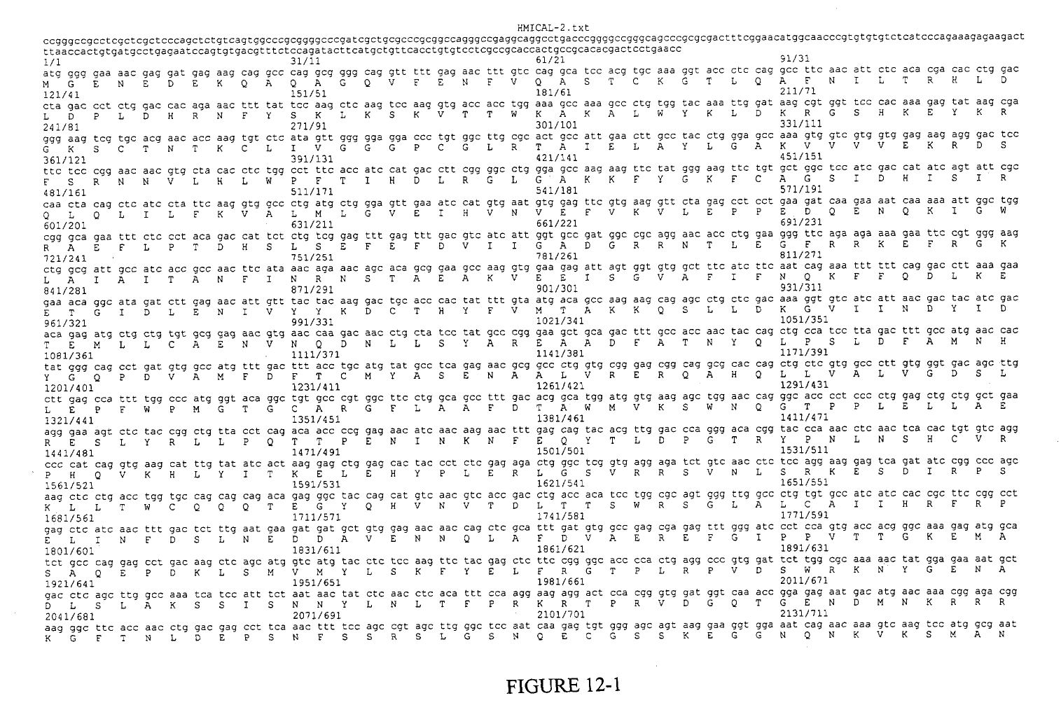

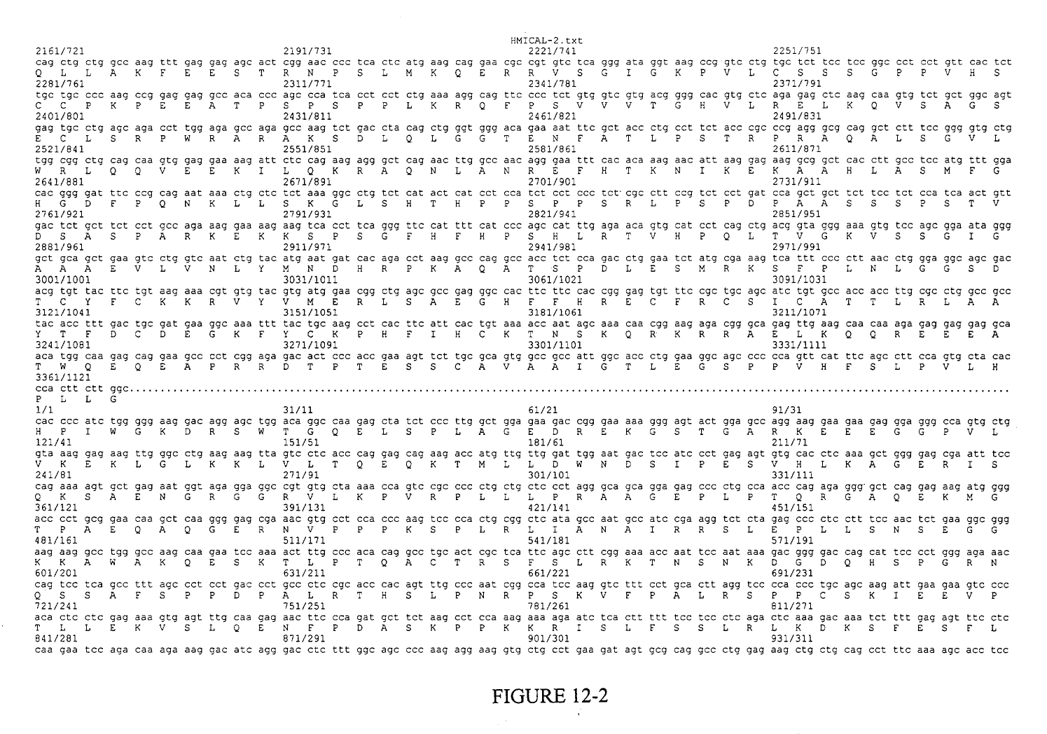

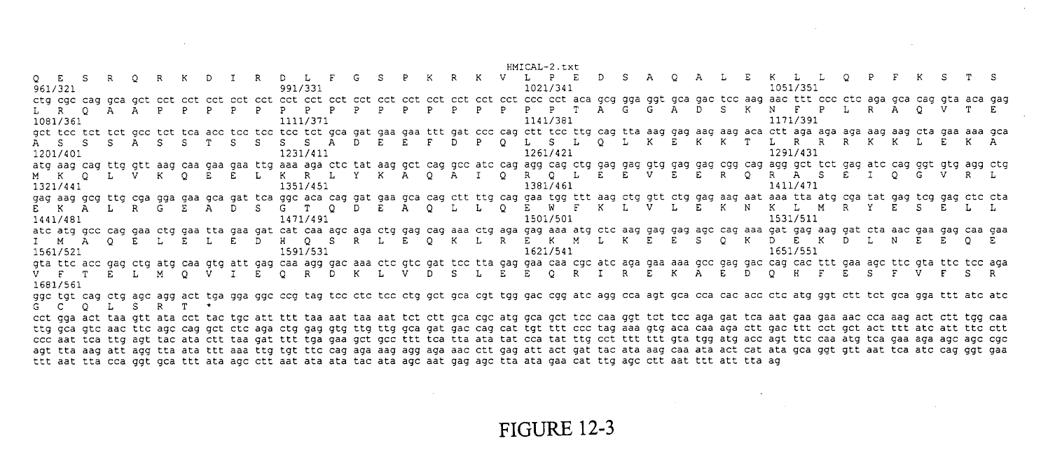

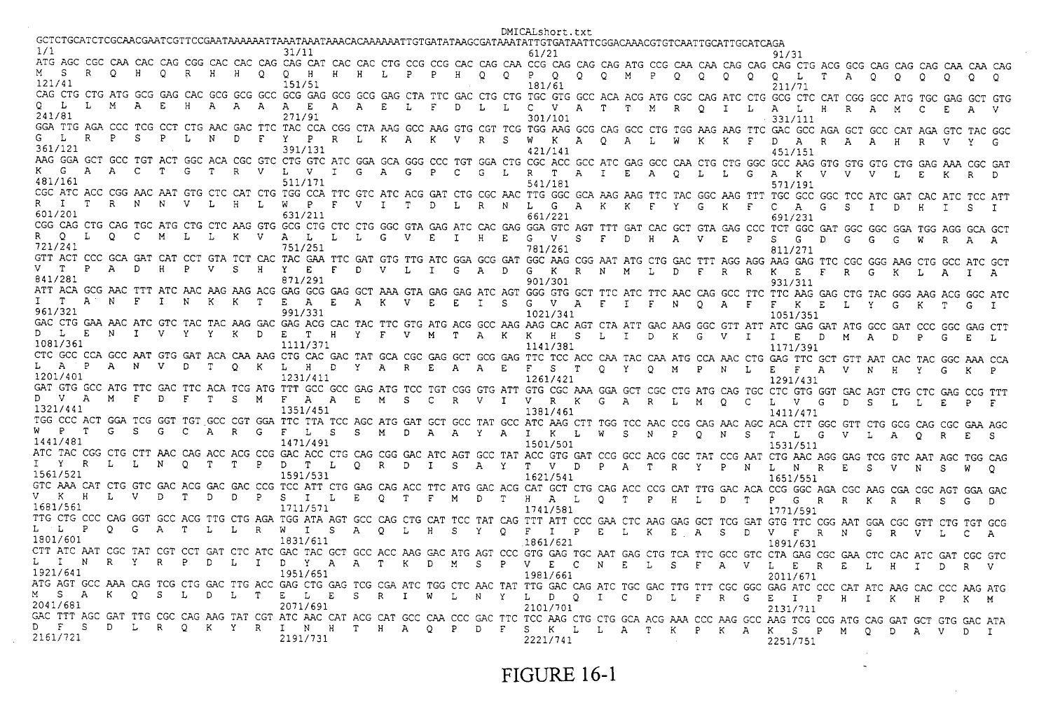

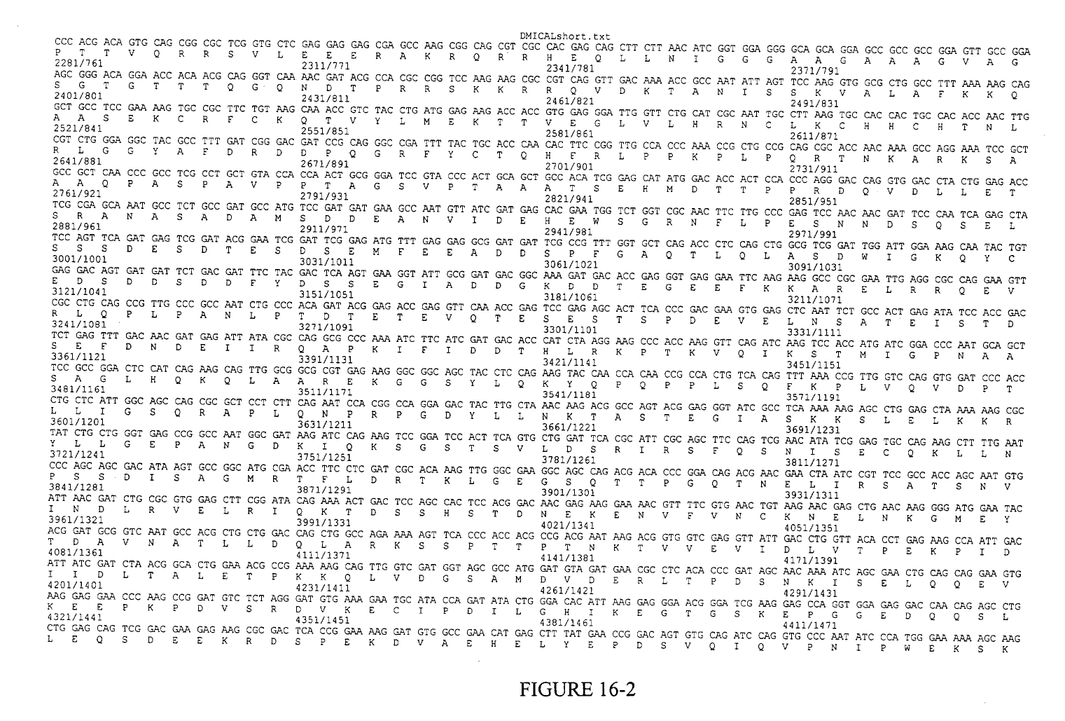

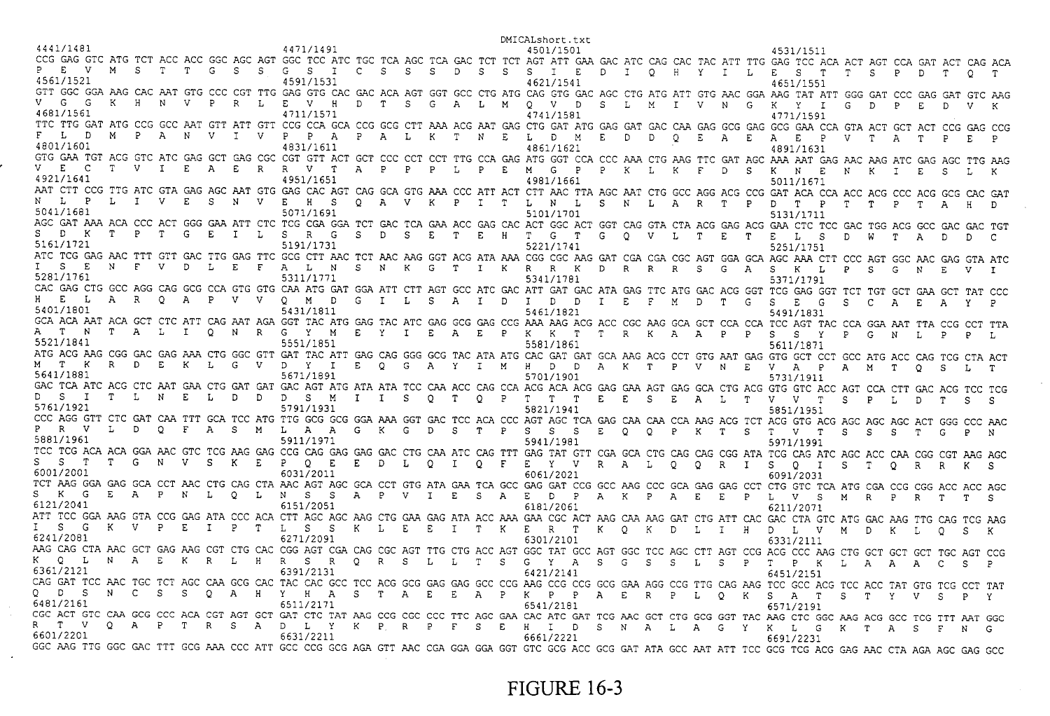

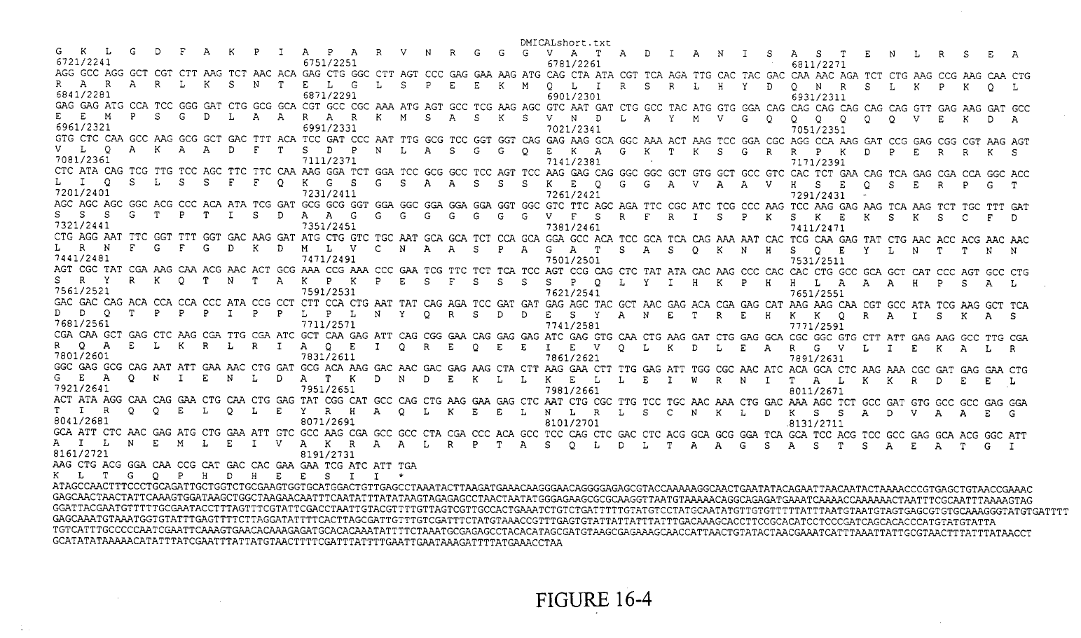

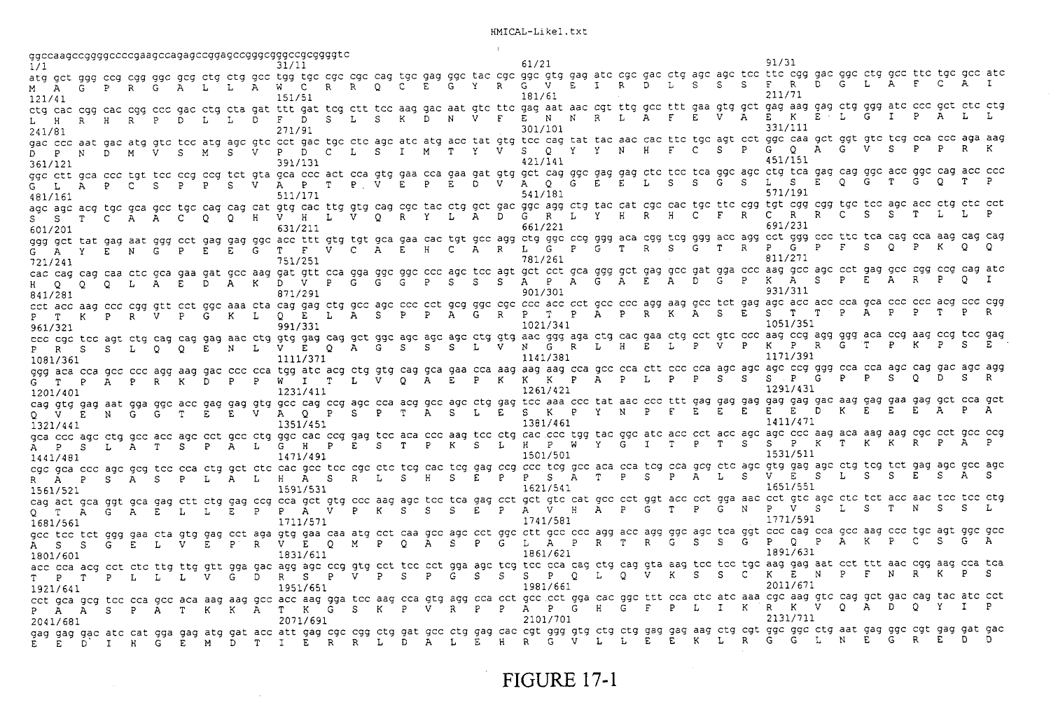

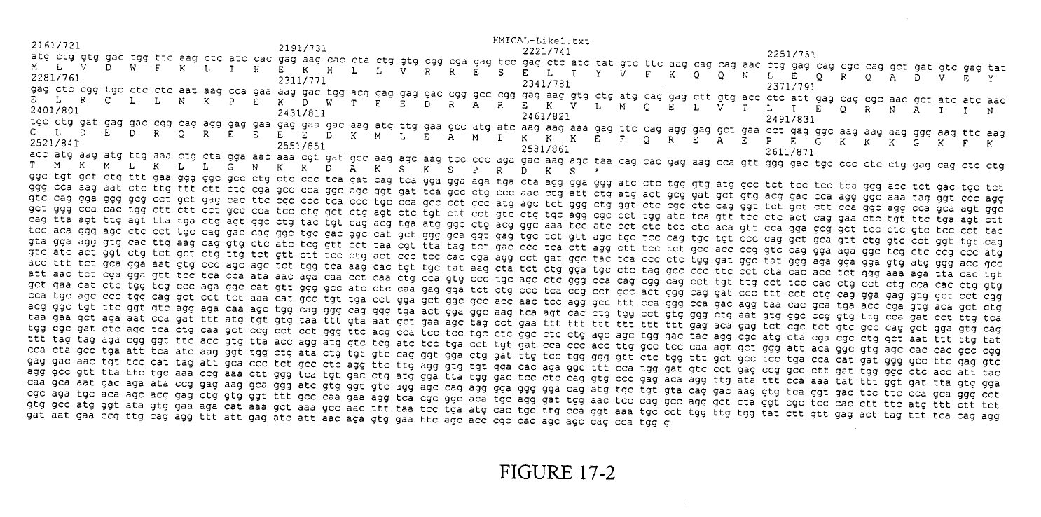

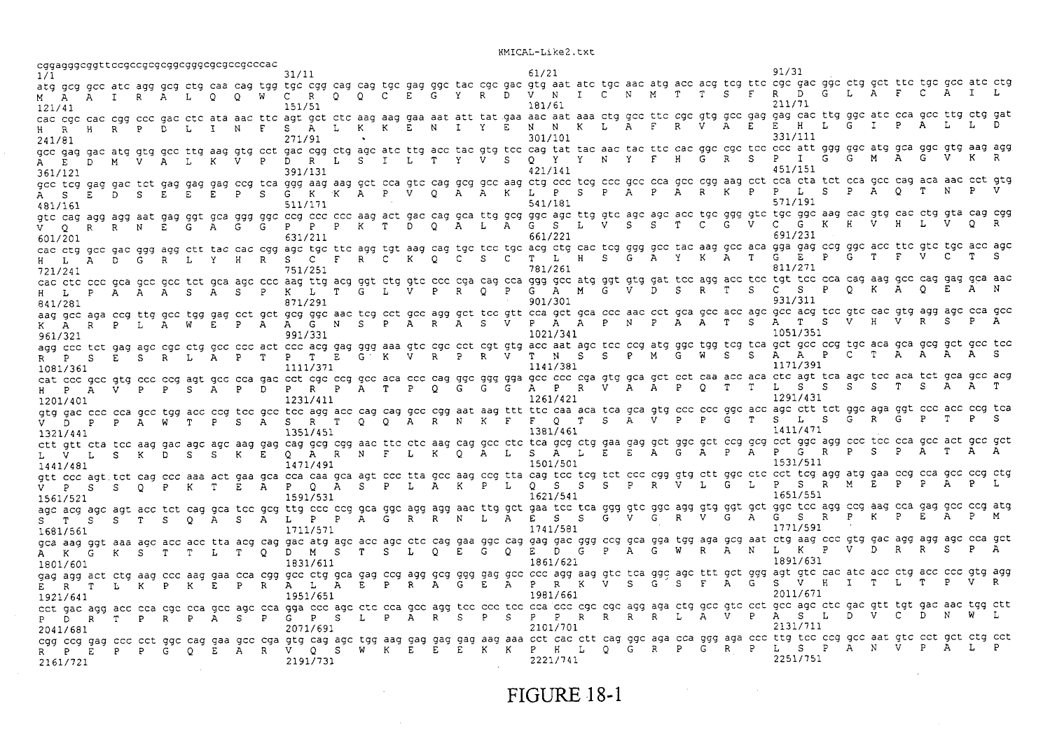

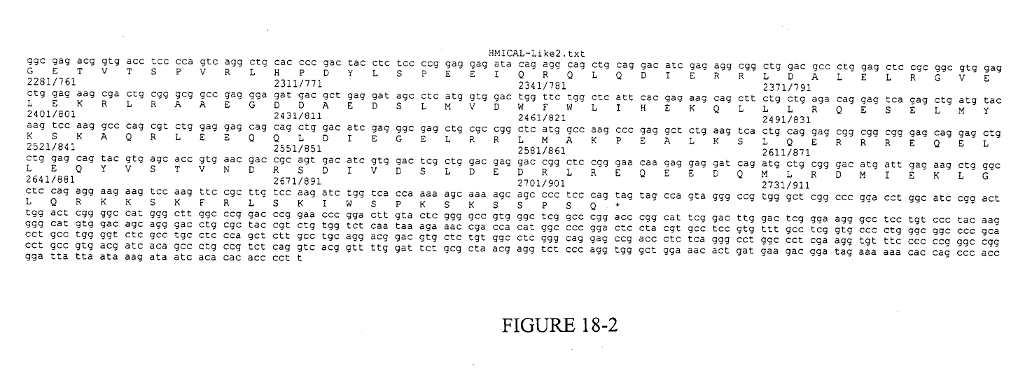

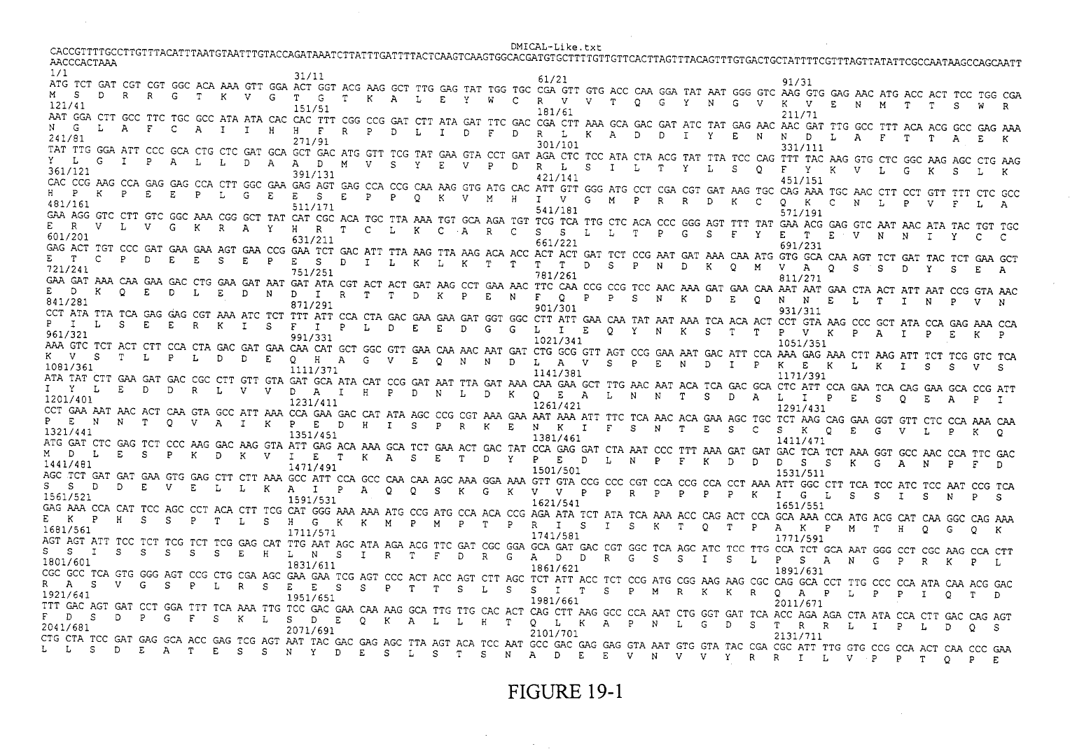

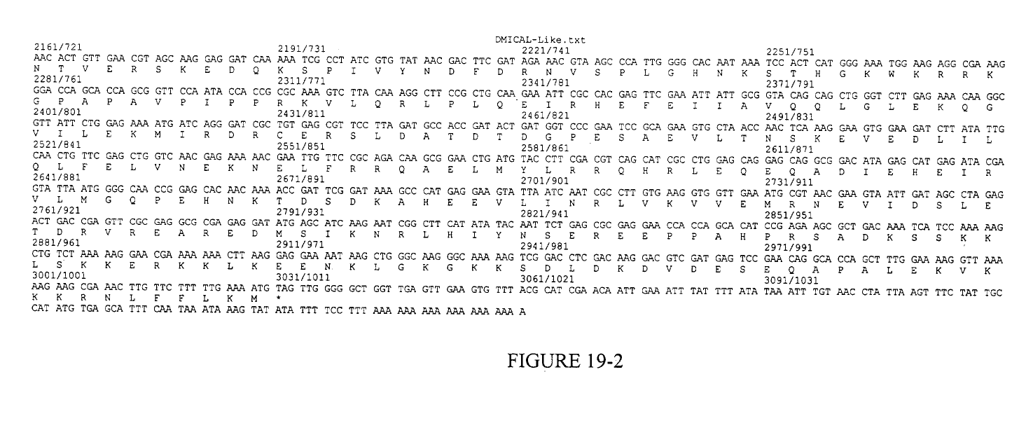

The polynucleotide can include a polynucleotide that is at least 50%, 75%, 80%, 85%, 90%, 95%, 98%, 99%, or 100% identical to a MICAL coding sequence as set forth in FIGS. 11 to 16. Accordingly, a polynucleotide of the present invention in certain aspects, includes a coding nucleotide portion of SEQ ID NO: 1 (human MICAL-1 cDNA), SEQ ID NO:3 (human MICAL-2 coding sequence), SEQ ID NO:5 (human MICAL-3 cDNA), SEQ ID NO:13 (human MICAL Like 1 cDNA), or SEQ ID NO:14 (human MICAL-Like 2 cDNA), or a portion thereof. The polynucleotide can include an entire MICAL cDNA or gene, or an entire MICAL-Like cDNA or gene, or a portion thereof.

A polynucleotide according to this embodiment of the invention can encode a Drosophila MICAL polypeptide or MICAL-Like polypeptide, for example a polypeptide having the sequence as set forth in SEQ ID NO:8 (Drosophila MICAL long isoform), SEQ ID NO: 10 (Drosophila MICAL medium isoform), SEQ ID NO: 12 (Drosophila MICAL short isoform), or SEQ ID NO: 18 (Drosophila MICAL-Like polypeptide). For example, the polynucleotide can be 50%, 75%, 80%, 85%, 90%, 95%, 98%, 99%, or 100% identical to a nucleotide sequence as set forth in SEQ ID NO:7 (Drosophila MICAL long isoform cDNA sequence), SEQ ID NO:9 (Drosophila MICAL medium isoform cDNA sequence), SEQ ID NO: 11 (Drosophila MICAL short isoform cDNA sequence), or SEQ ID NO: 17 (Drosophila MICAL-Like cDNA sequence), or a portion thereof.

Polynucleotides of the present invention are typically at least 15, 25, 50, 75, 100, 125, 150, 200, 250, 500, 1000, 2500, 5000, 10000, 25000, 5000, 10000, 15000, 20000, 25000, 30000, or 40,000 nucleotides in length.

In another embodiment, the present invention provides a polynucleotide that specifically hybridizes to a polynucleotide that encodes a MICAL polypeptide or a MICAL-Like polypeptide of the present invention. For example, the polynucleotide can specifically hybridize to a polynucleotide that encodes all or a portion of a polypeptide of SEQ ID NO:2, SEQ ID NO:4, SEQ ID NO:6, SEQ ID NO:8, SEQ ID NO: 10, SEQ ID NO:12, SEQ ID NO: 14, SEQ ID NO:16, or SEQ ID NO:18, or a complement thereof, that in certain aspects has monooxygenase activity.

A polynucleotide of the present invention can inhibit expression of a polynucleotide that encodes a MICAL polypeptide or a MICAL-Like polypeptide of the present invention. The polynucleotide can include a polynucleotide that is complementary to a nucleotide sequence that is at least 50%, 75%, 80%, 85%, 90%, 95%, 98%, 99%, or 100% identical to a all or a portion, such as a coding portion, of a nucleotide sequence as set forth in SEQ ID NO:1 (human MICAL-1 cDNA), SEQ ID NO:3 (human MICAL-2 cDNA), SEQ ID NO:5 (human MICAL-3 cDNA), SEQ ID NO: 13 (human MICAL-like I cDNA), or SEQ ID NO: 15 (human MICAL-like 2 cDNA), or a portion thereof.

Polynucleotides encoding MICAL or MICAL-Like polypeptides of various organisms in addition to those identified herein, can be identified using well known procedures and algorithms based on identity (or homology) to the disclosed sequences. Homology or identity is often measured using sequence analysis software such as the Sequence Analysis Software Package of the Genetics Computer Group (University of Wisconsin Biotechnology Center, 1710 University Avenue, Madison, Wis. 53705). Such software matches similar sequences by assigning degrees of homology to various deletions, substitutions and other modifications. The terms “homology” and “identity,” when used herein in the context of two or more nucleic acids or polypeptide sequences, refer to two or more sequences or subsequences that are the same or have a specified percentage of amino acid residues or of nucleotides that are the same when compared and aligned for maximum correspondence over a comparison window or designated region as measured using any number of sequence comparison algorithms or by manual alignment and visual inspection.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

The term “comparison window” is used broadly herein to include reference to a segment of any one of the number of contiguous positions, for example, about 20 to 600 positions, for example, amino acid or nucleotide position, usually about 50 to about 200 positions, more usually about 100 to about 150 positions, in which a sequence may be compared to a reference sequence of the same number of contiguous positions after the two sequences are optimally aligned. Methods of alignment of sequence for comparison are well-known in the art. Optimal alignment of sequences for comparison can be conducted, for example, by the local homology algorithm of Smith and Waterman (Adv. Appl. Math. 2:482, 1981), by the homology alignment algorithm of Needleman and Wunsch (J. Mol. Biol. 48:443, 1970), by the search for similarity method of Person and Lipman (Proc. Natl. Acad. Sci., USA 85:2444, 1988), each of which is incorporated herein by reference; by computerized implementations of these algorithms (GAP, BESTFIT, FASTA, and TFASTA in the Wisconsin Genetics Software Package, Genetics Computer Group, 575 Science Dr., Madison, Wis.); or by manual alignment and visual inspection. Other algorithms for determining homology or identity include, for example, in addition to a BLAST program (Basic Local Alignment Search Tool at the National Center for Biological Information), ALIGN, AMAS (Analysis of Multiply Aligned Sequences), AMPS (Protein Multiple Sequence Alignment), ASSET (Aligned Segment Statistical Evaluation Tool), BANDS, BESTSCOR, BIOSCAN (Biological Sequence Comparative Analysis Node), BLIMPS (BLocks IMProved Searcher), FASTA, Intervals & Points, BMB, CLUSTAL V, CLUSTAL W, CONSENSUS, LCONSENSUS, WCONSENSUS, Smith-Waterman algorithm, DARWIN, Las Vegas algorithm, FNAT (Forced Nucleotide Alignment Tool), Framealign, Framesearch, DYNAMIC, FILTER, FSAP (Fristensky Sequence Analysis Package), GAP (Global Alignment Program), GENAL, GIBBS, GenQuest, ISSC (Sensitive Sequence Comparison), LALIGN (Local Sequence Alignment), LCP (Local Content Program), MACAW (Multiple Alignment Construction & Analysis Workbench), MAP (Multiple Alignment Program), MBLKP, MBLKN, PIMA (Pattern-Induced Multi-sequence Alignment), SAGA (Sequence Alignment by Genetic Algorithm) and WHAT-IF. Such alignment programs can also be used to screen genome databases to identify polynucleotide sequences having substantially identical sequences.

A number of genome databases are available for comparison through the National Center for Biotechnology internet site (http://www.ncbi.nlm.nih.gov/). For example, the NCBI site provides access to the complete genomes of human (Ventor, J. C., et al., Science 291:1304-1351 (2001), M. genitalium, M. jannaschii, H. influenzae, E. coli, yeast (S. cerevisiae), and D. melanogaster (Adams et al., Science 287: 2185-2195 (2000)).

One example of a useful algorithm is BLAST and BLAST 2.0 algorithms, which are described by Altschul et al. (Nucleic Acids Res. 25:3389-3402, 1977; J Mol. Biol. 215:403-410, 1990, each of which is incorporated herein by reference). Software for performing BLAST analyses is publicly available through the National Center for Biotechnology Information (http://www.ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra, 1977, 1990). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=4 and a comparison of both strands. For amino acid sequences, the BLASTP program uses as defaults a wordlength of 3, and expectations (E) of 10, and the BLOSUM62 scoring matrix (see Henikoff and Henikoff, Proc. Natl. Acad. Sci., USA 89:10915, 1989) alignments (B) of 50, expectation (E) of 10, M=5, N=−4, and a comparison of both strands.

The BLAST algorithm also performs a statistical analysis of the similarity between two sequences (see, for example, Karlin and Altschul, Proc. Natl. Acad. Sci., USA 90:5873, 1993, which is incorporated herein by reference). One measure of similarity provided by BLAST algorithm is the smallest sum probability (P(N)), which provides an indication of the probability by which a match between two nucleotide or amino acid sequences would occur by chance. For example, a nucleic acid is considered similar to a references sequence if the smallest sum probability in a comparison of the test nucleic acid to the reference nucleic acid is less than about 0.2, more preferably less than about 0.01, and most preferably less than about 0.001.

In one embodiment, protein and nucleic acid sequence homologies are evaluated using the Basic Local Alignment Search Tool (“BLAST”). In particular, five specific BLAST programs are used to perform the following task:

(1) BLASTP and BLAST3 compare an amino acid query sequence against a protein sequence database;

(2) BLASTN compares a nucleotide query sequence against a nucleotide sequence database;

(3) BLASTX compares the six-frame conceptual translation products of a query nucleotide sequence (both strands) against a protein sequence database;

(4) TBLASTN compares a query protein sequence against a nucleotide sequence database translated in all six reading frames (both strands); and

(5) TBLASTX compares the six-frame translations of a nucleotide query sequence against the six-frame translations of a nucleotide sequence database.

The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix (Gonnet et al., Science 256:1443-1445, 1992; Henikoff and Henikoff, Proteins 17:49-61, 1993, each of which is incorporated herein by reference). Less preferably, the PAM or PAM250 matrices may also be used (Schwartz and Dayhoff, eds., “Matrices for Detecting Distance Relationships: Atlas of Protein Sequence and Structure” (Washington, National Biomedical Research Foundation 1978)). BLAST programs are accessible through the U.S. National Library of Medicine, for example, at www.ncbi.nlm.nih.gov.

The parameters used with the above algorithms may be adapted depending on the sequence length and degree of homology studied. In some embodiments, the parameters may be the default parameters used by the algorithms in the absence of instructions from the user.

Therefore, using MICAL sequences disclosed herein, and known methods and databases, a polynucleotide encoding a MICAL or MICAL-Like polypeptide, or the MICAL or MICAL-Like polypeptide or protein can be identified from any organism. Therefore, MICAL polynucleotides, polypeptides, and proteins of the present invention include, for example, mouse, rat, cow, pig, horse, dog, human, chicken, turkey, zebrafish, and other species.

It should also be recognized that reference is made herein to particular peptides or polypeptides beginning or ending at “about” a particular amino acid residue. The term “about” is used in this context because it is recognized that a particular protease can cleave a MICAL polypeptide at or immediately adjacent to a proteolytic cleavage recognition site, or one or a few amino acids from the recognition site. As such, reference, for example, to a MICAL polypeptide having a sequence of about amino acid residues 1 to 263 of SEQ ID NO:2 would include an amino terminal peptide portion of MICAL that has a carboxy terminus ending at amino acid residue 257 to amino acid residue 269, preferably at amino acid residue 260 to amino acid residue 266.

The term “peptide,” “peptide portion,” or polypeptide is used broadly herein to mean two or more amino acids linked by a peptide bond. The term “fragment” or “proteolytic fragment” also is used herein to refer to a product that can be produced by a proteolytic reaction on a polypeptide, i.e., a peptide produced upon cleavage of a peptide bond in the polypeptide. Although the term “proteolytic fragment” is used generally herein to refer to a peptide that can be produced by a proteolytic reaction, it should be recognized that the fragment need not necessarily be produced by a proteolytic reaction, but also can be produced using methods of chemical synthesis or methods of recombinant DNA technology, as discussed in greater detail below, to produce a synthetic peptide that is equivalent to a proteolytic fragment. In view of the disclosed homology of MICALs and MICAL-Like proteins with other proteins, it will be recognized that a polypeptide of the invention is characterized, in part, in that it is not present in previously disclosed members of this superfamily. Whether a polypeptide portion of a MICAL or MICAL-Like polypeptide is present in a previously disclosed protein readily can be determined using the computer algorithms described above.

Generally, a peptide or polypeptide of the invention contains at least about six amino acids, usually contains about ten amino acids, and can contain fifteen or more amino acids, particularly twenty or more amino acids. It should be recognized that the terms “peptide” and “polypeptide” is not used herein to suggest a particular size or number of amino acids comprising the molecule, and that a polypeptide of the invention can contain up to several amino acid residues or more.

As used herein, the term “substantially purified” or “substantially pure” or “isolated” means that the molecule being referred to, for example, a polypeptide or a polynucleotide, is in a form that is relatively free of proteins, nucleic acids, lipids, carbohydrates or other materials with which it is naturally associated. Generally, a substantially pure polypeptide, polynucleotide, or other molecule constitutes at least twenty percent of a sample, generally constitutes at least about fifty percent of a sample, usually constitutes at least about eighty percent of a sample, and particularly constitutes about ninety percent or ninety-five percent or more of a sample. A determination that a peptide or a polynucleotide of the invention is substantially pure can be made using well known methods, for example, by performing electrophoresis and identifying the particular molecule as a relatively discrete band. A substantially pure polynucleotide, for example, can be obtained by cloning the polynucleotide, or by chemical or enzymatic synthesis. A substantially pure peptide or polypeptide can be obtained, for example, by a method of chemical synthesis, or using methods of protein purification, followed by proteolysis and, if desired, further purification by chromatographic or electrophoretic methods.

A polypeptide of the invention can be identified by comparison to a MICAL or MICAL-Like sequence and determining that the amino acid sequence of the polypeptide is contained within the MICAL or MICAL-Like polypeptide sequence, respectively. It should be recognized, however, that a polypeptide of the invention need not be identical to a corresponding amino acid sequence of MICAL or a MICAL-Like polypeptide. Thus, a polypeptide of the invention can correspond to an amino acid sequence of a MICAL polypeptide, for example, but can vary from a naturally occurring sequence, for example, by containing one or more D-amino acids in place of a corresponding L-amino acid; or by containing one or more amino acid analogs, for example, an amino acid that has been derivatized or otherwise modified at its reactive side chain. Similarly, one or more peptide bonds in the polypeptide can be modified. In addition, a reactive group at the amino terminus or the carboxy terminus or both can be modified. Such polypeptides can be modified, for example, to have improved stability to a protease, an oxidizing agent or other reactive material the peptide may encounter in a biological environment, and, therefore, can be particularly useful in performing a method of the invention. Of course, the polypeptides can be modified to have decreased stability in a biological environment such that the period of time the polypeptide is active in the environment is reduced.

The sequence of a MICAL or MICAL-Like polypeptide of the invention also can be modified by incorporating a conservative amino acid substitution for one or a few amino acids in the polypeptide. Conservative amino acid substitutions include the replacement of one amino acid residue with another amino acid residue having relatively the same chemical characteristics, for example, the substitution of one hydrophobic residue such as isoleucine, valine, leucine or methionine for another, or the substitution of one polar residue for another, for example, substitution of arginine for lysine; or of glutamic for aspartic acid; or of glutamine for asparagine; or the like. Examples of positions of a MICAL polypeptide that can be modified are evident from examination of differences in the disclosed MICAL sequences.

The present invention also provides a substantially purified proteolytic fragment of a MICAL polypeptide or a functional peptide portion thereof. A peptide portion of a MICAL polypeptide that is equivalent to a proteolytic fragment of a MICAL can be produced by a chemical method or a recombinant DNA method.

The term “polynucleotide” is used broadly herein to mean a sequence of two or more deoxyribonucleotides or ribonucleotides that are linked together by a phosphodiester bond. As such, the term “polynucleotide” includes RNA and DNA, which can be a gene or a portion thereof, a CDNA, a synthetic polydeoxyribonucleic acid sequence, or the like, and can be single stranded or double stranded, as well as a DNA/RNA hybrid. Furthermore, the term “polynucleotide” as used herein includes naturally occurring nucleic acid molecules, which can be isolated from a cell, as well as synthetic molecules, which can be prepared, for example, by methods of chemical synthesis or by enzymatic methods such as by the polymerase chain reaction (PCR). In various embodiments, a polynucleotide of the invention can contain nucleoside or nucleotide analogs, or a backbone bond other than a phosphodiester bond (see above).

In general, the nucleotides that make up a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. However, a polynucleotide also can contain nucleotide analogs, including non-naturally occurring synthetic nucleotides or modified naturally occurring nucleotides. Such nucleotide analogs are well known in the art and commercially available, as are polynucleotides containing such nucleotide analogs (Lin et al., Nucl. Acids Res. 22:5220-5234 (1994); Jellinek et al., Biochemistry 34:11363-11372 (1995); Pagratis et al., Nature Biotechnol. 15:68-73 (1997), each of which is incorporated herein by reference).

The covalent bond linking the nucleotides of a polynucleotide generally is a phosphodiester bond. However, the covalent bond also can be any of numerous other bonds, including a thiodiester bond, a phosphorothioate bond, a peptide-like bond or any other bond known to those in the art as useful for linking nucleotides to produce synthetic polynucleotides (see, for example, Tam et al., Nucl. Acids Res. 22:977-986 (1994); Ecker and Crooke, BioTechnology 13:351360 (1995), each of which is incorporated herein by reference). The incorporation of non-naturally occurring nucleotide analogs or bonds linking the nucleotides or analogs can be particularly useful where the polynucleotide is to be exposed to an environment that can contain a nucleolytic activity, including, for example, a tissue culture medium or upon administration to a living subject, since the modified polynucleotides can be less susceptible to degradation.

A polynucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template (Jellinek et al., supra, 1995).

Where a polynucleotide encodes a polypeptide, for example, a polypeptide portion of a MICAL or a polypeptide agent, the coding sequence generally is contained in a vector and is operatively linked to appropriate regulatory elements, including, if desired, a tissue specific promoter or enhancer. The encoded peptide can be further operatively linked, for example, to a peptide tag such as a His-6 tag or the like, which can facilitate identification of expression of the agent in the target cell. A polyhistidine tag peptide such as His-6 can be detected using a divalent cation such as nickel ion, cobalt ion, or the like. Additional peptide tags include, for example, a FLAG epitope, which can be detected using an anti-FLAG antibody (see, for example, Hopp et al., BioTechnology 6:1204 (1988); U.S. Pat. No. 5,011,912, each of which is incorporated herein by reference); a c-myc epitope, which can be detected using an antibody specific for the epitope; biotin, which can be detected using streptavidin or avidin; and glutathione S-transferase, which can be detected using glutathione. Such tags can provide the additional advantage that they can facilitate isolation of the operatively linked peptide or peptide agent, for example, where it is desired to obtain a substantially purified peptide corresponding to a proteolytic fragment of a MICAL or MICAL-Like polypeptide.

As used herein, the term “operatively linked” or “operatively associated” means that two or more molecules are positioned with respect to each other such that they act as a single unit and effect a function attributable to one or both molecules or a combination thereof. For example, a polynucleotide sequence encoding a polypeptide of the invention can be operatively linked to a regulatory element, in which case the regulatory element confers its regulatory effect on the polynucleotide similarly to the way in which the regulatory element would effect a polynucleotide sequence with which it normally is associated with in a cell. A first polynucleotide coding sequence also can be operatively linked to a second (or more) coding sequence such that a chimeric polypeptide can be expressed from the operatively linked coding sequences. The chimeric polypeptide can be a fusion polypeptide, in which the two (or more) encoded peptides are translated into a single polypeptide, i.e., are covalently bound through a peptide bond; or can be translated as two discrete peptides that, upon translation, can operatively associate with each other to form a stable complex.

A polynucleotide of the invention, including a polynucleotide agent useful in performing a method of the invention, can be contacted directly with a target cell. For example, oligonucleotides useful as antisense molecules, ribozymes, or triplexing agents can be directly contacted with a target cell, whereupon they enter the cell and affect their function. A polynucleotide agent also can interact specifically with a polypeptide, for example, a MICAL polypeptide, thereby altering the ability of the MICAL to interact specifically with a plexin. Such polynucleotides, as well as methods of making and identifying such polynucleotides, are disclosed herein or otherwise well known in the art (see, for example, O'Connell et al., Proc. Natl. Acad. Sci., USA 93:5883-5887, 1996; Tuerk and Gold, Science 249:505-510, 1990; Gold et al., Ann. Rev. Biochem. 64:763-797, 1995; each of which is incorporated herein by reference).

A polynucleotide of the invention, which can encode a MICAL or MICAL-Like polypeptide or can encode a mutant MICAL or MICAL-Like polypeptide or functional peptide portion thereof, or can be a polynucleotide agent useful in performing a method of the invention, can be contained in a vector, which can facilitate manipulation of the polynucleotide, including introduction of the polynucleotide into a target cell. The vector can be a cloning vector, which is useful for maintaining the polynucleotide, or can be an expression vector, which contains, in addition to the polynucleotide, regulatory elements useful for expressing the polynucleotide and, where the polynucleotide encodes a peptide, for expressing the encoded peptide in a particular cell. An expression vector can contain the expression elements necessary to achieve, for example, sustained transcription of the encoding polynucleotide, or the regulatory elements can be operatively linked to the polynucleotide prior to its being cloned into the vector.

An expression vector, or the polynucleotide included on the vector generally contains or encodes a promoter sequence, which can provide constitutive or, if desired, inducible or tissue specific or developmental stage specific expression of the encoding polynucleotide, a poly-A recognition sequence, and a ribosome recognition site or internal ribosome entry site, or other regulatory elements such as an enhancer, which can be tissue specific. The vector also can contain elements required for replication in a prokaryotic or eukaryotic host system or both, as desired. Such vectors, which include plasmid vectors and viral vectors such as bacteriophage, baculovirus, retrovirus, lentivirus, adenovirus, vaccinia virus, semliki forest virus and adeno-associated virus vectors, are well known and can be purchased from a commercial source (Promega, Madison Wis.; Stratagene, La Jolla Calif.; GIBCO/BRL, Gaithersburg Md.) or can be constructed by one skilled in the art (see, for example, Meth. Enzymol., Vol. 185, Goeddel, ed. (Academic Press, Inc., 1990); Jolly, Canc. Gene Ther 1:51-64, 1994; Flotte, J. Bioenerg. Biomemb. 25:37-42, 1993; Kirshenbaum et al., J. Clin. Invest. 92:381-387, 1993; each of which is incorporated herein by reference).

A tetracycline (tet) inducible promoter can be particularly useful for driving expression of a polynucleotide of the invention, for example, a polynucleotide encoding a dominant negative form of a MICAL polypeptide. Upon administration of tetracycline, or a tetracycline analog, to a subject containing a polynucleotide operatively linked to a tet inducible promoter, expression of the encoded polypeptide is induced, whereby the polypeptide can effect its activity, for example, whereby a polypeptide agent can reduce or inhibit semaphorin mediated axonal repulsion. Such a method can be used, for example, to induce axon formation after spinal cord injury.

The polynucleotide also can be operatively linked to tissue specific regulatory element, for example, a neuron specific regulatory element, such that expression of an encoded peptide is restricted to the neurons in an individual, or to neurons in a mixed population of cells in culture. Neuron specific regulatory elements are well known in the art as illustrated in the Examples section (See also, e.g., Nelson, S. B., et al., Mol Endocrinol. 14:1509-22 (2000); and Navarro et al., Gene Ther. 6:1884-92 (1999)). For example, after a spinal cord injury, a vector that encodes a dominant negative MICAL polypeptide operatively linked to a neuron-specific promoter, can be delivered to the site of spinal cord injury. Expression of the dominant negative mutant in neurons is expected to inhibit MICAL regulatory activity, thereby inhibiting semaphorin-mediated axon repulsion. This inhibition permits axons to regrow and migrate to reach new targets

Viral expression vectors can be particularly useful for introducing a polynucleotide into a cell, particularly a cell in a subject. Viral vectors provide the advantage that they can infect host cells with relatively high efficiency and can infect specific cell types. For example, a polynucleotide encoding a MICAL polypeptide, or functional peptide portion thereof can be cloned into a baculovirus vector, which then can be used to infect an insect host cell, thereby providing a means to produce large amounts of the encoded MICAL or MICAL-Like protein. The viral vector also can be derived from a virus that infects cells of an organism of interest, for example, vertebrate host cells such as mammalian, avian or piscine host cells. Viral vectors can be particularly useful for introducing a polynucleotide useful in performing a method of the invention into a target cell. Viral vectors have been developed for use in particular host systems, particularly mammalian systems and include, for example, retroviral vectors, other lentivirus vectors such as those based on the human immunodeficiency virus (HIV), adenovirus vectors, adeno-associated virus vectors, herpesvirus vectors, vaccinia virus vectors, and the like (see Miller and Rosman, BioTechniques 7:980-990, 1992; Anderson et al., Nature 392:25-30 Suppl., 1998; Verma and Somia, Nature 389:239-242, 1997; Wilson, New Engl. J Med. 334:1185-1187 (1996), each of which is incorporated herein by reference).

When retroviruses, for example, are used for gene transfer, replication competent retroviruses theoretically can develop due to recombination of retroviral vector and viral gene sequences in the packaging cell line utilized to produce the retroviral vector. Packaging cell lines in which the production of replication competent virus by recombination has been reduced or eliminated can be used to minimize the likelihood that a replication competent retrovirus will be produced. All retroviral vector supernatants used to infect cells are screened for replication competent virus by standard assays such as PCR and reverse transcriptase assays. Retroviral vectors allow for integration of a heterologous gene into a host cell genome, which allows for the gene to be passed to daughter cells following cell division.

A polynucleotide, which can be contained in a vector, can be introduced into a cell by any of a variety of methods known in the art (Sambrook et al., Molecular Cloning: A laboratory manual (Cold Spring Harbor Laboratory Press 1989); Ausubel et al., Current Protocols in Molecular Biology, John Wiley and Sons, Baltimore, Md. (1987, and supplements through 1995), each of which is incorporated herein by reference). Such methods include, for example, transfection, lipofection, microinjection, electroporation and, with viral vectors, infection; and can include the use of liposomes, microemulsions or the like, which can facilitate introduction of the polynucleotide into the cell and can protect the polynucleotide from degradation prior to its introduction into the cell. The selection of a particular method will depend, for example, on the cell into which the polynucleotide is to be introduced, as well as whether the cell is isolated in culture, or is in a tissue or organ in culture or in situ.

Introduction of a polynucleotide into a cell by infection with a viral vector is particularly advantageous in that it can efficiently introduce the nucleic acid molecule into a cell ex vivo or in vivo (see, for example, U.S. Pat. No. 5,399,346, which is incorporated herein by reference). Moreover, viruses are very specialized and can be selected as vectors based on an ability to infect and propagate in one or a few specific cell types. Thus, their natural specificity can be used to target the nucleic acid molecule contained in the vector to specific cell types. As such, a vector based on an HIV can be used to infect T cells, a vector based on an adenovirus can be used, for example, to infect respiratory epithelial cells, a vector based on a herpesvirus can be used to infect neuronal cells, and the like. Other vectors, such as adeno-associated viruses can have greater host cell range and, therefore, can be used to infect various cell types, although viral or non-viral vectors also can be modified with specific receptors or ligands to alter target specificity through receptor mediated events.

Thus, a polynucleotide of the invention can be a naturally occurring, synthetic, or intentionally manipulated polynucleotide. For example, portions of the mRNA sequence can be altered due to alternate RNA splicing patterns or the use of alternate promoters for RNA transcription. As another example, the polynucleotide can be subjected to site directed mutagenesis. The polynucleotide also can be antisense nucleotide sequence. MICAL and MICAL-Like polynucleotides (i.e., polynucleotides that encode a MICAL polypeptide or a MICAL-Like polypeptide) of the invention include sequences that are degenerate as a result of the genetic code. There are 20 natural amino acids, most of which are specified by more than one codon. Therefore, all degenerate nucleotide sequences are included within the invention, provided the amino acid sequence of a MICAL polypeptide or a MICAL-Like encoded by the polynucleotide is functionally unchanged.

Oligonucleotide portions of a polynucleotide encoding a MICAL polypeptide or a MICAL-Like polypeptide of the invention also are encompassed within the present invention. Such oligonucleotides generally are at least about 15 bases in length, which is sufficient to permit the oligonucleotide to selectively hybridize to a polynucleotide encoding the MICAL or MICAL-Like polypeptide, and can be at least about 18 nucleotides or 21 nucleotides or more in length. As used herein, the term “selective hybridization” or “selectively hybridize” refers to hybridization under moderately stringent or highly stringent physiological conditions, which can distinguish related nucleotide sequences from unrelated nucleotide sequences.

In nucleic acid hybridization reactions, the conditions used to achieve a particular level of stringency will vary, depending on the nature of the nucleic acids being hybridized. For example, the length, degree of complementarity, nucleotide sequence composition (for example, relative GC:AT content), and nucleic acid type, i.e., whether the oligonucleotide or the target nucleic acid sequence is DNA or RNA, can be considered in selecting hybridization conditions. An additional consideration is whether one of the nucleic acids is immobilized, for example, on a filter. Methods for selecting appropriate stringency conditions can be determined empirically or estimated using various formulas, and are well known in the art (see, for example, Sambrook et al., supra, 1989).

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, for example, high stringency conditions, or each of the conditions can be used, for example, for 10 to 15 minutes each, in the order listed above, repeating any or all of the steps listed.

A MICAL or MICAL-Like polypeptide-encoding polynucleotide of the invention can be obtained by any of several methods. For example, the polynucleotide can be isolated using hybridization or computer based techniques, as are well known in the art. These methods include, but are not limited to, 1) hybridization of genomic or CDNA libraries with probes to detect homologous nucleotide sequences; 2) antibody screening of expression libraries to detect cloned DNA fragments with shared structural features; 3) polymerase chain reaction (PCR) on genomic DNA or cDNA using primers capable of annealing to the DNA sequence of interest; 4) computer searches of sequence databases for similar sequences (see above); 5) differential screening of a subtracted DNA library; and 6) two hybrid assays using, for example, a MICAL polypeptide in one of the hybrids.

A polynucleotide of the invention, for example, a polynucleotide encoding a MICAL, can be derived from a vertebrate species, including a mammalian, avian, or piscine species, or from an invertebrate species. Screening procedures that rely on nucleic acid hybridization allow the isolation any gene sequence from any organism, provided the appropriate probe is available. Oligonucleotide probes that correspond to a part of the sequence encoding the protein in question can be synthesized chemically. Hybridization is particularly useful in the detection of cDNA clones derived from sources where an extremely low amount of mRNA sequences relating to the polypeptide of interest are present. Thus, by using stringent hybridization conditions directed to avoid nonspecific binding, autoradiographic visualization can be used to identify a specific cDNA clone by the hybridization of the target DNA to an oligonucleotide probe in the mixture that is the complete complement of the target nucleic acid (Wallace et al., Nucl. Acid Res., 9:879, 1981, which is incorporated herein by reference). Alternatively, a subtractive library can be used, thereby eliminating nonspecific cDNA clones.

When the entire amino acid sequence of a desired polypeptide is not known, the direct synthesis of DNA sequences is not possible and the method of choice is the synthesis of cDNA sequences. Among the standard procedures for isolating cDNA sequences of interest is the formation of cDNA libraries prepared in plasmids or phage, wherein the libraries are derived from reverse transcription of mRNA that is abundant in donor cells having a high level of genetic expression. When used in combination with polymerase chain reaction technology, even rare expression products can be cloned. Where significant portions of the amino acid sequence of the polypeptide are known, the production of labeled single stranded or double stranded DNA or RNA probe sequences duplicating a sequence putatively present in the target cDNA can be employed in hybridization procedures carried out on cloned copies of the cDNA, which have been denatured into a single stranded form (Jay et al., Nucl. Acid Res., 11:2325, 1983, which is incorporated herein by reference).

A cDNA expression library, such as a lambda gt11 library, can be screened for MICAL or MICAL-Like polypeptides using an antibody specific for a MICAL or MICAL-Like polypeptide. The antibody can be polyclonal or monoclonal, and can be used to detect expression product indicative of the presence of a MICAL or MICAL-Like polypeptide encoding cDNA. Such an expression library also can be screened with a MICAL or MICAL-Like polypeptide to identify a clone encoding at least a portion of a MICAL or MICAL-Like polypeptide binding domain of a plexin or other protein that interacts with MICAL or the MICAL-Like polypeptide.

Polynucleotides encoding mutant MICAL and MICAL-Like polypeptides are also encompassed within the invention. An alteration in a polynucleotide encoding a MICAL or MICAL-Like protein can be an intragenic mutation such as point mutation, nonsense (STOP) mutation, missense mutation, splice site mutation or frameshift, or can be a heterozygous or homozygous deletion, and can be a naturally occurring mutation or can be engineered using recombinant DNA methods, for example. Such alterations can be detected using standard methods known to those of skill in the art, including, but not limited to, nucleotide sequence analysis, Southern blot analysis, a PCR based analysis such as multiplex PCR or sequence tagged sites (STS) analysis, or in situ hybridization analysis. MICAL and MICAL-Like polypeptides can be analyzed by standard SDS-PAGE, immunoprecipitation analysis, western blot analysis, or the like.

A polynucleotide encoding a MICAL or a MICAL-Like polypeptide can be expressed in vitro by introducing the polynucleotide into a suitable host cell. “Host cells” can be any cells in which the particular vector can be propagated, and, where appropriate, in which a polynucleotide contained in the vector can be expressed. The term “host cells” includes any progeny of an original host cell. It is understood that all progeny of the host cell may not be identical to the parental cell due, for example, to mutations that occur during replication. However, such progeny are included when the term “host cell” is used. Methods of obtaining a host cell that transiently or stably contains an introduced polynucleotide of the invention are well known in the art. In one aspect, the present invention provides host cell that includes a polynucleotide encoding a MICAL polypeptide according to the present invention, operably linked to a heterologous promoter.

In certain aspects of embodiments of the present invention, a cell is a mammalian cell, for example a human cell.

A polynucleotide encoding a MICAL or a MICAL-Like polypeptide of the invention can be inserted into a vector, which can be a cloning vector or a recombinant expression vector. The term “recombinant expression vector” refers to a plasmid, virus or other vehicle known in the art that has been manipulated by insertion or incorporation of a polynucleotide, particularly, with respect to the present invention, a polynucleotide encoding all or a peptide portion of a MICAL or a MICAL-Like polypeptide. Such expression vectors contain a promoter sequence, which facilitates the efficient transcription of the inserted genetic sequence of the host. The expression vector generally contains an origin of replication, a promoter, as well as specific genes which allow phenotypic selection of the transformed cells. Vectors suitable for use in the present invention include, but are not limited to, the T7-based expression vector for expression in bacteria (Rosenberg, et al., Gene 56:125, 1987), the pMSXND expression vector for expression in mammalian cells (Lee and Nathans, J. Biol. Chem. 263:3521, 1988) and baculovirus-derived vectors for expression in insect cells. The DNA segment can be present in the vector operably linked to regulatory elements, for example, a promoter, which can be a T7 promoter, metallothionein I promoter, polyhedrin promoter, or other promoter as desired, particularly tissue specific promoters or inducible promoters.

A polynucleotide sequence encoding a MICAL or a MICAL-Like polypeptide can be expressed in either prokaryotes or eukaryotes. Hosts can include microbial, yeast, insect and mammalian organisms. Methods of expressing polynucleotides having eukaryotic or viral sequences in prokaryotes are well known in the art, as are biologically functional viral and plasmid DNA vectors capable of expression and replication in a host. Methods for constructing an expression vector containing a polynucleotide of the invention are well known, as are factors to be considered in selecting transcriptional or translational control signals, including, for example, whether the polynucleotide is to be expressed preferentially in a particular cell type or under particular conditions (see, for example, Sambrook et al., supra, 1989).

A variety of host cell/expression vector systems can be utilized to express a MICAL or a MICAL-Like polypeptide coding sequence, including, but not limited to, microorganisms such as bacteria transformed with recombinant bacteriophage DNA, plasmid DNA or cosmid DNA expression vectors; yeast cells transformed with recombinant yeast expression vectors; plant cell systems infected with recombinant virus expression vectors such as a cauliflower mosaic virus or tobacco mosaic virus, or transformed with recombinant plasmid expression vector such as a Ti plasmid; insect cells infected with recombinant virus expression vectors such as a baculovirus; animal cell systems infected with recombinant virus expression vectors such as a retrovirus, adenovirus or vaccinia virus vector; and transformed animal cell systems genetically engineered for stable expression. Where the expressed MICAL or a MICAL-Like polypeptide is post-translationally modified, for example, by glycosylation, it can be particularly advantageous to select a host cell/expression vector system that can effect the desired modification, for example, a mammalian host cell/expression vector system.

Depending on the host cell/vector system utilized, any of a number of suitable transcription and translation elements, including constitutive and inducible promoters, transcription enhancer elements, transcription terminators, and the like can be used in the expression vector (Bitter et al., Meth. Enzymol. 153:516-544, 1987). For example, when cloning in bacterial systems, inducible promoters such as pL of bacteriophage λ, plac, ptrp, ptac (ptrp-lac hybrid promoter) and the like can be used. When cloning in mammalian cell systems, promoters derived from the genome of mammalian cells, for example, a human or mouse metallothionein promoter, or from mammalian viruses, for example, a retrovirus long terminal repeat, an adenovirus late promoter or a vaccinia virus 7.5K promoter, can be used. Promoters produced by recombinant DNA or synthetic techniques can also be used to provide for transcription of the inserted MICAL or a MICAL-Like polypeptide coding sequence.

In yeast cells, a number of vectors containing constitutive or inducible promoters can be used (see Ausubel et al., supra, 1987, see chapter 13; Grant et al., Meth. Enzymol. 153:516-544, 1987; Glover, DNA Cloning Vol. II (IRL Press, 1986), see chapter 3; Bitter, Meth. Enzymol. 152:673-684, 1987; see, also, The Molecular Biology of the Yeast Saccharomyces (Eds., Strathern et al., Cold Spring Harbor Laboratory Press, 1982), Vols. I and II). A constitutive yeast promoter such as ADH or LEU2 or an inducible promoter such as GAL can be used (Rothstein, DNA Cloning Vol. II (supra, 1986), chapter 3). Alternatively, vectors can be used which promote integration of foreign DNA sequences into the yeast chromosome.

Eukaryotic systems, particularly mammalian expression systems, allow for proper post-translational modifications of expressed mammalian proteins. Eukaryotic cells which possess the cellular machinery for proper processing of the primary transcript, glycosylation, phosphorylation, and advantageously, plasma membrane insertion of the gene product can be used as host cells for the expression of a MICAL or MICAL-Like polypeptide, or functional peptide portion thereof.

Mammalian cell systems which utilize recombinant viruses or viral elements to direct expression can be engineered. For example, when using adenovirus expression vectors, the MICAL or MICAL-Like polypeptide coding sequence can be ligated to an adenovirus transcription/translation control complex, e.g., the late promoter and tripartite leader sequence. Alternatively, the vaccinia virus 7.5K promoter can be used (Mackett et al., Proc. Natl. Acad. Sci., USA 79:7415-7419, 1982; Mackett et al., J. Virol. 49:857-864, 1984; Panicali et al., Proc. Natl. Acad. Sci., USA 79:4927-4931, 1982). Particularly useful are bovine papilloma virus vectors, which can replicate as extrachromosomal elements (Sarver et al., Mol. Cell. Biol. 1:486, 1981). Shortly after entry of this DNA into mouse cells, the plasmid replicates to about 100 to 200 copies per cell. Transcription of the inserted cDNA does not require integration of the plasmid into the host cell chromosome, thereby yielding a high level of expression. These vectors can be used for stable expression by including a selectable marker in the plasmid, such as, for example, the neo gene. Alternatively, the retroviral genome can be modified for use as a vector capable of introducing and directing the expression of the MICAL or MICAL-Like gene in host cells (Cone and Mulligan, Proc. Natl. Acad. Sci., USA 81:6349-6353, 1984). High level expression can also be achieved using inducible promoters, including, but not limited to, the metallothionein IIA promoter and heat shock promoters.

For long term, high yield production of recombinant proteins, stable expression is preferred. Rather than using expression vectors which contain viral origins of replication, host cells can be transformed with the MICAL or a MICAL-Like polypeptide encoding cDNA controlled by appropriate expression control elements such as promoter, enhancer, sequences, transcription terminators, and polyadenylation sites, and a selectable marker. The selectable marker in the recombinant plasmid can confer resistance to the selection, and allows cells to stably integrate the plasmid into their chromosomes and grow to form foci, which, in turn can be cloned and expanded into cell lines. For example, following the introduction of foreign DNA, engineered cells can be allowed to grow for 1 to 2 days in an enriched media, and then are switched to a selective media. A number of selection systems can be used, including, but not limited to, the herpes simplex virus thymidine kinase (Wigler et al., Cell 11:223, 1977), hypoxanthine-guanine phosphoribosyltransferase (Szybalska and Szybalski, Proc. Natl. Acad. Sci., USA 48:2026, 1982), and adenine phosphoribosyltransferase (Lowy, et al., Cell 22:817, 1980) genes can be employed in tk⁻, hgprt⁻ or aprt⁻ cells respectively. Also, antimetabolite resistance can be used as the basis of selection for dhfr, which confers resistance to methotrexate (Wigler, et al., Proc. Natl. Acad. Sci. USA 77:3567, 1980; O'Hare et al., Proc. Natl. Acad. Sci., USA 78: 1527, 1981); gpt, which confers resistance to mycophenolic acid (Mulligan and Berg, Proc. Natl. Acad. Sci., USA 78:2072, 1981); neo, which confers resistance to the aminoglycoside G-418 (Colberre-Garapin et al., J. Mol. Biol. 150:1, 1981); and hygro, which confers resistance to hygromycin (Santerre et al., Gene 30:147, 1984) genes. Additional selectable genes, including trpB, which allows cells to utilize indole in place of tryptophan; hisD, which allows cells to utilize histinol in place of histidine (Hartman and Mulligan, Proc. Natl. Acad. Sci., USA 85:8047, 1988); and ODC (ornithine decarboxylase) which confers resistance to the ornithine decarboxylase inhibitor, 2-(difluoromethyl)-DL-ornithine, DFMO (McConlogue, Curr. Comm. Mol. Biol. (Cold Spring Harbor Laboratory Press, 1987), also have been described.

When the host is a eukaryote, such methods of transfection of DNA as calcium phosphate coprecipitates, conventional mechanical procedures such as microinjection, electroporation, insertion of a plasmid encased in liposomes, or virus vectors can be used. Eukaryotic cells can also be cotransformed with DNA sequences encoding the MICAL or a MICAL-Like polypeptides of the invention, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein. (Gluzman, Eukaryotic Viral Vectors (Cold Spring Harbor Laboratory Press, 1982)).

The invention also provides stable recombinant cell lines, the cells of which express MICAL or a MICAL-Like polypeptides and contain DNA that encodes MICAL or a MICAL-Like polypeptides. Suitable cell types include, but are not limited to, NIH 3T3 cells (murine), C2C12 cells, L6 cells, and P19 cells. C2C12 and L6 myoblasts differentiate spontaneously in culture and form myotubes depending on the particular growth conditions (Yaffe and Saxel, Nature 270:725-727, 1977; Yaffe, Proc. Natl. Acad. Sci., USA 61:477-483, 1968). P19 is an embryonal carcinoma cell line. Such cells are described, for example, in the Cell Line Catalog of the American Type Culture Collection (ATCC). These cells can be stably transformed using well known methods (see, for example, Ausubel et al., supra, 1995, see sections 9.5.1-9.5.6).

A MICAL or a MICAL-Like polypeptide can be expressed from a recombinant polynucleotide of the invention using inducible or constitutive regulatory elements, as described herein. The desired protein encoding sequence and an operably linked promoter can be introduced into a recipient cell either as a non-replicating DNA (or RNA) molecule, which can either be a linear molecule or a covalently closed circular molecule. Expression of the desired molecule can occur due to transient expression of the introduced sequence, or the polynucleotide can be stably maintained in the cell, for example, by integration into a host cell chromosome, thus allowing a more permanent expression. Accordingly, the cells can be stably or transiently transformed (transfected) cells.

An example of a vector that can be employed is one which is capable of integrating the desired gene sequences into the host cell chromosome. Cells which have stably integrated the introduced DNA into their chromosomes can be selected by also introducing one or more markers which allow for selection of host cells which contain the expression vector. The marker can complement an auxotrophy in the host such as leu2, or ura3, which are common yeast auxotrophic markers; can confer a biocide resistance, for example, to an antibiotic or to heavy metal ions such as copper, or the like. The selectable marker gene can either be directly linked to the DNA gene sequences to be expressed, or can be introduced into the same cell by cotransfection.

The introduced sequence can be incorporated into a plasmid or viral vector capable of autonomous replication in the recipient host. Any of a variety of vectors can be employed for this purpose. Factors of importance in selecting a particular plasmid or viral vector include the ease with which recipient cells that contain the vector can be recognized and selected from those cells that do not contain the vector; the number of copies of the vector desired in a particular host cell; and whether it is desirable to be able to “shuttle” the vector between host cells of different species.

For a mammalian host, several vector systems are available for expression. One class of vectors utilizes DNA elements that provide autonomously replicating extra-chromosomal plasmids derived from animal viruses, for example, a bovine papilloma virus, polyoma virus, adenovirus, or SV40 virus. A second class of vectors includes vaccinia virus expression vectors. A third class of vectors relies upon the integration of the desired gene sequences into the host chromosome. Cells that have stably integrated the introduced DNA into their chromosomes can be selected by also introducing one or more markers genes (as described above), which allow selection of host cells that contain the expression vector. The selectable marker gene can be directly linked to the DNA sequences to be expressed, or introduced into the same cell by cotransfection. Additional elements can be included to provide for optimal synthesis of an encoded mRNA or peptide, including, for example, splice signals, transcription promoters or enhancers, and transcription or translation termination signals. cDNA expression vectors incorporating appropriate regulatory elements are well known in the art (see, for example, Okayama, Mol. Cell. Biol. 3:280, 1983).

Once the vector or DNA sequence containing the construct has been prepared for expression, the DNA construct can be introduced into an appropriate host. Various methods can be used for introducing the polynucleotide into a cell, including, for example, methods of transfection or transformation such as protoplast fusion, calcium phosphate precipitation, and electroporation or other conventional techniques, for example, infection where the vector is a viral vector.

The invention also provides transgenic non-human animals that have cells that constitutively express a recombinant MICAL or a MICAL-Like polypeptide or that have recombinantly inactivated MICAL or MICAL-Like function. In certain aspects, transgenic animals that constitutively express a recombinant MICAL or MICAL-Like protein can be expressed with a tag sequence that can be used to facilitate immunoprecipitation of the MICAL or MICAL-Like polypeptide. Such transgenic non-human animals can be used for example, to facilitate the identification of agents which bind the MICAL or MICAL-Like polypeptides. Alternatively, the transgenic non-human organism can express a mutant transgenic MICAL in the central nervous system or peripheral nervous system, to identify mutant MICAL polypeptides that affect axonal guidance.

Accordingly, in one aspect, the transgenic animal is a transgenic non-human organism whose genome includes a transgenic DNA sequence that includes a polynucleotide that encodes a mutant MICAL polypeptide operably linked to a promoter that is active in the central nervous system and/or peripheral nervous system, wherein the mouse expresses the transgenic polynucleotide in the central nervous system and/or peripheral nervous system, and wherein expression levels of transgenic polynucleotide are sufficient to effect an axonal guidance phenotype of the non-human organism.

In another aspect, the transgenic animal is a non-human transgenic animal having a genome comprising a transgene comprising a nucleotide sequence encoding a MICAL polypeptide operably linked to a heterologous promoter. The non-human transgenic animal expresses the transgenic polynucleotide in the central nervous system and/or peripheral nervous system at expression levels sufficient to effect an axonal guidance phenotype of the non-human organism. In one aspect, the MICAL polypeptide is ectopically expressed. The MICAL polypeptide is expressed in the transgenic animal at a greater level in one or more cells of the non-human transgenic animal than the MICAL polypeptide is expressed in comparable cells of a comparable non-human transgenic animal.

In another embodiment, the present invention provides a non-human transgenic animal having a genome comprising a recombinantly inactivated nucleotide sequence encoding a MICAL polypeptide that has been recombinantly inactivated. The non-human transgenic animal has an altered phenotype that results from inactivation of the MICAL polypeptide. For example, the altered phenotype can be an altered axon guidance phenotype.

The non-human transgenic animal of this aspect of the invention, in certain aspects is heterozygous for the nucleotide sequence that has been inactivated. Alternatively, the non-human transgenic animal of this aspect of the invention, can be homozygous for the nucleotide sequence that has been recombinantly inactivated.

As used herein, the term “transgenic,” when used in reference to an animal or an organism, means that cells of the animal or organism have been genetically manipulated to contain an exogenous polynucleotide sequence that is stably maintained with the cells. The manipulation can be, for example, microinjection of a polynucleotide or infection with a recombinant virus containing the polynucleotide. Thus, the term “transgenic” is used herein to refer to animals (organisms) in which one or more cells receive a recombinant polynucleotide, which can be integrated into a chromosome in the cell, or can be maintained as an extrachromosomally replicating polynucleotide, such as might be engineered into a yeast artificial chromosome. The term “transgenic animal” also includes a “germ cell line” transgenic animal. A germ cell line transgenic animal is a transgenic animal in which the genetic information has been taken up and incorporated into a germ line cell, therefore conferring the ability to transfer the information to offspring. If such offspring in fact possess some or all of that information, the offspring also are considered to be transgenic animals. The invention further encompasses transgenic organisms.

A transgenic organism can be any organism whose genome has been altered by in vitro manipulation of an early stage embryo or a fertilized egg, or by any transgenic technology to induce a specific gene knock-out. The term “gene knock-out” refers to the targeted disruption of a gene in a cell or in vivo that results in complete loss of function. Gene knock-outs is also referred to herein as inactivated genes, such as recombinantly inactivated genes. A target gene in a transgenic animal can be rendered nonfunctional by an insertion targeted to the gene to be rendered nonfunctional, for example, by homologous recombination, or by any other method for disrupting the function of a gene in a cell.

The transgene to be used in the practice of the subject invention can be a DNA sequence comprising a modified MICAL or MICAL-Like polypeptide coding sequence. Preferably, the modified MICAL or MICAL-Like gene is one that is disrupted by homologous targeting in embryonic stem cells. For example, the entire MICAL gene can be deleted (See Examples herein). Optionally, the disruption (or deletion) can be accompanied by insertion of or replacement with another polynucleotide, for example, a polynucleotide encoding a nonfunctional MICAL or MICAL-Like polypeptide. A “knock-out” phenotype also can be conferred by introducing or expressing an antisense MICAL or MICAL-Like polypeptide encoding polynucleotide in a cell in the organism, or by expressing an antibody or a dominant negative MICAL or MICAL-Like polypeptide in the cells.

Various methods are known for producing a transgenic animal. In one method, an embryo at the pronuclear stage (a “one cell embryo”) is harvested from a female and the transgene is microinjected into the embryo, in which case the transgene will be chromosomally integrated into the germ cells and somatic cells of the resulting mature animal. In another method, embryonic stem cells are isolated and the transgene is incorporated into the stem cells by electroporation, plasmid transfection or microinjection; the stem cells are then reintroduced into the embryo, where they colonize and contribute to the germ line. Methods for microinjection of polynucleotides into mammalian species are described, for example, in U.S. Pat. No. 4,873,191, which is incorporated herein by reference. In yet another method, embryonic cells are infected with a retrovirus containing the transgene, whereby the germ cells of the embryo have the transgene chromosomally integrated therein.

When the animals to be made transgenic are avian, microinjection into the pronucleus of the fertilized egg is problematic because avian fertilized ova generally go through cell division for the first twenty hours in the oviduct and, therefore, the pronucleus is inaccessible. Thus, the retrovirus infection method is preferred for making transgenic avian species (see U.S. Pat. No. 5,162,215, which is incorporated herein by reference). If microinjection is to be used with avian species, however, the embryo can be obtained from a sacrificed hen approximately 2.5 hours after the laying of the previous laid egg, the transgene is microinjected into the cytoplasm of the germinal disc and the embryo is cultured in a host shell until maturity (Love et al., Biotechnology 12, 1994). When the animals to be made transgenic are bovine or porcine, microinjection can be hampered by the opacity of the ova, thereby making the nuclei difficult to identify by traditional differential interference-contrast microscopy. To overcome this problem, the ova first can be centrifuged to segregate the pronuclei for better visualization.

Non-human transgenic animals of the invention can be an invertebrate or a vertebrate. For example, the transgenic organism can be Drosophila or a mammal such as a mouse or a rat. The transgene can be introduced into embryonal target cells at various developmental stages, and different methods are selected depending on the stage of development of the embryonal target cell. The zygote is the best target for microinjection. The use of zygotes as a target for gene transfer has a major advantage in that the injected DNA can incorporate into the host gene before the first cleavage (Brinster et al., Proc. Natl. Acad. Sci., USA 82:4438-4442, 1985). As a consequence, all cells of the transgenic non-human animal carry the incorporated transgene, thus contributing to efficient transmission of the transgene to offspring of the founder, since 50% of the germ cells will harbor the transgene.

A transgenic animal can be produced by crossbreeding two chimeric animals, each of which includes exogenous genetic material within cells used in reproduction. Twenty-five percent of the resulting offspring will be transgenic animals that are homozygous for the exogenous genetic material, 50% of the resulting animals will be heterozygous, and the remaining 25% will lack the exogenous genetic material and have a wild type phenotype.

In the microinjection method, the transgene is digested and purified free from any vector DNA, for example, by gel electrophoresis. The transgene can include an operatively associated promoter, which interacts with cellular proteins involved in transcription, and provides for constitutive expression, tissue specific expression, developmental stage specific expression, or the like. Such promoters include those from cytomegalovirus (CMV), Moloney leukemia virus (MLV), and herpes virus, as well as those from the genes encoding metallothionein, skeletal actin, Phosphenolpyruvate carboxylase (PEPCK), phosphoglycerate (PGK), dihydrofolate reductase (DHFR), and thymidine kinase (TK). Promoters from viral long terminal repeats (LTRs) such as Rous sarcoma virus LTR also can be employed. When the animals to be made transgenic are avian, preferred promoters include those for the chicken β-globin gene, chicken lysozyme gene, and avian leukosis virus. Constructs useful in plasmid transfection of embryonic stem cells will employ additional regulatory elements, including, for example, enhancer elements to stimulate transcription, splice acceptors, termination and polyadenylation signals, ribosome binding sites to permit translation, and the like.

In the retroviral infection method, the developing non-human embryo can be cultured in vitro to the blastocyst stage. During this time, the blastomeres can be targets for retroviral infection (Jaenich, Proc. Natl. Acad. Sci, USA 73:1260-1264, 1976). Efficient infection of the blastomeres is obtained by enzymatic treatment to remove the zona pellucida (Hogan et al., Manipulating the Mouse Embryo (Cold Spring Harbor Laboratory Press, 1986). The viral vector system used to introduce the transgene is typically a replication-defective retrovirus carrying the transgene (Jahner et al., Proc. Natl Acad. Sci., USA 82:6927-6931, 1985; Van der Putten et al., Proc. Natl. Acad. Sci, USA 82:6148-6152, 1985). Transfection is easily and efficiently obtained by culturing the blastomeres on a monolayer of virus producing cells (Van der Putten et al., supra, 1985; Stewart et al., EMBO J. 6:383-388, 1987). Alternatively, infection can be performed at a later stage. Virus or virus-producing cells can be injected into the blastocoele (Jahner et al., Nature 298:623-628, 1982). Most of the founders will be mosaic for the transgene since incorporation occurs only in a subset of the cells which formed the transgenic nonhuman animal. Further, the founder can contain various retroviral insertions of the transgene at different positions in the genome, which generally will segregate in the offspring. In addition, it is also possible to introduce transgenes into the germ line, albeit with low efficiency, by intrauterine retroviral infection of the mid-gestation embryo (Jahner et al., supra, 1982).

Embryonal stem cell (ES) also can be targeted for introduction of the transgene. ES cells are obtained from pre-implantation embryos cultured in vitro and fused with embryos (Evans et al. Nature 292:154-156, 1981; Bradley et al., Nature 309:255-258, 1984; Gossler et al., Proc. Natl. Acad. Sci., USA 83:9065-9069, 1986; Robertson et al., Nature 322:445-448, 1986). Transgenes can be efficiently introduced into the ES cells by DNA transfection or by retrovirus mediated transduction. Such transformed ES cells can thereafter be combined with blastocysts from a nonhuman animal. The ES cells thereafter colonize the embryo and contribute to the germ line of the resulting chimeric animal (see Jaenisch, Science 240:1468-1474, 1988).

The present invention also provides an antibody or antigen binding fragment thereof that specifically bind a MICAL polypeptide or a MICAL-Like polypeptide, or a functional peptide portion thereof. Particularly useful antibodies of the invention include antibodies that specifically bind a plexin interacting region of a MICAL, thereby inhibiting binding of the MICAL to plexins. Such antibodies can be useful, for example, for inhibiting semaphorin-mediate axonal repulsion and thus stimulating, for example, regeneration of axon connections after spinal cord injury. In certain aspects, the present invention provides an antibody or antigen binding fragment that binds an N-terminal MICAL domain, a MICAL calponin homology domain, a MICAL LIM domain, a MICAL proline rich region, or a MICAL plexin-interacting region.

A monoclonal antibody that binds specifically to a MICAL or MICAL-Like polypeptide can be used to treat a pathological condition involving, for example failure of axon regrowth. In a preferred embodiment, the MICAL or MICAL-Like polypeptide antibody is administered to a patient by intravenous, intramuscular subcutaneous injection, or direct injection to a site of spinal cord damage. A monoclonal antibody can be administered, for example, within a dose range between about 0.1 μg/kg to about 100 mg/kg; more preferably between about 1 μg/kg to 75 mg/kg; most preferably from about 10 mg/kg to 50 mg/kg. The antibody can be administered, for example, by bolus injunction or by slow infusion. Slow infusion over a period of 30 minutes to 2 hours is preferred. The anti-MICAL or anti-MICAL-Like polypeptide antibody, can be formulated in a formulation suitable for administration to a patient. Such formulations are known in the art.

The dosage regimen will be determined by the attending physician considering various factors which modify the action of the MICAL or MICAL-Like polypeptide protein or the plexin protein, for example, amount of tissue desired to be formed, the site of tissue damage, the condition of the damaged tissue, the size of a wound, type of damaged tissue, the patient's age, sex, and diet, the severity of any infection, time of administration and other clinical factors. The dosage can vary with the type of matrix used in the reconstitution and the types of agent, such as anti-MICAL or anti-MICAL-Like polypeptide antibodies, to be used in the composition. Generally, systemic or injectable administration, such as intravenous, intramuscular, subcutaneous injection, or injection to a site of damage of the nervous system is employed. Administration generally is initiated at a dose which is minimally effective, and the dose is increased over a preselected time course until a positive effect is observed. Subsequently, incremental increases in dosage are made limiting such incremental increases to such levels that produce a corresponding increase in effect, while taking into account any adverse affects that can appear. The addition of other agents that promote neuron process regrowth, can also affect the dosage.

As used herein, the term “antibody” is used in its broadest sense to include polyclonal and monoclonal antibodies, as well as antigen binding fragments of such antibodies. An antibody useful in a method of the invention, or an antigen binding fragment thereof, is characterized, for example, by having specific binding activity for an epitope of a MICAL or MICAL-Like polypeptide, or a Plexin. In addition, as discussed above, an antibody of the invention can be an antibody that specifically binds a peptide portion of a MICAL, a MICAL-Like polypeptide, or a plexin, particularly a plexin-interacting region of a MICAL or a MICAL-binding region of a plexin.

The term “binds specifically” or “specific binding activity,” when used in reference to an antibody means that an interaction of the antibody and a particular epitope has a dissociation constant of at least about 1×10⁻⁶, generally at least about 1×10⁻⁷, usually at least about 1×10⁻⁸, and particularly at least about 1×10⁻⁹ or 1×10⁻¹⁰ or less. As such, Fab, F(ab′)₂, Fd and Fv fragments of an antibody that retain specific binding activity for an epitope of a MICAL or MICAL-Like polypeptide, are included within the definition of an antibody.

The term “antibody” as used herein includes naturally occurring antibodies as well as non-naturally occurring antibodies, including, for example, single chain antibodies, chimeric, bifunctional, human, and humanized antibodies, intrabodies (i.e. intracellularly expressed antibodies, see e.g., Chen, S. Y., et al., Hum. Gene Ther 5:595-601 (1994)), as well as antigen-binding fragments thereof. Such non-naturally occurring antibodies can be constructed using solid phase peptide synthesis, can be produced recombinantly or can be obtained, for example, by screening combinatorial libraries consisting of variable heavy chains and variable light chains (see Huse et al., Science 246:1275-1281 (1989), which is incorporated herein by reference). These and other methods of making, for example, chimeric, humanized, CDR-grafted, single chain, and bifunctional antibodies are well known to those skilled in the art (Winter and Harris, Immunol. Today 14:243-246, 1993; Ward et al., Nature 341:544-546, 1989; Harlow and Lane, Antibodies: A laboratory manual (Cold Spring Harbor Laboratory Press, 1988); Hilyard et al., Protein Engineering: A practical approach (IRL Press 1992); Borrabeck, Antibody Engineering, 2d ed. (Oxford University Press 1995); each of which is incorporated herein by reference).

Antibodies that bind specifically with a MICAL or MICAL-Like polypeptide can be raised using the MICAL or MICAL-Like polypeptide, or a fragment thereof, as an immunogen. Where such a polypeptide or fragment thereof is non-immunogenic, it can be made immunogenic by coupling the hapten to a carrier molecule such as bovine serum albumin (BSA) or keyhole limpet hemocyanin (KLH), or by expressing the peptide portion as a fusion protein. Various other carrier molecules and methods for coupling a hapten to a carrier molecule are well known in the art (see, for example, by Harlow and Lane, supra, 1988).

If desired, a kit incorporating an antibody or other agent useful in a method of the invention can be prepared. Such a kit can contain, in addition to the agent, a pharmaceutical composition in which the agent can be reconstituted for administration to a subject. The kit also can contain, for example, reagents for detecting the antibody, or for detecting specific binding of the antibody to a MICAL or MICAL-Like polypeptide. Such detectable reagents useful for labeling or otherwise identifying the antibody are described herein and known in the art.

Methods for raising polyclonal antibodies, for example, in a rabbit, goat, mouse or other mammal, are well known in the art (see, for example, Green et al., “Production of Polyclonal Antisera,” in Immunochemical Protocols (Manson, ed., Humana Press 1992), pages 1-5; Coligan et al., “Production of Polyclonal Antisera in Rabbits, Rats, Mice and Hamsters,” in Curr. Protocols Immunol. (1992), section 2.4.1; each or which is incorporated herein by reference). In addition, monoclonal antibodies can be obtained using methods that are well known and routine in the art (Harlow and Lane, supra, 1988). For example, spleen cells from a mouse immunized with a MICAL or MICAL-Like polypeptide, or an epitopic fragment thereof, can be fused to an appropriate myeloma cell line such as SP/02 myeloma cells to produce hybridoma cells. Cloned hybridoma cell lines can be screened using labeled antigen to identify clones that secrete monoclonal antibodies having the appropriate specificity, and hybridomas expressing antibodies having a desirable specificity and affinity can be isolated and utilized as a continuous source of the antibodies. The antibodies can be further screened for the inability to bind specifically with the MICAL or MICAL-Like polypeptide. Such antibodies are useful, for example, for preparing standardized kits for clinical use. A recombinant phage that expresses, for example, a single chain anti-MICAL or MICAL-Like polypeptide antibody also provides an antibody that can used for preparing standardized kits.

Methods of preparing monoclonal antibodies well known (see, for example, Kohler and Milstein, Nature 256:495, 1975, which is incorporated herein by reference; see, also, Coligan et al., supra, 1992, see sections 2.5.1-2.6.7; Harlow and Lane, supra, 1988). Briefly, monoclonal antibodies can be obtained by injecting mice with a composition comprising an antigen, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures.

Monoclonal antibodies can be isolated and purified from hybridoma cultures by a variety of well established techniques, including, for example, affinity chromatography with Protein-A SEPHAROSE, size exclusion chromatography, and ion exchange chromatography (Coligan et al., supra, 1992, see sections 2.7.1-2.7.12 and sections 2.9.1-2.9.3; see, also, Barnes et al., “Purification of Immunoglobulin G (IgG),” in Meth. Molec. Biol. 10:79-104 (Humana Press 1992), which is incorporated herein by reference). Methods of in vitro and in vivo multiplication of monoclonal antibodies is well known to those skilled in the art. Multiplication in vitro can be carried out in suitable culture media such as Dulbecco's Modified Eagle Medium or RPMI 1640 medium, optionally replenished by a mammalian serum such as fetal calf serum or trace elements and growth sustaining supplements such as normal mouse peritoneal exudate cells, spleen cells, bone marrow macrophages. Production in vitro provides relatively pure antibody preparations and allows scale-up to yield large amounts of the desired antibodies. Large scale hybridoma cultivation can be carried out by homogenous suspension culture in an airlift reactor, in a continuous stirrer reactor, or in immobilized or entrapped cell culture. Multiplication in vivo can be carried out by injecting cell clones into mammals histocompatible with the parent cells, for example, syngeneic mice, to cause growth of antibody producing tumors. Optionally, the animals are primed with a hydrocarbon, especially oils such as pristane (tetramethylpentadecane) prior to injection. After one to three weeks, the desired monoclonal antibody is recovered from the body fluid of the animal.

Therapeutic applications for antibodies disclosed herein are also part of the present invention. For example, antibodies of the present invention can also be derived from subhuman primate antibody. General techniques for raising therapeutically useful antibodies in baboons can be found, for example, in Goldenberg et al., International Patent Publication WO 91/11465 (1991); and Losman et al., Int. J. Cancer 46:310, 1990, each of which is incorporated herein by reference. Accordingly, the present invention provides antibodies conjugated to a therapeutic moiety. For example, in certain aspects the present invention provides an anti-MICAL antibody conjugated to a monooxygenase inhibitor, for example EGCG.

A therapeutically useful anti-MICAL or MICAL-Like polypeptide antibody also can be derived from a “humanized” monoclonal antibody. Humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are known (see, for example, Orlandi et al., Proc. Natl. Acad. Sci., USA 86:3833, 1989, which is hereby incorporated in its entirety by reference). Techniques for producing humanized monoclonal antibodies also are known (see, for example, Jones et al., Nature 321:522, 1986; Riechmann et al., Nature 332:323, 1988; Verhoeyen et al., Science 239:1534, 1988; Carter et al., Proc. Natl. Acad. Sci., USA 89:4285, 1992; Sandhu, Crit. Rev. Biotechnol. 12:437, 1992; and Singer et al., J. Immunol. 150:2844, 1993; each of which is incorporated herein by reference).

Antibodies of the invention also can be derived from human antibody fragments isolated from a combinatorial immunoglobulin library (see, for example, Barbas et al., METHODS: A Companion to Methods in Immunology 2:119, 1991; Winter et al., Ann. Rev. Immunol. 12:433, 1994; each of which is incorporated herein by reference). Cloning and expression vectors that are useful for producing a human immunoglobulin phage library can be obtained, for example, from STRATAGENE Cloning Systems (La Jolla, Calif.).

An antibody of the invention also can be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody-secreting hybridomas. Methods for obtaining human antibodies from transgenic mice are described, for example, by Green et al., Nature Genet. 7:13, 1994; Lonberg et al., Nature 368:856, 1994; and Taylor et al., Int. Immunol. 6:579, 1994; each of which is incorporated herein by reference.

Antibody fragments of the present invention can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see, for example, Goldenberg, U.S. Pat. No. 4,036,945 and U.S. Pat. No. 4,331,647, each of which is incorporated by reference, and references contained therein; Nisonhoff et al., Arch. Biochem. Biophys. 89:230.1960; Porter, Biochem. J. 73:119, 1959; Edelman et al., Meth. Enzymol., 1:422 (Academic Press 1967), each of which is incorporated herein by reference; see, also, Coligan et al., supra, 1992, see sections 2.8.1-2.8.10 and 2.10.1-2.10.4).

Other methods of cleaving antibodies, such as separation of heavy chains to form monovalent light/heavy chain fragments, further cleavage of fragments, or other enzymatic, chemical, or genetic techniques can also be used, provided the fragments specifically bind to the antigen that is recognized by the intact antibody. For example, Fv fragments comprise an association of V_{H} and V_{L} chains. This association can be noncovalent (Inbar et al., Proc. Natl. Acad. Sci., USA 69:2659, 1972). Alternatively, the variable chains can be linked by an intermolecular disulfide bond or cross-linked by chemicals such as glutaraldehyde (Sandhu, supra, 1992). Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow et al., Methods: A Companion to Methods in Enzymology 2:97, 1991; Bird et al., Science 242:423-426, 1988; Ladner et al., U.S. Pat. No. 4,946,778; Pack et al., Bio/Technology 11: 1271-1277, 1993; each of which is incorporated herein by reference; see, also Sandhu, supra, 1992.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (see, for example, Larrick et al., Methods: A Companion to Methods in Enzymology 2:106, 1991, which is incorporated herein in its entirety by reference).

An intrabody comprises at least a portion of an antibody that is capable of immunospecifically binding an antigen and preferably does not contain sequences coding for its secretion. Such antibodies will bind antigen intracellularly. In one embodiment, the intrabody comprises a single-chain Fv (“sFv”). sFvs are antibody fragments comprising the VH and VL domains of antibody, wherein these domains are present in a single polypeptide chain. Generally, the sFv polypeptide further comprises a polypeptide linker between the VH and VL domains which enables the sFv to form the desired structure for antigen binding. For a review of sFvs see Pluckthun in The Pharmacology of Monoclonal Antibodies, vol. 113, Rosenburg and Moore eds. Springer-Verlag, New York, pp. 269-315 (1994). In a further embodiment, the intrabody preferably does not encode an operable secretory sequence and thus remains within the cell (see generally Marasco, Wash., 1998, “Intrabodies: Basic Research and Clinical Gene Therapy Applications” Springer:New York). Generation of intrabodies is well-known to the skilled artisan and is described, for example, in U.S. Pat. Nos. 6,004,940; 6,072,036; 5,965,371, which are incorporated by reference in their entireties herein. Further, the construction of intrabodies is discussed in Ohage and Steipe, 1999, J Mol. Biol. 291:1119-1128; Ohage et al., 1999, J. Mol. Biol. 291:1129-1134; and Wirtz and Steipe, 1999, Protein Science 8:2245-2250, which references are incorporated herein by reference in their entireties.

In another embodiment, the present invention provides a double-stranded RNA molecule that includes a first RNA strand that specifically hybridizes to an mRNA encoding a MICAL polypeptide. The double-stranded RNA molecule also includes a second RNA strand that is the reverse complement of the first strand. The double-stranded molecule is at least 15 base pairs in length. Double stranded RNA is involved in RNA interference, the process by which double-stranded RNA induces the silencing of homologous endogenous genes. (Hammond, S. M., et al., Nat. Rev. Genet. 2(2):110-9 (2001)). Accordingly, double stranded RNA of this embodiment inhibit expression of a MICAL polypeptide, thereby promoting axonal growth and target formation.

In another embodiment the present invention provides a method for identifying an agent that affects an activity of a MICAL or MICAL-Like protein. As such, the present invention provides screening methods for agents that affect MICAL protein or MICAL-Like protein activity. The method typically includes contacting a MICAL polypeptide, or a functional portion thereof or a MICAL-Like polypeptide, or a functional portion thereof, or a cell expressing at least one of these polypeptides, with a candidate agent, and determining whether the agent affects an activity of the polypeptide. The activity of the MICAL protein, can be any of the activities identified herein for a MICAL polypeptide.

For example, a method according to this embodiment can identify an agent that affects MICAL monooxygenase activity or plexin interacting activity. Methods for identifying monooxygenase activity and plexin interacting activity are known in the art. Examples of these methods are provided herein. For example, an immunoprecipitation experiment can be performed in the presence of plexA and a MICAL polypeptide, wherein the plexA and/or MICAL polypeptide are contacted with an on-test agent. It can then be determined whether the agent affected binding of PlexA and the MICAL polypeptide. Agents that affect binding are candidate agents for treatment of disorders such as spinal cord injury, since they are expected to inhibit semaphorin-mediated axonal repulsion.

In one aspect of this embodiment, the method can identify an agent that affects a semaphorin-mediated process. That is, the activity of a MICAL protein can be participation, typically regulation, of a semaphorin mediated process. For example, a semaphorin-mediated process can be semaphorin-mediated axonal repulsion. In particular embodiments, the semaphorin-mediated process is mediated by semaphorins 1A and 3A, 4A, or a class 7 semaphorin.

In aspects of this embodiment that include a cell, the cell can be virtually any cell. Recombinant cells can be produced using standard techniques as disclosed herein, that express a MICAL polypeptide using polynucleotides and vectors of the present invention. In embodiments where the method identifies an agent that affects a MICAL activity and/or a semaphorin-mediated process, the cell, for example, can be a cell of a type that is known to include a semaphorin-mediated process, as discussed hereinbelow, such as a cell of the immune system, for example a B cell or a T cell, a cell of neuronal origin, a cell with a transformed phenotype, a cardiac cell, or a neural crest precursor cell of a cardiac cell.

In another aspect of this embodiment, the present invention provides a method for identifying an agent that affects axonal guidance regulatory activity, monooxygenase activity, actin binding activity and/or plexin-interacting activity. The method typically includes contacting an isolated polypeptide of the present invention that has axonal guidance regulatory activity, monooxygenase activity, actin binding activity, and/or plexin-interacting activity, or a cell expressing the polypeptide, with a candidate agent. Next, axonal guidance regulatory activity, monooxygenase activity, actin binding activity, and/or plexin-interacting activity is compared in the presence versus absence of the agent. A difference in activity is indicative of an agent that affects axonal guidance regulatory activity, monooxygenase activity, and/or plexin-interacting activity. In this aspect of the invention, the cell is typically a cell of neuronal original, such as a neuron that recombinantly expresses the MICAL.

Methods for determining axonal guidance regulatory activity (i.e. axon guidance regulatory activity assays), for example semaphorin axonal repulsion activity, are known in the art. Some of these methods are disclosed herein. For example, an in vitro method such as a rat DRG growth cone repulsion assay using Sema 3A-secreting 293 cells, as disclosed above, can be employed (FIG. 4A; Messersmith et al., 1995). It will be recognized however, that virtually any assay of semaphorin mediated axon repulsion can be employed in the methods of the present invention for identifying an agent that affects axonal guidance regulatory activity. Methods for assessing plexin-interacting activity and monooxygenase activity are provided herein.

In another embodiment, the present invention provides a method for screening for an agent that modulates an activity of a MICAL or MICAL-Like polypeptide. The method includes contacting a cell that recombinantly expresses a MICAL or MICAL-Like polypeptide with a candidate agent. Then a phenotypic or physiological trait of the cell is compared in the presence or absence of the candidate agent. A difference in the phenotypic or physiological trait indicates that the agent modulates the activity of the MICAL polypeptide. The phenotypic or physiological trait can involve dynamics of the cytoskeleton, or can be axon guidance, cell proliferation or invasiveness, or an immune response.

In another embodiment, the present invention provides a method for screening for an agent that modulates the expression of a MICAL or MICAL-Like polypeptide. The method includes contacting a cell with a candidate agent. Then comparing the expression of the MICAL or MICAL-Like polypeptide, for example in the presence or absence of the candidate agent. A difference in the expression indicates that the agent modulates the expression of the MICAL polypeptide. In one aspect, the level of mRNA encoding MICAL is compared. In another aspect, the level of the MICAL polypeptide is compared.

Specificity can be further analyzed, for example, by assaying for an activity or phenotype in cells that recombinantly overexpress MICAL and comparable cells that do not recombinantly overexpress MICAL, or cells in which MICAL has been knocked out or reduced versus comparable cells in which MICAL is expressed at normal levels.

As illustrated in the Examples herein, MICALs are susceptible to small molecule inhibitors that affect their ability to oxidize their substrate. For example, gallic acid derivatives, including the green tea component (−)-epigallocatechin gallate (EGCG), appear to be potent and selective inhibitors of MICALs. It will be recognized that based on the identification of these small molecule inhibitors of semaphorin-mediated axonal repulsion, it will be recognized that other small molecule inhibitors can be identified.

All available evidence points to the plexin cytoplasmic domain as an essential signal transducing domain for signaling class 3 semaphorin repulsion (Cheng et al., 2001; Takahashi and Strittmatter, 2001). Sema3A appears to utilize neuropilin-1 in combination with A class plexins to signal repulsive axon guidance. As illustrated in the Examples herein, agents that affect MICAL axonal guidance regulatory activity can be identified in vitro. For example, a rat DRG growth cone repulsion assay using Sema 3A-secreting 293 cells, as disclosed above, can be employed (FIG. 4A; Messersmith et al., 1995). NGF-dependent DRG axons exhibit little to no outgrowth towards Sema3A-secreting 293 cell aggregates (FIG. 4C). However, this repulsion can be neutralized in a specific and dose-dependent manner by inclusion of an inhibitor such as EGCG or EC, in the growth media (FIG. 4C). As illustrated in the Example, like EGCG, EC is capable of completely neutralizing Sema-3A-dependent repulsion in a dose-dependent manner, but a much higher EC concentration is required (FIG. 4C).

As will be understood, typically a method according to this embodiment of the invention includes a control sample wherein the polypeptide or cell is not contacted with the agent. However, known control values or qualitative results can also be used, such that a control sample does not need to be included each time the method is performed. For example, a visual microscopic comparison of axonal outgrowth can be performed using methods of the present invention that utilize the DRG rat growth cone repulsion assay. In addition, results of the rat DRG growth cone repulsion assay can be quantitated as illustrated in FIG. 4C. For example, results can be scored as the ratio of the axon lengths on the proximal and distal sides of the explant (P/D ratio), and on Sema3A-mediated growth cone collapse indicated as % collapsed growth cones. Therefore, known values for controls can be calculated and used to establish a baseline using known statistical methods, above which significant inhibition of the repulsion is established, thereby identifying an agent that affects axon guidance regulatory activity, most particular semaphorin-mediated axon repulsion.

The agent can affect axonal guidance regulatory activity, monooxygenase activity, and/or plexin interacting activity by enhancing or inhibiting this activity. The agent can be a small molecule, such as an antioxidant flavonoid, an antisense polynucleotide, a MICAL-like polypeptide or fragment thereof, a mutant MICAL polypeptide, a mutant MICAL-Like polypeptide, an anti-MICAL antibody, a double stranded RNA, or a peptidomimetic.

As indicated herein, anti-oxidant flavonoids such as ECGC and EC are inhibitors of semaphorin-mediated axonal repulsion. Therefore, particularly important candidate agents include those with anti-oxidant activity, especially anti-oxidant flavonoids or other green tea polyphenols. A variety of antioxidant flavonoids have been identified and can be analyzed to determine whether they affect semaphorin-mediated axonal repulsion. For example, the antioxidant flavonoid can be a gallic acid derivative or another flavoprotein monooxygenase inhibitor. Examples of gallic acid derivatives that can be analyzed using methods of the present invention include, but are not limited to, (−)-gallocatechin-3-O-gallate (GCG), (−)-epicatechin-3-O-gallate (ECG), (−)-epigallocatechin (EGC), (+)-gallocatechin (GC), theasinensin A, 3″-O-methyl-EGCG, 3″-O-methyl-ECG, 3″-O-methyl-GCG, (−)-epigallocatechin (EGC), (1)-gallocatechin (GC), gallic acid, catechin, n-octyl gallate, and n-cetyl gallate.

Additionally, it will be recognized that new antioxidant flavonoids, for example new gallic acid derivatives can be synthesized and tested using the methods of this aspect of the present invention for the ability to affect axonal guidance regulatory activity. Furthermore, flavoprotein monooxygenase inhibitors, which include but are not limited to certain anti-oxidant flavonoids can be analyzed for an affect on axonal guidance regulatory activity using methods of this embodiment of the present invention.

An agent tested by the screening embodiments of the present invention can include a known oxidase inhibitor. For example, an oxidase inhibitor outlined in Cross, Free Radical Biology and Med., 8:71-93 (1990) including, for example, DPI, ibuprofen, and aspirin. Furthermore, the screening assay can be used to test other agents including inhibitors of similar flavoprotein monooxygenase family members (See e.g., Cross (supra.); and Arnould and Camadro, Proc. Nat. Acad. Sci., 95:10553-10558 (1998)).

In certain embodiments, a screening method of the invention can be performed, for example, by contacting under suitable conditions a MICAL, or a functional peptide portion thereof, a plexin such as plexin A, and an agent to be tested. The MICAL, the plexin and the agent can be contacted in any order as desired. As such, the screening method can be used to identify agents that can competitively or non-competitively inhibit MICAL binding to plexin, agents that can mediate or enhance MICAL binding to the plexin, agents that can induce dissociation of specifically bound MICAL from the plexin, and agents that otherwise affect the ability of MICAL to regulate axon guidance, such agents having agonist or antagonist activity. Appropriate control reactions are performed to confirm that the action of the agent is specific with respect to the MICAL.

Suitable conditions for performing a screening method of the invention can be any conditions that allow MICAL to specifically interact with plexin, that provide a semaphorin-plexin repulsive axon guidance activity, and/or that support MICAL monooxygenase activity, including methods as disclosed herein or otherwise known in the art. Thus, suitable conditions for performing the screening assay can be, for example, in vitro conditions using a substantially purified MICAL and/or plexin; cell culture conditions, utilizing a cell that normally expresses a semaphorin-plexin A mediated repulsive axon activity and a MICAL polypeptide, for example, a neuron, or a cell that has been genetically modified to express a semaphorin-plexin A mediated repulsive axon activity including expression of a MICAL polypeptide; or in situ conditions as occur in an organism.

A screening method of the invention also can be performed using the methods of molecular modeling. The utilization of a molecular modeling method provides a convenient, cost effective means to identify those agents, among a large population such as a combinatorial library of potential agents, that are most likely to interact specifically with MICAL or a plexin, thereby reducing the number of potential agents that need to be screened using a biological assay. Upon identifying agents that interact specifically with a MICAL or a plexin, for example Plexin A, using a molecular modeling method, the selected agents can be examined for the ability to modulate an effect of a MICAL on a cell, such as regulation of axon guidance, using the methods disclosed herein.

The ability of a test agent to modulate an effect of MICAL can be detected using methods as disclosed herein or otherwise known in the art. The term “test agent” or “test molecule” is used broadly herein to mean any agent that is being examined for agonist or antagonist activity in a method of the invention. Although the method generally is used as a screening assay to identify previously unknown molecules that can act as agonist or antagonist agents as described herein, the methods also can be used to confirm that a agent known to have a particular activity in fact has the activity, for example, in standardizing the activity of the agent.

Further assays for testing the specificity of a candidate agent for MICAL, for example, test for lack of inhibition of steroid 5 alpha-reductase, NADPH-cytochrome P450 reductase, telomerase, MMP-2, MMP-9, and phenol sulfotransferase. These enzymes are known to be inhibited by EGCG in addition to its inhibition of flavoprotein monooxygenases. Therefore, by using these assays, agents can be identified that are more specific inhibitors of MICALs than EGCG.

A screening method of the invention provides the advantage that it can be adapted to high throughput analysis and, therefore, can be used to screen combinatorial libraries of test agents in order to identify those agents that can modulate an effect of MICAL on a cell, including those agents that can alter a specific interaction of MICAL and a plexin, and those that otherwise modulate MICAL axon guidance regulatory activity. Methods for preparing a combinatorial library of molecules that can be tested for a desired activity are well known in the art and include, for example, methods of making a phage display library of peptides, which can be constrained peptides (see, for example, U.S. Pat. No. 5,622,699; U.S. Pat. No. 5,206,347; Scott and Smith, Science 249:386-390, 1992; Markland et al., Gene 109:13-19, 1991; each of which is incorporated herein by reference); a peptide library (U.S. Pat. No. 5,264,563, which is incorporated herein by reference); a peptidomimetic library (Blondelle et al., Trends Anal. Chem. 14:83-92, 1995; a nucleic acid library (O'Connell et al., supra, 1996; Tuerk and Gold, supra, 1990; Gold et al., supra, 1995; each of which is incorporated herein by reference); an oligosaccharide library (York et al., Carb. Res., 285:99-128, 1996; Liang et al., Science, 274:1520-1522, 1996; Ding et al., Adv. Expt. Med. Biol., 376:261-269, 1995; each of which is incorporated herein by reference); a lipoprotein library (de Kruif et al., FEBS Lett., 399:232-236, 1996, which is incorporated herein by reference); a glycoprotein or glycolipid library (Karaoglu et al., J. Cell Biol., 130:567-577, 1995, which is incorporated herein by reference); or a chemical library containing, for example, drugs or other pharmaceutical agents (Gordon et al., J. Med. Chem., 37:1385-1401, 1994; Ecker and Crooke, Bio/Technology, 13:351-360, 1995; each of which is incorporated herein by reference). Polynucleotides can be particularly useful as agents that can modulate a specific interaction of MICAL and a plexin because nucleic acid molecules having binding specificity for cellular targets, including cellular polypeptides, exist naturally, and because synthetic molecules having such specificity can be readily prepared and identified (see, for example, U.S. Pat. No. 5,750,342, which is incorporated herein by reference).

In view of the present disclosure, it will be recognized that various animal model systems can be used as research tools to identify agents useful for practicing a method of the invention. For example, as discussed above, transgenic flies, mice or other experimental animals can be prepared and the transgenic non-human organism can be examined directly to determine the effect produced by expressing various levels of a particular agent in the organism.

As disclosed herein, MICALs can exert their activity, at least in part, through a Semaphorin-Plexin mediated pathway which can be associated with various pathological conditions. As such, the present invention provides new targets for the treatment of various conditions, especially regeneration of damaged neurological tissue, such as damage resulting from spinal cord injury, abnormal immune cell function, and abnormal cell motility such as cancer cell motility. Accordingly, the present invention provides methods for ameliorating the severity of a pathological condition in a subject, wherein the pathologic condition is characterized, for example, by an inability of neurons to regenerate proper axonal connections.

In another embodiment, the present invention provides a method for affecting axonal guidance regulatory activity. The method includes contacting a cell, typically a neuron, that expresses a polypeptide of the invention such as a MICAL polypeptide, with an agent that affects axonal guidance regulatory activity or monooxygenase activity. Typically, the method is performed in vivo and includes inhibiting axonal guidance regulatory activity by contacting the cell with an antioxidant. The cell can be contacted chronically with the antioxidant. The axonal guidance activity is typically semaphorin-mediated axonal repulsion. As such, in another embodiment, the present invention provides a method for affecting a semaphorin-mediated process by contacting a cell that expresses a MICAL polypeptide of the invention with an effective amount of an agent that affects axonal guidance regulatory activity. The agent can be, for example, a small molecule, a polypeptide or fragment thereof, a peptidomimetic, or an antisense polynucleotide, as discussed herein.

Not to be limited by theory, as disclosed herein MICALs regulate semaphorin-mediated axonal repulsion through a mechanism that requires their monooxygenase activity. Therefore, as illustrated in the Examples section, antioxidants such as flavonoids inhibit semaphorin-mediated axonal repulsion, apparently by overcoming the effects of MICAL monooxygenase activity.

As mentioned above, following spinal cord injury in humans, axons fail to reestablish their connections, which results in paralysis and loss of sensation of the affected area. During development, inhibition of axon growth plays a role in forming the nervous system. Axons are guided to their targets by molecules that attract them as well as by those that inhibit (i.e., repel) them. The inhibition of semaphorin-mediated axonal repulsion can allow axons to reach new targets. Therefore, methods for treating spinal cord injuries can focus on inhibiting axonal repulsion (See e.g., Schwab, M. E., Science, 295:1029 (2002); and Fournier, A. E., and Strittmatter, S. M., Current Opinion in Neurobiol., 11:89 (2001)). Accordingly, methods of this embodiment of the invention are useful for example, for inducing regeneration of axons after spinal cord injury.

In another embodiment, the present invention provides a method for treating a neurological condition in a subject, that includes contacting in the subject, a cell of the central nervous system and/or peripheral nervous system having a disrupted axonal connection, or a cell that affects axonal growth of the central nervous system and/or peripheral nervous system cell, or surrounding tissue, with an amount of an agent that modulates the activity or expression of a MICAL polypeptide, the amount being effect to modulate axonal guidance regulatory activity, axon out-growth, monooxygenase activity, or plexin-interacting activity. The subject in certain aspects is a human patient in need of the treatment. The neurological condition is any neurological condition in which a treatment strategy includes promoting axon growth. For example, neurological conditions treatable using methods of the present invention include a spinal cord injury, traumatic brain injury, neuropathic pain, Parkinson's Disease, Amyotrophic Lateral Sclerosis (ALS), ischemic injury, Alzheimer's Disease, Multiple Sclerosis, Huntington's chorea, multiple system atrophy, progressive supranuclear palsy, traumatic brain injury, neuropathic pain, ischemic injury, a neuropathy resulting from a stroke, a peripheral neuropathy resulting from chemotherapy, or a peripheral neuropathy resulting from diabetes. The neurodegenerative disease may be associated with a bacterial, viral or other infection, such as damage caused by HIV or herpes viral infections, encephalitis, and Creutzfeldt-Jacob disease and kuru or may be due to the effects of a drug or toxin. Surrounding tissue is any substrate through which an axon need to re-grow or re-establish its connections.

In certain aspects of this embodiment of the invention, the agent contacts a site in need of axonal growth or regrowth chronically. For example, the agent is applied for a length of time sufficient to promote neurorestoration. In general, the length of time of chronic application of an agent for the present invention is longer than the time period required to protect injured neurons (i.e. neuroprotection) from the harmful cascade of secondary events that follow injury, for example detrimental inflammatory responses and death of neurons and glia (i.e. secondary death due to reactive oxidative species (lipid peroxidation)). The length of time for chronic agent contact in the present invention is the time necessary to continue to stimulate axonal growth, to guide axons to their targets, and/or to establish new functional synapses. In certain aspects of the present invention, an agent can be applied initially to save neurons (neuroprotection), and continued over longer periods to promote neurorestoration. Therefore, the present invention in certain aspects, couples neuroprotection and neurorestoration through delivery of agents a short time after neurological damage, followed by long-term administration of the agent.

In one aspect of the present invention, the agent is applied for at least 1, 2, 7, or 14 days, or 1, 2, 3, 4, 5, 6, 12, 24, 36, 48, 60 months, either continually or repeatedly, for example by use of gene therapy, after identification or suspicion of a neurological condition. Other embodiments of the invention that include beneficial aspects directed at chronic application of an agent include for example, a method for affecting axon growth, a method for affecting a plexin mediated process, a method for treating a neurological disorder involving failure of axon regrowth, and a method for inducing regrowth and preventing inhibition of an injured process of a neuron.

As indicated herein, monooxygenase inhibitors and anti-oxidant flavonoids, including those that are monooxygenase inhibitors such as ECGC and EC, are inhibitors of semaphorin-mediated axonal repulsion. Accordingly, monooxygenase inhibitors and anti-oxidant flavonoids can be used as the agent in methods of various embodiments of the present invention, to inhibit axonal guidance regulatory activity. These embodiments of the invention include methods for treating a neurological condition, methods for affecting axonal guidance regulatory activity, methods for affecting axon growth, methods for affecting a plexin-mediated process, methods for treating a neurological disorder involving a failure of axon regrowth, and methods for inducing regrowth of an injured process of a neuron.

As mentioned above, a variety of monooxygenase inhibitors and anti-oxidant flavonoids have been identified and can be used to inhibit axonal guidance regulatory activity, such as semaphorin-mediated axonal repulsion. The anti-oxidant flavonoid can be a gallic acid derivative such as ECGC or EC, another flavoprotein monooxygenase inhibitor, another green tea polyphenol. Examples of gallic acid derivatives that can be used to affect axonal guidance regulatory activity in embodiments of the present invention include, but are not limited to, ECGC, EC, (−)-gallocatechin-3-O-gallate (GCG), (−)-epicatechin-3-O-gallate (ECG), (−)-epigallocatechin (EGC), (+)-gallocatechin (GC), theasinensin A, 3″-O-methyl-EGCG, 3″-O-methyl-ECG, 3″-O-methyl-GCG, (−)-epigallocatechin (EGC), (1)-gallocatechin (GC), gallic acid, catechin, n-octyl gallate, and n-cetyl gallate.

An agent used in the methods of the present invention for affecting axonal guidance regulatory activity or monooxygenase activity, in certain aspects, is a known oxidase inhibitor. For example, an oxidase inhibitor reported in Cross, Free Radical Biology and Med., 8:71-93 (1990) including DPI, ibuprofen, and/or aspirin. Furthermore, the screening assay can be used to test other agents including inhibitors of similar flavoprotein monooxygenase family members (See e.g., Cross (supra.); and Arnould and Camadro, Proc. Nat. Acad. Sci., 95:10553-10558 (1998)).

Various embodiments of the present invention can further include contacting the cell with an agent that modulates MICAL activity and affects axon regeneration. The embodiments include methods for treating a neurological condition, methods for treating a neurological disorder involving a failure of axon regrowth, and methods for inducing regrowth of an injured process of a neuron, as described herein. In certain aspects, the agent that affects axon regeneration promotes axon regeneration. Accordingly, the agent can be a neurotrophic factor, a mechanical bridge, and/or a stem cell (see e.g., Blesch A., et al., Brain Res. Bulletin, 57:83, 2002; and Cao, Q., et al., J. Neurosci. Res., 68: 501 (2002)).

Mechanical bridges include, for example, genetically engineered cells, stem cells, fetal tissue, Schwann cells, olfactory ensheathing glia, as discussed below. Neurotrophins are molecules with closely related structures that are known to support the survival of different classes of embryonic neurons. Virtually any neurotrophin can be used in methods of the present invention, including, for example, nerve growth factor (NGF), brain-derived neurotrophic factor (BDNF), neurotrophin 3 (NT 3), neutortrophin-4/5 (NT-4/5), glia cell line-derived neurotrophic factor (GDNF), and leukemia inhibitory factor (LIF).

Neurotrophins can be delivered to a site of severed axons, for example, by use of gene therapy. For example, viral vectors, including retroviral vectors, as described herein, capable of infecting neurons or glia can provide a localized source of trophic factors, such as neurotrophins to stimulate axonal outgrowth. Genetically modified cells grafted to a lesion site in the spinal cord can provide not only augmented amounts of trophic molecules at the injury site, but also a potential axonal growth substrate. Thus, genetically modified cells can provide mechanical bridges for growing axons to potentially connect injured spinal cord regions (Blesch et al., 2002). Furthermore, genetically modified cells can be used for long-term delivery of a neurotrophin, in a regulated manner. Furthermore, gene therapy can be used to deliver MICAL inhibitors, such as mutant MICALs or MICAL-Like proteins, chronically to a site of spinal cord injury.

Cell types used for grafting in gene therapy, can include for example fibroblasts, Schwann cells, and neural stem cells (see e.g., Brecknell J. E., et al., Neuroscience, 74(3):775-84 (1996)). Furthermore, autologous cells can be used to avoid immune responses and graft rejection. Gene therapy for use in the present invention can include in vivo or ex vivo gene therapy.

Methods of the present invention that include providing a stem cell as well as an anti-oxidant typically involve providing both the stem cell and the anti-oxidant to a site of neuronal damage, such as a site of spinal cord injury. For example, the stem cell can be grafted to a site of spinal cord injury.

A variety of stem cells can be used with the methods of the present invention. The stem cells are typically neural stems cells (i.e., stem cells that give rise to cells of neuronal lineage). The stem cell can be an embryonic stem cell or an adult stem cell and can be isolated, for example from blood or bone marrow (See e.g., Kabos, P., et al., Exp Neurol., 178(2):288-93 (2002)). Neural stem cells typically express the marker, nestin. The stem cells are capable of giving rise to neurons and glial cells.

Neural stem cells could be induced towards neuronal phenotypes to allow the replacement of spinal neurons lost after injury, toward astrocytes to restore the non-neuronal milieu of the pre-injured spinal cord, or towards oligodendroglia to allow remyelination. Either neuronal or glial lineages could be useful for reconstituting a permissive substrate for regenerating axons to extend over sites of injury (Blesch et al., 2002). The cells can be modified to produce a recombinant neurotrophic factor.

Other mechanical bridges of the invention in certain aspects, include an axon outgrowth promoting molecule such as netrin, laminin, collagen, or artificial polymer-based substrates. In another embodiment, the present invention provides a method for affecting axon growth, that includes contacting a neuronal lineage cell with an agent that inhibits axonal guidance regulatory activity of a polypeptide as set forth in SEQ ID NO:2 (human MICAL-1), SEQ ID NO:4 (human MICAL-2), SEQ ID NO:6 (human MICAL-3), or SEQ ID NO: 8 (Drosophila MICAL long isoform), SEQ ID NO:10 (Drosophila MICAL medium isoform), SEQ ID NO: 12 (Drosophila MICAL short isoform).

The neuronal lineage cell can be for example, a neuron or an oligodendrocyte (J. Neurosci. Jul 15;22(14):5992-6004 (2002)). Furthermore, the neuronal lineage cell can be a neural stem cell, or a neuronal cell derived from an isolated stem cell. In this aspect, the stem cell can be introduced into a subject at the site of a spinal cord injury, and contacted with the agent before introduction into the subject, and/or at the site of the spinal cord injury. Accordingly, the cell can be contacted with the agent in vivo or in vitro.

In another embodiment, the present invention provides a method for affecting a plexin-mediated process or a semaphorin-mediated process, that includes contacting a cell that carries out the plexin-mediate process or semaphorin-mediated process, such as a cell expressing a MICAL-polypeptide of the present invention, with an effective amount of an agent, for example an antioxidant such as a monooxygenase inhibitor, that affects a MICAL polypeptide activity. The agent affects the plexin-mediated process or the semaphorin-mediated process. For example, where the plexin-mediated process is axonal regrowth, the agent is chronically contacted with the cell.

For this embodiment of the invention, virtually any cell can be used. For example a recombinant cell that expresses a MICAL polypeptide can be used. The recombinant cell can be obtained using a vector of the present invention and transfection or transformation methods well known in the art. The cell in this embodiment of the invention can be a cell of the nervous system, such as a neuron, an immune cell, a cancer cell, and a cardiac cell, particularly a cardiac neural crest.

As is indicated herein by the analysis of MICAL proteins in a model invertebrate system (Drosophila) and in the vertebrate nervous system, the interactions between all semaphorins, both secreted and transmembrane, with plexin family members are likely to involve essential interactions and functions provided by MICAL proteins. The present disclosure suggests that the N-terminal MICAL monooxygenase domain is essential for semaphorin/plexin-mediated neuronal repulsion. Therefore, MICALs and the subclass of monooxygenase they belong to are targets with regard to neuronal regeneration following injury and various strategies designed to promote neuronal regeneration following neurodegeneration. Additionally, since plexins bind homophilically to other plexins (Neuron, 14: 1189-99 (1995)) they may signal in a semaphorin-independent, but MICAL-dependent, manner.

The cell included in methods of the present invention, including methods for affecting a plexin-mediated or semaphorin-mediated process, in certain aspects is a cell of the immune system. For example, the cell of the immune system can be a lymphocyte, such as a B-cell, a T-cell, or precursor thereof, a monocyte, or a phagocyte. Other cell types for methods of the present invention include cancer cells, particularly metastatic cancer cells, and cardiac cells, particularly cardiac cells from the neural crest. In certain aspects the plexin-mediated process is mediated by Plexin A or Plexin B. In certain aspects the semaphorin-mediated process is mediated by Sema 1a, Sema 3a, Sema 7a, or a class 4 semaphorin.

Semaphorins and plexins have been shown to provide important functions in the immune system (See e.g., Ventura, A., and Pelicci, P. G., “Semaphorins: Green Light for Redox Signaling?” Science 's STKE, pgs. 1-3 (2002)). In addition to what appear to be a plexin-independent function of the class 4 semaphorin Sema4D (i.e. CD100) in the enhancement of B-cell responses via the inactivation of CD72, viral semaphorin interactions with plexin C1 (aka VESPR) induce robust responses in human monocytes (i.e. cell aggregation and the expression of pro-inflammatory cytokines). The mammalian orthologue of one of these viral semaphorins, called Sema7A, may therefore also be involved in immunomodulation events. Further, the interactions between class 4 semaphorins such as CD100 with B class plexins, interactions which are well-characterized in the vertebrate neurons, may function in the immune system. The expression of all class B plexins in the immune system has not been exhaustively determined, and it is likely that neuronal class 4 semaphorin/Plexin B functions have immune system counterparts.

Finally, the initial characterization of MICAL as a CasL interacting protein was carried out using human thymus cells as a starting source of tissue. Cas family members are important for TCR and b1 integrin-induced immunological reactions in lymphocytes, including interleukin-2 production and various migratory responses. Thus, though at present it is difficult to predict exact roles in immune system function, it is likely that MICALs will be involved in plexin immune system function, that modulation of Cas protein function will follow, and that the disclosure herein with respect to characterization of flavonoids which are likely MICAL antagonists will at the least provide a set of compounds with potent effects on immune system function.

The plexin-mediated process or the semaphorin-mediated process can be semaphorin 1a-PlexA-mediated repulsive axon guidance. In another aspect, however, the plexin-mediated process or semaphorin-mediated process affected by antioxidants in the methods of the present invention can include cell migration, for example the migration of cancer cells (see e.g., Trusolino, L., and Comoglio, P. M., Nat., Rev. Cancer, 2:289 (2002)). Studies by J. Mina and colleagues, for example, have implicated class 3 semaphorins in certain small cell lung carcinomas. Sema3F, and more recently the closely linked gene for Sema3B, have both been associated with genetic lesions in lung cancer cell lines, and the most recent evidence suggests that Sema3B may be a key determinant in these lung cancers. Given demonstrations of class 3 semaphorin mediated effects on neural crest cell morphology and GABAergic cortical neuronal migration, coupled with the in vitro semaphorin collapse assays recently developed in tissue culture cell lines (COS cells, Cell, 99:59-69 (1999)), if seems reasonable to think that non-neuronal cell adhesivity and migration are influenced by these secreted repellents. Based upon the present disclosure, which implicate MICAL monooxygenase function in Sema3A repulsion in vertebrates, it is expected that class 3 semaphorin function can be either attenuated or enhanced by using anti-oxidants that affect MICAL function.

Class 4 semaphorins, which as described are likely to play key roles in the immune system, have also recently been implicated in regulating invasive growth by Comoglio and colleagues. Sema3F in a liver cell line apparently can mediate invasive growth via a coupling of PlexinB1 and Met receptor signaling. This requirement for a plexin and an associated membrane bound co-receptor component for a functional semaphorin response is reminiscent in certain respects of observations in Drosophila from Goodman and colleagues showing that Plexin A requires offtrack (OTK), a protein related to receptor tyrosine kinases, for plexin-mediated semaphorin repulsion. This work suggests that Plexin B1, and perhaps many other plexins, can regulate cell migration, either positively or negatively. It is interesting to note that recent work by T. Hunter and colleagues demonstrates a requirement for both FAK and CasL function for Ephrin/Eph receptor mediated attractive responses.

A role has also been established for plexinA2 and semaphorin signaling in cardiac neural crest cells. For example, studies have shown that PlexinA2 is expressed in migrating and postmigrating cardiac neural crest cells in the mouse (Brown et al., Development 128:3071 (2001)). Furthermore, it has been shown that PlexinA2-expressing cardiac neural crest cells are patterned abnormally in several mutant mouse lines with congenital heart disease including those lacking Semaphorin 3C. (Id.).

Additionally, reports have suggested a role of plexins in numerous other diseases. For example, studies have revealed that at least certain semaphorins are dysregulated and/or downregulated in patients with Alzheimer's Disease and Down's Syndrome. (Andorn, A. C., and Kalaria, R. N., Acta. Neurochir. Suppl. (Wien) 70:212-5 (1997); Lubec, G., J Neural Transm Suppl., 57:161-77 (1999); and Hirsch et al., Brain Res., 27:67-79 (1999)). Other studies have identified a plexin family member as involved in polycystic kidney and hepatic disease (Onuchic, L. F., et al., Pediatr. Res. 52:830 (2002)). Furthermore, single nucleotide polymorphisms (SNPs) related to Rett syndrome have been identified in a Plexin gene (Dahle, A. R., et al., Am. J. Med. Genet. 3:69 (2000)).

Finally, the very well documented roles of Cas family members in regulating non-neuronal cell morphology and various growth characteristics also suggest understanding that MICALs are linked to CasL could be invaluable for controlling certain cellular behaviors. p130Cas was first identified and a target of hyperphosphorylation by oncogenic Src and Crk. Since then, many in vitro experiments have implicated Cas proteins in the maintenance of the transformed cell state, however to date in vivo support for Cas function in cell transformation is still lacking. Nevertheless, the demonstrated roles for Cas proteins in cell cycle progression, the regulation of cell shape, and for the induction of cell migration, make this protein an attractive target for controlling a myriad of cellular functions, from osteoclast activation to vasculogenesis and angiogenesis. How MICAL regulates Cas is at present unknown and is a major focus for our own research. However, given well-established interactions between Cas family members and a variety of proteins essential for the establishment and maintenance of cell shape, cell-cell interactions, and morphological changes including migration (including, but not limited to, FAK, RAFTK, Crk, Fyn, Yes, Abl, Grb2, several phosphatases including PTP1B, Nck, and Src), it is likely that MICALs play a role in these processes through Cas interactions.

A method of the invention for the various embodiments that include contacting a cell or a MICAL with an agent such as an antioxidant, can be performed, for example, by contacting under suitable conditions a target cell and an agent that affects a MICAL function such as MICAL axon guidance regulatory activity. Suitable conditions can be provided by placing the cell, which can be an isolated cell or can be a component of a tissue or organ, in an appropriate culture medium, or by contacting the cell in situ in an organism. For example, a medium containing the cell can be contacted with an agent the affects the ability of a MICAL to specifically interact with a plexin expressed on the cell, or with an agent that affects MIXAL axon guidance regulatory activity in the cell. In general, the cell is a component of a tissue or organ in a subject, in which case contacting the cell can comprise administering the agent to the subject. However, the cell also can be manipulated in culture, then can be maintained in culture, administered to a subject, or used to produce a transgenic nonhuman animal.

An agent useful in a method of the invention can be any type of molecule, for example, a polynucleotide, a peptide, a peptidomimetic, peptoids such as vinylogous peptoids, a small organic molecule, or the like, and can act in any of various ways to affect a MICAL function such as axon guidance regulatory activity. The agent can act to alter a semaphorin-mediated pathway in the cell. In addition, the agent can be an agonist, which mimics or enhances the effect of MICAL on a cell, for example, the ability of MICAL to specifically interact with a plexin, thereby increasing MICAL axon guidance regulatory activity in the cell; or can be an antagonist, which reduces or inhibits the effect of MICAL on a cell, thereby reducing or inhibiting MICAL axon guidance regulatory activity. For example, administration of an antagonist can result in axon growth and new positioning to allow formation of new targets by inhibiting semaphorin-mediated axonal repulsion activity.

As used herein, the term “specific interaction” or “specifically binds” or the like means that two molecules form a complex that is relatively stable under physiologic conditions. The term is used herein in reference to various interactions, including, for example, the interaction of MICAL and a plexin such as plexin A, the interaction of the intracellular components of a semaphorin-mediated pathway, and the interaction of an antibody and its antigen. A specific interaction can be characterized by a dissociation constant of at least about 1×10⁻⁶ M, generally at least about 1×10⁻⁷ M, usually at least about 1×10⁻⁸ M, and particularly at least about 1×10⁻⁹ M or 1×10⁻¹⁰ M or greater. A specific interaction generally is stable under physiological conditions, including, for example, conditions that occur in a living individual such as a human or other vertebrate or invertebrate, as well as conditions that occur in a cell culture such as used for maintaining mammalian cells or cells from another vertebrate organism or an invertebrate organism. Methods for determining whether two molecules interact specifically are well known and include, for example, equilibrium dialysis, surface plasmon resonance, and the like.

An agent that alters a specific interaction of a MICAL with a plexin can act, for example, by binding to a MICAL such that it cannot interact specifically with the plexin, by competing with MICAL for binding to the plexin, or by otherwise by-passing the requirement that MICAL specifically interact with a plexin in order to regulate axonal guidance. A mutant plexin that retains its ability to bind to a MICAL but not other plexin functions is an example of an agent that can bind a MICAL, thereby sequestering it and reducing or inhibiting its ability to interact specifically with a functional plexin. A MICAL mutant that includes only its plexin interacting region is an example of an agent that can compete with MICAL for plexin binding, thereby reducing or inhibiting the ability of the MICAL to interact specifically with a plexin. Such MICAL antagonists are useful in practicing a method of the invention, particularly for reducing or inhibiting MICAL axon guidance regulatory activity.

An agent useful in a method of the invention an antibody that specifically binds a MICAL, including all or a portion of the plexin interacting region, thereby preventing MICAL from interacting specifically with a plexin. Alternatively, the agent can be an antibody that specifically binds to a plexin, including all or a portion of the MICAL interacting region, thereby preventing the plexin from interacting specifically with a MICAL. Such an anti-MICAL or anti-plexin antibody can be selected for its ability to specifically bind MICAL or plexin, respectively, without activating MICAL axon guidance regulatory activity, and can be useful as a MICAL antagonist for reducing or inhibiting MICAL axon guidance regulatory activity; or can be selected for its ability to specifically bind MICAL or plexin, respectively and activate axon guidance regulatory activity, thus acting as a MICAL agonist. The antibody can be raised using a MICAL or a plexin, including plexin A as an immunogen, or can be an anti-idiotype antibody, which is raised against an anti-MICAL antibody and mimics MICAL.

An agent useful in a method of the invention also can be an agent that reduces MICAL monooxygenase activity, thereby reducing or inhibiting MICAL axon guidance regulatory activity.

In addition, an agent useful in a method of the invention can be a mutant plexin, which, for example, lacks semaphorin signal transduction activity in response to MICAL binding, or has constitutive semaphorin signal transduction activity. For example, a mutant plexin can have a point mutation, a deletion, or the like in a functional domain other than the MICAL binding domain. Such a dominant negative mutant plexin lacks the ability to transmit a semaphorin and/or MICAL signal despite the fact that it can specifically bind a MICAL.

An agent useful in a method of the invention also can modulate the level or activity of a MICAL.

The specific interaction of MICAL with plexin A indicates that MICAL axonal guidance regulatory activity can involve components of the semaphorin-plexin mediated repulsive axon guidance pathway. Thus, the Semaphorin repulsive axon guidance pathway provides a target for modulating the effect of MICAL on a cell, and agents that affect the Semaphorin pathway can be useful for modulating MICAL axon guidance regulatory activity.

Antagonist agents that can reduce or inhibit MICAL axon guidance regulatory activity are exemplified by dominant negative MICAL polypeptides in which the a functional domain other than the plexin-interacting region has been mutated. The mutants include polypeptides that include a plexin-interacting region and no other

Where the agent that acts intracellularly is a peptide or a polypeptide, it can be contacted with the cell directly, or a polynucleotide encoding the peptide (or polypeptide) can be introduced into the cell and the peptide can be expressed in the cell. It is recognized that some of the peptides useful in a method of the invention are relatively large and, therefore, may not readily traverse a cell membrane. However, various methods are known for introducing a peptide into a cell. The selection of a method for introducing such a peptide into a cell will depend, in part, on the characteristics of the target cell, into which the polypeptide is to be provided. For example, where the target cells, or a few cell types including the target cells, express a receptor, which, upon binding a particular ligand, is internalized into the cell, the peptide agent can be operatively associated with the ligand. Upon binding to the receptor, the peptide is translocated into the cell by receptor-mediated endocytosis. The peptide agent also can be encapsulated in a liposome or formulated in a lipid complex, which can facilitate entry of the peptide into the cell, and can be further modified to express a receptor (or ligand), as above. The peptide agent also can be introduced into a cell by engineering the peptide to contain a protein transduction domain such as the human immunodeficiency virus TAT protein transduction domain, which facilitates translocation of the peptide into the cell (see Schwarze et al., Science 285:1569-1572 (1999), which is incorporated herein by reference; see, also, Derossi et al., J. Biol. Chem. 271:18188 (1996)). The target cell also can be contacted with a polynucleotide encoding the peptide or polypeptide agent, which can be expressed in the cell.

An agent useful in a method of the invention can be a polynucleotide, which can be contacted with or introduced into a cell as described above. Generally, but not necessarily, the polynucleotide is introduced into the cell, where it effects its function either directly, or following transcription or translation or both. For example, as discussed above, the polynucleotide can encode a polypeptide agent, which is expressed in the cell and modulates MICAL activity. Such an expressed polypeptide can be, for example, a mutant MICAL polypeptide, which does not have monooxygenase activity; or can be a mutant plexin. Methods for introducing a polynucleotide into a cell are exemplified below or otherwise known in the art.

A polynucleotide agent useful in a method of the invention also can be, or can encode, an antisense molecule, a ribozyme or a triplexing agent. For example, the polynucleotide can be (or can encode) an antisense nucleotide sequence such as an antisense MICAL, plexin, or semaphorin sequence, which can act as an antagonist to reduce or inhibit MICAL axon guidance regulatory activity, thereby inhibiting semaphorin-mediated repulsive axon guidance. Such polynucleotides can be contacted directly with a target cell and, upon uptake by the cell, can effect their antisense, ribozyme or triplexing activity; or can be encoded by a polynucleotide that is introduced into a cell, whereupon the polynucleotide is expressed to produce, for example, an antisense RNA molecule or ribozyme, which effects its activity.

An antisense polynucleotide, ribozyme or triplexing agent is complementary to a target sequence, which can be a DNA or RNA sequence, for example, messenger RNA, and can be a coding sequence, a nucleotide sequence comprising an intron-exon junction, a regulatory sequence such as a Shine-Delgarno sequence, or the like. The degree of complementarity is such that the polynucleotide, for example, an antisense polynucleotide, can interact specifically with the target sequence in a cell. Depending on the total length of the antisense or other polynucleotide, one or a few mismatches with respect to the target sequence can be tolerated without losing the specificity of the polynucleotide for its target sequence. Thus, few if any mismatches would be tolerated in an antisense molecule consisting, for example, of 20 nucleotides, whereas several mismatches will not affect the hybridization efficiency of an antisense molecule that is complementary, for example, to the full length of a target mRNA encoding a cellular polypeptide. The number of mismatches that can be tolerated can be estimated, for example, using well known formulas for determining hybridization kinetics (see Sambrook et al., supra, 1989) or can be determined empirically using methods as disclosed herein or otherwise known in the art, particularly by determining that the presence of the antisense polynucleotide, ribozyme, or triplexing agent in a cell decreases the level of the target sequence or the expression of a polypeptide encoded by the target sequence in the cell.

A polynucleotide useful as an antisense molecule, a ribozyme or a triplexing agent can inhibit translation or cleave the nucleic acid molecule, thereby modulating MYCAL axon guidance regulatory activity in a cell. An antisense molecule, for example, can bind to an mRNA to form a double stranded molecule that cannot be translated in a cell. Antisense oligonucleotides of at least about 15 to 25 nucleotides are preferred since they are easily synthesized and can hybridize specifically with a target sequence, although longer antisense molecules can be expressed from a polynucleotide introduced into the target cell. Specific nucleotide sequences useful as antisense molecules can be identified using well known methods, for example, gene walking methods (see, for example, Seimiya et al., J. Biol. Chem. 272:4631-4636 (1997), which is incorporated herein by reference). Where the antisense molecule is contacted directly with a target cell, it can be operatively associated with a chemically reactive group such as iron-linked EDTA, which cleaves a target RNA at the site of hybridization. A triplexing agent, in comparison, can stall transcription (Maher et al., Antisense Res. Devel. 1:227 (1991); Helene, Anticancer Drug Design 6:569 (1991)). Thus, a triplexing agent can be designed to recognize, for example, a sequence of a MICAL gene regulatory element, thereby reducing or inhibiting the expression of a MICAL polypeptide in the cell, and modulating MICAL axon guidance regulatory activity in a target cell.

The agent to be administered to the subject is administered under conditions that facilitate contact of the agent with the target cell and, if appropriate, entry into the cell. Entry of a polynucleotide agent into a cell, for example, can be facilitated by incorporating the polynucleotide into a viral vector that can infect the cells. If a viral vector specific for the cell type is not available, the vector can be modified to express a receptor (or ligand) specific for a ligand (or receptor) expressed on the target cell, or can be encapsulated within a liposome, which also can be modified to include such a ligand (or receptor). A polypeptide agent can be introduced into a cell by various methods, including, for example, by engineering the peptide to contain a protein transduction domain such as the human immunodeficiency virus TAT protein transduction domain, which can facilitate translocation of the peptide into the cell (see Schwarze et al., supra, 1999; Derossi et al., supra, 1996).

The presence of the agent in the target cell can be identified directly, for example, by operatively linking a detectable label to the agent, by using an antibody specific for the agent, particularly a polypeptide agent, or by detecting a downstream effect due to the agent, for example, decreased semaphorin-mediated axon repulsion in the cell. An agent can be labeled so as to be detectable using methods well known in the art (Hermanson, “Bioconjugate Techniques” (Academic Press 1996), which is incorporated herein by reference; see, also, Harlow and Lane, supra, 1988). For example, a peptide or polynucleotide agent can be labeled with various detectable moieties including a radiolabel, an enzyme such as alkaline phosphatase, biotin, a fluorochrome, and the like. Where the agent is contained in a kit, the reagents for labeling the agent also can be included in the kit, or the reagents can be purchased separately from a commercial source.

An agent useful in a method of the invention can be administered to the site of the pathologic condition, or can be administered by any method that provides the target cells with the polynucleotide or peptide. As used herein, the term “target cells” typical means an immune cell, a transformed eukaryotic cell, or a cell of the nervous system, for example a neuron, that are to be contacted with the agent. For administration to a living subject, the agent generally is formulated in a pharmaceutical composition suitable for administration to the subject. Thus, the invention provides pharmaceutical compositions containing an agent, which is useful for modulating MICAL axonal guidance regulatory activity in a cell, in a pharmaceutically acceptable carrier. As such, the agents are useful as medicaments for treating a subject suffering from a pathological condition as defined herein.

Pharmaceutically acceptable carriers are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the conjugate. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the physico-chemical characteristics of the therapeutic agent and on the route of administration of the composition, which can be, for example, orally or parenterally such as intravenously, and by injection, intubation, or other such method known in the art. The pharmaceutical composition also can contain a second reagent such as a diagnostic reagent, nutritional substance, toxin, or therapeutic agent, for example, a cancer chemotherapeutic agent.

The agent can be incorporated within an encapsulating material such as into an oil-in-water emulsion, a microemulsion, micelle, mixed micelle, liposome, microsphere or other polymer matrix (see, for example, Gregoriadis, Liposome Technology, Vol. 1 (CRC Press, Boca Raton, Fla. 1984); Fraley, et al., Trends Biochem. Sci., 6:77 (1981), each of which is incorporated herein by reference). Liposomes, for example, which consist of phospholipids or other lipids, are nontoxic, physiologically acceptable and metabolizable carriers that are relatively simple to make and administer. “Stealth” liposomes (see, for example, U.S. Pat. Nos. 5,882,679; 5,395,619; and 5,225,212, each of which is incorporated herein by reference) are an example of such encapsulating materials particularly useful for preparing a pharmaceutical composition useful for practicing a method of the invention, and other “masked” liposomes similarly can be used, such liposomes extending the time that the therapeutic agent remain in the circulation. Cationic liposomes, for example, also can be modified with specific receptors or ligands (Morishita et al., J. Clin. Invest., 91:2580-2585 (1993), which is incorporated herein by reference). In addition, a polynucleotide agent can be introduced into a cell using, for example, adenovirus-polylysine DNA complexes (see, for example, Michael et al., J. Biol. Chem. 268:6866-6869 (1993), which is incorporated herein by reference).

The route of administration of a pharmaceutical composition containing an agent that alters MICAL axon guidance regulatory activity such as semaphorin-mediated axon repulsion activity, will depend, in part, on the chemical structure of the molecule. Polypeptides and polynucleotides, for example, are not particularly useful when administered orally because they can be degraded in the digestive tract. However, methods for chemically modifying polypeptides, for example, to render them less susceptible to degradation by endogenous proteases or more absorbable through the alimentary tract are well known (see, for example, Blondelle et al., supra, 1995; Ecker and Crook, supra, 1995). In addition, a peptide agent can be prepared using D-amino acids, or can contain one or more domains based on peptidomimetics, which are organic molecules that mimic the structure of peptide domain; or based on a peptoid such as a vinylogous peptoid.

A pharmaceutical composition as disclosed herein can be administered to an individual by various routes including, for example, orally or parenterally, such as intravenously, intramuscularly, subcutaneously, intraorbitally, intracapsularly, intraperitoneally, intrarectally, intracisternally or by passive or facilitated absorption through the skin using, for example, a skin patch or transdermal iontophoresis, respectively. Furthermore, the pharmaceutical composition can be administered by injection, intubation, orally or topically, the latter of which can be passive, for example, by direct application of an ointment, or active, for example, using a nasal spray or inhalant, in which case one component of the composition is an appropriate propellant. Furthermore, the agent can be delivered by intrathecal administration using a pump to administer the agent over a period of time.

A pharmaceutical composition also can be administered to the site of a pathologic condition, for example, intravenously or intra-arterially into a blood vessel supplying a tumor, or by direct injection into the central nervous system., or a portion thereof such as the spinal cord. In aspects of the invention wherein the agent is intended to be delivered to the spinal cord, the agent can be an agent that is capable of crossing into the spinal cord from the blood stream. Such agents include anti-oxidant flavonoids discussed herein.

The total amount of an agent to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of the pharmaceutical composition to treat a pathologic condition in a subject depends on many factors including the age and general health of the subject as well as the route of administration and the number of treatments to be administered. In view of these factors, the skilled artisan would adjust the particular dose as necessary. In general, the formulation of the pharmaceutical composition and the routes and frequency of administration are determined, initially, using Phase I and Phase II clinical trials.

In general, in methods of the present invention, an agent is administered in an amount that is sufficient to modulate axonal guidance regulatory activity, monooxygenase activity, or plexin-interacting activity. It will be recognized that routine methods can be used to identify effective amounts.

The pharmaceutical composition can be formulated for oral formulation, such as a tablet, or a solution or suspension form; or can comprise an admixture with an organic or inorganic carrier or excipient suitable for enteral or parenteral applications, and can be compounded, for example, with the usual non-toxic, pharmaceutically acceptable carriers for tablets, pellets, capsules, suppositories, solutions, emulsions, suspensions, or other form suitable for use. The carriers, in addition to those disclosed above, can include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening or coloring agents and perfumes can be used, for example a stabilizing dry agent such as triulose (see, for example, U.S. Pat. No. 5,314,695).

In certain embodiments, the present invention provides detection methods. For example, in one embodiment, the present invention provides a method of detecting an immune disease or disorder in a subject. The method includes determining in the subject, the level of expression of a polynucleotide and/or polypeptide of the present invention, such as a MICAL polynucleotide or a MICAL polypeptide. An increased or a decreased level of expression or activity can be indicative of the immune disease or disorder.

In another embodiment, the present invention provides a method of detecting cancer in a subject. The method includes determining in the subject, the level of expression of a polynucleotide and/or polypeptide of the present invention, such as a MICAL polynucleotide or a MICAL polypeptide. An increased level of expression or activity is indicative of the cancer.

In another method, the present invention provides a method of assessing the invasiveness of cancer cells in a subject. The method includes determining in the subject, or, more particularly, in cancer cells from the subject, the level of expression of a polynucleotide and/or polypeptide of the present invention, such as a MICAL polynucleotide or a MICAL polypeptide. An increased level of expression or activity indicates that the cancer cells are invasive.

In another embodiment, the present invention provides a method of detecting central nervous system injury and/or peripheral nervous system injury in a subject, that includes determining the level of expression of a polynucleotide of the present invention, or a polypeptide of the present invention or a MICAL activity thereof, in a sample from the central nervous system or peripheral nervous system of the subject. It has been found that MICAL expression is increased upon injury to the nervous system. Accordingly, an increased level of expression or activity identified by a method of the invention is indicative of the central nervous system injury and/or peripheral nervous system.

A sample of the central nervous system can be obtained using known methods. The sample can include for example, spinal fluid or neurons from the spinal cord at a suspected site of injury. The subject can be a human. The increased level can be identified by comparing the determined level to a level of a subject not suspected of suffering from spinal cord injury.

The MICAL activity can be any of the MICAL activities, including, for example, monooxygenase activity, axon guidance regulatory activity, plexin interacting activity, or binding to SH-3 domain-containing proteins. Methods for detecting these activity, some of which are provided herein, are known in the art. Furthermore, methods for determining the level of expression of a polynucleotide or a polypeptide of the present invention, examples of which are provided below, are known in the art.

Cells obtained in the sample for any of the methods for detecting or assessing of the present invention can be contacted with a lysis buffer. The sample obtained can then be further processed, for example to isolate nucleic acids or polypeptides.

Nucleic can be isolated from the lysed cells and cellular material by any number of means well known in the art. For example, a number of commercial products are available for isolating polynucleotides, including but not limited to, TriReagent (Molecular Research Center, Inc, Cincinnati, Ohio). The isolated polynucleotides can then be assayed for the presence of a polynucleotide that encodes a MICAL or MICAL-Like polypeptide.

Analyzing expression of a MICAL polypeptide or a nucleotide encoding a MICAL polypeptide includes any qualitative or quantitative method for detecting expression of a gene, many of which are known in the art. Non-limiting methods for analyzing polynucleotides and polypeptides are discussed below.

The methods of analyzing expression of MICAL or a MICAL-Like polypeptide of the present invention can utilize a biochip, or other miniature high-throughput technology. The manufacture and use of biochips such as those involving bioarrays, are known in the art and commercially available (See e.g., bioarrays available from Sigma-Genosys (The Woodlands, Tex.); Affymetrix (Santa Clara, Calif.), and Full Moon Biosystems (Sunnyvale, Calif.)) (For reviews of Biochips and bioarrays see, e.g., Kallioniemi O. P., “Biochip technologies in cancer research,” Ann Med, Mar; 33(2):142-7 (2001); and Rudert F., “Genomics and proteomics tools for the clinic,” Curr Opin. Mol. Ther., Dec;2(6):633-42 (2000)).

Such bioarrays can be analyzed using blotting techniques similar to those discussed below for conventional techniques of detecting polynucleotides and polypeptides. Other microfluidic devices and methods for analyzing gene expression can be used for the methods of the present invention.

Quantitative measurement of expression levels using bioarrays is also known in the art, and typically involve a modified version of a traditional method for measuring expression as described herein. For example, such quantitation can be performed by measuring a phosphor image of a radioactive-labeled probe binding to a spot of a microarray, using a phospohor imager and imaging software.

A method of the present invention in certain aspects, employs RNA, including messenger RNA (mRNA), isolated from a CNS sample. The RNA may be single stranded or double stranded. Enzymes and conditions optimal for reverse transcribing the template to DNA well known in the art can be used. Alternatively, the RNA can be subjected to RNAse protection assays. A DNA-RNA hybrid that contains one strand of each can also be used. A mixture of polynucleotides can also be employed, or the polynucleotides produced in a previous amplification reaction, using the same or different primers may be so used. In the instance where the polynucleotide sequence is to be amplified the polynucleotide sequence may be a portion of a MICAL, or can be present initially as a discrete molecule, such that the specific sequence is the entire nucleic acid. It is not necessary that the sequence to be amplified be present initially in a pure form; it may be a minor fraction of a complex mixture.

In addition, RNAse protection assays can be used if RNA is the polynucleotide obtained from the sample. In this procedure, a labeled antisense RNA probe is hybridized to the complementary polynucleotide in the sample. The remaining unhybridized single-stranded probe is degraded by ribonuclease treatment. The hybridized, double stranded probe is protected from RNAse digestion. After an appropriate time, the products of the digestion reaction are collected and analyzed on a gel (see for example Ausubel et al., CURRENT PROTOCOLS IN MOLECULAR BIOLOGY, section 4.7.1 (1987)). As used herein, “RNA probe” refers to a ribonucleotide capable of hybridizing to RNA in a sample of interest. Those skilled in the art will be able to identify and modify the RNAse protection assay specific to the polynucleotide to be measured, for example, probe specificity may be altered, hybridization temperatures, quantity of nucleic acid etc. Additionally, a number of commercial kits are available, for example, RiboQuant™ Multi-Probe RNAse Protection Assay System (Pharmingen, Inc., San Diego, Calif.).

In another embodiment, the polynucleotide in the sample may be analyzed by a blotting procedure, typically a Northern blot procedure, as illustrated in the Examples herein. For blotting procedures polynucleotides are separated on a gel and then probed with a complementary polynucleotide to the sequence of interest. For example, RNA is separated on a gel transferred to nitrocellulose and probed with complementary DNA that is derived from a MICAL gene. The complementary probe may be labeled radioactively, chemically etc. Hybridization of the probe is indicative of the expression of the MICAL.

Detection of a polynucleotide encoding a MICAL can be performed by standard methods such as size fractionating the nucleic acid. Methods of size fractionating the DNA and RNA are well known to those of skill in the art, such as by gel electrophoresis, including polyacrylamide gel electrophoresis (PAGE). For example, the gel may be a denaturing 7 M or 8 M urea-polyacrylamide-formamide gel. Size fractionating the nucleic acid may also be accomplished by chromatographic methods known to those of skill in the art.

The detection of polynucleotides may optionally be performed by using radioactively labeled probes. Any radioactive label may be employed which provides an adequate signal. Other labels include ligands, colored dyes, and fluorescent molecules, which can serve as a specific binding pair member for a labeled ligand, and the like. The labeled preparations are used to probe for a polynucleotide by the Southern or Northern hybridization techniques, for example. Nucleotides obtained from samples are transferred to filters that bind polynucleotides. After exposure to the labeled polynucleotide probe, which will hybridize to nucleotide fragments containing target nucleic acid sequences, the binding of the radioactive probe to target nucleic acid fragments is identified by autoradiography (see Genetic Engineering, 1 ed. Robert Williamson, Academic Press (1981), pp. 72-81). The particular hybridization technique is not essential to the invention. Hybridization techniques are well known or easily ascertained by one of ordinary skill in the art. As improvements are made in hybridization techniques, they can readily be applied in the method of the invention.

Probes according to the present invention and used in a method of the present invention selectively hybridize to a polynucleotide encoding a MICAL polypeptide. In preferred aspects, the probes are spotted on a bioarray using methods known in the art.

The polynucleotides encoding a MICAL may be amplified before they are detected. The term “amplified” refers to the process of making multiple copies of the nucleic acid from a single polynucleotide molecule. The amplification of polynucleotides can be carried out in vitro by biochemical processes known to those of skill in the art. The amplification agent may be any compound or system that will function to accomplish the synthesis of primer extension products, including enzymes. Suitable enzymes for this purpose include, for example, E. coli DNA polymerase I, Taq polymerase, Klenow fragment of E. coli DNA polymerase I, T4 DNA polymerase, other available DNA polymerases, polymerase muteins, reverse transcriptase, ligase, and other enzymes, including heat-stable enzymes (i.e., those enzymes that perform primer extension after being subjected to temperatures sufficiently elevated to cause denaturation). Suitable enzymes will facilitate combination of the nucleotides in the proper manner to form the primer extension products that are complementary to each mutant nucleotide strand. Generally, the synthesis will be initiated at the 3′-end of each primer and proceed in the 5′-direction along the template strand, until synthesis terminates, producing molecules of different lengths. There may be amplification agents, however, that initiate synthesis at the 5′-end and proceed in the other direction, using the same process as described above. In any event, the method of the invention is not to be limited to the embodiments of amplification described herein.

One method of in vitro amplification, which can be used according to this invention, is the polymerase chain reaction (PCR) described in U.S. Pat. Nos. 4,683,202 and 4,683,195. The term “polymerase chain reaction” refers to a method for amplifying a DNA base sequence using a heat-stable DNA polymerase and two oligonucleotide primers, one complementary to the (+)-strand at one end of the sequence to be amplified and the other complementary to the (−)-strand at the other end. Because the newly synthesized DNA strands can subsequently serve as additional templates for the same primer sequences, successive rounds of primer annealing, strand elongation, and dissociation produce rapid and highly specific amplification of the desired sequence. The polymerase chain reaction is used to detect the presence of polynucleotides encoding cytokines in the sample. Many polymerase chain methods are known to those of skill in the art and may be used in the method of the invention. For example, DNA can be subjected to 30 to 35 cycles of amplification in a thermocycler as follows: 95° C. for 30 sec, 52° to 60° C. for 1 min, and 72° C. for 1 min, with a final extension step of 72° C. for 5 min. For another example, DNA can be subjected to 35 polymerase chain reaction cycles in a thermocycler at a denaturing temperature of 95° C. for 30 sec, followed by varying annealing temperatures ranging from 54-58° C. for 1 min, an extension step at 70° C. for 1 min and a final extension step at 70° C.

The primers for use in amplifying the polynucleotides of the invention may be prepared using any suitable method, such as conventional phosphotriester and phosphodiester methods or automated embodiments thereof so long as the primers are capable of hybridizing to the polynucleotides of interest. One method for synthesizing oligonucleotides on a modified solid support is described in U.S. Pat. No. 4,458,066. The exact length of primer will depend on many factors, including temperature, buffer, and nucleotide composition. The primer must prime the synthesis of extension products in the presence of the inducing agent for amplification.

Primers used according to the method of the invention are complementary to each strand of nucleotide sequence to be amplified. The term “complementary” means that the primers must hybridize with their respective strands under conditions, which allow the agent for polymerization to function. In other words, the primers that are complementary to the flanking sequences hybridize with the flanking sequences and permit amplification of the nucleotide sequence. Preferably, the 3′ terminus of the primer that is extended has perfectly base paired complementarity with the complementary flanking strand. Primers and probes for polynucleotides encoding MICALs of the present invention, can be developed using known methods combined with the present disclosure.

Those of ordinary skill in the art will know of various amplification methodologies that can also be utilized to increase the copy number of target nucleic acid. The polynucleotides detected in the method of the invention can be further evaluated, detected, cloned, sequenced, and the like, either in solution or after binding to a solid support, by any method usually applied to the detection of a specific nucleic acid sequence such as another polymerase chain reaction, oligomer restriction (Saiki et al., Bio/Technology 3:1008-1012 (1985)), allele-specific oligonucleotide (ASO) probe analysis (Conner et al., Proc. Natl. Acad. Sci. USA 80: 278 (1983), oligonucleotide ligation assays (OLAs) (Landegren et al., Science 241:1077 (1988)), RNAse Protection Assay and the like. Molecular techniques for DNA analysis have been reviewed (Landegren et al, Science 242: 229-237 (1988)). Following DNA amplification, the reaction product may be detected by Southern blot analysis, without using radioactive probes. In such a process, for example, a small sample of DNA containing the polynucleotides obtained from the tissue or subject are amplified, and analyzed via a Southern blotting technique. The use of non-radioactive probes or labels is facilitated by the high level of the amplified signal. In one embodiment of the invention, one nucleoside triphosphate is radioactively labeled, thereby allowing direct visualization of the amplification product by autoradiography. In another embodiment, amplification primers are fluorescently labeled and run through an electrophoresis system. Visualization of amplified products is by laser detection followed by computer assisted graphic display, without a radioactive signal.

The methods of the present invention can involve a real-time quantitative PCR assay, such as a Taqman® assay (Holland et al., Proc. Natl. Acad. Sci. USA, 88(16):7276 (1991)). The assays can be performed on an instrument designed to perform such assays, for example those available from Applied Biosystems (Foster City, Calif.). Primers and probes for such an assay can be designed according to known procedures in the art.

Simple visualization of a gel containing the separated products may be utilized to analyze polynucleotides encoding MICALs according to the methods of the present invention. For example, staining of a gel to visualize separated polynucleotides, a number of stains are well known to those skilled in the art. However, other methods known to those skilled in the art may also be used, for example scanning densitometry, computer aided scanning and quantitation as well as others.

The method for detecting MICAL expression can alternatively employ the detection of a polypeptide product of one of these genes. The method for detecting a MICAL polypeptide in a cell is useful for detecting spinal cord injury by measuring the level of the MICAL polypeptide, in cells obtained from a subject suspected of having, or at risk of having spinal cord injury. The levels of MICALs are indicative of spinal cord injury when compared to a MICAL levels in a subject without spinal cord injury

In this regard, the sample, as described herein, can be used as a source to isolate polypeptides. The MICAL polypeptide can then be quantified using methods known to those of skill in the art, for example by ELISA.

Monoclonal antibodies to a particular polypeptide can be used in immunoassays, such as in liquid phase or bound to a solid phase carrier, to detect the MICAL polypeptide In addition, the monoclonal antibodies in these immunoassays can be detectably labeled in various ways. Examples of types of immunoassays that can utilize monoclonal antibodies of the invention are competitive and non-competitive immunoassays in either a direct or indirect format. Examples of such immunoassays are the radioimmunoassay (RIA) and the sandwich (immunometric) assay. Detection of the polypeptide antigens using the monoclonal antibodies of the invention can be done utilizing immunoassays, which are run in either the forward, reverse, or simultaneous modes, including immunohistochemical assays on physiological samples. Those of skill in the art will know, or can readily discern, other immunoassay formats without undue experimentation. In addition, there are a number of commercially available antibodies to cytokines of interest.

The term “immunometric assay” or “sandwich immunoassay” includes simultaneous sandwich, forward sandwich and reverse sandwich immunoassays. These terms are well understood by those skilled in the art. Those of skill will also appreciate that antibodies according to the present invention will be useful in other variations and forms of assays which are presently known or which may be developed in the future. These are intended to be included within the scope of the present invention.

Monoclonal antibodies can be bound to many different carriers and used to detect the presence of a MICAL polypeptide. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such using routine experimentation.

In performing the assays it may be desirable to include certain “blockers” in the incubation medium (usually added with the labeled soluble antibody). The “blockers” are added to assure that non-specific proteins, proteases, or anti-heterophilic immunoglobulins to anti-cytokine immunoglobulins present in the experimental sample do not cross-link or destroy the antibodies on the solid phase support, or the radiolabeled indicator antibody, to yield false positive or false negative results. The selection of “blockers” therefore may add substantially to the specificity of the assays.

Alternatively, the level of MICAL protein or nucleic acid can be determined in vivo using known imaging techniques. For example, an anti-MICAL antibody can be labeled with a radioactive marker whose presence and location in a subject can be detected by standard imaging techniques.

The results present herein reveal a series of embodiments directed at methods of treating neurological disorders involving a failure of axon regrowth and methods for inducing regrowth of injured processes of neurons that include altering the oxidative state of an affected cell. The present disclosure identifies a novel gene family, the MICALs (also referred to as the zeyphyrins, and the 151 family), whose protein domains suggest a novel means by which nerve growth is regulated. MICALs are characterized by a flavoprotein monooxygenases region (i.e., oxidoreducatase family) that has not been previously shown to function in axon guidance. The presence and necessity of this flavoprotein monooxygenase domain indicates that this gene family regulates repulsive axon guidance through a novel means-oxidation/reduction (redox) mechanisms. The present disclosure indicates that MICALs use redox mechanisms to regulate axon growth by directly or indirectly destabilizing the cellular machinery (i.e., the actin cytoskeleton) necessary for axon outgrowth.

The discovery of MICALs and their mechanism of action (i.e., redox mechanisms) illuminates a novel general means through which widespread inhibition of axon growth can occur-actin oxidation. In particular, the inability of axons to regrow after spinal cord injury may be a consequence of the presence of high amounts of reactive oxygen species and other oxidative mechanisms in the spinal cord milieu after injury. These oxidants may directly alter the structure of the actin cytoskeleton-in effect, acting non-specifically like members of the MICAL protein family. In total, these discoveries indicate novel and immediate treatments for many neurological disorders, targeting both general oxidants as well as the MICALs. In particular, these novel treatments include novel therapeutic strategies (e.g., antioxidants and other redox active compounds) and novel agents (e.g., EGCG, EC, and other flavonoids and antioxidants) to promote axonal regrowth (e.g., following spinal cord injury and similar neurological disorders) as well as novel strategies (e.g., oxidants) to limit abnormal and excessive axonal growth (e.g., following certain neuropathies, and increased sensitizations). In summary, the discoveries disclosed herein, implicate oxidation mechanisms in limiting axon growth and preventing axon regeneration in general; these mechanisms have not previously been suggested or shown to be involved in limiting axon outgrowth after spinal cord injury or after other neurological disorders.

Accordingly, in another aspect, the present invention provides a method for treating a neurological disorder involving a failure of axon regrowth, comprising contacting a neuron having axons that fail to regrow, or surrounding tissue, with an agent that neutralizes oxidants, thereby treating the neurological disorder. The surrounding tissue can include any tissue whose components, typically cells, can produce factors that affect axonal growth.

The agent that neutralizes oxidants can include virtually any anti-oxidant. Especially preferred are anti-oxidants that can be delivered orally and that can enter the central nervous system or peripheral nervous system. The agent for example, can be applied directly to a neuron having axons that fail to regrow. For example, the agent can be directly injected to the site of spinal cord injury. A method according to this aspect of the invention can be performed in vitro or in vivo.

For example, anti-oxidant vitamins can be used. These vitamins include vitamins E, C and beta carotene. Other useful antioxidants for the present invention include, for example, methylprednisolone, Tirilazad, lazaroids (21-aminosteroids) and similar steroids, alpha tocophenol, lycopene, gamma tocophenol, mannitol, catalase, and glutathione, superoxide dismutase. Also useful for the present invention is the anti-oxidant compound H 290/5 (See e.g., Thornwall M., et al., Acta Neurochir Suppl (Wien) 70:212-5 (1997)), and the anti-oxidant AM-36 (Callaway, J. K., J. Alzheimers Dis. 2(2):69-78 (2000)).

As indicated herein, monooxygenase inhibitors and anti-oxidant flavonoids, including those that are monooxygenase inhibitors such as ECGC and EC, can be used as anti-oxidants for this embodiment and embodiments aimed at inducing regrowth of an injured process of a neuron. The anti-oxidant flavonoid can be a gallic acid derivative such as ECGC or EC. Examples of other gallic acid derivatives that can be used to affect axonal guidance regulatory activity in embodiments of the present invention include, but are not limited to, (−)-gallocatechin-3-O-gallate (GCG), (−)-epicatechin-3-O-gallate (ECG), (−)-epigallocatechin (EGC), (+)-gallocatechin (GC), theasinensin A, 3″-O-methyl-EGCG, 3″-O-methyl-ECG, 3″-O-methyl-GCG, (−)-epigallocatechin (EGC), (1)-gallocatechin (GC), gallic acid, catechin, n-octyl gallate, and n-cetyl gallate.

In another embodiment, the present invention provides a method for inducing regrowth and/or preventing inhibition of an injured process of a neuron, that includes altering the levels of reactive oxygen species in the milieu of the neuron. The method can include identifying a site that includes the neuron suspected of having an injured process, before altering the levels of reactive oxygen species or other oxidation products in the milieu of the neuron. The neuronal process can be an axon or a dendrite. The levels of reactive oxygen species or other oxidation products are typically decreased by the method.

In certain aspects, levels of reactive oxygen species are altered chronically, as discussed herein for application of an agent. For example, the levels can be altered for a period of time that is sufficient to permit axon regrowth (i.e. neurorestoration) and the establishment of synaptic connections with new targets. This chronic alteration of the level of reactive oxygen species, in certain aspects is for at least 1, 2, 7, or 14 days, or 1, 2, 3, 4, 5, 6, 12, 24, 36, 48, or 60 months after identification or suspicion of the injured process of the neuron.

In one aspect, oxygen species can be decreased chronically by delivery of a recombinant cell that expresses a recombinant enzyme that lowers reactive oxygen species to a site of neurological damage or other site in need of regrowth of neural processes. Such recombinant enzymes include, for example, catalase and superoxide dismutase. Alternatively, enzymes that lower reactive oxygen species can be delivered to a site in need of regrowth of neural processes.

The method can further include adding an agent that promote neuron process regrowth, such as a neurotrophic factor or a neural stem cell, as discussed above, to the milieu of the neuron. The milieu of the neuron includes fluids, molecules, and tissues that surround a neuron. As discussed hereinabove, the agent that promotes neuron process regrowth can be, for example, a neurotrophin, a mechanical bridge, or a stem cell.

In another embodiment, the present invention provides a method for limiting abnormal axon outgrowth, that includes contacting a neuron or the milieu of the neuron with an agent that affects oxidation state. The abnormal axon outgrowth can be excessive axon outgrowth.

In another embodiment, the present invention provides a method for improving sperm function, that includes contacting a sperm cell, or progenitor thereof, with an antioxidant agent in an amount sufficient to modulate MICAL activity. In certain aspects, the method includes reducing levels of reactive oxygen species in the milieu of the sperm or progenitor thereof. Human spermatozoa exhibit a capacity to generate ROS and initiate peroxidation of the unsaturated fatty acids in the sperm plasma membrane, which plays a key role in the etiology of male infertility (Sharma R. K., and Agarwal A., Urology, 48:835 (1996)). Accordingly, MICAL expression can be involved in sperm malfunction through oxidation of components of sperm cells. Therefore, agents such as antioxidants, for example, antioxidant flavonoids, and particularly monooxygenase inhibitors, such as those disclosed herein can be used to improve sperm function and to treat male infertility.

In another embodiment, the present invention provides a method for modulating cardiac development in a subject, for example a human subject such as a patient in need of the method. The method includes contacting a cardiac neural crest cell with an amount of an agent that modulates MICAL activity, the amount being effective to modulate cardiac development.

In another embodiment, the present invention provides a method for treating, managing, and/or ameliorating the symptoms of a cardiovascular disease in a human subject. The method includes contacting a cardiac cell in the human subject with an amount of an agent that modulates MICAL activity. The amount is effective to treat, manage, and/or ameliorate the symptoms of the cardiovascular disease. The agent can modulate any of the activities of a MICAL included, for example, axon guidance regulatory activity, plexin interacting activity, actin binding, and/or monooxygenase activity.

In another embodiment, the present invention provides a method for modulating an immune response in a human subject in need thereof. The method includes contacting an immune cell in the human subject with an amount of an agent that modulates MICAL activity. The amount is effective for modulating said immune response. In certain aspects, the immune response is inflammation. MICALs through their involvement in semaphorin-mediated pathways, are predicted to be involved in semaphorin-mediated processes of the immune system. For example, using a differential display technique, upregulation of semaphorin E in rheumatic synovial fibroblasts has been observed. Accordingly, in certain aspects, the human subject for the method for modulating an immune response has rheumatoid arthritis. In other embodiments, the human subject for the method of modulating an immune response has another inflammatory disease, such as, but not limited to, asthma, encephilitis, inflammatory bowel disease, chronic obstructive pulmonary disease (COPD), allergic disorders, septic shock, pulmonary fibrosis, undifferentitated spondyloarthropathy, undifferentiated arthropathy, inflammatory osteolysis, and chronic inflammation resulting from chronic viral or bacteria infections.

In another embodiment, the present invention provides a method for inhibiting cancer cell proliferation or metastasis in a human subject. The method includes contacting a cancer cell in the human subject with an amount of an agent that modulates MICAL activity. The amount is effective to inhibit MICAL axon guidance regulatory activity, monooxygenase activity, or plexin-interacting activity, thereby being effective to inhibit proliferation or metastasis of the cancer cell. As discussed herein, MICALs through their involvement in semphorin-mediated pathways are predicted to be involved in semaphorin-mediated processes in cancer cells, including metastatic cancer cells.

In another aspect, the present invention includes kits that are useful for carrying out the methods of the present invention. The components contained in the kit depend on a number of factors, including the type of method being carried out.

Accordingly, the present invention provides a kit for modulating the activity of a MICAL polypeptide. The kit includes an agent in an effective amount and formulation to be effectively delivered to a subject. The agent can be any of the agents disclosed herein. In certain aspects, the agent is a monooxygenase inhibitor, such as a flavonoid, for example a gallic acid derivative. The gallic acid derivative in certain aspects is ECGC or EC. The kit also includes instructions, either as a pamphlet provided with the kit, or in an on-line site that provides instructions, for performing the method for modulating activity of the MICAL polypeptide.

In another aspect of a kit embodiment, the present invention provides one or more containers that include a MICAL or MICAL-Like polypeptide or polynucleotide of the present invention, a vector that includes the MICAL or MICAL-like polypeptide operably linked to a heterologous promoter, and/or a recombinant cell that includes the vector. The kits can include instructions for performing any of the methods provided herein.

In another aspect, the kit can provide a container that includes a MICAL detection molecule. A MICAL detection molecule is for example, an antibody, an oligonucleotide probe, or any of the other known types of molecules that can be used to detect expression or activity of MICAL, as disclosed herein. The kit in certain aspects includes an oligonucleotide probe, primer, or primer pair, or combination thereof for carrying out a detection method of the present invention, as discussed above. For example, the probe, primer, or primer pair, can be capable of selectively hybridizing to a MICAL polynucleotide. The kit can further include one or more detectable labels.

The following examples are intended to illustrate but not limit the invention.

### EXAMPLE 1

### MICAL is a Large, Cytosolic, Multidomain Protein that Interacts with Drosophila Plexin A

This example illustrates that Drosophila MICAL is a multi-domain protein that interacts with Plexin A.

Yeast Two-Hybrid Screening

Yeast protocols were conducted using standard techniques (Golemis et al., 1994). Portions of the intracellular domains of PlexA (amino acids 1702-1945; EST LD13083), PlexB (amino acids 1785-2051; EST CK00213), and the corresponding intracellular regions of human Plexin A3, and mouse Plexin A4 (gifts of L. Tamagnone, and H. Fujisawa, respectively) were inserted into the yeast bait vector, as described in more detail as follows:

The terminal “C2” portion of the PlexA cytoplasmic domain (amino acids 1702-1945), which is highly conserved among all plexin family members, was used to search for interacting proteins encoded by a Drosophila embryonic (0-24 hrs.) cDNA library. The PCR-amplified PlexA C2 domain (the bait) was inserted into the yeast expression vector pEG202 (bait vector). Following sequencing of both strands, the bait was introduced into the yeast strain EGY48 containing the β-galactosidase expressing plasmid pJK103. Western analysis of transformed yeast using an antibody to LexA (Invitrogen) confirmed appropriate sized expression and an activation assay showed that the bait could not activate transcription on its own. A 0-24 hr. Drosophila embryonic cDNA library was cloned into the yeast expression vector pJG4-5 (generated by H. Araj). >2×10⁶ clones were screened and interactions assessed with a visual p-galactosidase assay and a test of growth in the absence of leucine. Yeast clones exhibiting varying degrees of interaction were selected and standard protocols were used to recover the library vector and sequence these clones on both strands.

cDNAs containing the C-terminal of human MICAL-1 (DFkzp434B517) and mouse MICAL-2 (BB481898) were used to amplify portions homologous to the last 200 amino acids of Drosophila MICAL and cloned into the library vector.

Molecular Analysis

Proteins, domains, and alignments were identified using Web-based protein domain searching and alignment tools (PFAM, BLAST, PRINTS, JALVIEW, and ClustalX) and our own molecular analysis. Human MICAL-1 (EST FLJ 1937), Human MICAL-2 (ESTs BF815128, KIAA1364, KIAA0819), and Human MICAL-3 (ESTs KIAA0750, and FLJ14966) were identified by BLAST searches on publicly available cDNA and genomic sequence and in some cases overlapping ESTs were assembled virtually. Drosophila MICAL-L (EST LD45758) and human MICAL-L1 (EST XM001070) and MICAL-L2 (ESTS FL00139 and FLJ23471) were identified by searching publicly available cDNA and genomic sequence databases.

Results

To identify mediators of semaphorin-dependent repulsive axonal guidance the terminal highly conserved “C2” portion of the PlexA cytoplasmic domain was used to search for interacting proteins encoded by a Drosophila embryonic (0-24 hrs.) yeast two-hybrid cDNA library (FIGS. 5A-C). Fifty-two interactors encoded by cDNAs derived from four different genes were identified. Over half of these interactors were encoded by a single gene and two overlapping cDNAs encoded by this gene were selected for further study (clones 23 and 151).

Yeast interactions using the C2 domain of PlexA as the bait (Plexin A C2) were assessed and the strongest interactors (e.g., clones 23, and 151), as determined by a β-galactosidase assay (Beta Gal Activity), were derived from MICAL (FIG. 5B). Further, the bait construct (Plexin A C2) was cloned into the library vector and clone 151 was inserted into the bait vector and an interaction assay demonstrated the vector independence of these interactions. Clones 23 and 151 do not interact with the C2 domain of Drosophila PlexB. Though GOF experiments suggest that PlexB functions like PlexA to signal semaphorin-mediated motor axon repulsion (Hu et al., 2001), we have thus far not observed associations between MICAL and PlexB proteins. The C2 domains of human Plexin A3 (HPlexin A3 C2) and mouse Plexin A4 (MPlexin A4 C2) interact strongly with the plexin interacting regions (PIR) of human MICAL-1 (HMICAL 1 PIR) and mouse MICAL-2 (MMICAL2 PIR), respectively.

DNA sequence analysis suggested that the overlapping cDNA clones we identified in our yeast screen did not encode a full-length gene product. These cDNAs have an open reading frame (ORF) at their 5′ ends and a stop codon near their 3′ ends, indicating we had identified the C terminal 255 amino acids of this novel protein. Northern analysis using standard techniques (Sambrook et al, 1989) on 0-24 hr Drosophila embryonic total RNA and a portion of clone 151 as a probe showed that the full-length transcript from this gene is greater than 10 kilobases (Kb) in length (Clone 151, 3′ MICAL) (FIG. 5C). Given a lack of publicly available expressed sequence tags (ESTs) extending our cDNA further 5′, we used one of our initial cDNA clones (clone 23; FIG. 1D) to screen a Drosophila embryonic lambda gt11 phage CDNA library (a generous gift from K. Zinn) for full-length cDNAs using standard techniques (Sambrook et al., 1989). The longest clones were selected and sequenced on both strands. We were unable to identify a single full-length transcript, so we conducted an extended cDNA walk to obtain full-length MICAL transcripts. Isolated cDNAs were assembled to identify full-length MICAL isoforms. Northern analysis using a probe derived from the 5′ end of an assembled full-length cDNA detected a large transcript of greater than 10 kb (5′ MICAL). This transcript is similar in size to the transcript detected with a probe from the 3′ portion of this assembled cDNA, providing further evidence that the 3′ and 5′ portions of the assembled full-length cDNAs are from the same transcript or group of large transcripts.

The genomic organization of the MICAL locus was determined using the Sequencer 2.1 program (Gene Codes Corp.), the identified cDNAs, and publicly available Drosophila genomic DNA sequences. This extensive molecular analysis demonstrated that the Drosophila gene defined by clones 23 and 151, is Drosophila MICAL, and covers >41 kb of genomic sequence and has at least 25 exons (FIG. 1A; see FIG. 5). Based on analysis of isolated cDNAs and western analysis (see FIG. 6D), there are at least three MICAL isoforms (“long,” “medium,” and “short” variants; FIG. 1A).

At the MICAL C-terminus is the plexin interacting region that was identified in the yeast screen. Within the plexin interacting region there is a predicted heptad-repeat, coiled-coil structure (FIG. 1B), a motif thought to be involved in protein-protein interactions (Burkhard et al., 2001). Interestingly, this region of MICAL shares amino acid similarity with several other coiled-coil domain-containing proteins including a portion of the alpha domain found in the Ezrin, Radixin, and Moesin (ERM) proteins (˜22% identity; Bretscher et al., 2000). The last four amino acids of MICAL (ESII) are a PDZ protein binding motif (Harris and Lim, 2001). N-terminal to the plexin interacting region of MICAL there is a proline rich region. MICAL has two regions of varying length, variable regions (1) and (2), which have no significant similarity to any other proteins and which appear to determine the size of the different MICAL proteins (FIG. 1B). MICAL has a single LIM domain (FIG. 1B), a protein-protein interaction module found in a variety of proteins involved in signal transduction cascades and in cytoskeletal organization (Bach, 2000), and also a single calponin homology (CH) domain (FIG. 1B), a domain also found in cytoskeletal and signal transduction proteins and known to be involved in actin filament binding (Gimona et al., 2002). The MICAL N-terminal ˜500 amino acid domain is highly conserved among MICAL-related proteins (see below), but is unique over its entire length in comparison to other proteins.

### EXAMPLE 2

### MICAL is Expressed on Drosophila Embryonic Motor and CNS Axons and Coimmunoprecipitates with Plex A

This example illustrates that MICAL is expressed in axons and that MICAL interacts with PlexA.

In Situ Hybridization

RNA in situ analysis of whole-mount Drosophila embryos and cryosections of E15 and E18 rat spinal cords were as described (Kolodkin et al., 1993; Pasterkamp et al., 1998).

Development of HA-PlexA Transgenic Flies

The HA-PlexA construct was created by inserting in the correct orientation an in-frame PCR amplified HA sequence into the EcoRI site that links the artificial signal sequence and the extracellular domain of PlexA in a PlexA pSectag B construct generously provided by C. Goodman. Following sequencing of the insert, the entire HA-PlexA cDNA was inserted into the pUAST vector; one transgenic fly was obtained.

MICAL Antibody Generation, Western Analysis, Immunohistochemistry, and Immunoprecipitation

Antibodies were generated and characterized as described (Yu et al, 1998). cDNAs corresponding to the last 359 amino acid of MICAL (MICAL-CT antibody) were inserted into the pTrcHisA vector (Invitrogen). MICAL-CT antibodies were used for Western analysis at a 1:2000 dilution, and on Drosophila embryos at 1:3000 dilution.

Embryos generated by crossing UAS-HAPlexA and Elav-GAL4/Cyo adults were collected, and co-immunoprecipitations were performed using standard techniques, and an HA monoclonal antibody (12CA5; Roche). Western analysis was performed using an HA antibody (1:3000, rat mAb Clone 3F10, Roche), our MICAL-CT antibody (1:2000), and an Enabled antibody (1:500; IG6C10; gift from D. Van Vactor).

Immunoprecipitations

Embryos generated by crossing UAS-HAPlexA and Elav-GAL4/Cyo adults were collected, dechlorinated with 50% bleach, and 100-200 mg of embryos were lysed in either 1) 1 mL of RIPA buffer (150 mM NACl, 1.0% NP-40, 0.5% deoxycholate, 0.1% SDS, 50 mM Tris, pH 8.0, 0.2 mM NaVO₄, 10 mM NaF, and protease inhibitor cocktail (Sigma) and 20 μg/mL PMSF), 2) 1 mL of 1% Triton buffer (150 mM NaCl; 50 mM Tris-HCl, pH 8.0), or 3) 1% NP-40 (150 mM NaCl, 5 mM Tris-HCl, pH 8.0) using a tight 2 mL Dounce homogenizer at 4° C. Similar results were observed with each buffer. Extracts were cleared by ultra centrifugation at 100,000×g for 15 min. at 4° C. and added to 50 μL of a 50% slurry of Gammabind G beads (Amersham) for 30 min with rocking at 4° C. Lysates with beads were then centrifuged for 30 minutes and supernatants were immunoprecipitated for 30 minutes with 2 μL anti-HA per sample (mouse monoclonal antibody (Clone12CA5, Roche). 100 μL of Gammabind G beads were then added to the sample, and the samples were incubated for 90 min. at 4° C. with rocking, washed 6 times with lysis buffer and resuspended in 50 μL of Laemmli loading buffer. Western analysis was performed using an HA antibody (1:3000, rat mAb Clone 3F10, Roche), our MICAL-CT antibody (1:2000), and an Enabled antibody (1:500; IG6C10; a generous gift from D. Van Vactor).

Results

In situ hybridization analysis using RNA probes corresponding to the N- or C-terminal of MICAL shows that MICAL and PlexA have similar patterns of embryonic mRNA expression. During early Drosophila development (stages 7-8), both MICAL and PlexA are expressed in the ventral neurogenic region and in many non-neuronal tissues (including developing mesoderm, cells surrounding the cephalic furrow and amnioproctodeal invagination, and in gut primordia). This non-neuronal expression is also seen later in embryonic development (stages 11-17), where both MICAL and PlexA are present within the anterior and posterior midgut primordia, the visceral musculature, and weakly in somatic musculature. During axonal pathfinding (stage 13 onward) both MICAL and PlexA are expressed within the developing brain and ventral nerve cord in most, if not all, CNS neurons but MICAL, like Sema1a and PlexA, is not highly expressed in peripheral sensory neurons.

Western blot analysis using a polyclonal antibody directed against the MICAL C-terminus (MICAL-CT) revealed prominent bands at 530 kD, 330 kD, 300 kD, 200 kDa, and 125 kDa in lysates from wild-type embryos which are seen at greater intensity in lysates from embryos harboring a chromosomal duplication that includes the MICAL locus (see FIG. 6). The three largest protein bands are in agreement with the molecular weights of the three MICAL isoforms predicted from our analysis of MICAL cDNAs (FIG. 1A). MICAL immunoreactivity was not observed in embryonic lysates obtained from mutant embryos harboring a deficiency which includes the MICAL locus (see below), showing these products identified by Western analysis are derived from MICAL and that our antibodies are MICAL-specific (see FIG. 6D).

MICAL protein is present in neuronal cell bodies, along axons, and in growth cones. MICAL immunostaining first appears in the nervous system at stage 13 and labels motor and CNS projections. At later embryonic stages, MICAL immunostaining is present on axons that make up all motor axon pathways: the intersegmental nerve (ISN); the intersegmental nerves (ISNb and ISNd); and the segmental nerves a and c (SNa, and SNc). MICAL immunostaining is also present in segment boundaries at the position of muscle attachment sites and at low levels in the lateral cluster of chordotonal organs.

To ask whether PlexA and MICAL directly interact in neurons, transgenic flies were generated that contain a transgene encoding epitope-tagged PlexA (HA-PlexA) under the control of an upstream activator sequence (UAS) (Brand and Perrimon, 1993) and crossed with flies that express the GAL4 transcription factor in all neurons (Elav-GAL4). Lysates from embryos containing both HA-PlexA and Elav-GAL4 elements were subjected to immunoprecipitation using HA antibodies and then Western blotting with MICAL-CT antibodies. Robust co-immunoprecipitation of MICAL was observed using HA antibodies and also reciprocal co-immunoprecipitation of HA-PlexA using MICAL-CT antibodies. The “large” MICAL isoform is the predominant variant observed to be associated with neuronally-expressed HA-PlexA, which may reflect tissue-specific expression of this isoform in neurons. MICAL co-immunoprecipitation appears specific since enabled (Ena), a neuronally expressed cytosolic protein, was not co-immunoprecipitated by HA antibodies in similar experiments and Unc5, a neuronally-expressed transmembrane receptor was not co-immunoprecipitated by MICAL-CT antibodies.

### EXAMPLE 3

### A MICAL Loss-of-Function Mutant Demonstrates that MICAL is Required for Motor Axon Pathfinding

This example illustrates that MICAL is required for motor axon pathfinding.

Drosophila Genetics and Phenotypic Characterization

Drosophila genetics, transformations, and preparation and analyses of Drosophila embryos was performed as described (Winberg et al., 1998b; Yu et al., 1998). The cytological location of MICAL was determined by hybridizing a radiolabeled cDNA probe corresponding to either the 5′ or the 3′ regions of the MICAL ORF on a Drosophila genomic P1 clone filter (Genome Systems) and following the manufacturer's instructions. MICAL is located on the third chromosome of Drosophila in the 85F3-6 chromosomal location. Unfortunately, this region was devoid of any small, publicly available, deficiencies and candidate MICAL mutations.

To generate a MICAL LOF mutant we identified two P transposable elements closely flanking the MICAL locus and used a P element transposase-mediated mutagenesis strategy to delete the region between these P elements (Cook et al., 2001; Cooley et al., 1990; Preston et al., 1996). A search of public databases revealed two transposable elements that flanked the MICAL locus and were separated by ˜165 kbs. Our molecular analysis confirmed that one (1(3)s2681; Bloomington Stock Center) was located in a separate gene <3 kb from the putative 5′ end of MICAL and the other (EP(3)3681; Berkeley Drosophila Genome Project) was 120 kb from the MICAL 3′ end. A third P element (1(3)10477) was situated between these two elements and served as a genetic marker. Our molecular analysis shows that 1(3)10477 (Bloomington Stock Center) is situated in a novel gene ˜70 kb from the 3′ end of MICAL, between 1(3)s2681 and EP(3)3681.1(3)10477 adult flies hold their wings out-stretched at a 45° angle from their bodies. Molecular analysis, genetic complementation analysis, and identification of additional 1(3)10477 alleles (including on the TM3 balancer) show that the wing phenotype associated with 1(3)10477 is recessive and due to a mutation of a gene located ˜70 kb from the 3′ end of MICAL (Terman and Kolodkin, unpublished). We have called this new gene stretched out (stretch) and used it as a genetic marker to identify MICAL deletions (submitted to Flybase). (1) Using standard genetic techniques, we generated adult flies containing each starting P element in trans (EP(3)3681|1(3)s2681). (2) A source of P element transposase was introduced as a mutagen and screened for the appearance of adult progeny containing a stretch wing phenotype (Screened through ˜25,000 flies).

80 adults were identified with a stretch-like wing phenotype and individual fly stocks were made for each. Each stock was then re-scored for the stretch phenotype. Five candidate MICAL deficiency lines were identified and the extent of the deletion was mapped using standard techniques by asking whether these lines complemented (“+”) or failed to complement (“−”) (i.e., showed lethality or the wing phenotype) when crossed to flies containing chromosomal aberrations flanking the MICAL locus. The fly stocks used in the mapping (complementation analysis) experiments were as follows: Df(3R)by10 (85D8;85E10-13; Bloomington Stock Center), Df(3R)by62 (85D11-14;85F6, 041h; 85F6(T); Bloomington Stock Center), Df(3R)segG16 (segregant Dp(2;2)G16[2D]TE35B-3[2P]; breakpoints=Df(3R)85F6-8;85F12-86A2+Dp(2;2)35B2;35D7 was made from T(2;3)G16; for simplicity referred to as Df(3R)segG16; kindly provided by John Roote and Michael Ashburner), and two small deficiencies generated in our lab (Df(3R)mr73 and Df(3R)mrO1; Terman and Kolodkin, unpublished). One of the five lines which exhibited a stretched out wing phenotype and removes MICAL (Df(3R)sWp2^{MICAL}) was used to characterize the MICAL loss-of-function phenotype, and the extent of this deletion is indicated in FIG. 6C.

Western Blot Analysis

Late stage 16/17 Drosophila embryos were genotyped and homogenized in lysis buffer (1% NP-40, 50 mM Tris-HCl pH 8.0, 2 mM EDTA, protease inhibitor cocktail), run on 4% SDS-PAGE gel, and subjected to western blotting. Lysates from 5 wild-type embryos, 5 embryos carrying a duplication of the MICAL locus (Dp(3;3)M86D[+]2 (85DI-4;87A5; Bloomington Stock Center), and 5 Df(3R)swp2MICAL embryos were blotted with either MICAL polyclonal antisera (MICAL-CT) or as a loading control an enabled monoclonal antibody (IG6C10). Prominent bands are observed at 530 kD, 330 kD, 300 kD, 200 kDa, and 125 kDa in wild type and at stronger intensity in MICAL duplication embryo; none of these bands are observed in Df(3R)swp2^{MICAL} embryos.

Drosophila Transformation Constructs

A MICAL rescue construct (UASMICAL) was physically assembled from isolated cDNAs and cloned into the pUAST vector for Drosophila germline transformation (Yu et al, 1998). Three independent transgenics were obtained. In addition to using the rescue construct to attempt to rescue the LOF and the genetic interaction phenotypes, this construct was used to examine the effects of overexpression of MICAL in all neurons. Expressing one copy of UASMICAL in all neurons in a wild-type background resulted in less penetrant phenotypes than expressing 2 copies.

Results

To determine whether MICAL functions in vivo to propagate Sema-1a-mediated motor axon guidance, detailed genetic analyses were performed of MICAL gain- and loss-of-function mutants. A small, tractable, deficiency (called Df(3R)swp2MICAL; see FIG. 6) was generated that removes ˜170 Kb that includes the MICAL locus and ˜six other genes. Western blot analysis on lysates from embryos homozygous for Df(3R)swp2MICAL demonstrates a loss of all MICAL protein (FIG. 6D), and no MICAL immunostaining is observed in these embryos. These data, in combination with rescue experiments using a MICAL CDNA (see below), define the small deficiency Df(3R)swp2MICAL as a MICAL null allele. Df(3R)swp2MICAL homozygotes survive into larval stages and have no overt morphological abnormalities (see FIG. 6).

If MICAL functions in Sema-l a/PlexA-dependent repulsive axon guidance, then MICAL LOF mutants should exhibit motor axon guidance defects similar to the distinct and highly penetrant defects seen in Sema1a and PlexA LOF mutants. The development of the stereotypic pattern of neuromuscular connectivity in embryonic Drosophila abdominal segments is observed with anti-fasciclin II (mAb 1D4) staining of stage 16/17 embryos (VanVactor et al., 1993). Motor axons initially exit the CNS as part of either the intersegmental nerve (ISN) or the segmental nerve (SN). They are then guided into five major nerve branches (the ISN, ISNb, ISNd, SNa, and SNc), each of which targets different muscle groups such that individual motor axons eventually innervate individual target muscles (Landgraf et al., 1997).

In wild-type embryos, the ISNb is formed by ISN axons defasciculating and extending dorsally through the ventral musculature to innervate muscles 6 and 7 and muscles 12 and 13. Axons within the ISNb pathway in Sema1a or PlexA mutants often fail to defasciculate and innervate their muscle targets (Table 1; Winberg et al., 1998b; Yu et al., 1998). In the absence of MICAL, axons within the ISNb show similar highly penetrant ISNb phenotypes (Table 1). These phenotypes include the failure of some or all axons to defasciculate from the ISN, stalling of axons within the ISNb following defasciculation from the ISN, ISNb axons bypassing their target muscle groups, and greatly reduced or absent innervation of target muscles.

Axons within the SNa pathway in MICAL mutants also exhibit highly penetrant defects similar to those observed in both Sema1a mutants and PlexA mutants. In wild-type embryos, SNa axons defasciculate from the SN and extend through the ventral musculature as a single tightly fasciculated bundle. At the dorsal edge of muscle 12, SNa axons defasciculate to give rise to a dorsal (D) and lateral (L) branch. Axons within the dorsal branch extend dorsally between muscles 22 and 23 and then make two characteristic turns, continuing further dorsally between muscles 23 and 24. In Sema1a and PlexA mutants, SNa axons within the dorsal branch often stall near muscle 12 and fail to reach the dorsal-most portion of their trajectory (Table 1; Winberg et al., 1998b; Yu et al., 1998). MICAL mutants exhibit similar, highly penetrant, SNa stall phenotypes (Table 1). MICAL mutants also exhibit prominent guidance defects in axons that give rise to the ISNd, SNc, TN, and the third most lateral fasciclin II-positive CNS longitudinal connective-defects, which have been observed in Sema1a and PlexA mutants (Winberg et al, 1998b; Yu et al, 1998). In MICAL LOF mutant embryos additional phenotypes beyond those seen in PlexA and Sema1a mutants were not observed, suggesting that MICAL primarily functions during Drosophila neural development in PlexA signaling events.

MICAL expression was restored in homozygous Df(₃R)swp2^{MICAL} embryos using one copy of the transgenic construct UAS-^{MICAL} under the control of the neuron-specific driver Elav-GAL4. Due to the large size of the MICAL protein, rescue was attempted using the smallest MICAL isoform—the 300 kD “small” form (FIG. 1A). The level of neuronal MICAL expression observed by immunostaining with MICAL antibodies in three independent MICAL transformants was somewhat lower than that seen in wild type embryos. Neuronal MICAL expression did not rescue the adult lethality in Df(3R)swp2 MICAL homozygotes, suggesting a requirement for the MICAL “long” form, other genes within the Df(3R)swp2 MICAL deficiency, and/or MICAL in non-neuronal cells for adult viability. We did, however, observe that neuronal MICAL expression in homozygous Df(3R)swp2^{MICAL} embryos almost completely rescues embryonic ISNb and SNa motor axon guidance defects (Table 1) and CNS longitudinal connective defects. Therefore, axon guidance phenotypes observed in the MICAL deficiency Df(3R)swp2^{MICAL} result from a lack of neuronal MICAL.

TABLE 1
Axon guidance phenotypes (ISNb and SNa Phenotypes)

| | Abnormal | Abnormal | Abnormal | |
|---|---|---|---|---|
| | ISNb | Muscle 6/7 | Muscle 12/13 | SNa |
| Genotype (hemisegments) | Bypass^{a}(^{x}) | Innervation^{b}(^{y}) | Innervation | Pathway^{c}(^{z}) |
| CONTROLS: | | | | |
| +/+ (wild type) (n = 120)) | 0% | 1.7% | 2.5% | 10.0% |
| Elav-GAL4/+ (n = 130) | 0% (0%) | 1.5% (0%) | 4.6% | 8.5% (0%) |
| Elav-GAL4/Elav-GAL4 (n = 109) | 0% (0%) | 2.8% (0%) | 8.2% | 12.8% (0%) |
| Df(3R)swp2^{MICAL}/+ (n = 110) | 0% | 3.6% | 7.2% | 7.3% |
| Semala^{P1}/+ (n = 110) | 0% | 2.7% | 8.1% | 9.1% |
| Df(4)C3^{PlexA}/+ (n = 100) | 0% | 1.0% | 3.0% | 12.0% |
| LOSS OF FUNCTION: | | | | |
| Df(3R)swp2^{MICAL}/Df(3R)swp2^{MICAL}(n = 103) | 1.0% | 68.9% | 57.3% | 81.2% |
| Semala^{P1}/Semala^{P1} (n = 97) | 5.2% | 47.4% | 77.3% | 85.7% |
| Df(4)C3^{PlexA}/Df(4)C3^{PlexA} (n = 148) | 4.1% | 60.8% | 47.3% | 74.3% |
| Elav-GAL4/UASMICAL; | 0 | 5.8% | 13.0% | 20.3% |
| Df(3R)swp2^{MICAL}/Df(3R)swp2^{MICAL} (n = 138) | | | | |
| UASMICAL^{G→W}/+; Elav-GAL4/+; | 0% | 67.2% | 68.6% | 69.7% |
| Df(3R)swp2^{MICAL}/Df(3R)swp2^{MICAL} (n = 137) | | | | |
| GENETIC INTERACTIONS: | | | | |
| Semala^{P1}/+; Df(3R)swp2^{MICAL}/+ (n = 110) | 3.6% | 32.7% | 37.3% | 51.8% |
| Df(3R)swp2^{MICAL}/+; Df(4)C3^{PlexA}/+ (n = 105) | 0% | 41.9% | 33.0% | 44.6% |
| Semala^{P1}/+; Df(4)C3^{PlexA}/+ (n = 108) | 0% | 32.4% | 39.8% | 68.5% |
| Semala^{P1}, Elav-GAL4/+; | 0% | 6.1% | 6.1% | 9.2% |
| UASMICAL/Df(3R)swp2^{MICAL} (n = 99) | | | | |
| GAIN OF FUNCTION: | | | | |
| Elav-GAL4/Elav-GAL4; | 2.1% (50%) | 32.0% (3.2%) | 48.5% | 46.4% (2.2%) |
| UASMICAL^{Myr-CT}/UASMICAL^{Myr-CT} (n = 97) | | | | |
| UASMICAL/UASMICAL; | 0.8% (100%) | 44.2% (59.6%) | 50.0% | 38.0% (71.4%) |
| Elav-GAL4/Elav-GAL4 (n = 130) | | | | |
| UASMICAL^{G→W}/+; | 2.4% (100%) | 36.7% (43.5%) | 44.4% | 48.5% (37.8%) |
| Elav-GAL4/+ (n = 170) | | | | |
| Description of phenotypes: ^{a}failure of all ISNb axons to defasiculate from the ISN; ^{b}ISNb axons stalling, bypassing targets, absent or decreased muscle innervation; ^{c}failure to make the two characteristic turns between muscles 22 and 23 and muscles 23 and 24; ^{x}all ISNb axons follow the ISNd or SNc, or ISNb axons remain fasciculated with the ISN but ultimately wander in the lateral muscle fields | | | | |
| # or project back to innervate ventral muscles; ^{y}increased (long or excessively thick) muscle innervation, excessive branching, projecting into abnormal target fields; ^{z}fasciculation with the ISN, premature branching, following abnormal pathways, termination on wrong muscles. (^{x, y, z} indicate percent (%) of defects in ^{a, b,} and ^{c}, respectively). | | | | |

### EXAMPLE 4

### MICAL Genetically Interacts with Sema1a and PlexA

This example illustrates that MICAL and PlexA function in the same signaling pathway to guide motor axons.

Results

To address whether MICAL functions in the same signaling pathway with PlexA to mediate Sema-1a-mediated repulsive axon guidance classical genetic interaction analysis was employed by asking whether heterozygosity at both MICAL and PlexA, or MICAL and Sema1a, resulted in phenotypes not observed in either heterozygote alone. MICAL, PlexA, or Sema1a heterozygous embryos show no motor axon guidance defects (Table 1). Embryos heterozygous for both Sema1a and PlexA (Sema1a/+;Df(4)C3PlexA/+) show highly penetrant axon guidance defects similar to those observed in homozygous Sema1a or PlexA mutants (Winberg et al., 1998b; Table 1). Embryos heterozygous for both MICAL and Sema1a, or heterozygous for both MICAL and PlexA, exhibit axon guidance phenotypes similar to those seen in Sema1a/+;Df(4)C3^{PlexA}/+embryos, and these are seen at approximately equal penetrance (Table 1). For example, the ISNb and SNa of Sema1a/+; Df(3R)swp2^{MICAL}/+or Df(3R)swp2^{MICAL}/+;Df(4)C₃^{PlexA}/+embryos exhibit guidance errors at specific choice points similar to those seen in homozygous PlexA, Sema1a, or MICAL mutant embryos. One copy each of both the UAS-MICAL and Elav-GAL4 transgenes was introduced into the Sema1a/+; Df(3R)swp2^{MICAL}/+background and observed that neuronal MICAL expression rescues both the ISNb and SNa phenotypes in these transheterozygous embryos (Table 1). These results support the idea that MICAL and PlexA function in the same signaling pathway to guide motor axons.

### EXAMPLE 5

### MICAL Gain-of-Function Axon Guidance Phenotypes

This example provides results that further establish that MICAL participates in PlexA-mediated motor neuron guidance, and illustrates that dominant negative MICAL mutants can be generated.

The MICAL^{Myr-CT} construct was constructed by PCR amplification of the plexin interacting region of MICAL with PCR primers containing a myristoylation sequence (base pairs corresponding to the first 14 amino acids of Drosophila src; Simon et al, 1985), cloned into the pUAST vector, sequenced on both strands, and one Drosophila transgenic was obtained as described above. Embryos expressing 1 copy of UASMICAL^{Myr-CT} in all neurons using the GAL4-UAS system (Elav-GAL4) exhibited phenotypes less penetrant then when 2 copies were expressed.

Results

To complement MICAL LOF analysis, it was determined whether MICAL GOF mutants exhibit motor axon guidance phenotypes similar to those observed in PlexA GOF mutants (Winberg et al., 1998b). MICAL was overexpressed in all neurons in a wild type background using the GAL4-UAS system and our rescue construct. Neuronal overexpression using one or two copies of our MICAL rescue construct leads to highly penetrant motor axon guidance phenotypes (Table 1). GOF phenotypes resulting from one copy of the MICAL rescue construct in a wild type background can be suppressed in a Df(3R)swp2^{MICAL} genetic background (Table 1). These defects in some cases are quite similar to the defects observed in MICAL mutants and defects reported in PlexA GOF mutants (Winberg et al., 1998b). However, a large fraction of these MICAL GOF motor axon guidance phenotypes are consistent with increased defasciculation (Table 1), as similarly described for the PlexA GOF mutants. For example, ISNb axons were often seen to abnormally leave the ISNb and project incorrectly within the ventral musculature (Table 1). Likewise, some SNa axons defasciculated at incorrect locations and projected to inappropriate areas (Table 1). Therefore, MICAL GOF mutants exhibit phenotypes similar to PlexA GOF mutants, again suggesting that MICAL participates in PlexA-mediated motor axon guidance.

Additional support for MICAL's role in PlexA signaling was obtained by expressing in all neurons a truncated MICAL protein consisting only of the MICAL PlexA-interacting region (FIG. 1B). This protein was targeted to the cell membrane by introducing an N-terminal myristoylation sequence (MICALMyr-CT) and found that neuronally expressed MICALMyr-CT acts in a dominant-negative fashion, resulting in axon guidance phenotypes similar to those observed in MICAL mutants (Table 1). Prominent GOF phenotypes like those resulting from MICAL or PlexA overexpression were not observed, indicating that neuronal MICALMyr-CT is likely occluding normal MICAL-PlexA associations and therefore MICAL signaling. This also suggests that the MICAL protein contains domains distinct from the PlexA-interacting domain that function to regulate axonal guidance.

### EXAMPLE 6

### The MICALS are a Family of Neuronally Expressed, Plexin-Interacting Proteins Conserved from Flies to Mammals

This example illustrates that MICAL proteins have conserved protein domains with identical organization in all family members.

Results

MICAL proteins have conserved protein domains with identical organization in all family members and a high degree of amino acid identity among these domains in different MICALs (FIG. 2A). Suzuki et al. (2002) identified MICAL-1 and a partial sequence of MICAL-2. One MICAL was identified in Drosophila and three mammalian MICALs were identified. The MICALs appear unique with respect to containing both calponin homology (CH) and LIM domains, in addition to their conserved N- and C-terminal regions. A family of MICAL-like (MICAL-L) proteins were also identified, members of which have a similar organization to MICALs but lack the region N-terminal to the CH domain (FIG. 2B). There is one MICAL-L protein in Drosophila (D-MICAL-L) and at least two family members in humans. D-MICAL-L cDNA and genomic DNA sequence information suggest that D-MICAL-L begins just N-terminal to the CH domain. Analysis of publicly available mammalian cDNA and genomic sequences suggests that human MICAL-L1 and MICAL-L2 are similar in overall domain organization to D-MICAL-L and do not contain the highly conserved 500 amino acid MICAL N-terminal domain.

To address whether the function of MICALs is conserved in vertebrates, expression patterns and interactions with plexins were analyzed. It was found that the mRNA of all three rat MICALs shows specific neuronal and non-neuronal expression during development. For example, MICAL1, MICAL2, and MICAL3 are expressed in the rat spinal cord, dorsal root ganglia (DRG), and sympathetic ganglia at embryonic days 15 (E15) and E18 in patterns which appear overlapping but distinct. Interestingly, the neuronal expression patterns of individual MICALs are similar to those observed for several plexins, as can be seen for PlexA3 and MICAL1. In addition, results presented herein indicate that the plexin interacting domains of human MICAL-1 and mouse MICAL-2 specifically interact with the C2 domains of human PlexA3 and mouse PlexA4, respectively, and do so as strongly as the autologous domains of Drosophila MICAL and PlexA (see FIG. 5B).

### EXAMPLE 7

### MICALS Contain an N-Terminal Flavoprotein Monooxygenase Domain

This example illustrates that MICAL proteins include a highly conserved an N-terminal monooxygenases domain.

### MICAL Flavoprotein Monooxygenase Fusion Protein Purification and FAD Binding

A His-tagged bacterial fusion protein was constructed that included the MICAL flavoprotein monooxygenase domain (MICAL FM) by inserting amino acids 1-526 of Drosophila MICAL into the bacterial expression vector pET 43. lb containing a hexahistidine tag (Novagen). The plasmid was transformed into E. coli BL21 (DE3) and the hexahistidine tagged recombinant protein was expressed by IPTG induction and MICAL FM was isolated with the inclusion bodies, denatured with 6M GdmHCl (Gibco) and purified under denaturing conditions over a Ni²⁺ column (Novagen). MICAL FM was renatured at 25° C. for 3 hours by diluting the purified protein 100× into a solution containing a 5-fold molar excess (to MICAL FM present in the solution) of free FAD (Sigma), 10 mM DTT, and 10 μg/mL BSA as described (Lindsay et al., 2000). Renatured protein was dialyzed for 48 hours at 4° C. into 5 changes of His binding buffer (5 mM imidazole, 0.5 M NaCl, 20 mM Tris-HCl, pH 7.9) to remove free FAD, and DTT. Ni²⁺ purification beads were then incubated with the MICAL FM sample in batch for 5 hours. at 4° C. The solution was then repurified through a Ni²⁺ column. Fractions containing MICAL FM were pooled and subjected to dialysis into a more stable buffer containing 5 mM DTT, and then subjected to Coomassie staining and Western analysis to confirm the purity of the sample. Spectral analysis was done using a Perkin-Elmer UV/VIS Lambda-12 spectrophotometer scanning from 300 to 550 nm.

Results

The high degree of conservation of the MICAL N-terminus among family members (up to 62% identical between flies and humans; FIG. 2A) suggests that this domain is functionally important. Upon closer examination of this conserved region, we noted a consensus dinucleotide binding sequence, GXGXXG (FIGS. 1B and 3A), which is distinct from the sequence present in classical mononucleotide binding motifs (Eggink et al., 1990; Eppink et al., 1997; Schulz, 1992; Wierenga et al., 1986). Further, the amino acid sequence in this 500 amino acid region reveals that MICALs contain three separate sequence motifs spaced throughout this domain that define them as flavoprotein monooxygenases (also called hydroxylases), a subclass of oxidoreductases (Eggink et al., 1990; Eppink et al., 1997; Wierenga et al., 1986). The amino acid sequence surrounding the GXGXXG motif matches perfectly the consensus sequence for the ADP binding region of flavin adenine dinucleotide (FAD) binding proteins (Rossmann fold or FAD Fingerprint 1, FIGS. 1B and 3A), and distinguishes this region from consensus NAD, or NADP binding folds (Vallon, 2000; Wierenga et al., 1986). MICALs also have a well-conserved GD motif (FAD Fingerprint 2; FIGS. 1B and 3A) C-terminal to the FAD Fingerprint 1 region, which is important for binding the ribose moiety of FAD (Eggink et al., 1990; Eppink et al., 1997). Finally, MICALs have the conserved DG motif (“Conserved Motif”; FIGS. 1B and 3A) between the FAD Fingerprint 1 and 2 motifs that has been reported to be involved in binding the pyrophospate moiety of FAD (Eppink et al., 1997). Proteins with these consensus FAD binding regions bind FAD and use FAD in the catalysis of oxidation-reduction reactions. Flavoprotein monooxygenases are oxidoreductases (enzymes that catalyze oxidation and reduction reactions) and catalyze the insertion of one atom of molecular oxygen into their substrate using nucleotides as electron donors (Massey, 1995). These monooxygenases are also defined by their use of FAD as a co-enzyme. Apart from these three consensus regions, monooxygenases vary significantly, reflecting the wide range of enzymes in this family and their variable substrate binding pockets also encompassed within this domain (Eppink et al., 1997). However, MICALs and other monooxygenases show significant similarity within these three FAD binding regions and also similar spacing of these regions within the monooxygenase domain.

Does MICAL bind FAD? A solution of the purified MICAL-flavoprotein monooxygenase (FM) domain (expressed in bacteria) is yellow in color, a characteristic of flavoproteins. Spectral analysis of purified MICAL-FM shows that it has an absorption peak at 452 nm and a shoulder at ˜358 nm (FIG. 3B). This is similar to the absorption spectra of FAD itself (˜450 nm and ˜360 nm; Macheroux, 1999), and to other related flavoproteins (e.g., p-Hydroxybenzoate Hydroxylase, Hosokawa and Stanier, 1966; and GidA, White et al., 2001), suggesting that MICAL-FM binds FAD. These results, in combination with the sequence homology, raises the possibility that MICAL enzymatic activity within the N-terminal conserved domain serves an integral function in plexin signaling.

### EXAMPLE 8

### An Intact FAD Binding Motif is Required for MICAL Motor Axon Guidance Functions

This example illustrates that an intact flavoprotein monooxygenases domain is necessary for MICAL function in repulsive motor axon guidance.

To make the MICAL^{G→W} mutant (SEQ ID NO:20), the dinucleotide binding region of Drosophila MICAL was mutated from GXGXXG to WXWXXW, such that the glycines were changed to tryptophans. Oligonucleotides containing terminal endogenous restriction sites (Mlu I) and base pair substitutions (GGA GCA GGG CCC TGT GGA (SEQ ID NO:37) changed to TGG GCA TGG CCC TGT TGG (SEQ ID NO:38)) were used to amplify a 1.4 kb fragment that was cloned in the correct orientation into the full length MICAL rescue construct. The region was sequenced on both stands and substitutions also disrupted a restriction site (Apa I) so the mutated construct could also be confirmed by restriction analysis. One transgenic, located on the X chromosome, was obtained. RESULTS

The glycine residues within the GXGXXG motif of FAD binding proteins are essential for allowing the FAD binding and enzymatic activity (Wierenga et al, 1986; Dym and Eisenberg, 2001). To test the necessity of MICAL FAD binding, and potential enzymatic activity, for plexin-mediated repulsive axon guidance the three glycine residues within the FAD Fingerprint 1 motif of MICAL were mutated to tryptophan (GAGPCGL (SEQ ID NO:39)→WAWPCWL (SEQ ID NO:40): mutations which in related flavin-containing monooxygenases disrupt FAD binding but do not alter the overall structure of the protein (Kubo et al., 1997; Lawton and Philpot, 1993; Wierenga et al., 1986). The resulting construct, MICAL^{G→W}, was used for in vivo neuronal expression in Drosophila. Transgenic flies containing the UAS-MICAL^{G→W} transgene were generated and immunohistochemical and Western analyses confirmed that MICAL^{G→W} was expressed at levels comparable to those of our wild-type “short” MICAL variant that was used to rescue MICAL mutant motor axon guidance phenotypes. Unlike neuronal expression of MICAL in a homozygous Df(3R)swp2MICAL mutant background, which rescues all ISNb and SNa defects, one copy of the neuronal MICAL^{G→W} rescues none of these defects (Table 1). This strongly suggests that activity of the MICAL monooxygenase domain is necessary for normal MICAL function.

Since MICAL^{G→W} contains an intact plexin interacting domain but is functionally inactive, we predicted that it would exert a dominant-negative effect on motor axon projections in a wild-type genetic background, binding to PlexA but blocking signaling in a manner similar to the MICALMyr-CT construct. When one copy of the MICAL^{G→W} reporter construct was used to express MICAL^{G→W} in all neurons in a wild-type genetic background we observed highly penetrant ISNb, SNa, and CNS longitudinal connective defects (Table 1) providing further evidence that MICAL^{G→W} is being expressed neuronally and is likely able to bind PlexA. However, though many of these defects resemble phenotypes observed in Sema1a, PlexA, or MICAL LOF mutants (Table 1), a significant fraction (ISNb: >44%, SNa: 38%) were strikingly distinct (Table 1). For example, though it was observed that ISNb and SNa axon guidance phenotypes consistent with MICAL LOF phenotypes (Table 1), these phenotypes were often more severe. They include defects in which axons bypass their muscle targets but then appear to defasciculate in inappropriate places and project into adjacent segments. Interestingly, also observed were ISNb and SNa axon guidance phenotypes consistent with MICAL GOF, but these phenotypes also appeared more severe and included, severely defasciculated and tangled axons. Finally, phenotypes were observed that were unlike MICAL or PlexA LOF or GOF mutants, including axons projecting along the entire length of the muscle 6/7 cleft and dramatic axonal wandering within muscle fields. These phenotypes suggest that expression of MICAL^{G→W} leads to defects not explained by a simple elevation or diminution of PlexA signaling activity.

In summary, these results show the necessity of an intact flavoprotein monooxygenase domain for MICAL function in repulsive motor axon guidance.

### EXAMPLE 9

### Flavoprotein Monooxygenase Inhibitors Neutralize Vertebrate Sema3a Axonal Repulsion

This example identifies the gallic acid derivatives EGCG and EC as inhibitors of semaphorin-mediated axon repulsion.

Vertebrate In vitro Repulsion and Collapse Assays

DRG repulsion assays were performed as described (Messersmith et al., 1995). (EGCG), (EC), L-NAME, allopurinol, (Sigma) were dissolved in vehicle (PBS), protected from light, and then added to the culture media to final concentrations. Rotenone (Sigma) was dissolved in 95% EtOH and then added to the culture media (final EtOH concentration was below 0.1% and had no effect on axon outgrowth).

Inhibitors specific for nitric oxide synthase (N-nitro-L-arginine methylester (L-NAME); Comoletti et al., 2001), xanthine oxidase (allopurinol (Allo); Jonakait et al., 2000), or mitochondrial electron transport (NADH dehydrogenase; rotenone (Rote); Frantseva et al., 2001) were used at concentrations previously shown to be effective in cell culture conditions (see FIG. 7). The effect of EGCG on growth cone collapse was performed by culturing DRGs using standards techniques (Fan et al, 1993). After 48 hours in culture, DRGs were incubated in media containing EGCG or vehicle (PBS) for 3 hours prior to a 1 hour application of 1 nM AP-Sema3A or non-AP-Sema3A containing growth media. Scoring was done by splitting explants into quadrants and scoring all growth cones as either collapsed or not-collapsed.

Results

The MICALs may be susceptible to small molecule inhibitors that affect their ability to oxidize their substrate. Some gallic acid derivatives, including the green tea component (−)-epigallocatechin gallate (EGCG), are potent and selective inhibitors of two flavoprotein monooxygenases: squalene epoxidase (SE) and p-hydroxybenzoate hydroxylase (pHBH) (Abe et al., 2000a; Abe et al., 2000b).

All available evidence points to the plexin cytoplasmic domain as an essential signal transducing domain for signaling class 3 semaphorin repulsion (Cheng et al., 2001; Takahashi and Strittmatter, 2001). Sema3A appears to utilize neuropilin-1 in combination with A class plexins to signal repulsive guidance. To ask whether selective flavoprotein monooxygenase inhibitors can neutralize semaphorin-mediated repulsion in vertebrates, in vitro rat DRG growth cone repulsion assays were employed using Sema 3A-secreting 293 cells (FIG. 7A; Messersmith et al., 1995). NGF-dependent DRG axons exhibit little to no outgrowth towards Sema3A-secreting 293 cell aggregates (FIG. 4C). However, when 25 mM EGCG is added to the growth media Sema3A repulsion was completely neutralized (FIG. 4C). EGCG attenuation of Sema3A-mediated repulsion is dose-dependent (FIG. 4C). We also asked whether (−)-epicatechin (EC), a compound structurally similar to EGCG but a poor inhibitor of SE (Abe et al., 2000b), had a similar effect on Sema-3A-mediated repulsion. Like EGCG, EC was capable of completely neutralizing Sema-3A-dependent repulsion in a dose-dependent manner, but a much higher EC concentration was required (FIG. 4C). To address the possibility that a general inhibition of oxidation-reduction mechanisms by these reagents underlies this attenuation of Sema3A repulsion, selective inhibitors of other redox enzymes present in neurons were analyzed for an effect on Sema3A-mediated repulsion. No attenuation of Sema 3A mediated axonal repulsion was observed using inhibitors specific for nitric oxide synthase (N-nitro-L-arginine methylester (L-NAME)), xanthine oxidase (allopurinol (Allo), or mitochondrial electron transport (NADH dehydrogenase; rotenone (Rote), at concentrations previously shown to be effective in cell culture conditions (FIG. 4C). DRG axons and Sema3A-secreting 293 cells appeared normal following growth in the presence of all but one of these inhibitors. In some explants we noticed an adverse effect on survival of DRGs treated with rotenone, but axons in those rotenone-treated explants that survived, although somewhat thinner than normal, were robustly repelled (FIG. 4C). The amount and biological activity of Sema3A produced by 293 cells in the presence of all inhibitors was similar as assessed using a DRG growth cone collapse assay (FIG. 4B), showing that none of these inhibitors had an adverse effect on the ability of 293 cells to produce active Sema 3A. It was also determined in separate experiments that 25 mM EGCG dramatically abrogates Sema3A-mediated growth cone collapse in NGF-dependent DRG neurons. Taken together, our results support a role for flavoenzymes and oxidation-reduction mechanisms in semaphorin-mediated axon guidance.

### EXAMPLE 10

### Further Considerations Regarding the Association of MICALS and Semaphorin-Mediated Axonal Guidance

This example provides further insight into the association of MICALs and semaphorin-mediated axonal guidance.

Neuronal growth cone guidance depends on the ability of various guidance cue receptors to regulate cytoskeletal dynamics in response to the local presentation of ligands. It was shown herein that proteins belonging to the MICAL family of cytosolic, multi-domain, flavoprotein monooxygenases are required for certain plexin-mediated semaphorin axon guidance events. MICALs associate with plexins and contain several conserved domains that provide the potential for interactions with both growth cone cytoskeletal components and many signaling proteins intimately involved in their regulation. Our results suggest that MICALs directly participate in plexin signaling through the action of their flavoprotein monooxygenase domain. These observations provide a framework for dissecting the molecular basis of semaphorin-meditated neuronal guidance and also a potential target for attenuating their repulsive action.

Genetic and biochemical results provided herein support an essential role for Drosophila MICAL in mediating PlexA/Sema-l a repulsive guidance events required for motor axon pathfinding. Future experiments will establish whether MICAL mediates PlexB signaling, and if so, whether this occurs directly or indirectly. Drosophila MICAL is an orthologue of a mammalian MICAL-1 protein. It is shown herein that that there are at least three vertebrate MICAL orthologues (MICALs 1, 2, and 3). We also identify here a family of MICAL-like proteins that lack the conserved N-terminal MICAL monooxygenase domain. Expression and interaction data herein support the idea that MICALs mediate plexin signaling in vertebrates. In addition, flavoprotein monooxygenase inhibitors block Sema3A-mediated repulsion and collapse of NGF-dependent DRG axons-repulsive interactions dependent on A class plexins including Plexin A3. Future genetic and biochemical analysis will establish the role of vertebrate MICALs in neuronal and non-neuronal plexin signaling.

The highly conserved ˜500 amino acid N-terminal MICAL domain contains signature amino acid sequences of the flavoprotein monooxygenase family of oxidoreductases. Biochemical and genetic analyses herein strongly suggest that MICALs contain functional FAD binding monooxygenase domains required for mediating plexin signaling. In support of this idea, it was observed that inhibition of flavoprotein monooxygenase enzymatic activity dramatically attenuates semaphorin-mediated axon repulsion and growth cone collapse. However, though the inhibitors we used, ECGC and EC, have a high degree of selectivity for flavoprotein monooxygenases, similar concentrations of EGCG inhibit other enzymes including steroid 5a-reductase, NADPH-cytochrome P450 reductase, telomerase, matrix metalloproteinases MMP-2 and MMP-9, and phenol sulfotransferase (Abe et al., 2000a; Abe et al., 2000b). Although most of these enzymes are unlikely to be expressed in the growth cones of DRG axons, potential non-specific effects of these inhibitors cannot be ruled out despite their demonstrated selectivity for monooxygenases. Taken together with our in vivo Drosophila experiments showing a requirement for the MICAL FAD binding region in Sema-1a mediated axon repulsion, these data suggest redox signaling plays an important role in vertebrate semaphorin-mediated axonal repulsion.

Flavoprotein monooxygenases specifically catalyze the oxidation of a number of substrates, and in some contexts they can function as oxidases and generate reactive oxygen species (Massey, 1994). Results herein suggest that MICALs are flavoproteins most similar to the flavoprotein monooxygenase family of oxidoreductases, but a complete understanding of the chemical nature of the reactions catalyzed by MICALs awaits future study and identification of substrates. The redox regulation of amino acid residues within signaling proteins (including kinases, phosphatases, small GTPases, guanylate cyclases, and adapter proteins) and cytoskeletal proteins (including actin, actin binding proteins, intermediate filament proteins, and GAP-43) has been shown to modulate their function (Finkel, 1998; Kim et al., 2002; Meng et al., 2002; Rhee et al., 2000; Stamler et al., 2001; Thannickal and Fanburg, 2000). In addition, oxidation of actin leads to disassembly of actin filaments, instability and collapse of actin networks, reduced ability of actin to interact with actin cross-linking proteins, and a decrease in the ability of actin monomers to form polymers (Dalle-Donne et al., 2001a; Dalle-Donne et al., 2001b; Milzani et al., 1997). Finally, it is also interesting that MICALs have a putative actin filament binding domain (CH domain) and that MICAL-1 interacts with vimentin, an intermediate filament protein (Suzuki et al., 2002).

It was recently reported that the proline rich region of vertebrate MICAL-1 interacts with the SH3 domain of the adaptor protein CasL (HEF 1) in non-neuronal cells (Suzuki et al., 2002). CasL, along with the related proteins p130Cas, and Efs (Sin), make up the Cas family of proteins (O'Neill et al., 2000), which assemble and transduce intracellular signals that stimulate cell migration and axon outgrowth. These proteins have numerous protein-protein interaction domains, including a Src-homology 3 (SH3) domain, multiple SH2-binding sites in their substrate domain, several proline-rich motifs, and a C-terminal dimerization module. This structure suggests a role for Cas family proteins as docking molecules, and numerous interacting proteins have been identified, including kinases (e.g. FAK, Src, and Abl), phosphatases (e.g. PTP-1B, and SHP2), GEFs (e.g. C3G), and adaptor proteins (e.g., Nck, Crk, Grb2, and 14-3-3) (O'Neill et al., 2000). Studies indicate that Cas proteins localize mainly to focal adhesions and stress fibers, and that they are required in integrin-dependent cell migration and actin filament assembly. Cas proteins, therefore, may play an important role in plexin-mediated repulsive and attractive guidance events.

In conclusion, characterized herein is a gene family conserved from invertebrates to vertebrates, with proteins whose structure and function strongly suggest that redox signaling is important for semaphorin-mediated axonal repulsion. The results herein also suggest that protein oxidation could be a general means for inhibiting axonal growth. Given the presence of high amounts of reactive oxygen species and other oxidants in the spinal cord after injury (Juurlink and Paterson, 1998) regulation of redox signaling using antioxidants and specific enzyme inhibitors may be a powerful approach for encouraging axonal regeneration.

TABLE 2
List of sequences

| SEQ ID NO: | Sequence |
|---|---|
| 1 | Human MICAL 1 cDNA |
| 2 | Human MICAL 1 polypeptide |
| 3 | Human MICAL 2 cDNA |
| 4 | Human MICAL 2 polypeptide |
| 5 | Human MICAL 3 cDNA |
| 6 | Human MICAL 3 polypeptide |
| 7 | Drosophila MICAL long variant cDNA |
| 8 | Drosophila polypeptide (long variant) |
| 9 | Drosophila MICAL medium variant cDNA |
| 10 | Drosophila polypeptide (medium variant) |
| 11 | Drosophila short variant cDNA |
| 12 | Drosophila polypeptide (short variant) |
| 13 | Human MICAL-Like 1 cDNA |
| 14 | Human MICAL-Like 1 polypeptide |
| 15 | Human MICAL-Like 2 cDNA |
| 16 | Human MICAL-Like 2 polypeptide |
| 17 | Drosophila MICAL-Like cDNA |
| 18 | Drosophila MICAL-Like polypeptide |
| 19 | Drosophila truncated mutant polypeptide |
| 20 | Drosophila G to W mutant polypeptide |
| 21 | Mouse MICAL 1 polypeptide |
| 22 | Mouse MICAL 2 polypeptide |
| 23 | Mouse MICAL 3 polypeptide |
| 24 | Anopheles gambiae MICAL polypeptide fragment |
| 25 | Ciona inetstinalis MICAL polypeptide fragment |
| 26 | Danio rerio MICAL 1 polypeptide fragment |
| 27 | Danio rerio MICAL 2 polypeptide fragment |
| 28 | Gallus gallus MICAL 1 polypeptide fragment |
| 29 | Gallus gallus MICAL 2 polypeptide fragment |
| 30 | Rattus norvegicus MICAL 1 polypeptide fragment |
| 31 | Rattus norvegicus MICAL 2 polypeptide fragment |
| 32 | Rattus norvegicus MICAL 3 polypeptide fragment |
| 33 | Bos taurus MICAL 1 polypeptide fragment |
| 34 | Bos taurus MICAL 2 polypeptide fragment |
| 35 | Sus scrofa MICAL polypeptide fragment |
| 36 | Pan troglodytes MICAL polypeptide fragment |
| 37 | Amplification primer for MICAL |
| 38 | Amplification primer for mutant MICAL |
| 39 | FAD binding domain |
| 40 | mutated FAD binding domain |

### REFERENCES

Abe, I., Kashiwagi, K., and Noguchi, H. (2000a). Antioxidative galloyl esters as enzyme inhibitors of p-hydroxybenzoate hydroxylase, FEBS Lett. 483, 131-4.

Abe, I., Seki, T., Umehara, K., Miyase, T., Noguchi, H., Sakakibara, J., and Ono, T. (2000b). Green tea polyphenols: novel and potent inhibitors of squalene epoxidase. Biochem. Biophys. Res. Commun. 268, 767-71.

Bach, I. (2000). The LIM domain: regulation by association. Mech. Dev. 91, 5-17.

Brand, A. H., and Perrimon, N. (1993). Targeted gene expression as a means of altering cell fates and generating dominant phenotypes. Development. 118, 401-415.

Bretscher, A., Chambers, D., Nguyen, R., and Reczek, D. (2000). ERM-Merlin and EBP50 protein families in plasma membrane organization and function. Annu. Rev. Cell Dev. Biol. 16, 113-43.

Burkhard, P., Stetefeld, J., and Strelkov, S. V. (2001). Coiled coils: a highly versatile protein folding motif. Trends Cell Biol. 11, 82-8.

Cheng, H. J., Bagri, A., Yaron, A., Stein, E., Pleasure, S. J., and Tessier-Lavigne, M. (2001). Plexin-A3 mediates semaphorin signaling and regulates the development of hippocampal axonal projections. Neuron. 32, 249-63.

Comoletti, D., Muzio, V., Capobianco, A., Ravizza, T., and Mennini, T. (2001). Nitric oxide produced by non-motoneuron cells enhances rat embryonic motoneuron sensitivity to excitotoxins: comparison in mixed neuron/glia or purified cultures. J Neurol Sci. 192, 61-9.

Cook, K., Coulson, D., Roote, J., Morley, T., Bogart, K., Deal, J., Kaufman, T., and Gubb, D. (2001). Chromosomal deletions and inversions with well-defined endpoints can be generated at high frequencies by transposase-induced recombination between pairs of P-element insertions. Drosophila. Research Conference 42, 19W.

Cooley, L., Thompson, D., and Spradling, A. C. (1990). Constructing deletions with defined endpoints in Drosophila. Proc Natl Acad Sci U S A 87, 3170-3.

Dalle-Donne, I., Rossi, R., Giustarini, D., Gagliano, N., Lusini, L., Milzani, A., Di Simplicio, P., and Colombo, R. (2001a). Actin carbonylation: from a simple marker of protein oxidation to relevant signs of severe functional impairment. Free Radic Biol Med. 31, 1075-83.

Dalle-Donne, I., Rossi, R., Milzani, A., Di Simplicio, P., and Colombo, R. (200 lb). The actin cytoskeleton response to oxidants: from small heat shock protein phosphorylation to changes in the redox state of actin itself. Free Radic Biol Med. 31, 1624-32.

Dym, O., and Eisenberg, D. (2001). Sequence-structure analysis of FAD-containing proteins. Protein Sci. 10, 1712-28.

Eggink, G., Engel, H., Vriend, G., Terpstra, P., and Witholt, B. (1990). Rubredoxin reductase of Pseudomonas oleovorans. Structural relationship to other flavoprotein oxidoreductases based on one NAD and two FAD fingerprints. J Mol Biol. 212, 135-42.

Golemis, E. A., Gyuris, J., and Brent, R. (1994). Interaction trap/two hybrid system to identify interacting proteins. In Current Protocols in Molecular Biology. (New York:Wiley) 27, pp. 13.14.1-13.14.17.

Eppink, M. H., Schreuder, H. A., and Van Berkel, W. J. (1997). Identification of a novel conserved sequence motif in flavoprotein hydroxylases with a putative dual function in FAD/NAD(P)H binding. Protein Sci. 6, 2454-8.

Fan, J., Mansfield, S. G., Redman, T., Phillip, R., Gordon-Weeks, P. R., and Raper, J. A. (1993). The organization of F-actin and microtubules in growth cones exposed to a brain-derived collapsing factor. J Cell Biol. 121, 867-878.

Finkel, T. (1998). Oxygen radicals and signaling. Curr Opin Cell Bio.l 10, 248-53.

Frantseva, M. V., Carlen, P. L., and Perez Velazquez, J. L. (2001). Dynamics of intracellular calcium and free radical production during ischemia in pyramidal neurons. Free Radic Biol Med. 31, 1216-27.

Gimona, M., Djinovic-Carugo, K., Kranewitter, W. J., and Winder, S. J. (2002). Functional plasticity of CH domains. FEBS Lett. 513, 98-106.

Harris, B. Z., and Lim, W. A. (2001). Mechanism and role of PDZ domains in signaling complex assembly. J. Cell Sci. 114, 3219-31.

He, Z., Wang, K. C., Koprivica, V., Ming, G., and Song, H. J. (2002). Knowing how to navigate: mechanisms of semaphorin signaling in the nervous system. Sci STKE. 2002, RE1.

Hosokawa, K., and Stanier, R. Y. (1966). Crystallization and properties of p-hydroxybenzoate hydroxylase from Pseudomonas putida. J Biol Chem. 241, 2453-60.

Hu, H., Marton, T. F., and Goodman, C. S. (2001). Plexin B mediates axon guidance in Drosophila by simultaneously inhibiting active Rac and enhancing RhoA signaling. Neuron. 32, 39-51.

Jonakait, G. M., Wen, Y., Wan, Y., and Ni, L. (2000). Macrophage cell-conditioned medium promotes cholinergic differentiation of undifferentiated progenitors and synergizes with nerve growth factor action in the developing basal forebrain. Exp Neurol. 161, 285-96.

Juurlink, B. H., and Paterson, P. G. (1998). Review of oxidative stress in brain and spinal cord injury: suggestions for pharmacological and nutritional management strategies. J Spinal Cord Med. 21, 309-34.

Kim, S. O., Merchant, K., Nudelman, R., Beyer, J., W. F., Keng, T., DeAngelo, J., Hausladen, A., and Stamler, J. S. (2002). OxyR: A molecular code for redox-related signaling. Cell. 109, 383-396.

Kolodkin, A. L., Matthes, D., and Goodman, C. S. (1993). The semaphorin genes encode a family of transmembrane and secreted growth cone guidance molecules. Cell. 75, 1389-1399.

Kubo, A., Itoh, S., Itoh, K., and Kamataki, T. (1997). Determination of FAD-binding domain in flavin-containing monooxygenase 1 (FMO1). Arch Biochem Biophys. 345, 271-7.

Landgraf, M., Bossing, T., Technau, G. M., and Bate, M. (1997). The origin, location, and projections of the embryonic abdominal motorneurons of Drosophila. J Neurosci. 17, 9642-55.

Lawton, M. P., and Philpot, R. M. (1993). Functional characterization of flavin-containing monooxygenase 1B1 expressed in Saccharomyces cerevisiae and Escherichia coli and analysis of proposed FAD- and membrane-binding domains. J Biol Chem. 268, 5728-34.

Lindsay, H., Beaumont, E., Richards, S. D., Kelly, S. M., Sanderson, S. J., Price, N.C., and Lindsay, J. G. (2000). FAD insertion is essential for attaining the assembly competence of the dihydrolipoamide dehydrogenase (E3) monomer from Escherichia coli. J Biol Chem. 275, 36665-70.

Liu, B. P., and Strittmatter, S. M. (2001). Semaphorin-mediated axonal guidance via Rho-related G proteins. Curr Opin Cell Biol. 13, 619-26.

Macheroux, P. (1999). UV-visible spectroscopy as a tool to study flavoproteins. Methods Mol Biol. 131, 1-7.

Massey, V. (1994). Activation of molecular oxygen by flavins and flavoproteins. J Biol Chem. 269, 22459-62.

Massey, V. (1995). Introduction: flavoprotein structure and mechanism. Faseb. J 9, 473-5.

Matthes, D. J., Sink, H., Kolodkin, A. L., and Goodman, C. S. (1995). Semaphorin II can function as a selective inhibitor of specific synaptic arborizations in Drosophila. Cell. 81, 631-639.

Meng, T. C., Fukada, T., and Tonks, N. K. (2002). Reversible oxidation and inactivation of protein tyrosine phosphatases in vivo. Mol Cell. 9, 387-99.

Messersmith, E. K., Leonardo, E. D., Shatz, C. J., Tessier-Lavigne, M., Goodman, C. S., and Kolodkin, A. L. (1995). Semaphorin III can function as a selective chemorepellent to pattern sensory projections in the spinal cord. Neuron. 14, 949-959.

Milzani, A., DalleDonne, I., and Colombo, R. (1997). Prolonged oxidative stress on actin. Arch Biochem Biophys. 339, 267-74.

O'Neill, G. M., Fashena, S. J., and Golemis, E. A. (2000). Integrin signalling: a new Cas(t) of characters enters the stage. Trends Cell Biol. 10, 111-9.

Pasterkamp, R. J., De Winter, F., Holtmaat, A. J. G. D., and Verhaagen, J. (1998). Evidence for a role of the chemorepellent semaphorin III and its receptor neuropilin-1 in the regeneration of primary olfactory axons. J Neurosci. 18, 9962-9976.

Preston, C. R., Sved, J. A., and Engels, W. R. (1996). Flanking duplications and deletions associated with P-induced male recombination in Drosophila. Genetics 144, 1623-38.

Raper, J. A. (2000). Semaphorins and their receptors in vertebrates and invertebrates. Curr Opin Neurobiol. 10, 88-94.

Rhee, S. G., Bae, Y. S., Lee, S. R., and Kwon, J. (2000). Hydrogen peroxide: a key messenger that modulates protein phosphorylation through cysteine oxidation. Sci STKE. 2000, E1.

Schulz, G. E. (1992). Binding of nucleotides by proteins. Curr Opin Struct Biol. 2, 61-67.

Sambrook, J., Fritsch, E. F., and Maniatis, T. (1989). Molecular Cloning: A Laboratory Manual. Second Edition (Cold Spring Harbor, N.Y., Cold Spring Harbor Laboratory).

Simon, M. A., Drees, B., Kornberg, T., and J. M. Bishop. (1985) The Nucleotide Sequence and the Tissue-Specific Expression of Drosophila c-src. Cell 42, 831-840.Semaphorin Nomenclature Committee (1999). Unified nomenclature for the semaphorins/collapsins. Semaphorin Nomenclature Committee, Cell. 97, 551-2.

Stamler, J. S., Lamas, S., and Fang, F. C. (2001). Nitrosylation the prototypic redox-based signaling mechanism. Cell. 106, 675-83.

Suzuki, T., Nakamoto, T., Ogawa, S., Seo, S., Matsumura, T., Tachibana, K., Morimoto, C., and Hirai, H. (2002). MICAL, a novel CasL interacting molecule, associates with vimentin. J Biol Chem. 277, 14933-41.

Takahashi, T., and Strittmatter, S. M. (2001). PlexinA1 Autoinhibition by the Plexin Sema Domain. Neuron. 29, 429-439.

Tamagnone, L., and Comoglio, P. M. (2000). Signalling by semaphorin receptors: cell guidance and beyond. Trends Cell Biol. 10, 377-83.

Thannickal, V. J., and Fanburg, B. L. (2000). Reactive oxygen species in cell signaling. Am J Physiol Lung. Cell Mol Physiol. 279, L1005-28.

Vallon, O. (2000). New sequence motifs in flavoproteins: evidence for common ancestry and tools to predict structure. Proteins. 38, 95-114.

VanVactor, D., Sink, H., Fambrough, D., Tsoo, R., and Goodman, C. S. (1993). Genes that control neuromuscular specificity in Drosophila. Cell. 73, 1137-1153.

White, D. J., Merod, R., Thomasson, B., and Hartzell, P. L. (2001). GidA is an FAD-binding protein involved in development of Myxococcus xanthus. Mol Microbiol. 42, 503-17.

Whitford, K. L., and Ghosh, A. (2001). Plexin signaling via off-track and rho family GTPases. Neuron. 32, 1-3.

Wierenga, R. K., Terpstra, P., and Hol, W. G. (1986). Prediction of the occurrence of the ADP-binding beta alpha beta-fold in proteins, using an amino acid sequence fingerprint. J Mol Biol 187, 101-7.

Winberg, M. L., Mitchell, K. J., and Goodman, C. S. (1998a). Genetic analysis of the mechanisms controlling target selection: complementary and combinatorial functions of netrins, semaphorins, and IgCAMs. Cell. 93, 581-591.

Winberg, M. L., Noordermeer, J. N., Tamagnone, L., Comoglio, P. M., Spriggs, M. K., Tessier-Lavigne, M., and Goodman, C. S. (1998b). Plexin A is a neuronal semaphorin receptor that controls axon guidance. Cell. 95, 903-916.

Xu, X. M., Fisher, D. A., Zhou, L., White, F. A., Ng, S., Snider, W. D., and Luo, Y. (2000). The transmembrane protein semaphorin 6A repels embryonic sympathetic axons. J. Neurosci. 20, 2638-48.

Yu, H. H., Araj, H. H., Ralls, S. A., and Kolodkin, A. L. (1998). The transmembrane Semaphorin Sema1 is required in Drosophila for embryonic motor and CNS axon guidance. Neuron 20, 207-20.

Although the invention has been described with reference to the above example, it will be understood that modifications and variations are encompassed within the spirit and scope of the invention. Accordingly, the invention is limited only by the following claims.

What is claimed is:

1. An isolated polypeptide comprising an N-terminal MICAL domain, a calponin homology domain, a LIM domain, a proline rich region, and a plexin interacting region, wherein the polypeptide has monooxygenase activity.

2. An isolated polypeptide of claim 1, wherein the polypeptide is a mammalian MICAL polypeptide.

3. An isolated polypeptide of claim 2, wherein the isolated polypeptide is human MICAL-1, human MICAL-2, or human MICAL-3.

4. An isolated polypeptide of claim 3, wherein the polypeptide comprises an amino acid sequence as set forth in SEQ ID NO:2, SEQ ID NO:4, or SEQ ID NO:6.

5. An isolated polypeptide of claim 1, wherein the polypeptide comprises an N-terminal MICAL domain having at least about 50% sequence identity to the N-terminal amino acids 1-500 of SEQ ID NO:2, SEQ ID NO:4, or SEQ ID NO:6.

6. An isolated polypeptide of claim 1, wherein the polypeptide is a Drosophila MICAL polypeptide.

7. An isolated polypeptide of claim 6, wherein the polypeptide is set forth in SEQ ID NO:8.

8. An isolated polypeptide of claim 1, wherein the polypeptide is a MICAL isoform.

9. An isolated polypeptide of claim 1, wherein the isolated polypeptide is at least 90% identical to SEQ ID NO:2, SEQ ID NO:4, SEQ ID NO:6, SEQ ID NO:8, SEQ ID NO:10, or SEQ ID NO: 12.

10. An isolated polypeptide of claim 1, wherein the polypeptide comprises from N-terminal to C-terminal, an N-terminal MICAL domain, a calponin homology domain, a first variable MICAL region, a LIM domain, a proline rich region, and a plexin interacting region.

11. An isolated polypeptide comprising a plexin interacting region at least 90% identical to a plexin interacting region of SEQ ID NO:2, SEQ ID NO:4, SEQ ID NO:6, SEQ ID NO:8, SEQ ID NO:10, or SEQ ID NO:12, wherein the polypeptide has plexin interacting activity.

12. An isolated polypeptide of claim 11, wherein the polypeptide comprises a plexin interacting region of SEQ ID NO:2, SEQ ID NO:4, SEQ ID NO:6, SEQ ID NO:8, SEQ ID NO:10, or SEQ ID NO:12, or a conservative variant thereof.

13. An isolated polypeptide of claim 11, with the proviso that the polypeptide does not have monooxygenase activity.

14. An isolated polypeptide of claim 11, wherein the polypeptide comprises the plexin interacting region of Drosophila MICAL-like polypeptide, or the plexin interacting region of human MICAL-like polypeptide 1, or 2, or a conservative variant thereof.

15. An isolated polypeptide of claim 14, wherein the polypeptide has the amino acid sequence of SEQ ID NO:14, SEQ ID NO:16, or SEQ ID NO:18.

16. An isolated polypeptide comprising an N-terminal MICAL domain of Drosophila MICAL 1, or the N-terminal MICAL domain of human MICAL 1, 2, or 3, or a conservative variant thereof.

17. An isolated polypeptide of claim 16, wherein the polypeptide has monooxygenase activity.

18. An isolated polypeptide comprising a calponin homology domain at least 90% identical to the calponin homology domain of SEQ ID NO:2, SEQ ID NO:4, SEQ ID NO:6, SEQ ID NO:8, SEQ ID NO:10, or SEQ ID NO:12, and wherein the polypeptide is involved in actin filament binding.

19. An isolated polypeptide comprising a LIM domain at least 90% identical to the LIM domain of SEQ ID NO:2, SEQ ID NO:4, SEQ ID NO:6, SEQ ID NO:8, SEQ ID NO:10, or SEQ ID NO:12, and wherein the polypeptide specifically interacts with a LIM-binding protein.

20. An isolated polypeptide comprising a proline rich region at least 90% identical to the proline rich region of SEQ ID NO:2, SEQ ID NO:4, SEQ ID NO:6, SEQ ID NO:8, SEQ ID NO:10, or SEQ ID NO:12, and wherein the polypeptide interacts with a polypeptide comprising an SH3-domain

21. An isolated polynucleotide comprising a nucleotide sequence encoding a polypeptide of claim 1.

22. An isolated polynucleotide of claim 21, wherein the polynucleotide encodes a mammalian MICAL polypeptide.

23. An isolated polynucleotide of claim 22, wherein the polynucleotide encodes a polypeptide that is at least 90% identical to SEQ ID NO:2, SEQ ID NO:4, or SEQ ID NO:6.

24. An isolated polynucleotide of claim 23, wherein the polynucleotide encodes a polypeptide as set forth in SEQ ID NO:2, SEQ ID NO:4, or SEQ ID NO:6.

25. An isolated polynucleotide of claim 21, wherein the polynucleotide encodes a MICAL polypeptide comprising an N-terminal MICAL domain having monooxygenase activity, and at least 50% sequence identity to the N-terminal 500 amino acids of human MICAL 1 polypeptide.

26. An isolated polynucleotide of claim 21, wherein the polynucleotide encodes a Drosophila MICAL polypeptide.

27. An isolated polynucleotide of claim 26, wherein the polynucleotide encodes an amino acid sequence as set forth in SEQ ID NO:8, SEQ ID NO:10, or SEQ ID NO:12.

28. A vector comprising a polynucleotide of claim 15.

29. A vector of claim 28, wherein the vector is a recombinant expression vector.

30. A vector of claim 28, wherein the vector is a viral vector.

31. A host cell comprising a polynucleotide encoding the polypeptide of claim 1 operably linked to a heterologous promoter.

32. A host cell comprising a vector of claim 28.

33. The host cell of claim 32, wherein the host cell is a stem cell.

34. The host cell of claim 32, wherein the host cell is a neuronal lineage cell.

35. An antibody or antigen binding fragment thereof that specifically binds the polypeptide of claim 1.

36. The antibody or antigen binding fragment thereof of claim 35 which is human or humanized.

37. The antibody or antigen binding fragment thereof of claim 35 which is an intrabody.

38. The antibody or antigen binding fragment thereof of claim 35 which specifically binds the N-terminal MICAL domain of said polypeptide.

39. The antibody or antigen binding fragment thereof of claim 35 which specifically binds plexin interacting region of said polypeptide.

40. A method for identifying an agent that affects axonal guidance regulatory activity, comprising contacting an isolated polypeptide of claim 1, or a cell recombinantly expressing a polypeptide of claim 1, with a candidate agent, and comparing the axonal guidance regulatory activity in the presence and absence of the agent, wherein a difference in activity is indicative of an agent that affects axonal guidance regulatory activity.

41. The method of claim 40, wherein the agent inhibits axonal guidance regulatory activity.

42. The method of claim 40, wherein the agent is a small molecule, an antisense polynucleotide, a MICAL-like polypeptide or fragment thereof, a mutant MICAL polypeptide, an anti-MICAL antibody, a double stranded RNA, or a peptidomimetic.

43. The method of claim 40, wherein the agent is a monooxygenase inhibitor.

44. The method of claim 43, wherein the anti-oxidant is a flavonoid.

45. The method of claim 44, wherein the flavonoid is a gallic acid derivative.

46. A method for screening for an agent that modulates an activity of a MICAL polypeptide, said method comprising (a) contacting the isolated polypeptide of claim 1 with a candidate agent and (b) comparing said activity of the polypeptide of claim 1 in the presence or absence of said candidate agent, wherein a difference in said activity indicates that the agent modulates the activity of the MICAL polypeptide.

47. The method of claim 46, wherein said activity is monooxygenase activity.

48. The method of claim 46, wherein said activity is plexin A-binding activity.

49. The method of claim 46, wherein the method is a cell-free assay.

50. A method for screening for an agent that modulates an activity of a MICAL polypeptide, said method comprising (a) contacting a cell expressing the polypeptide of claim 1 with a candidate agent and (b) comparing said activity of the polypeptide of claim 1 in the presence or absence of said candidate agent, wherein a difference in said activity indicates that the agent modulates the activity of the MICAL polypeptide.

51. The method of claim 50, wherein the activity is monooxygenase activity.

52. The method of claim 50, wherein the activity is plexin A-binding activity.

53. The method of claim 50, wherein the cell is a neuron.

54. The method of claim 50, wherein the cell is an immune cell.

55. The method of claim 50, wherein the cell has a transformed phenotype.

56. The method of claim 50, wherein the cell is a cardiac cell.

57. A method for screening for an agent that modulates an activity of a MICAL polypeptide, said method comprising (a) contacting a cell that recombinantly expresses the polypeptide of claim 1 with a candidate agent and (b) comparing a phenotypic or physiological trait of said cell in the presence or absence of said candidate agent, wherein a difference in said phenotypic or physiological trait indicates that the agent modulates the activity of the MICAL polypeptide.

58. The method of claim 57, wherein the phenotypic or physiological trait involves dynamics of the cytoskeleton.

59. The method of claim 57, wherein the phenotypic or physiological trait is axon guidance.

60. The method of claim 57, wherein the phenotypic or physiological trait is cell proliferation or invasiveness.

61. The method of claim 57, wherein the phenotypic or physiological trait is an immune response.

62. A method for screening for an agent that modulates the expression of a MICAL polypeptide, the method comprising (a) contacting a cell with a candidate agent; and (b) comparing the expression of the polypeptide of claim 1 in the presence or absence of the candidate agent, wherein a difference in the expression indicates that the agent modulates the expression of the MICAL polypeptide.

63. The method of claim 62, wherein the level of mRNA encoding MICAL is compared.

64. The method of claim 62, wherein the level of the MICAL polypeptide is compared.

65. A polynucleotide that specifically hybridizes to a polynucleotide of claim 15, wherein the polynucleotide is at least 15 nucleotides in length.

66. A polynucleotide of claim 65, wherein the polynucleotide inhibits expression of a polynucleotide that encodes a polypeptide of claim 1.

67. A polynucleotide of claim 65, wherein the polynucleotide is at least 90% identical to a complementary polynucleotide of a polynucleotide encoding a polypeptide of SEQ ID NO:2, SEQ ID NO:4, SEQ ID NO:6, SEQ ID NO:8, SEQ ID NO:10, or SEQ ID NO:12.

68. A polynucleotide of claim 65, wherein the polynucleotide specifically hybridizes to a polynucleotide encoding a polypeptide of SEQ ID NO:2, SEQ ID NO:4, SEQ ID NO:6, SEQ ID NO:8, SEQ ID NO:10, or SEQ ID NO:12.

69. A double-stranded RNA molecule comprising a first RNA strand that specifically hybridizes to an mRNA encoding a MICAL polypeptide and a second RNA strand that is the reverse complement of said first strand, wherein said double-stranded RNA molecule is at least 15 base pairs in length.

70. An isolated polypeptide or a functional peptide portion thereof, comprising a calponin homology domain, a LIM domain, a proline rich region, and a plexin interacting region, and having plexin-interacting activity.

71. An isolated polypeptide of claim 70, wherein the polypeptide comprises a calponin homology domain, followed by a first variable region, followed by a LIM domain, followed by a proline rich region, and followed by a plexin interacting region.

72. An isolated polypeptide of claim 70, wherein the polypeptide is at least 90% identical to an amino acid sequence as set forth in SEQ ID NO: 14, SEQ ID NO: 16, or SEQ ID NO:18.

73. An isolated polypeptide of claim 72, wherein the polypeptide has an amino acid sequence as set forth in SEQ ID NO: 14, SEQ ID NO: 16, or SEQ ID NO: 18.

74. An isolated polypeptide of claim 70, wherein the polypeptide is a mammalian polypeptide.

75. An isolated polypeptide of claim 70, wherein the polypeptide is a human polypeptide.

76. An isolated polypeptide of claim 70, wherein the polypeptide is a Drosophila polypeptide.

77. An isolated polynucleotide encoding a polypeptide according to claim 70, or a functional peptide portion thereof.

78. An isolated polynucleotide of claim 77, wherein the polynucleotide encodes a mammalian MICAL-like polypeptide.

79. An isolated polynucleotide of claim 77, wherein the polynucleotide encodes a polypeptide that is at least 90% identical to an amino acid sequence as set forth in SEQ ID NO:14, SEQ ID NO:16, or SEQ ID NO:18.

80. An isolated polynucleotide of claim 77, wherein the polynucleotide encodes a polypeptide comprising a calponin homology domain, followed by a first non-conserved region, followed by a LIM domain, followed by a second non-conserved region, followed by a proline rich region, and followed by a plexin interacting region.

81. An isolated polynucleotide of claim 79, wherein the polynucleotide encodes a polypeptide having an amino acid sequence of SEQ ID NO:14, SEQ ID NO:16, or SEQ ID NO:18.

82. A vector comprising a polynucleotide of claim 77.

83. A vector of claim 75, wherein the vector is a recombinant expression vector.

84. A recombinant host cell comprising the polynucleotide of claim 77 operably linked to a heterologous promoter.

85. An isolated polynucleotide of claim 77, wherein the polynucleotide encodes a human polypeptide.

86. An isolated polynucleotide of claim 77, wherein the polynucleotide encodes a human polypeptide.

87. A non-human transgenic animal having a genome comprising a transgene comprising a nucleotide sequence encoding a MICAL polypeptide operably linked to a heterologous promoter, wherein the non-human transgenic animal expresses the transgenic polynucleotide in the central nervous system, and wherein expression levels of the transgenic polynucleotide are sufficient to effect an axonal guidance phenotype of the non-human organism.

88. The non-human transgenic animal of claim 87, wherein the non-human transgenic animal is a mouse.

89. The non-human transgenic animal of claim 87, wherein the MICAL polypeptide is ectopically expressed.

90. The non-human transgenic animal of claim 87, wherein the MICAL polypeptide is expressed at a greater level in one or more cells of the non-human transgenic animal than the MICAL polypeptide is expressed in comparable cells of a comparable non-human transgenic animal.

91. A non-human transgenic animal having a genome comprising a recombinantly inactivated nucleotide sequence encoding a MICAL polypeptide that has been recombinantly inactivated, wherein the non-human transgenic animal has an altered axon guidance phenotype.

92. The non-human transgenic animal of claim 91, wherein the non-human transgenic animal is a mouse.

93. The non-human transgenic animal of claim 91, wherein the non-human transgenic animal is heterozygous for the nucleotide sequence that has been inactivated.

94. The non-human transgenic animal of claim 91, wherein the non-human transgenic animal is homozygous for the nucleotide sequence that has been recombinantly inactivated.

95. A method for inhibiting axonal guidance regulatory activity, comprising contacting a cell that expresses a polypeptide of claim 1 with a monooxygenase inhibitor, thereby inhibiting axonal guidance regulatory activity.

96. The method of claim 95, wherein the monooxygenase inhibitor is a flavonoid.

97. The method of claim 96, wherein the flavonoid is a gallic acid derivative.

98. The method of claim 97, wherein the gallic acid derivative is (−)-epigallocatechin gallate (EGCG), (−)-epicatachin (EC), (−)-gallocatechin-3-O-gallate (GCG), (−)-epicatechin-3-O-gallate (ECG), (−)-epigallocatechin (EGC), (+)-gallocatechin (GC), theasinensin A, 3″-O-methyl-EGCG, 3″-O-methyl-ECG, 3″-O-methyl-GCG, (−)-epigallocatechin (EGC), (1)-gallocatechin (GC), gallic acid, catechin, n-octyl gallate, or n-cetyl gallate.

99. The method of claim 95, wherein the contacting is performed in vitro.

100. The method of claim 95, wherein the contacting is performed in vivo.

101. A method for affecting a semaphorin-mediated process, comprising contacting a cell that expresses a polypeptide of claim 1 with an effective amount of a monooxygenase inhibitor, thereby affecting the semaphorin-mediated process.

102. The method of claim 101, wherein the agent inhibits semaphorin la-PlexA-mediated repulsive axon guidance.

103. The method of claim 102, wherein the cell is a neuron.

104. The method of claim 102, wherein the cell is an immune cell, a cancer cell, or a cardiac cell.

105. The method of claim 101, wherein the monooxygenase inhibitor is a flavonoid.

106. The method of claim 105, wherein the flavonoid is a gallic acid derivative.

107. The method of claim 106, wherein the gallic acid derivative is (−)-epigallocatechin gallate (EGCG), (−)-epicatachin (EC), (−)-gallocatechin-3-O-gallate (GCG), (−)-epicatechin-3-O-gallate (ECG), (−)-epigallocatechin (EGC), (+)-gallocatechin (GC), theasinensin A, 3″-O-methyl-EGCG, 3″-O-methyl-ECG, 3″-O-methyl-GCG, (−)-epigallocatechin (EGC), (1)-gallocatechin (GC), gallic acid, catechin, n-octyl gallate, or n-cetyl gallate.

108. A method for treating a neurological condition in a subject in need thereof, the method comprising contacting in the subject, a cell of the central nervous system (CNS) or peripheral nervous system (PNS) having a disrupted axonal connection or a cell that affects axonal growth of the CNS cell or the PNS cell, with an amount of an agent that modulates the activity or expression of a MICAL polypeptide, the amount being effective to modulate axonal guidance or axon out-growth regulatory activity.

109. The method of claim 108, wherein the cell is contacted with the agent for a length of time effective for allowing axon regrowth.

110. The method of claim 108, wherein the agent is applied for a an length of time sufficient to promote neurorestoration.

111. The method of claim 108, wherein the agent is applied chronically.

112. The method of claim 108, wherein the neurological condition is a spinal cord injury.

113. The method of claim 108, wherein the neurological condition is traumatic brain injury.

114. The method of claim 108, wherein the neurological condition is neuropathic pain.

115. The method of claim 108, wherein the neurological condition is Parkinson's Disease.

116. The method of claim 108, wherein the neurological condition is Amyotrophic Lateral Sclerosis.

117. The method of claim 108, wherein the neurological condition is ischemic injury.

118. The method of claim 108, wherein the neurological condition is Alzheimer's Disease.

119. The method of claim 108, wherein the neurological condition is Multiple Sclerosis.

120. The method of claim 108, wherein the neurological condition is a neuropathy resulting from a stroke.

121. The method of claim 108, wherein the agent is an anti-oxidant.

122. The method of claim 121, wherein the anti-oxidant is a flavonoid.

123. The method of claim 122, wherein the flavonoid is a gallic acid derivative.

124. The method of claim 123, wherein the gallic acid derivative is (−)-epigallocatechin gallate (EGCG), (−)-epicatachin (EC), (−)-gallocatechin-3-O-gallate (GCG), (−)-epicatechin-3-O-gallate (ECG), (−)-epigallocatechin (EGC), (+)-gallocatechin (GC), theasinensin A, 3″-O-methyl-EGCG, 3″-O-methyl-ECG, 3″-O-methyl-GCG, (−)-epigallocatechin (EGC), (1)-gallocatechin (GC), gallic acid, catechin, n-octyl gallate, or n-cetyl gallate.

125. The method of claim 108, wherein the method further comprises contacting the cell with an agent that affects axon regeneration.

126. The method of claim 125, wherein the effect is promotion of axon regeneration.

127. The method of claim 126, wherein the agent is a neurotrophin, a mechanical bridge, or a stem cell.

128. The method of claim 127, wherein the mechanical bridge is a cell engineered to express a neurotrophic factor, a growth factor, an axon outgrowth promoting molecule, or an artificial polymer-based substrate.

129. The method of claim 127, wherein the mechanical bridge comprises fetal tissue.

130. The method of claim 127, wherein the mechanical bridge comprises a schwann cell or an olfactory ensheathing glia.

131. The method of claim 127, wherein the agent is a neurotrophic factor, a growth factor, an axon outgrowth promoting molecule, or an artificial polymer-based substrate.

132. The method of claim 131, wherein the agent is NGF, BDNF, NT-3, or NT-4/5.

133. The method of claim 131, wherein the agent is CNTF, GDNF, FGF, EGF, or PDGF.

134. The method of claim 131, wherein the agent is netrin, laminin, or collagen.

135. A method for treating a neurological disorder involving a failure of axon regrowth, comprising contacting a neuron having axons that fail to regrow, or surrounding tissue, with an agent that neutralizes oxidants for a length of time effective for allowing axon regrowth, thereby treating the neurological disorder.

136. The method of claim 135, wherein the agent is applied for a length of time sufficient to promote neurorestoration.

137. The method of claim 135, wherein the agent is applied for a length of time to diminish free radicals chronically after spinal cord injury.

138. The method of claim 135, wherein the agent is an anti-oxidant.

139. The method of claim 138, wherein the anti-oxidant is a flavonoid.

140. The method of claim 139, wherein the flavonoid is a gallic acid derivative.

141. The method of claim 140, wherein the gallic acid derivative is (−)-epigallocatechin gallate (EGCG) or (−)-epicatachin (EC).

142. A method for inducing regrowth and preventing inhibition of an injured process of a neuron, comprising altering the levels of reactive oxygen species in the milieu of the neuron for an length of time effective for allowing the injured process to reach a target.

143. The method of claim 142, wherein the levels of reactive oxygen species are altered for a length of time sufficient to promote neurorestoration.

144. The method of claim 142, further comprising identifying a site that includes the neuron suspected of having an injured process, before altering the levels of reactive oxygen species in the milieu of the neuron.

145. The method of claim 142, wherein the neuronal process is an axon.

146. The method of claim 142, wherein the neuronal process is a dendrite.

147. The method of claim 142, wherein levels of reactive oxygen species or other oxidation products are decreased.

148. The method of claim 142, wherein the method further comprises adding an agent that promote neuron process regrowth to the milieu of the neuron.

149. The method of claim 148, wherein the agent is a neurotrophin, a mechanical bridge, or a stem cell.

150. The method of claim 148, wherein levels of reactive oxygen species are lowered for at least 1 month.

151. The method of claim 148, wherein levels of reactive oxygen species are lowered for at least 3 months.

152. A method for limiting abnormal axon outgrowth, comprising contacting a neuron or the milieu of the neuron with an agent that affects oxidation state.

153. The method of claim 150, wherein the abnormal axon outgrowth is excessive axon outgrowth.
